SEQUENCE LISTING (160 sequences)

SEQ ID NO: 1 - DNA, 333 bp, Artificial Sequence
  Description of Artificial Sequence Synthetic polynucleotide
tgataataggctggagcctcggtggcctagcttcttgccccttgggcctccccccagcccctcctccccttcctgcacccgtacccccgtggtctttgaataaagtctgagtgggcggccaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggag

SEQ ID NO: 2 - DNA, 333 bp, Artificial Sequence
  Description of Artificial Sequence Synthetic polynucleotide
tgataatagcaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggaggctggagcctcggtggcctagcttcttgccccttgggcctccccccagcccctcctccccttcctgcacccgtacccccgtggtctttgaataaagtctgagtgggcggc

SEQ ID NO: 3 - DNA, 223 bp, Artificial Sequence
  Description of Artificial Sequence Synthetic polynucleotide
tgataatagcaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggag

SEQ ID NO: 4 - DNA, 119 bp, Artificial Sequence
  Description of Artificial Sequence Synthetic polynucleotide
tgataataggctggagcctcggtggcctagcttcttgccccttgggcctccccccagcccctcctccccttcctgcacccgtacccccgtggtctttgaataaagtctgagtgggcggc

SEQ ID NO: 5 - DNA, 333 bp, Artificial Sequence; identical to SEQ ID NO: 1
  Description of Artificial Sequence Synthetic polynucleotide

SEQ ID NO: 6 - DNA, 57 bp, Artificial Sequence
  Description of Artificial Sequence Synthetic oligonucleotide
gggaaataagagagaaaagaagagtaagaagaaatataagaccccggcgccgccacc

SEQ ID NO: 7 - RNA, 121 nt, Artificial Sequence
  Description of Artificial Sequence Synthetic polynucleotide
ggauccgugaucggaaacgugagauccaccucagauccgcuaggacacccgcagaucgagaagaaggcgaauuaagagagaaaagaagaguaagaagaaauauaagacaccggucgccacc

SEQ ID NO: 8 - RNA, 236 nt, Artificial Sequence
  Description of Artificial Sequence Synthetic polynucleotide
gggaaacggcaagcagcagcugccgcuguuuugucuuaaccgguugaaagaccauuaggcuuucuaacggacgcucacgagagaggggcaccaauagggccccacggcacuucaaagguuuggcuuggaguaguaacccaagcagcaacaguuuugacuuucggaccaccaucaggggucccacguugggaacacguaacucuccuacuaacaagaggagccccggcgccgccacc

SEQ ID NO: 9 - RNA, 73 nt, Artificial Sequence
  Description of Artificial Sequence Synthetic oligonucleotide
gggaaauaagagagaaaaagagagagggagaggagaagaguaagaagaaauauaagaccccggcgccgccacc

SEQ ID NO: 10 - RNA, 73 nt, Artificial Sequence
  Description of Artificial Sequence Synthetic oligonucleotide
gggaaauaagagagaaaagaagaguaagaaagagagagggagaggagaaauauaagaccccggcgccgccacc

SEQ ID NO: 11 - RNA, 73 nt, Artificial Sequence
  Description of Artificial Sequence Synthetic oligonucleotide
gggaaauaagagagaaaagaagaguaagaagaaauauaagacagagagagggagaggacccggcgccgccacc

SEQ ID NO: 12 - RNA, 105 nt, Artificial Sequence
  Description of Artificial Sequence Synthetic polynucleotide
gggaaauuuuuuuuugauauuauaagaguuuuuuuuugauauuaagaaaauuuuuuuuugauauuagaagaguaagaagaaauauaagaccccggcgccgccacc

SEQ ID NO: 13 - RNA, 105 nt, Artificial Sequence
  Description of Artificial Sequence Synthetic polynucleotide
gggaaauaagaguuuuuuuuugauauuaagaaaauuuuuuuuugauauuagaagaguuuuuuuuugauauuauaagaagaaauauaagaccccggcgccgccacc

SEQ ID NO: 14 - RNA, 105 nt, Artificial Sequence
  Description of Artificial Sequence Synthetic polynucleotide
gggaaauaagagagaaaauuuuuuuuugauauuagaagaguuuuuuuuugauauuauaagaauuuuuuuuugauauuagaaauauaagaccccggcgccgccacc

SEQ ID NO: 15 - RNA, 105 nt, Artificial Sequence
  Description of Artificial Sequence Synthetic polynucleotide
gggaaauaagagagaaaagaagaguuuuuuuuugauauuauaagaauuuuuuuuugauauuagaaauauuuuuuuuugauauuauaagaccccggcgccgccacc

SEQ ID NO: 16 - RNA, 105 nt, Artificial Sequence
  Description of Artificial Sequence Synthetic polynucleotide
gggaaauaagagagaaaagaagaguaagaauuuuuuuuugauauuagaaauauuuuuuuuugauauuauaagacuuuuuuuuugauauuacccggcgccgccacc

SEQ ID NO: 17 - RNA, 105 nt, Artificial Sequence
  Description of Artificial Sequence Synthetic polynucleotide
gggaaauaagagagaaaagaagaguaagaagaaauauuuuuuuuugauauuauaagacuuuuuuuuugauauuacccggcuuuuuuuuugauauuagccgccacc

SEQ ID NO: 18 - RNA, 19 nt, Artificial Sequence
  Description of Artificial Sequence Synthetic oligonucleotide
gggcccugaagaagggccc

SEQ ID NO: 19 - RNA, 21 nt, Artificial Sequence
  Description of Artificial Sequence Synthetic oligonucleotide
aauccauugcacuccggauuu

SEQ ID NO: 20 - DNA, 244 bp, Artificial Sequence
  Description of Artificial Sequence Synthetic polynucleotide
ggatgtgtggccagttggtgctgtctgtcttcttggtgaccaggagtaatttggaccatctgggaaaccagacctcttttcacccaggctgtttcgtgtgcatcacttctgaggctgtgtgctcagtcaagaccttgccagagagttgggggattagccttaggtcgagaatattctgagcagtagtaagaaatttaaaaatcatccataatttcatcactcttattttaagagattagtttat

SEQ ID NO: 21 - protein, 117 aa, Unknown
  Description of Unknown MBP sequence
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 22 - DNA, 351 bp, Unknown
  Description of Unknown MBP sequence
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctac

SEQ ID NO: 23 - protein, 977 aa, Unknown
  Description of Unknown eIF4GdN sequence
IFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 24 - DNA, 2931 bp, Unknown
  Description of Unknown eIF4GdN sequence
atattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 25 - protein, 1099 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 26 - DNA, 3297 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 27 - protein, 1720 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSNKAPQSTGPPPAPSPGLPQPAFPPGQTAPVVFSTPQATQMNTPSQPRQHFYPSRAQPPSSAASRVQSAAPARPGPAAHVYPAGSQVMMIPSQISYPASQGAYYIPGQGRSTYVVPTQQYPVQPGAPGFYPGASPTEFGTYAGAYYPAQGVQQFPTGVAPTPVLMNQPPQIAPKRERKTIRIRDPNQGGKDITEEIMSGARTASTPTPPQTGGGLEPQANGETPQVAVIVRPDDRSQGAIIADRPGLPGPEHSPSESQPSSPSPTPSPSPVLEPGSEPNLAVLSIPGDTMTTIQMSVEESTPISRETGEPYRLSPEPTPLAEPILEVEVTLSKPVPESEFSSSPLQAPTPLASHTVEIHEPNGMVPSEDLEPEVESSPELAPPPACPSESPVPIAPTAQPEELLNGAPSPPAVDLSPVSEPEEQAKEVTASMAPPTIPSATPATAPSATSPAQEEEMEEEEEEEEGEAGEAGEAESEKGGEELLPPESTPIPANLSQNLEAAAATQVAVSVPKRRRKIKELNKKEAVGDLLDAFKEANPAVPEVENQPPAGSNPGPESEGSGVPPRPEEADETWDSKEDKIHNAENIQPGEQKYEYKSDQWKPLNLEEKKRYDREFLLGFQFIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 28 - DNA, 5160 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaacaaggcccctcagagcaccggccctcctcctgcccctagccctggcctgcctcagcctgccttccctcctggccagaccgcccctgtggtgttcagcacccctcaggccacccagatgaacacacctagccagcctagacagcacttctaccctagcagagcccagcctcctagcagcgccgccagcagagtgcagtccgctgcacctgccagacctggccctgccgcccacgtgtaccctgccggcagccaggtgatgatgatcccgagtcaaatcagctaccctgcaagccagggcgcctactacatcccgggccagggcagaagcacctacgtggtgcctacccagcagtaccctgtgcagcctggcgcccctggtttctatcctggcgcaagccctaccgagttcggaacttacgccggcgcttactatccagctcagggcgtgcagcagttccctaccggcgtggcccctacccctgtgctgatgaatcagccacctcagatcgcccctaagcgcgagcgcaagaccatcagaatccgcgatcctaaccagggcggcaaggacatcaccgaggagatcatgagcggcgccagaacagcaagtactccaaccccgccacaaaccggcggcggcctggagcctcaagccaacggcgagacgccacaagtggccgtgatcgtacgccctgacgaccggagccaaggtgcaatcatcgccgataggcctggcctcccaggtccggagcacagccctagcgagtcccagccgtcttcaccatcaccaacccctagtccatcccctgttctcgaaccaggcagcgagcctaacctggccgtgctgagcataccaggtgacaccatgaccaccatccagatgagcgtggaggagagcaccccaatcagcagagaaactggagagccttacagactgtccccagagccgaccccactggccgagccaatactggaggtggaggtgaccctgagcaagcctgtgcctgagagcgagttcagctcctctccactgcaggccccaactcctctcgcaagccacaccgtggagatccacgaacctaacggcatggtaccaagcgaagatcttgagccagaggtcgaatcaagcccagaactggcccctccacctgcctgcccgtctgaatctccggtccctatcgctcctacggcacagcctgaggagctgctgaacggtgccccgagccctccagcagtggacttatccccagtatcagagcctgaagaacaggccaaggaggtaactgcctctatggcgccacctaccataccttcggcaacaccggctacagcaccatctgcgactagtccggctcaggaggaggagatggaggaagaagaagaggaagaggagggcgaggccggagaggccggtgaagccgagtccgagaagggcggcgaagaacttctccctccagagtcaactcctatccctgccaaccttagtcagaatctggaggccgccgccgctactcaggttgcagtgagcgtgccaaagagacgtcgcaagatcaaggagctgaacaagaaggaggccgtgggcgacctgctggacgccttcaaggaggcaaacccggcggtgcctgaagtggagaatcagcctccggccggatcaaaccctggtcctgagagtgaaggcagcggcgtcccaccaagacctgaagaggctgacgaaacttgggacagcaaggaggacaagatccacaacgccgagaacatccagccaggcgagcagaagtacgagtacaagagcgaccagtggaagcctcttaaccttgaagagaagaagagatacgacagagagttcctgctgggcttccagttcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 29 - protein, 1099 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSIANKATPQMAQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 30 - DNA, 3297 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcgcgaacaaggcgacccctcagatggcgcagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 31 - protein, 1069 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 32 - DNA, 3207 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 33 - protein, 921 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLL

SEQ ID NO: 34 - DNA, 2763 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctc

SEQ ID NO: 35 - protein, 528 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSID

SEQ ID NO: 36 - DNA, 1584 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgac

SEQ ID NO: 37 - protein, 600 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 38 - DNA, 1800 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 39 - protein, 541 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 40 - DNA, 1623 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 41 - protein, 642 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 42 - DNA, 1926 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 43 - protein, 494 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLL

SEQ ID NO: 44 - DNA, 1482 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctc

SEQ ID NO: 45 - protein, 719 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSENSSAASASSEAGSSRSQEIEELERFIDSYVLEYQVQGLLADKTEGDGESERTQSHISQWTADCSEPLDSSCSFSRGRAPPQQNGSKDNSLDMLGTDIWAANTFDSFSGATWDLQPEKLDFTQFHRKVRHTPKQPLPHIDREGCGKGKLEDGDGINLNDIEKVLPAWQGYHPMPHEVEIAHTKKLFRRRRNDRRRQQRPPGGNKPQQHGDHQPGSAKHNRDHQKSYQGGSAPHPSGRPTHHGYSQNRRWHHGNMKHPPGDKGEAGAHRNAKETMTIENPKLEDTAGDTGHSSLEAPRSPDTLAPVASERLPPQQSGGPEVETKRKDSILPERIGERPKITLLQSSKDRLRRRLKEKDEVAVETTTPQQNKMDKLIEILNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWNPLTPPITQYYNRTIQKLTA

SEQ ID NO: 46 - DNA, 2157 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgagaacagtagcgccgctagcgcgagcagcgaagccgggagcagccggtctcaggagatcgaggagctggagcggttcatcgacagctacgtgctggagtaccaggtgcagggcctgctggccgacaagaccgagggcgacggcgagagcgagcggacccagagccacatcagccagtggaccgccgactgcagcgagcccctggactctagctgctcattcagtcgtggacgggcccctccacagcagaacggcagcaaggacaacagcctggacatgctgggcaccgacatctgggccgccaacaccttcgacagcttcagcggcgccacctgggatctgcagcccgagaagctggactttacccagttccaccggaaggtgcggcacactcccaagcagcccctgccccacatcgatcgggagggctgcggcaagggcaagctggaagacggcgacggcatcaacctgaacgacatcgagaaggtgctgcctgcctggcagggctaccaccccatgccccacgaggtggagatcgcccacaccaagaagctgttccggcgacgacgcaacgaccggcgtaggcagcaacggccgcctggagggaacaagccccagcagcacggagaccaccagcccggtagcgccaagcacaaccgggaccaccagaagagctaccagggcggaagcgcaccacacccctcgggcagacccacccaccacggctacagccagaaccggcggtggcatcacggtaacatgaagcacccacccggcgacaaaggagaggccggcgctcaccgtaacgccaaggagaccatgaccatcgagaaccccaagctggaggataccgccggcgatacgggtcacagcagcctggaggcaccgcggtctcccgacaccctggcacccgtggccagcgaacggctgccaccccaacagagcggcggccctgaggttgagaccaagcggaaggacagcatcctgcccgaacggatcggtgagcggcccaagatcaccttactgcagagtagcaaggaccggctgagacggcggctgaaggagaaggacgaggtggccgtggagacaaccactccccagcagaacaagatggacaagctgatcgagatcctgaacagcatgcggaacaacagcagcgacgtggacaccaagctgaccaccttcatggaggaggcccagaacagcaccaacagcgaggagatgctgggcgagatcgtgcggaccatctaccagaaggccgtgagcgaccggagcttcgccttcaccgccgccaagctgtgcgacaagatggccctgttcatggtggagggcaccaagttccggagcttactgctgaatatgctgcagaaggacttcaccgtgcgggaggagctgcagcagcaggacgtggagcggtggctgggcttcatcaccttcctgtgcgaggtgttcggcaccatgcggagcagcaccggcgaacccttccgggtgctggtgtgccccatctacacctgcctgcgggagttgctgcagagccaggacgtgaaggaggacgccgtgctgtgctgcagcatggaactgcagagcactggccggctgctggaggagcagctgcccgagatgatgaccgagctgctcgctagcgcccgggacaagatgctgtgccccagcgagagcatgctgacccggagcctgcttctggaggtgatcgagctgcacgccaacagctggaatcccctgacccctcccatcacccagtactacaaccggaccatccagaagctgaccgcc

SEQ ID NO: 47 - protein, 323 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSLNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWN

SEQ ID NO: 48 - DNA, 969 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcctgaacagcatgagaaacaacagcagcgacgtggacaccaagctgaccaccttcatggaggaggcccagaacagcaccaacagcgaggagatgctgggcgagatcgtgagaaccatctaccagaaggccgtgagcgacagaagcttcgccttcaccgccgccaagctgtgcgacaagatggccctgttcatggtggagggcaccaagttcagaagcctgctgctgaacatgctgcagaaggacttcaccgtgagagaggagctgcagcagcaggacgtggagcgatggctgggcttcatcaccttcctgtgcgaggtgttcggcaccatgagaagcagcaccggcgagcctttcagagtgctggtgtgccctatctacacctgcctgagagagctgctgcagagccaggacgtgaaggaggacgccgtgctgtgctgcagcatggagctgcagagcaccggcagactgctggaggagcagctgcctgagatgatgaccgagctgctggccagcgccagagacaagatgctgtgccctagcgagagcatgctgactagatccctactgttggaggtgatcgagctgcacgccaacagctggaac

SEQ ID NO: 49 - protein, 356 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSTTPQQNKMDKLIEILNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWNPLTPPITQYYNRTIQKLTA

SEQ ID NO: 50 - DNA, 1068 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaccacccctcagcagaacaagatggacaagctgatcgagatcctgaacagcatgagaaacaacagcagcgacgtggacaccaagctgaccaccttcatggaggaggcccagaacagcaccaacagcgaggagatgctgggcgagatcgtgagaaccatctaccagaaggccgtgagcgacagaagcttcgccttcaccgccgccaagctgtgcgacaagatggccctgttcatggtggagggcaccaagttcagaagcctgctgctgaacatgctgcagaaggacttcaccgtgagagaggagctgcagcagcaggacgtggagaggtggctgggcttcatcaccttcctgtgcgaggtgttcggcaccatgagaagcagcaccggcgagcctttcagagtgctggtgtgccctatctacacctgcctgagagagctgctgcagagccaggacgtgaaggaggacgccgtgctgtgctgcagcatggagctgcagagcaccggcagactgctggaggagcagctgcctgagatgatgaccgagctgctggccagcgccagagacaagatgctgtgccctagcgagagcatgctgacgcgcagcctcctgctggaggtgatcgagctgcacgccaacagctggaaccctctgacccctcctatcacccagtactacaacagaaccatccagaagctgaccgcc

SEQ ID NO: 51 - protein, 364 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPR

SEQ ID NO: 52 - DNA, 1092 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgccgacggcagcaagacccaggacctgttcagaagagtgagaagcatcctgaacaagctgacccctcagatgttccagcagctgatgaagcaggtgacccagctggccatcgacaccgaggagagactgaagggcgtgatcgacctgatcttcgagaaggccatcagcgagcctaacttcagcgtggcctacgccaacatgtgccggtgcctgatggccctgaaggtgcctaccaccgagaagcctaccgtgaccgtgaacttcagaaagctgctgctgaaccggtgccagaaggagttcgagaaggacaaggacgacgacgaggtgttcgagaagaagcagaaggagatggacgaggccgccaccgccgaggagagaggcagactgaaggaggagctggaggaggccagagacatcgccagaagaagaagcctgggcaacatcaagttcatcggcgagctgttcaagctgaagatgctgaccgaggccatcatgcacgactgcgtggtgaagctgctgaagaaccacgacgaggagagcctggagtgcctgtgcagactgctgaccaccatcggcaaggacctggatttcgagaaggcgaagcctagaatggaccagtacttcaaccagatggagaagatcatcaaggagaagaagaccagcagcagaatcagattcatgctgcaggacgtgctggacctgagaggcagcaactgggtgcctaga

SEQ ID NO: 53 - protein, 1168 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSFTLTNKNVIFVAGLGGIGLDTSKELLKRDPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAELQLSAGRYHYQLVWCQK

SEQ ID NO: 54 - DNA, 3504 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagctcgttcacgctaacgaacaagaacgtcatcttcgtagcgggacttggcggtatcggcctagacacgtcgaaggaactactaaagcgtgacccggtagtcctccaacgtcgcgattgggagaacccgggcgtaacgcaactaaaccgtcttgcggcgcacccgccgtttgcgtcgtggcgtaactcggaggaggcgcgaacggatcgtccgtcgcaacaactacgttcgctcaacggggagtggcgcttcgcgtggttcccggcgccggaggcggtaccggagtcgtggctcgagtgcgatctaccggaggcggacacggtcgtcgtaccgtcgaactggcaaatgcacggttacgacgcgccgatatacacgaacgtcacgtacccgataacggtaaacccgccgttcgtcccgacggagaacccgacggggtgctactcgctaacgttcaacgttgacgagtcgtggttgcaagagggtcaaacgcgtatcatattcgacggtgtaaactcggcgttccacctgtggtgcaacgggcgctgggtagggtacggccaagactcgcgtctaccgtcggagttcgacctatcggcgttcctacgagcgggtgagaaccggctagcggtcatggtcctacgttggtcggacggttcgtacctcgaggaccaagacatgtggcgaatgtcgggtatcttccgcgacgtatcgctcctacacaagccgacgacgcaaatctcggacttccacgtcgcgacgcgtttcaacgacgatttctcgcgggcagtcctagaggcggaggtccaaatgtgcggggagctacgtgactacctccgtgtcacggtatcgctctggcaaggtgagacgcaagtagcgtcgggtacggcgccgttcggcggtgagatcatcgacgagcgtggtgggtacgcggaccgtgtaacgctacgtctaaacgtcgagaacccgaagctctggtcggcggagatcccgaacctataccgtgcggtcgtcgagctacatacggcggacgggacgctaatagaggcggaagcgtgcgacgtcgggtttcgagaggttcgtatagagaacgggctgctacttctaaacgggaagccgttgctcatacgtggtgtcaaccgtcacgagcaccacccgctacacggtcaagtaatggacgagcaaacgatggtacaagacatcctactaatgaagcagaacaacttcaacgcggtacgctgttcgcattacccgaaccatccgttgtggtacacgctttgcgaccgatacggtctatacgtcgtagacgaggcgaacatagagacgcacgggatggtaccgatgaatcgcctaacggacgacccgcgttggctaccggcgatgtcggagcgagtcacgcgtatggtccaacgggaccgtaaccacccgtcggtaataatctggtcgctaggcaacgaatcggggcacggggcgaaccacgacgcgctataccgttggatcaagtcggtagacccgtcgcgtccggtacaatacgaaggtggcggtgcggacacgacggcgacggacatcatctgcccgatgtacgcgcgcgtcgacgaagaccaaccgttcccggcggtaccgaagtggtcgatcaagaagtggctctcgttgccgggtgaaacgcgtccgttgatactttgcgagtacgcgcacgcgatgggcaactcgttgggtgggttcgcgaagtactggcaggcgttccgtcaatacccgcgtctacagggtgggttcgtctgggactgggtagaccaatcgctaatcaagtacgacgagaacggcaacccgtggtcggcgtacggtggggacttcggggacacgccgaacgaccgccaattctgtatgaacggcctagtcttcgcggaccgaacgccgcacccggcgttgacggaggcgaagcatcaacaacaattcttccaattccgtctatcggggcaaacgatcgaggtaacgtcggagtacttgttccggcactcggacaacgagctactacactggatggtagcactagacggcaagccgctagcgtcgggagaagtccctttggacgtcgcgccgcaaggtaagcaactaatcgagctaccggagctaccgcaaccggagtcggcgggtcaactgtggttgacggtccgtgtcgttcaaccgaacgcgacggcgtggtcggaggcgggtcacatctcggcgtggcagcagtggcgtctagcggagaacctctcggtcacgctaccggcggcgtcgcacgcgataccgcatctaacgacgtcggagatggacttctgcatcgagttggggaacaagaggtggcagttcaaccgtcaatcgggattcctatcgcaaatgtggataggtgacaagaagcaactactaacgccgctacgtgatcagttcacgcgtgctccgctagacaacgacataggtgtttcggaggcgacgcgtatagacccgaacgcgtgggtggagcggtggaaggcggcggggcactaccaagcggaggcggcgctactacagtgcacggcggacacgctagcggacgcggtattgatcacgacggcgcacgcgtggcaacaccaggggaagacgctattcatctcgcgtaagacgtaccgtatcgacggttcgggccaaatggcgatcacggtcgacgtagaggtagcgtcggacacgccgcatccggcgcgcatcggtctaaactgccaactagcgcaagtagcggagcgtgtaaactggctagggctagggccgcaagagaactatccggaccgcctaacggcggcgtgcttcgaccgttgggacctaccgctttcggacatgtataccccgtacgtcttcccgtcggagaacgggttgaggtgcgggacgcgcgagctaaactacgggccgcaccagtggcgaggggacttccaattcaacatatcgcgttactcgcaacaacaactaatggagacgtcgcaccgtcacctactacacgcggaggaggggacgtggctaaacatcgacgggttccacatgggcataggtggggacgactcgtggtcgccgtcggtctcggcggagctccaactctcggcgggtcgttaccattaccaactagtttggtgccagaag

SEQ ID NO: 55 - protein, 119 aa, Unknown
  Description of Unknown 50S ribosomal L7Ae sequence
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 56 - DNA, 378 bp, Unknown
  Description of Unknown 50S ribosomal L7Ae sequence
atgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagagatctcatatgcatctcgag

SEQ ID NO: 57 - protein, 264 aa, Artificial Sequence
  Description of Artificial Sequence Synthetic polypeptide
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGGGGSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 58 - DNA, 792 bp, Artificial Sequence
  Description of Artificial Sequence Synthetic polynucleotide
atggccagcaattttacccagttcgtgctggtcgacaacggcgggaccggtgacgttaccgtggcccctagcaatttcgctaacggcgtggctgagtggatctccagcaacagccggagccaagcttacaaggtgacctgtagcgtgcggcagagcagcgcccagaaccggaagtacaccatcaaggtagaagtgcctaaggtggccacacagactgtgggcggtgtcgagctgcccgtggcagcttggaggagctatctgaatatggaactgaccatccccatcttcgccactaacagcgattgtgagctgattgttaaggccatgcaaggcctgctaaaggacgggaaccccattcccagcgccatcgctgccaacagcggcatctacggaggaggcggaagcgctagcaacttcacccagtttgtgctggtggacaacggtggcaccggcgacgtgaccgtggctcccagcaactttgccaacggtgtggccgagtggatcagcagcaattctcggagccaggcctacaaggtcacgtgcagcgtgcgccaaagcagcgctcagaatcggaagtatactatcaaggtggaggtgcccaaagtggccacccagaccgtgggtggcgtggagctgcctgtggccgcttggcgatcatacctgaacatggagctcaccatccctatcttcgcaaccaacagcgactgcgagctgatcgtgaaagccatgcagggcctgctgaaggacggcaatcccatccccagcgctatcgccgccaatagcggaatctac

SEQ ID NO: 59 - protein, 600 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKAIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 60 - DNA, 1800 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaaggcgataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 61 - protein, 128 aa, Unknown
  Description of Unknown PCP sequence
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVVQATSEDLVVNLVPLGR

SEQ ID NO: 62 - DNA, 384 bp, Unknown
  Description of Unknown PCP sequence
atgagcaagaccatcgtgctgagcgtgggcgaggccaccagaaccctgaccgagatccagagcaccgccgacagacagatcttcgaggagaaggtgggccctctggtgggcagactgagactgaccgccagcctgagacagaacggcgccaagaccgcctacagagtgaacctgaagctggaccaggccgacgtggtggactgcagcaccagcgtgtgcggcgagctgcctaaggtgagatacacccaggtgtggagccacgacgtgaccatcgtggccaacagcaccgaggccagcagaaagagcctgtacgacctgaccaagagcctggtggtgcaggccaccagcgaggacctggtggtgaacctggtgcctctgggcaga

SEQ ID NO: 63 - protein, 667 aa, Artificial Sequence
  Description of Artificial Sequence Synthetic polypeptide
MGSSHHHHHHSQDLEVLFQGPHMGRSRLLEDFRNNRYPNLQLREIAGHIMEFSQDQHGSRFIQLKLERATPAERQLVFNEILQAAYQLMVDVFGSYVIEKFFEFGSLEQKLALAERIRGHVLSLALQMYGSRVIEKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFASNVVEKCVTHASRTERAVLIDEVCTMNDGPHSALYTMMKDQYASYVVEKMIDVAEPGQRKIVMHKIRPHIMEFSQDQHGSRFIELKLERATPAERQLVFNEILQAAYQLMVDVFGCYVIQKFFEFGSLEQKLALAERIRGHVLSLALQMYGSRVIEKALEFIPSDQQNEMVRELDGHVLKCVKDQNGCHVVQKCIECVQPQSLQFIIDAFKGQVFALSTHPYGSRVIERILEHCLPDQTLPILEELHQHTEQLVQDQYGCYVIQHVLEHGRPEDKSKIVAEIRGNVLVLSQHKFASNVVEKCVTHASRTERAVLIDEVCTMNDGPHSALYTMMKDQYACYVVQKMIDVAEPGQRKIVMHKIRPHIATLRKYTYGKHILAKLEKYYMKNGVDLG

SEQ ID NO: 64 - DNA, 2001 bp, Artificial Sequence
  Description of Artificial Sequence Synthetic polynucleotide
atgggcagcagccaccatcaccaccaccacagccaggacctggaggtgctgttccagggcccacacatgggccgtagccggctgctggaggacttccggaacaaccggtaccccaacctgcagctgcgggagatcgccggccacatcatggagttcagccaggaccagcacggcagccgcttcatccagctgaagttggagcgggctacacccgctgagcggcagctggtgttcaacgagatcctgcaggccgcctatcaactgatggtagacgtgttcggcagctacgtgatcgagaagttctttgagttcggcagtctggagcagaagctcgccctggccgaacggatacggggccacgtgcttagcctggccctacagatgtacggcagcagggtgatcgagaaagccctggagttcatccccagcgaccaacagaacgaaatggtgcgggagctcgacggccacgtcctgaagtgcgtgaaggaccagaacggttgccacgtggtgcagaagtgcatcgagtgcgtccagccccagtctctgcagttcattatcgacgccttcaagggccaagtgttcgccctgagtacccacccctacggaagccgggtgatcgaacggattctggagcattgcctgcccgatcagaccctgcccatcctggaggagctgcatcaacacaccgaacagcttgtgcaagaccagtacggctgctacgtgatccagcacgtgttggagcacggacggcccgaggacaagagcaagattgtggccgagatcaggggtaacgtgctggtgctgtctcagcacaaattcgccagcaacgtggtagagaagtgcgtgacccacgccagccggactgaacgggccgtgctgatcgacgaggtgtgcactatgaacgacgggccccactctgccctgtacacaatgatgaaagaccaatacgccagctacgtggtggagaagatgatcgacgtggccgagccaggccaacggaagatcgtgatgcataagatccggcctcatatcatggagtttagccaggatcaacacggcagccggttcatcgagctgaagctggaacgggccacccctgccgaacggcagctggtctttaacgagatactgcaagccgcctaccagctgatggtggacgtgtttggttgctacgttatccagaagttcttcgagttcggaagcctggagcagaaactggccctggccgagcgtatccggggccacgttctgagcttagccctgcagatgtacgggagccgggttatcgagaaggccctggaattcatccctagcgatcagcagaacgagatggtcagagagctggacggccacgtgctcaagtgcgtgaaagaccagaacggctgccacgtggtacagaagtgtatcgagtgtgtgcagccccagagcctccagtttatcattgacgcctttaagggccaggtgtttgccctgagcacacacccctacggcagccgggtaatcgagcggatcctggagcactgcctgcccgaccagaccttgcccatcctcgaggaactgcaccagcacaccgagcagctggtgcaggaccaatacggctgttacgtcatccagcacgttctggagcacggccggcctgaggacaagtctaagatcgtggccgaaatccggggcaacgtgttggtgctgagccagcacaagttcgcctcaaacgtcgtggagaagtgtgtgactcacgcctctcggaccgagcgggcagtgctgattgacgaggtgtgtaccatgaacgacggcccacacagcgccctgtacaccatgatgaaggaccagtacgcctgctacgttgtgcagaagatgattgacgtggccgaacccggtcagcgcaagattgtgatgcacaagatccggccccacatcgccaccctgcggaagtacacctacggcaagcacatcctggccaagctggagaagtactacatgaagaacggcgtggacctgggc

SEQ ID NO: 65 - protein, 667 aa, Artificial Sequence
  Description of Artificial Sequence Synthetic polypeptide
MGSSHHHHHHSQDLEVLFQGPHMGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGNRVIQKALEFIPSDQQNEMVRELDGHVLKCVKDQNGCHVVQKCIECVQPQSLQFIIDAFKGQVFALSTHPYGNRVIQRILEHCLPDQTLPILEELHQHTEQLVQDQYGCYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIMEFSQDQHGNRFIQLKLERATPAERQLVFNEILQAAYQLMVDVFGNYVIQKFFEFGSLEQKLALAERIRGHVLSLALQMYGNRVIQKALEFIPSDQQNEMVRELDGHVLKCVKDQNGNHVVQKCIECVQPQSLQFIIDAFKGQVFALSTHPYGNRVIQRILEHCLPDQTLPILEELHQHTEQLVQDQYGNYVIQHVLEHGRPEDKSKIVAEIRGNVLVLSQHKFANNVVQKCVTHASRTERAVLIDEVCTMNDGPHSALYTMMKDQYANYVVQKMIDVAEPGQRKIVMHKIRPHIATLRKYTYGKHILAKLEKYYMKNGVDLG

SEQ ID NO: 66 - DNA, 2001 bp, Artificial Sequence
  Description of Artificial Sequence Synthetic polynucleotide
atgggcagctcccatcaccaccaccatcattcgcaggaccttgaagtcctgttccaaggtcctcatatgggtagatcaaggcttttggaagattttcgaaataaccggtatcctaacctgcagctcagggagatagcaggacacatcatggagttcagccaggaccaacacggaagcaggtttatccaactgaaactcgagagagcaacgcccgcagagcgtcagctggtgtttaacgagatcctgcaggcagcctaccagctgatggttgacgtgttcggtaactacgtaattcagaagttctttgagttcggttctctggaacagaaactcgctctggctgagcgaattcggggtcacgtgctctcactggcgctccagatgtacggcaatagagtgattcagaaggctctggaatttattccatcggaccagcagaacgaaatggtacgagaactggacggtcacgtccttaagtgtgtcaaagatcagaacggctgtcacgtagtacagaagtgcatcgagtgtgtgcagccgcagagcctccagtttatcatcgacgcctttaaagggcaggtcttcgccttgtccacccatccgtacggaaaccgagtcattcagcgcatcctggaacattgcctcccggatcagaccctgccgattctggaggagcttcaccagcacaccgagcaactagtgcaggatcagtacggctgttacgtgatacagcacgtcctggagcacggtagaccagaggacaaatcgaagattgtggccgaaatcagaggaaacgtgctggtcctttcccagcacaagttcgcttccaacgtggtggagaagtgcgtgacacacgcaagccgcaccgagagggcagtcttaatcgacgaagtgtgcacaatgaacgacggtccacactctgccttatacacaatgatgaaggaccagtacgcaaactacgtggtgcagaagatgattgacgtagctgagcctggccagcgaaagattgtcatgcataagatcaggcctcatattatggagttttctcaggatcaacacggcaatcggttcatccaattaaagctggagagggccacacctgcggagaggcagttggtctttaacgaaattctacaagccgcttaccaattaatggtggacgtctttggcaactacgtgatccagaaattcttcgagtttggaagtcttgaacagaagctggcgctggctgaaagaatcagaggtcacgtcctcagcctggcattgcaaatgtacggaaatcgagtcatccagaaagcccttgagtttatccccagcgatcaacagaacgagatggttcgcgaattggacgggcacgtgctgaagtgtgttaaggaccagaacgggaaccacgtcgtgcagaagtgcatagagtgcgtccagccacagtccctgcagtttattattgacgcatttaagggacaagtgttcgccttatctacccacccttacggcaatagggtcattcagaggatcttggaacactgtcttcccgaccagacactgccaatattggaagaactgcatcaacataccgaacagctcgtacaagatcagtacggtaattacgtcattcaacacgttctcgaacacggaaggcccgaagacaagtctaagatcgtcgcagagatacgtgggaacgtcctcgtcttaagtcagcataagttcgcaaacaacgttgtgcagaagtgtgtaacacacgcgagcagaacagagagagccgtgctgatcgacgaggtgtgtaccatgaacgacgggccacacagcgccctctacaccatgatgaaagaccaatacgctaactacgtggttcagaagatgatcgacgtcgccgaaccaggtcaacggaagatcgtgatgcacaagatcaggccacacatagccaccctgagaaagtatacgtacggtaaacatatcctggcaaagttagagaagtactatatgaagaacggcgtggacttagga

SEQ ID NO: 67 - protein, 23 aa, Unknown
  Description of Unknown Lambda sequence
MGNARTRRRERRAEKQAQWKAAN

SEQ ID NO: 68 - DNA, 69 bp, Unknown
  Description of Unknown Lambda sequence
atgggcaacgcccggaccaggagaagagagcggcgggccgagaagcaggcccagtggaaggccgccaac

SEQ ID NO: 69 - protein, 101 aa, Unknown
  Description of Unknown U1A sequence
MAVPETRPNHTIYINNLNEKIKKDELKKSLYAIFSQFGQILDILVSRSLKMRGQAFVIFKEVSSATNALRSMQGFPFYDKPMRIQYAKTDSDIIAKMKGTF

SEQ ID NO: 70 - DNA, 303 bp, Unknown
  Description of Unknown U1A sequence
atggccgtgcctgagaccagacctaaccacaccatctacatcaacaacctgaacgagaagatcaagaaggacgagctgaagaagagcctgtacgccatcttcagccagttcggccagatcctggacatcctggtgagcagaagcctgaagatgagaggccaggccttcgtgatcttcaaggaggtgagcagcgccaccaacgccctgagaagcatgcagggcttccctttctacgacaagcctatgagaatccagtacgccaagaccgacagcgacatcatcgccaagatgaagggcaccttc

SEQ ID NO: 71 - protein, 128 aa, Unknown
  Description of Unknown 15.5kd sequence
MTEADVNPKAYPLADAHLTKKLLDLVQQSCNYKQLRKGANEATKTLNRGISEFIVMAADAEPLEIILHLPLLCEDKNVPYVFVRSKQALGRACGVSRPVIACSVTIKEGSQLKQQIQSIQQSIERLLV

SEQ ID NO: 72 - DNA, 384 bp, Unknown
  Description of Unknown 15.5kd sequence
atgaccgaggccgacgtgaaccctaaggcctaccctctggccgacgcccacctgaccaagaagctgctggacctggtgcagcagagctgcaactacaagcagctgagaaagggcgccaacgaggccaccaagaccctgaacagaggcatcagcgagttcatcgtgatggccgccgacgccgagcctctggagatcatcctgcacctgcctctgctgtgcgaggacaagaacgtgccttacgtgttcgtgagaagcaagcaggccctgggcagagcctgcggcgtgagcagacctgtgatcgcctgcagcgtgaccatcaaggagggcagccagctgaagcagcagatccagagcatccagcagagcatcgagagactgctggtg

SEQ ID NO: 73 - protein, 582 aa, Unknown
  Description of Unknown LARP7 sequence
METESGNQEKVMEEESTEKKKEVEKKKRSRVKQVLADIAKQVDFWFGDANLHKDRFLREQIEKSRDGYVDISLLVSFNKMKKLTTDGKLIARALRSSAVVELDLEGTRIRRKKPLGERPKDEDERTVYVELLPKNVNHSWIERVFGKCGNVVYISIPHYKSTGDPKGFAFVEFETKEQAAKAIEFLNNPPEEAPRKPGIFPKTVKNKPIPALRVVEEKKKKKKKKGRMKKEDNIQAKEENMDTSNTSISKMKRSRPTSEGSDIESTEPQKQCSKKKKKRDRVEASSLPEVRTGKRKRSSSEDAESLAPRSKVKKIIQKDIIKEASEASKENRDIEISTEEEKDTGDLKDSSLLKTKRKHKKKHKERHKMGEEVIPLRVLSKSEWMDLKKEYLALQKASMASLKKTISQIKSESEMETDSGVPQNTGMKNEKTANREECRTQEKVNATGPQFVSGVIVKIISTEPLPGRKQVRDTLAAISEVLYVDLLEGDTECHARFKTPEDAQAVINAYTEINKKHCWKLEILSGDHEQRYWQKILVDRQAKLNQPREKKRGTEKLITKAEKIRLAKTQQASKHIRFSEYD

SEQ ID NO: 74 - DNA, 1746 bp, Unknown
  Description of Unknown LARP7 sequence
atggagaccgagagcggcaaccaggagaaggtgatggaggaggagagcaccgagaagaagaaggaggtggagaagaagaagagaagcagagtgaagcaggtgctggccgacatcgccaagcaggtggacttctggttcggcgacgccaacctgcacaaggacagattcctgagagagcagatcgagaagagcagagacggctacgtggacatcagcctgctggtgagcttcaacaagatgaagaagctgaccaccgacggcaagctgatcgccagagccctgagaagcagcgccgtggtggagctggacctggagggcaccagaatcagaagaaagaagcctctgggcgagagacctaaggacgaggacgagagaaccgtgtacgtggagctgctgcctaagaacgtgaaccacagctggatcgagagagtgttcggcaagtgcggcaacgtggtgtacatcagcatccctcactacaagagcaccggcgaccctaagggcttcgccttcgtggagttcgagaccaaggagcaggccgccaaggccatcgagttcctgaacaaccctcctgaggaggcccctagaaagcctggcatcttccctaagaccgtgaagaacaagcctatccctgccctgagagtggtggaggagaagaagaagaagaagaagaagaagggcagaatgaagaaggaggacaacatccaggccaaggaggagaacatggacaccagcaacaccagcatcagcaagatgaagagaagcagacctaccagcgagggcagcgacatcgagagcaccgagcctcagaagcagtgcagcaagaagaagaagaagagagacagagtggaggccagcagcctgcctgaggtgagaaccggcaagagaaagagaagcagcagcgaggacgccgagagcctggcccctagaagcaaggtgaagaagatcatccagaaggacatcatcaaggaggccagcgaggccagcaaggagaacagagacatcgagatcagcaccgaggaggagaaggacaccggcgacctgaaggacagcagcctgctgaagaccaagagaaagcacaagaagaagcacaaggagagacacaagatgggcgaggaggtgatccctctgagagtgctgagcaagagcgagtggatggacctgaagaaggagtacctggccctgcagaaggccagcatggccagcctgaagaagaccatcagccagatcaagagcgagagcgagatggagaccgacagcggcgtgcctcagaacaccggcatgaagaacgagaagaccgccaacagagaggagtgcagaacccaggagaaggtgaacgccaccggccctcagttcgtgagcggcgtgatcgtgaagatcatcagcaccgagcctctgcctggcagaaagcaggtgagagacaccctggccgccatcagcgaggtgctgtacgtggacctgctggagggcgacaccgagtgccacgccagattcaagacccctgaggacgcccaggccgtgatcaacgcctacaccgagatcaacaagaagcactgctggaagctggagatcctgagcggcgaccacgagcagagatactggcagaagatcctggtggacagacaggccaagctgaaccagcctagagagaagaagagaggcaccgagaagctgatcaccaaggccgagaagatcagactggccaagacccagcaggccagcaagcacatcagattcagcgagtacgac

SEQ ID NO: 75 - DNA, 22 bp, Unknown
  Description of Unknown Target sequence
caaacaccattgtcacactcca

SEQ ID NO: 76 - DNA, 23 bp, Unknown
  Description of Unknown Target sequence
tccataaagtaggaaacactaca

SEQ ID NO: 77 - DNA, 22 bp, Unknown
  Description of Unknown Target sequence
cgcattattactcacggtacga

SEQ ID NO: 78 - DNA, 22 bp, Unknown
  Description of Unknown Target sequence
cactggtacaagggttgggaga

SEQ ID NO: 79 - DNA, 23 bp, Unknown
  Description of Unknown Target sequence
acccctatcacgattagcattaa

SEQ ID NO: 80 - DNA, 22 bp, Unknown
  Description of Unknown Target sequence
tggggtatttgacaaactgaca

SEQ ID NO: 81 - RNA, 133 nt, Artificial Sequence
  Description of Artificial Sequence Synthetic polynucleotide
gcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 82 - RNA, 141 nt, Artificial Sequence
  Description of Artificial Sequence Synthetic polynucleotide
ugauaauaggcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 83 - RNA, 119 nt, Artificial Sequence
  Description of Artificial Sequence Synthetic polynucleotide
ugauaauaggcuggagccucgguggccaugcuucuugccccuugggccuccccccagccccuccuccccuuccugcacccguacccccguggucuuugaauaaagucugagugggcggc

SEQ ID NO: 84 - RNA, 142 nt, Artificial Sequence
  Description of Artificial Sequence Synthetic polynucleotide
ugauaauaggcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 85 - RNA, 164 nt, Artificial Sequence
  Description of Artificial Sequence Synthetic polynucleotide
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 86 - RNA, 188 nt, Artificial Sequence
  Description of Artificial Sequence Synthetic polynucleotide
ugauaauaguccauaaaguaggaaacacuacagcuggagccucgguggccaugcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 87 - RNA, 140 nt, Artificial Sequence
  Description of Artificial Sequence Synthetic polynucleotide
ugauaauaggcuggagccucgguggccaugcuucuugccccuugggccuccccccagccccuccuccccuuccugcacccguacccccaguagugcuuucuacuuuaugguggucuuugaauaaagucugagugggcggc

SEQ ID NO: 88 - RNA, 182 nt, Artificial Sequence
  Description of Artificial Sequence Synthetic polynucleotide
ugauaauagaguagugcuuucuacuuuauggcuggagccucgguggccaugcuucuugccccuugggccaguagugcuuucuacuuuauguccccccagccccuccuccccuuccugcacccguacccccaguagugcuuucuacuuuaugguggucuuugaauaaagucugagugggcggc

SEQ ID NO: 89 - RNA, 184 nt, Artificial Sequence
  Description of Artificial Sequence Synthetic polynucleotide
ugauaauagaguagugcuuucuacuuuauggcuggagccucgguggccaugcuucuugccccuugggccuccauaaaguaggaaacacuacauccccccagccccuccuccccuuccugcacccguacccccaguagugcuuucuacuuuaugguggucuuugaauaaagucugagugggcggc

SEQ ID NO: 90 - RNA, 142 nt, Artificial Sequence
  Description of Artificial Sequence Synthetic polynucleotide
ugauaauaggcuggagccucgguggccaugcuucuugccccuugggccuccccccagccccuccuccccuuccugcacccguacccccaccccuaucacaauuagcauuaaguggucuuugaauaaagucugagugggcggc

SEQ ID NO: 91 - RNA, 188 nt, Artificial Sequence
  Description of Artificial Sequence Synthetic polynucleotide
ugauaauagaccccuaucacaauuagcauuaagcuggagccucgguggccaugcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 92 - RNA, 188 nt, Artificial Sequence
  Description of Artificial Sequence Synthetic polynucleotide
ugauaauagaccccuaucacaauuagcauuaagcuggagccucgguggccaugcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 93 - RNA, 142 nt, Artificial Sequence
  Description of Artificial Sequence Synthetic polynucleotide
ugauaauaguccauaaaguaggaaacacuacagcuggagccucgguggccaugcuucuugccccuugggccuccccccagccccuccuccccuuccugcacccguacccccguggucuuugaauaaagucugagugggcggc

SEQ ID NO: 94 - RNA, 142 nt, Artificial Sequence
  Description of Artificial Sequence Synthetic polynucleotide
ugauaauaggcuggagccucgguggcuccauaaaguaggaaacacuacacaugcuucuugccccuugggccuccccccagccccuccuccccuuccugcacccguacccccguggucuuugaauaaagucugagugggcggc

SEQ ID NO: 95 - RNA, 142 nt, Artificial Sequence
  Description of Artificial Sequence Synthetic polynucleotide
ugauaauaggcuggagccucgguggccaugcuucuugccccuugggccuccauaaaguaggaaacacuacauccccccagccccuccuccccuuccugcacccguacccccguggucuuugaauaaagucugagugggcggc

SEQ ID NO: 96 - RNA, 181 nt, Artificial Sequence
  Description of Artificial Sequence Synthetic polynucleotide
ugauaauagaguagugcuuucuacuuuauggcuggagccucgguggccaugcuucuugccccuugggccaguagugcuuucuacuuuauguccccccagccccucuccccuuccugcacccguacccccaguagugcuuucuacuuuaugguggucuuugaauaaagucugagugggcggc

SEQ ID NO: 97 - RNA, 142 nt, Artificial Sequence
  Description of Artificial Sequence Synthetic polynucleotide
ugauaauaggcuggagccucgguggccaugcuucuugccccuuccauaaaguaggaaacacuacaugggccuccccccagccccuccuccccuuccugcacccguacccccguggucuuugaauaaagucugagugggcggc

SEQ ID NO: 98 - RNA, 142 nt, Artificial Sequence
  Description of Artificial Sequence Synthetic polynucleotide
ugauaauaggcuggagccucgguggccaugcuucuugccccuugggccuccccccaguccauaaaguaggaaacacuacaccccuccuccccuuccugcacccguacccccguggucuuugaauaaagucugagugggcggc

SEQ ID NO: 99 - RNA, 142 nt, Artificial Sequence
  Description of Artificial Sequence Synthetic polynucleotide
ugauaauaggcuggagccucgguggccaugcuucuugccccuugggccuccccccagccccuccuccccuucuccauaaaguaggaaacacuacacugcacccguacccccguggucuuugaauaaagucugagugggcggc

SEQ ID NO: 100 - RNA, 142 nt, Artificial Sequence
  Description of Artificial Sequence Synthetic polynucleotide
ugauaauaggcuggagccucgguggccaugcuucuugccccuugggccuccccccagccccuccuccccuuccugcacccguacccccguggucuuugaauaaaguuccauaaaguaggaaacacuacacugagugggcggc

SEQ ID NO: 101 - RNA, 164 nt, Artificial Sequence
  Description of Artificial Sequence Synthetic polynucleotide
ugauaauaguccauaaaguaggaaacacuacagcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 102 - RNA, 119 nt, Artificial Sequence
  Description of Artificial Sequence Synthetic polynucleotide
ugauaauaggcuggagccucgguggccuagcuucuugccccuugggccuccccccagccccuccuccccuuccugcacccguacccccguggucuuugaauaaagucugagugggcggc

SEQ ID NO: 103 - RNA, 142 nt, Artificial Sequence
  Description of Artificial Sequence Synthetic polynucleotide
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 104 - RNA, 141 nt, Artificial Sequence
  Description of Artificial Sequence Synthetic polynucleotide
ugauaauaggcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 105 - RNA, 188 nt, Artificial Sequence
  Description of Artificial Sequence Synthetic polynucleotide
ugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 106 - RNA, 142 nt, Artificial Sequence
  Description of Artificial Sequence Synthetic polynucleotide
ugauaauaguccauaaaguaggaaacacuacagcuggagccucgguggccuagcuucuugccccuugggccuccccccagccccuccuccccuuccugcacccguacccccguggucuuugaauaaagucugagugggcggc

SEQ ID NO: 107 - RNA, 142 nt, Artificial Sequence
  Description of Artificial Sequence Synthetic polynucleotide
ugauaauaggcuggagccucgguggcuccauaaaguaggaaacacuacacuagcuucuugccccuugggccuccccccagccccuccuccccuuccugcacccguacccccguggucuuugaauaaagucugagugggcggc

SEQ ID NO: 108 - RNA, 142 nt, Artificial Sequence
  Description of Artificial Sequence Synthetic polynucleotide
ugauaauaggcuggagccucgguggccuagcuucuugccccuugggccuccauaaaguaggaaacacuacauccccccagccccuccuccccuuccugcacccguacccccguggucuuugaauaaagucugagugggcggc

SEQ ID NO: 109 - RNA, 142 nt, Artificial Sequence
  Description of Artificial Sequence Synthetic polynucleotide
ugauaauaggcuggagccucgguggccuagcuucuugccccuugggccuccccccagccccuccuccccuuccugcacccguacccccaccccuaucacaauuagcauuaaguggucuuugaauaaagucugagugggcggc

SEQ ID NO: 110 - RNA, 188 nt, Artificial Sequence
  Description of Artificial Sequence Synthetic polynucleotide
ugauaauagaccccuaucacaauuagcauuaagcuggagccucgguggccuagcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 111 - RNA, 188 nt, Artificial Sequence
  Description of Artificial Sequence Synthetic polynucleotide
ugauaauagaccccuaucacaauuagcauuaagcuggagccucgguggccuagcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 112 - RNA, 141 nt, Artificial Sequence
  Description of Artificial Sequence Synthetic polynucleotide
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 113 - RNA, 141 nt, Artificial Sequence
  Description of Artificial Sequence Synthetic polynucleotide
ugauaauaggcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 114 - RNA, 141 nt, Artificial Sequence; identical to SEQ ID NO: 104
  Description of Artificial Sequence Synthetic polynucleotide

SEQ ID NO: 115 - RNA, 47 nt, Artificial Sequence
  Description of Artificial Sequence Synthetic oligonucleotide
gggaaauaagagagaaaagaagaguaagaagaaauauaagagccacc

SEQ ID NO: 116 - RNA, 70 nt, Artificial Sequence
  Description of Artificial Sequence Synthetic oligonucleotide
gggaaauaagaguccauaaaguaggaaacacuacaagaaaagaagaguaagaagaaauauaagagccacc

SEQ ID NO: 117 - RNA, 70 nt, Artificial Sequence
  Description of Artificial Sequence Synthetic oligonucleotide
gggaaauaagagagaaaagaagaguaauccauaaaguaggaaacacuacagaagaaauauaagagccacc

SEQ ID NO: 118 - RNA, 70 nt, Artificial Sequence
  Description of Artificial Sequence Synthetic oligonucleotide
gggaaauaagagagaaaagaagaguaagaagaaauauaauccauaaaguaggaaacacuacagagccacc

SEQ ID NO: 119 - RNA, 47 nt, Artificial Sequence
  Description of Artificial Sequence Synthetic oligonucleotide
aggaaauaagagagaaaagaagaguaagaagaaauauaagagccacc

SEQ ID NO: 120 - RNA, 57 nt, Artificial Sequence
  Description of Artificial Sequence Synthetic oligonucleotide
gggaaauaagagagaaaagaagaguaagaagaaauauaagaccccggcgccgccacc

SEQ ID NO: 121 - RNA, 57 nt, Artificial Sequence
  Description of Artificial Sequence Synthetic oligonucleotide
aggaaauaagagagaaaagaagaguaagaagaaauauaagaccccggcgccgccacc

SEQ ID NO: 122 - RNA, 47 nt, Artificial Sequence
  Description of Artificial Sequence Synthetic oligonucleotide
gggagaucagagagaaaagaagaguaagaagaaauauaagagccacc

SEQ ID NO: 123 - RNA, 42 nt, Artificial Sequence
  Description of Artificial Sequence Synthetic oligonucleotide
gggagacaagcuuggcauuccgguacuguugguaaagccacc

SEQ ID NO: 124 - RNA, 47 nt, Artificial Sequence
  Description of Artificial Sequence Synthetic oligonucleotide
gggaauuaacagagaaaagaagaguaagaagaaauauaagagccacc

SEQ ID NO: 125 - RNA, 47 nt, Artificial Sequence
  Description of Artificial Sequence Synthetic oligonucleotide
gggaaauuagacagaaaagaagaguaagaagaaauauaagagccacc

SEQ ID NO: 126 - RNA, 47 nt, Artificial Sequence
  Description of Artificial Sequence Synthetic oligonucleotide
gggaaauaagagaguaaagaacaguaagaagaaauauaagagccacc

SEQ ID NO: 127 - RNA, 47 nt, Artificial Sequence
  Description of Artificial Sequence Synthetic oligonucleotide
gggaaaaaagagagaaaagaagacuaagaagaaauauaagagccacc

SEQ ID NO: 128 - RNA, 47 nt, Artificial Sequence
  Description of Artificial Sequence Synthetic oligonucleotide
gggaaauaagagagaaaagaagaguaagaagauauauaagagccacc

SEQ ID NO: 129 - RNA, 47 nt, Artificial Sequence
  Description of Artificial Sequence Synthetic oligonucleotide
gggaaauaagagacaaaacaagaguaagaagaaauauaagagccacc

SEQ ID NO: 130 - RNA, 47 nt, Artificial Sequence
  Description of Artificial Sequence Synthetic oligonucleotide
gggaaauuagagaguaaagaacaguaaguagaauuaaaagagccacc

SEQ ID NO: 131 - RNA, 47 nt, Artificial Sequence
  Description of Artificial Sequence Synthetic oligonucleotide
gggaaauaagagagaauagaagaguaagaagaaauauaagagccacc

SEQ ID NO: 132 - RNA, 47 nt, Artificial Sequence
  Description of Artificial Sequence Synthetic oligonucleotide
gggaaauaagagagaaaagaagaguaagaagaaaauuaagagccacc

SEQ ID NO: 133 - RNA, 47 nt, Artificial Sequence
  Description of Artificial Sequence Synthetic oligonucleotide
gggaaauaagagagaaaagaagaguaagaagaaauuuaagagccacc

SEQ ID NO: 134 - RNA, 92 nt, Artificial Sequence
  Description of Artificial Sequence Synthetic oligonucleotide
ucaagcuuuuggacccucguacagaagcuaauacgacucacuauagggaaauaagagagaaaagaagaguaagaagaaauauaagagccacc

SEQ ID NO: 135 - RNA, 236 nt, Artificial Sequence; identical to SEQ ID NO: 8
  Description of Artificial Sequence Synthetic polynucleotide

SEQ ID NO: 136 - protein, 1019 aa, Artificial Sequence
  Description of Artificial Sequence Synthetic polypeptide
MALAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAELQLSAGRYHYQLVWCQK

SEQ ID NO: 137 - DNA, 3057 bp, Artificial Sequence
  Description of Artificial Sequence Synthetic polynucleotide
atggccttggctgtcgtcctgcaaagaagagattgggaaaatcctggagttacgcaactgaatagactcgccgcacatccaccgttcgcgtcctggcgaaatagcgaagaagcgcggaccgacagaccttcgcagcagctgcgctctctcaacggggaatggcggttcgcatggtttccggctcctgaggcagtcccggaaagctggctcgagtgcgacctcccggaagccgatacggtggtggtgccgtcaaattggcaaatgcatggatacgacgcccccatctacaccaacgtcacttaccctatcaccgtgaatcccccattcgtcccgactgagaacccgactggatgctacagcctgacctttaacgtggacgagtcgtggctgcaagaagggcagactcgcatcattttcgacggagtcaactccgcgttccatctttggtgtaacggacggtgggtgggatacgggcaggactccaggctgccgagcgaattcgacttgtcagccttcctgcgcgccggcgaaaaccgcctggctgtcatggtccttagatggtcggatggctcgtacctggaggatcaggacatgtggaggatgtcagggatcttccgggatgtctcgctgctccacaagccaactacccagatctccgactttcatgtggccacccgcttcaacgatgacttcagcagggcggttctggaagccgaggtgcaaatgtgcggagaactgagggactacctccgcgtgactgtctcgctctggcagggtgaaacccaagtggcttcaggcactgcaccgttcggaggagaaatcatcgacgaacggggaggatacgccgatcgcgtcaccctgcgcctcaatgtggaaaatccgaaactgtggtcggcagaaatccctaatttgtaccgggccgtggtggagctgcacaccgccgacggaactctgatcgaggccgaggcatgcgatgtgggattccgcgaggtccgcatcgaaaatggactgcttctgcttaatggcaaaccgctgctcatccgcggagtgaacagacacgagcatcacccgctgcacggtcaggtcatggatgaacagactatggtgcaagacatcctgctgatgaaacaaaacaacttcaacgccgttcggtgctcccattaccctaatcacccgttgtggtataccctttgcgatcggtacggcctctacgtggtggacgaagcgaacatcgagactcacggaatggtccctatgaaccgcctcactgacgacccgaggtggctcccggcaatgtcggaacgagtgactcggatggtgcagagggaccgcaaccatccgtcggtgataatctggtcgctggggaacgaatctggccacggagctaaccacgatgcgctgtaccgctggattaagtccgtggacccaagccggcccgtccagtacgaaggaggtggtgctgataccactgcaaccgacatcatctgcccaatgtatgcgcgggtggatgaggaccaacctttcccggcggtgccaaagtggtccatcaagaaatggctctcgctgcccggagaaacgcgcccgctgatcctgtgcgaatatgcgcacgctatgggaaattcactggggggatttgcgaagtactggcaggcttttcgacagtacccgagactccagggtggcttcgtgtgggactgggttgaccagagcctcatcaaatacgatgaaaacggcaacccatggtccgcgtacggcggagactttggagacacccctaacgatcgccagttctgcatgaacggcctggtgttcgccgacagaactccgcatccagcccttactgaggctaagcaccaacaacagttcttccagttcagactgtcggggcaaacgatcgaagtgacttccgaatacctcttccggcattcggacaacgagttgctgcactggatggtcgccctggatggaaagcccctcgcctccggagaagtgccgctcgacgtggcgccgcagggaaagcagttgatcgagttgccggaactgccacagcccgagtcagcaggacagctctggcttaccgtcagagtcgtgcagccaaatgccaccgcctggtcggaggcaggacatatttcagcctggcagcagtggcgcctcgccgagaatctgagcgtgaccttgccagcagcctcacacgccattccgcatctgaccacgtccgaaatggacttttgtatcgaactggggaataagcgctggcagttcaatcggcaatcaggcttcctgtcccagatgtggattggtgacaagaagcagctcctgaccccgctgcgcgatcagttcactcgcgccccacttgacaacgacattggcgtgagcgaggccacgcgtatcgatccaaacgcttgggtggagcgctggaaggcggctggccactatcaggcggaggccgcgctcctgcagtgtaccgcggataccctcgcagacgccgttctgattaccacggcgcatgcctggcaacaccagggaaagaccctgtttatcagccgcaaaacttaccggatcgatggcagcggccaaatggcgatcactgtggacgtcgaggtggcatcagacactccacacccagcacggatcggactcaattgccaactggctcaagtggctgagagagtcaattggctgggcctcggcccccaagagaactaccctgatcggcttactgccgcatgctttgaccggtgggatctgcctctgtcggatatgtacaccccctacgtgttcccatccgagaacggtctgagatgcggtactagggagttgaactacggaccgcaccaatggaggggggactttcagttcaacatctcaagatacagccagcagcaattgatggaaacctcgcaccggcatctcttgcatgcagaggaagggacctggctgaacatcgatggattccacatgggaattggtggggatgactcctggtcccctagcgtgtccgcggaacttcagctgtccgccggccggtaccactaccagctcgtgtggtgtcaaaag

SEQ ID NO: 138 - protein, 239 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 139 - DNA, 717 bp, Artificial Sequence
  Description of Artificial Sequence Synthetic polynucleotide
atggtgtccaagggtgaggaattgtttaccggggtggtgcctattctcgtcgaacttgacggggatgtgaatggacacaagttttcggtatccggagaaggagagggtgacgccacatacggaaagcttacactcaaattcatctgtacgacggggaaactgcccgtaccctggcctacgctcgtaaccacgctgacttatggagtgcagtgctttagcagataccccgaccatatgaagcagcacgacttcttcaagtcggcgatgcccgaggggtacgtgcaagagaggaccattttcttcaaagacgatggcaattacaaaacacgcgcagaagtcaagtttgagggcgatactctggtcaatcggatcgaattgaagggaatcgatttcaaagaagatggaaacatccttggccataagctcgagtacaactataactcgcataatgtctatatcatggctgacaagcagaaaaacggtatcaaagtcaactttaagatccgacacaatattgaggacggttcggtgcagcttgcggaccactatcaacagaatacgccgattggggatggtccggtccttttgccggataaccattatctctcaacccagtcagccctgagcaaagatccaaacgagaagagggaccacatggtcttgctcgaattcgtgacagcggcagggatcactctgggaatggacgagttgtacaag

SEQ ID NO: 140 - protein, 699 aa, Artificial Sequence
  Description of Artificial Sequence Synthetic polypeptide
MGKPIPNPLLGLDSTQGQEQKLISEEDLAGKPIPNPLLGLDSTQGQEQKLISEEDLAGKPIPNPLLGLDSTQGQEQKLISEEDLAGKPIPNPLLGLDSTQGQEQKLISEEDLAGKPIPNPLLGLDSTQGQEQKLISEEDLAPAAKRVKLDEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 141 - DNA, 2097 bp, Artificial Sequence
  Description of Artificial Sequence Synthetic polynucleotide
atggggaaacctatcccaaaccccttgctcgggctcgattcaacacaagggcaggaacagaagcttatttccgaggaggacttagcaggaaagcccattcctaatccattgctgggtttggacagtacgcaggggcaagagcagaaattaatttcagaggaggatctcgccggaaaacccatacccaacccccttcttggcctagacagtactcagggacaagaacagaagttaatctcagaagaggacctagcagggaagcctatccccaacccccttctagggctagattccacccaggggcaggagcagaaactgattagcgaggaggatttggcaggcaaacctatccctaaccccctgctgggactcgatagcactcaagggcaggaacagaagctgatctccgaggaagacctcgccccggctgcgaagcgcgtgaagctggatgaagatgcgaagaacatcaagaagggacctgccccgttttaccctttggaggacggtacagcaggagaacagctccacaaggcgatgaaacgctacgccctggtccccggaacgattgcgtttaccgatgcacatattgaggtagacatcacatacgcagaatacttcgaaatgtcggtgaggctggcggaagcgatgaagagatatggtcttaacactaatcaccgcatcgtggtgtgttcggagaactcattgcagtttttcatgccggtccttggagcacttttcatcggggtcgcagtcgcgccagcgaacgacatctacaatgagcgggaactcttgaatagcatgggaatctcccagccgacggtcgtgtttgtctccaaaaaggggctgcagaaaatcctcaacgtgcagaagaagctccccattattcaaaagatcatcattatggatagcaagacagattaccaagggttccagtcgatgtatacctttgtgacatcgcatttgccgccagggtttaacgagtatgacttcgtccccgagtcatttgacagagataaaaccatcgcgctgattatgaattcctcgggtagcaccggtttgccaaagggggtggcgttgccccaccgcactgcttgtgtgcggttctcgcacgctagggatcctatctttggtaatcagatcattcccgacacagcaatcctgtccgtggtaccttttcatcacggttttggcatgttcacgactctcggctatttgatttgcggtttcagggtcgtacttatgtatcggttcgaggaagaactgtttttgagatccttgcaagattacaagatccagtcggccctccttgtgccaacgcttttctcattctttgcgaaatcgacacttattgataagtatgacctttccaatctgcatgagattgcctcagggggagcgccgcttagcaaggaagtcggggaggcagtggccaagcgcttccaccttcccggaattcggcagggatacgggctcacggagacaacatccgcgatccttatcacgcccgagggtgacgataagccgggagccgtcggaaaagtggtccccttctttgaagccaaggtcgtagacctcgacacgggaaaaaccctcggagtgaaccagaggggcgagctctgcgtgagagggccgatgatcatgtcaggttacgtgaataaccctgaagcgacgaatgcgctgatcgacaaggatgggtggttgcattcgggagacattgcctattgggatgaggatgagcacttctttatcgtagatcgacttaagagcttgatcaaatacaaaggctatcaggtagcgcctgccgagctcgagtcaatcctgctccagcaccccaacattttcgacgccggagtggccgggttgcccgatgacgacgcgggtgagctgccagcggccgtggtagtcctcgaacatgggaaaacaatgaccgaaaaggagatcgtggactacgtagcatcacaagtgacgactgcgaagaaactgaggggaggggtagtctttgtggacgaggtcccgaaaggcttgactgggaagcttgacgctcgcaaaatccgggaaatcctgattaaggcaaagaaaggcgggaaaatcgctgtc

SEQ ID NO: 142 - protein, 284 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKRSRDISHGFPPAVAAQDDGTLPMSCAQESGMDRHPAACASARINV

SEQ ID NO: 143 - DNA, 852 bp, Artificial Sequence
  Description of Artificial Sequence Synthetic polynucleotide
atggtgtccaagggtgaggaattgtttaccggggtggtgcctattctcgtcgaacttgacggggatgtgaatggacacaagttttcggtatccggagaaggagagggtgacgccacatacggaaagcttacactcaaattcatctgtacgacggggaaactgcccgtaccctggcctacgctcgtaaccacgctgacttatggagtgcagtgctttagcagataccccgaccatatgaagcagcacgacttcttcaagtcggcgatgcccgaggggtacgtgcaagagaggaccattttcttcaaagacgatggcaattacaaaacacgcgcagaagtcaagtttgagggcgatactctggtcaatcggatcgaattgaagggaatcgatttcaaagaagatggaaacatccttggccataagctcgagtacaactataactcgcataatgtctatatcatggctgacaagcagaaaaacggtatcaaagtcaactttaagatccgacacaatattgaggacggttcggtgcagcttgcggaccactatcaacagaatacgccgattggggatggtccggtccttttgccggataaccattatctctcaacccagtcagccctgagcaaagatccaaacgagaagagggaccacatggtcttgctcgaattcgtgacagcggcagggatcactctgggaatggacgagttgtacaagagatctcgagatatcagccatggcttcccgccggcggtggcggcgcaggatgatggcacgctgcccatgtcttgtgcccaggagagcgggatggaccgtcaccctgcagcctgtgcttctgctaggatcaatgtg

SEQ ID NO: 144 - protein, 550 aa, Artificial Sequence
  Description of Artificial Sequence Synthetic polypeptide
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 145 - DNA, 1650 bp, Artificial Sequence
  Description of Artificial Sequence Synthetic polynucleotide
atggaagatgcgaagaacatcaagaagggacctgccccgttttaccctttggaggacggtacagcaggagaacagctccacaaggcgatgaaacgctacgccctggtccccggaacgattgcgtttaccgatgcacatattgaggtagacatcacatacgcagaatacttcgaaatgtcggtgaggctggcggaagcgatgaagagatatggtcttaacactaatcaccgcatcgtggtgtgttcggagaactcattgcagtttttcatgccggtccttggagcacttttcatcggggtcgcagtcgcgccagcgaacgacatctacaatgagcgggaactcttgaatagcatgggaatctcccagccgacggtcgtgtttgtctccaaaaaggggctgcagaaaatcctcaacgtgcagaagaagctccccattattcaaaagatcatcattatggatagcaagacagattaccaagggttccagtcgatgtatacctttgtgacatcgcatttgccgccagggtttaacgagtatgacttcgtccccgagtcatttgacagagataaaaccatcgcgctgattatgaattcctcgggtagcaccggtttgccaaagggggtggcgttgccccaccgcactgcttgtgtgcggttctcgcacgctagggatcctatctttggtaatcagatcattcccgacacagcaatcctgtccgtggtaccttttcatcacggttttggcatgttcacgactctcggctatttgatttgcggtttcagggtcgtacttatgtatcggttcgaggaagaactgtttttgagatccttgcaagattacaagatccagtcggccctccttgtgccaacgcttttctcattctttgcgaaatcgacacttattgataagtatgacctttccaatctgcatgagattgcctcagggggagcgccgcttagcaaggaagtcggggaggcagtggccaagcgcttccaccttcccggaattcggcagggatacgggctcacggagacaacatccgcgatccttatcacgcccgagggtgacgataagccgggagccgtcggaaaagtggtccccttctttgaagccaaggtcgtagacctcgacacgggaaaaaccctcggagtgaaccagaggggcgagctctgcgtgagagggccgatgatcatgtcaggttacgtgaataaccctgaagcgacgaatgcgctgatcgacaaggatgggtggttgcattcgggagacattgcctattgggatgaggatgagcacttctttatcgtagatcgacttaagagcttgatcaaatacaaaggctatcaggtagcgcctgccgagctcgagtcaatcctgctccagcaccccaacattttcgacgccggagtggccgggttgcccgatgacgacgcgggtgagctgccagcggccgtggtagtcctcgaacatgggaaaacaatgaccgaaaaggagatcgtggactacgtagcatcacaagtgacgactgcgaagaaactgaggggaggggtagtctttgtggacgaggtcccgaaaggcttgactgggaagcttgacgctcgcaaaatccgggaaatcctgattaaggcaaagaaaggcgggaaaatcgctgtc

SEQ ID NO: 146 - protein, 193 aa, Artificial Sequence
  Description of Artificial Sequence Synthetic polypeptide
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 147 - DNA, 579 bp, Artificial Sequence
  Description of Artificial Sequence Synthetic polynucleotide
atgggagtgcacgagtgtcccgcgtggttgtggttgctgctgtcgctcttgagcctcccactgggactgcctgtgctgggggcaccacccagattgatctgcgactcacgggtacttgagaggtaccttcttgaagccaaagaagccgaaaacatcacaaccggatgcgccgagcactgctccctcaatgagaacattactgtaccggatacaaaggtcaatttctatgcatggaagagaatggaagtaggacagcaggccgtcgaagtgtggcaggggctcgcgcttttgtcggaggcggtgttgcggggtcaggccctcctcgtcaactcatcacagccgtgggagcccctccaacttcatgtcgataaagcggtgtcggggctccgcagcttgacgacgttgcttcgggctctgggcgcacaaaaggaggctatttcgccgcctgacgcggcctccgcggcacccctccgaacgatcaccgcggacacgtttaggaagctttttagagtgtacagcaatttcctccgcggaaagctgaaattgtatactggtgaagcgtgtaggacaggggatcgc

SEQ ID NO: 148 - RNA, 9 nt, Artificial Sequence
  Description of Artificial Sequence Synthetic oligonucleotide
ccrccaugg

SEQ ID NO: 149 - RNA, 100 nt, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 150 - RNA, 120 nt, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 151 - protein, 479 aa, Artificial Sequence
  Description of Artificial Sequence Synthetic polypeptide
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQ

SEQ ID NO: 152 - DNA, 1437 bp, Artificial Sequence
  Description of Artificial Sequence Synthetic polynucleotide
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggagggagcgcagagaacggcgacaacgagaagatggccgccctggaggccaagatctgccaccagatcgagtactacttcggcgactttaacctgccccgggacaagttcctgaaggagcagatcaagctggacgagggctgggtgcccctggagattatgatcaagttcaaccggctgaatcggctgaccaccgacttcaacgtgatcgtggaggctctgagcaagagcaaggccgagctgatggagatcagcgaggacaagacaaagatccggcggagcccatccaagcccctgcccgaggttaccgacgagtacaagaacgacgtgaagaaccggagcgtgtacatcaagggcttccccaccgacgccaccctggacgatatcaaggagtggctggaagacaagggccaggtgctgaacatccagatgcggcggaccctgcacaaggccttcaagggcagcatcttcgtggtgttcgacagcatcgagagcgccaagaaattcgtggagacacccggccagaagtacaaagagaccgacctgctgatcctgttcaaggacgactactttgccaagaagaacgaggagcggaagcagaacaaggtcgaagccaagctgcgggccaagcaggagcaggaggctaagcagaagctggaggaggacgccgagatgaagagcttggaggagaagatcggctgcctgctgaagttcagcggcgatctggacgaccagacctgccgggaggacctgcacatccttttcagcaaccacggcgagatcaagtggatcgacttcgtgcggggcgccaaggagggcatcatcctgtttaaggagaaggccaaagaagccctgggcaaggccaaggacgccaacaacggcaacctgcagctgcggaacaaggaggtgacctgggaggtgctggagggcgaggtggagaaggaggccctgaagaagatcatcgaggaccagcaggagagcctgaacaagtggaagtccaagggccggcggttcaaggggaagggtaagggcaacaaggcagcccagcccggctctgggaagggcaaggtgcagttccag

SEQ ID NO: 153 - RNA, 76 nt, Artificial Sequence
  Description of Artificial Sequence Synthetic oligonucleotide
gggaaaucgcaaaauuugcucuucgcguuagauuucuuuuaguuuucucgcaacuagcaagcuuuuuguucucgcc

SEQ ID NO: 154 - protein, 699 aa, Artificial Sequence; identical to SEQ ID NO: 140
  Description of Artificial Sequence Synthetic polypeptide

SEQ ID NO: 155 - DNA, 2097 bp, Artificial Sequence
  Description of Artificial Sequence Synthetic polynucleotide
atgggcaagcctattcccaacccgctgctgggcttagacagcacccaaggccaagagcagaagctcataagcgaagaggatctcgccggcaagccgatccccaacccactgctgggccttgacagtacccagggccaggaacagaaactgatcagcgaagaggacttagccgggaagcccatccccaaccctctgctgggtctggactccacccagggacaagaacagaagcttatatccgaggaagacctggccgggaagcctatccccaatcctctgctgggcctggatagcacccagggtcaggagcagaagctgatctccgaagaggaccttgcgggcaagcccatcccaaatcccctgttaggcctggacagcacccaggggcaggaacagaagctgataagcgaggaagatctggctcccgctgccaaacgggtgaagctggacgaggacgccaagaacatcaagaaggggcccgctcccttctacccgctggaggacggcaccgctggcgagcagctgcacaaggccatgaagaggtacgcactggtgcccggcacaatcgccttcaccgacgcccacatcgaggtggacatcacctacgccgagtacttcgagatgagcgtgcggctggccgaagccatgaagcggtacggcctgaacaccaaccaccgcatcgtggtgtgctccgagaactccctgcagttcttcatgcccgttctgggcgccctgttcattggggtggccgtggcaccagccaacgacatctacaacgagagggagctgctgaacagcatgggcatcagccagcccaccgtggtgttcgtgtccaagaagggcctgcagaagatcctgaacgtgcagaagaagctgcccatcatccagaagatcatcatcatggacagcaagaccgactaccagggcttccagtccatgtacaccttcgtgaccagccacctgcctcccggcttcaacgagtacgacttcgtgcccgagtccttcgacagggacaagaccatcgccctgatcatgaactccagcggaagcaccggcctgcccaagggtgtggctctaccccaccggaccgcctgtgtacggttcagccacgcccgtgaccccatcttcgggaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagtccgccctgctggtgcccaccctgttctccttcttcgccaagtccaccctgatcgacaagtacgacctgagcaacctgcacgagattgcctctggcggcgcacctctgtccaaggaggtcggggaggccgtagccaagcggttccacctgcccggcatccggcaaggctacggcctgaccgagaccaccagcgccatcctgatcacccctgagggcgacgacaagcccggcgcagtggggaaggtggtgccattcttcgaggccaaggtggtggacctggacaccggcaagaccctgggcgtgaaccagcggggcgagctgtgcgtgcggggacccatgatcatgtccggctacgtgaacaaccccgaggccaccaacgccctgattgacaaggacgggtggctgcacagcggcgacatcgcctactgggacgaggacgagcacttcttcatcgtggaccgcctgaagtccctgatcaagtacaaggggtaccaggtggctcccgccgagctggagtccatcctgctgcagcaccccaacatctttgacgccggcgttgccggactgcctgacgacgacgctggggaactccccgcagctgtggtggtgctggagcacggcaagaccatgaccgagaaggagatcgtggactacgtcgcctcccaggtgaccaccgccaagaagttacgcggcggcgtcgtgttcgtggacgaggtgcccaagggcctgaccggcaagctggacgcccgcaagatccgggagatcctgatcaaggccaagaagggcgggaagatcgccgtg

SEQ ID NO: 156 - DNA, 312 bp, Artificial Sequence
  Description of Artificial Sequence Synthetic polynucleotide
tgataataggctggagcctcggtggcctagcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctaggggtataattctcatttccacctcttttcatctgattcacgactacggaatcgttgttctaggacacttccgaaaccaaaatctcgcc

SEQ ID NO: 157 - protein, 407 aa, Artificial Sequence
  Description of Artificial Sequence Synthetic polypeptide
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGD

SEQ ID NO: 158 - protein, 358 aa, Artificial Sequence
  Description of Artificial Sequence Synthetic polypeptide
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQ

SEQ ID NO: 159 - protein, 50 aa, Artificial Sequence
  Description of Artificial Sequence Synthetic polypeptide
GKKTKFASDDEHDEHDENGATGPVKRAREETDKEEPASKQQKTENGAGDQ

SEQ ID NO: 160 - DNA, 21 bp, Artificial Sequence
  Description of Artificial Sequence Synthetic oligonucleotide
  Description of Combined DNA/RNA Molecule Synthetic oligonucleotide
  modified_base (21)..(21): Inverted deoxythymidine
aaaaaaaaaaaaaaaaaaaat

(19) United States
(12) Patent Application Publication
(10) Pub. No.: US 2024/0384277 A1
(43) Pub. Date: Nov. 21, 2024

(54) ENGINEERED POLYNUCLEOTIDES FOR CELL-TYPE OR MICROENVIRONMENT-SPECIFIC EXPRESSION

(71) Applicant: ModernaTX, Inc., Cambridge, MA (US)

(72) Inventors: Ruchi Jain, Brookline, MA (US); Brian Robert Fritz, Norwood, MA (US); Mihir Metkar, Arlington, MA (US)

(21) Appl. No.: 18/569,888

(22) PCT Filed: Jun. 14, 2022
(86) PCT No.: PCT/US2022/033411

§ 371 Date: Dec. 13, 2023

Related U.S. Application Data
(60) Provisional application No. 63/210,613, filed on Jun. 15, 2021.

(51) Int. Cl. C12N 15/63; C12N 9/22; C12N 15/88

(52) U.S. Cl. CPC C12N 15/635; C12N 9/22; C12N 15/88

(57) ABSTRACT

The disclosure features compositions or systems and uses thereof, comprising a first polynucleotide encoding a target molecule, optionally a second polynucleotide encoding an effector, repressor, or endonuclease molecule; optionally a recognition or cleavage site in the first or second polynucleotide, and optionally a repressor/effector binding site in the first polynucleotide. The compositions or systems of the present disclosure can increase the level and/or activity of the target molecule in desired cells and/or microenvironments while suppressing the level and/or activity of the target molecule in off-target cells and/or microenvironments.

### CROSS-REFERENCE TO RELATED APPLICATIONS

This application claims the benefit of priority of U.S. Provisional Appl. No. 63/210,613 filed Jun. 15, 2021, the contents of which are incorporated herein by reference in their entirety.

### BACKGROUND

Off-target effects are common and limit the utility of many drug candidates (Chartier, M., et al. BMC Pharmacol Toxicol 18, 18 (2017). While target-cell specificity is desirable in general for enhancing the safety of therapeutics, limiting off-target expression is especially important for some applications, e.g., gene editing where the genetic material of cells is permanently altered and/or the expression of toxic cargo in certain cell types, e.g., myeloid cells that release perforin (Dufait I. et al., Cancers (Basel). 2019 Jun. 11; 11(6):808) or cancer cells that make the p53 upregulated modulator of apoptosis (PUMA) (Yu J, Zhang L. Oncogene. 2008; 27 Suppl 1(Suppl 1):S71-S83). This is particularly relevant in the case of gene therapy and mRNA therapeutics which have the potential to revolutionize vaccination, protein replacement therapies, and the treatment of genetic diseases (Kowalski P. S. et al., Mol Ther. 2019 Apr. 10; 27(4): 710-728). Therefore, there is a need to reduce off-target expression of therapeutic nucleic acids and ensure specific expression in intended cells, intended cell-states and/or specific microenvironments, with wide-ranging benefits for immune-oncology therapeutics, auto-immunity therapeutics, in vivo gene editing, etc.

### SUMMARY

The present disclosure provides, inter alia, compositions or systems comprising a first polynucleotide encoding a target molecule, optionally a second polynucleotide encoding an effector, repressor, or endonuclease molecule; optionally a recognition site or cleavage site in the first or second polynucleotide, and optionally a repressor/effector binding site in the first polynucleotide, and uses thereof. The instant disclosure features a first polynucleotide (e.g., a therapeutic mRNA) encoding a first protein (i.e., a target molecule) with minimal or no translation ability (turned OFF) unless the first polynucleotide is in a specific cell and/or microenvironment, i.e., translation of the first protein can only be selectively turned ON by a trigger in a specific cell and/or microenvironment. The first polynucleotide may be, for example, a non-canonical mRNA. The trigger may be, for example, a promoter element, a microRNA (miRNA) or endonuclease target site recognition, and/or binding of a ligand and/or change of environment such as pH change or oxygen availability (e.g., hypoxia).

In some embodiments, the first polynucleotide that is turned OFF is under the control of a co-delivered second polynucleotide encoding a second protein (e.g., a repressor). For example, the first polynucleotide has a repressor binding site upstream of the target encoding region. The translation of the first protein is turned ON in the presence of a trigger in a specific cell and/or microenvironment that can either (a) repress the translation of the repressor or (b) eliminate the repressor binding site on the first polynucleotide.

In other embodiments, the first polynucleotide that is turned OFF is sterically occluded from translation. Further, the first polynucleotide is co-delivered with a second polynucleotide encoding a second protein (e.g., an effector) under the control of a tissue-specific promoter. The translation of the first protein is turned ON when (a) the effector is expressed in that specific tissue, or (b) a trigger in a specific cell and/or microenvironment facilitates the removal of that steric occlusion, thereby turning ON translation of the first polynucleotide.

In other embodiments, the first polynucleotide that is turned OFF is inherently unstable due to the presence of a destabilizing sequence in the first polynucleotide. Further, the first polynucleotide is co-delivered with a second polynucleotide encoding a second protein (e.g., an endonuclease) under the control of a tissue-specific promoter. The translation of the first protein is turned ON when (a) the endonuclease is expressed in that specific tissue, and (b) the endonuclease cleaves off the destabilizing sequence from the first polynucleotide.

In one aspect, the compositions or systems of the present disclosure comprise: (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein modification of the recognition site reduces translation of the repressor from the second polynucleotide, wherein binding of the repressor to the repressor binding element reduces translation of the polypeptide from the first polynucleotide.

In a second aspect, the compositions or systems of the present disclosure comprise: (a) a first polynucleotide comprising (i) an open reading frame encoding a first polypeptide, (ii) an effector binding element, and (iii) optionally a recognition site, wherein the first polynucleotide is mRNA; and (b) a second polynucleotide comprising a sequence encoding a second polypeptide, wherein the second polypeptide comprises an effector, wherein binding of the effector to the effector binding element increases translation of the first polypeptide from the first polynucleotide.

In a third aspect, the compositions or systems of the present disclosure comprise: (a) an RNA molecule comprising in order from the 5′ to 3′ end of the RNA (i) an open reading frame encoding a polypeptide, (ii) a polyA tail, (iii) a cleavage site, and (iv) a destabilizing sequence; and (b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide.

### Additional Aspects of the Disclosure are Described in Further Detail Below.

In a fourth aspect, the disclosure features a composition comprising: (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein modification of the recognition site reduces translation of the repressor from the second polynucleotide, wherein binding of the repressor to the repressor binding element reduces translation of the polypeptide from the first polynucleotide.

In some embodiments, the first polynucleotide is mRNA and comprises a polyA tail or is a DNA. In some embodiments, the second polynucleotide is (a) an mRNA and comprises a 5′ cap, a start codon, and a polyA tail or (b) a DNA. In some embodiments, when the first and second polynucleotides are DNA, they are encoded in a single plasmid. In some embodiments, the recognition site in the second polynucleotide which is an mRNA is (a) positioned between the sequence encoding the repressor and the polyA tail; or (b) positioned between the 5′ cap and the start codon. In some embodiments, the repressor binding element comprises a kink-turn forming sequence. In some embodiments, the repressor binding element is selected from the group consisting of PRE, PRE2, MS2, PP7, BoxB, U1A hairpin, and 7SK. In some embodiments, the repressor is selected from the group consisting of 50S ribosomal L7Ae protein, Pumilio and FBF (PUF) protein, PUF2 protein, MBP-LacZ, MBP, PCP, Lambda N, U1A, 15.5 kd, LARP7, and other RNA-binding proteins. In some embodiments, the recognition site is a microRNA target site or an endonuclease recognition site. In some embodiments, the microRNA target site comprises a sequence selected from a group consisting of SEQ ID NOs: 75-80.

In some embodiments, the polypeptide is selected from the group consisting of a secreted protein, a membrane-bound protein, an intercellular protein, or peptides, polypeptides or biologically active fragments thereof.

In some embodiments, the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

In some embodiments, (a) an mRNA and comprises a 5′ cap, a start codon, and a polyA tail and (b) a DNA are in separate dosage forms packaged together. In some embodiments, (a) an mRNA and comprises a 5′ cap, a start codon, and a polyA tail and (b) a DNA are in a unit dosage form.

In some embodiments, the disclosure features a method of expressing a polypeptide in a cell, the method comprising contacting the cell with any of the above compositions, wherein the cell expresses a microRNA or endonuclease that binds to the recognition site and reduces translation of the repressor from the second polynucleotide.

In a fifth aspect, the disclosure features a method of expressing a polypeptide in a cell, the method comprising contacting the cell with (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein the cell expresses a microRNA or an endonuclease that binds to the recognition site and reduces translation of the repressor from the second polynucleotide.

In a sixth aspect, the disclosure features a method of expressing a polypeptide in a cell in a subject, the method comprising administering to the subject: (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein the cell expresses a microRNA or an endonuclease that binds to the recognition site and reduces translation of the repressor from the second polynucleotide.

In some embodiments of the above methods, the cell is a liver cell. In some embodiments, the first polynucleotide is DNA or RNA and the second polynucleotide is DNA or RNA.

In a seventh aspect, the disclosure features a composition comprising: (a) a first polynucleotide comprising (i) an open reading frame encoding a first polypeptide, (ii) an effector binding element, and optionally (iii) a recognition site, wherein the first polynucleotide is mRNA; and (b) a second polynucleotide comprising a sequence encoding a second polypeptide, wherein the second polypeptide comprises an effector, wherein binding of the effector to the effector binding element enables and/or increases translation of the first polypeptide from the first polynucleotide.

In some embodiments, the second polynucleotide is an mRNA and comprises a polyA tail or is a DNA. In some embodiments, the effector binding element comprises a sequence selected from the group consisting of MS2, PRE, PRE2, PP7, BoxB, U1A hairpin and 7SK. In some embodiments, the second polypeptide comprises a sequence selected from the group consisting of Pumilio and FBF (PUF) protein, PUF2 protein, MBP-LacZ, 50S ribosomal L7Ae protein, PCP, Lambda N, U1A, 15.5 kd, LARP7, and an MBP polypeptide set forth in Table 4, wherein the second polypeptide binds to the respective effector binding element set forth in Table 1. In some embodiments, the recognition site is (a) between the open reading frame encoding the first polypeptide and the effector binding element in the first polynucleotide; or (b) located after the effector binding element in the first polynucleotide that (i) is linear and 5′-3′ oriented, or (ii) has a circular clockwise orientation. In some embodiments, the first polynucleotide (a) has no polyA tail; (b) is circular; (c) has no 5′ cap; (d) has no 5′ cap and no polyA tail; or (e) has a 5′ and/or 3′ end that is unsuitable for canonical translation. In some embodiments, the effector is the Eukaryotic translation initiation factor 4 G (eIF4G) or a fragment thereof. In some embodiments, the effector is an internal ribosome entry site (IRES)-Trans activating factor (ITAF) protein or a variant thereof. In some embodiments, the effector is La protein or a variant thereof. In some embodiments, the recognition site is a microRNA target site or an endonuclease cleavage site. In some embodiments, the microRNA target site is selected from a group consisting of SEQ ID NOs: 75-80. In some embodiments, the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

In some embodiments, the first and second polynucleotides are in separate dosage forms packaged together. In some embodiments, the first and second polynucleotides are in a unit dosage form.

In some embodiments, the disclosure features a method of expressing a polypeptide in a cell, the method comprising contacting the cell with the composition of the seventh aspect, wherein the cell expresses a microRNA or an endonuclease that binds to the recognition site of the first polynucleotide and enhances the ability of the effector to bind to the effector binding element and promote translation of the first polynucleotide.

In some embodiments, the method comprising contacting the cell with (a) a first polynucleotide comprising (i) an open reading frame encoding a first polypeptide, and (ii) an effector binding element, and optionally (iii) a recognition site, wherein the first polynucleotide is mRNA; and (b) a second polynucleotide comprising a sequence encoding a second polypeptide, wherein the second polypeptide comprises an effector, wherein binding of the effector to the effector binding element increases translation of the first polypeptide from the first polynucleotide, optionally wherein the cell expresses a microRNA or an endonuclease that binds to the recognition site of the first polynucleotide and enhances the ability of the effector to bind to the effector binding element and promote translation of the first polynucleotide.

In an eighth aspect, the disclosure features a composition comprising: (a) a first polynucleotide comprising (i) an open reading frame encoding a first polypeptide, and (ii) an effector binding element, wherein the first polynucleotide is mRNA which lacks a polyA tail; and (b) a second polynucleotide comprising a sequence encoding a second polypeptide, wherein the second polypeptide comprises an effector, wherein binding of the effector to the effector binding element enables and/or increases translation of the first polypeptide from the first polynucleotide.

In some embodiments, the second polynucleotide is an mRNA and comprises a polyA tail or is a DNA. In some embodiments, when the second polynucleotide is a DNA, the sequence encoding the effector is under the control of a tissue-specific promoter. In some embodiments, the expression and/or recruitment of the effector is under the control of a trigger in a specific microenvironment or specific cell-type. In some embodiments, the trigger is microRNA, receptor-mediated activation, and/or a change in pH and/or hypoxia. In some embodiments, the effector is a stabilizer and effector binding element is a destabilizing sequence or structure.

In a ninth aspect, the disclosure features a composition comprising: (a) an RNA molecule comprising in order from the 5′ to 3′ end of the RNA (i) an open reading frame encoding a polypeptide, (ii) a polyA tail, (iii) a cleavage site, and (iv) a destabilizing sequence; and (b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide.

Additional features of any of the aforesaid compositions or methods of using said compositions, include one or more of the following enumerated embodiments. Those skilled in the art will recognize or be able to ascertain using no more than routine experimentation, many equivalents to the specific embodiments of the invention described herein. Such equivalents are intended to be encompassed by the following enumerated embodiments.

### OTHER EMBODIMENTS OF THE DISCLOSURE

Embodiment 1(E1). A composition comprising:
(a) a first polynucleotide comprising (i) a repressor binding element and (ii) an open reading frame encoding a polypeptide; and
(b) a second polynucleotide comprising (i) a sequence encoding a repressor that binds to the repressor binding element and (ii) a recognition site, wherein modification of the recognition site reduces translation of the repressor from the second polynucleotide, wherein binding of the repressor to the repressor binding element reduces translation of the polypeptide from the first polynucleotide.
   E2. The composition of embodiment 1, wherein the first polynucleotide is mRNA and comprises a polyA tail or is a DNA.
   E3. The composition of embodiment 1 or 2, wherein the second polynucleotide is (a) an mRNA and comprises a 5′ cap, a start codon, and a polyA tail or (b) a DNA.
   E4. The composition of embodiment 2 or 3, wherein when the first and second polynucleotides are DNA, they are encoded in a single plasmid.
   E5. The composition of embodiment 3, wherein the recognition site in the second polynucleotide which is an mRNA is (a) positioned between the sequence encoding the repressor and the polyA tail; or (b) positioned between the 5′ cap and the start codon.
   E6. The composition of any one of embodiments 1-5, wherein the repressor binding element comprises a kink-turn forming sequence.
   E7. The composition of embodiment 6, wherein the repressor binding element is selected from the group consisting of PRE, PRE2, MS2, PP7, BoxB, U1A hairpin, and 7SK.
   E8. The composition of any one of embodiments 1-7, wherein the repressor is selected from the group consisting of 50S ribosomal L7Ae protein, Pumilio and FBF (PUF) protein, PUF2 protein, MBP-LacZ, MBP, PCP, Lambda N, U1A, 15.5 kd, LARP7, and other RNA-binding proteins.
   E9. The composition of any one of embodiments 1-8, wherein the recognition site is a microRNA target site or an endonuclease recognition site.
   E10. The composition of embodiment 9, wherein the microRNA target site comprises a sequence selected from a group consisting of SEQ ID NOs: 75-80.
   E11. The composition of any one of embodiments 1-10, wherein the polypeptide is selected from the group consisting of a secreted protein, a membrane-bound protein, an intercellular protein, or peptides, polypeptides or biologically active fragments thereof.
   E12. The composition of any one of embodiments 1-11, wherein the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.
   E13. The composition of any one of embodiments 1-12, wherein (a) and (b) are in separate dosage forms packaged together.
   E14. The composition of any one of embodiments 1-12, wherein (a) and (b) are in a unit dosage form.
   E15. A method of expressing a polypeptide in a cell, the method comprising contacting the cell with the composition of any one of embodiments 1-14, wherein the cell expresses a microRNA or endonuclease that binds to the recognition site and reduces translation of the repressor from the second polynucleotide.
   E16. A method of expressing a polypeptide in a cell, the method comprising contacting the cell with
(a) a first polynucleotide comprising (i) a repressor binding element and (ii) an open reading frame encoding a polypeptide; and
(b) a second polynucleotide comprising (i) a sequence encoding a repressor that binds to the repressor binding element and (ii) a recognition site, wherein the cell expresses a microRNA or an endonuclease that binds to the recognition site and reduces translation of the repressor from the second polynucleotide.
   E17. A method of expressing a polypeptide in a cell in a subject, the method comprising administering to the subject:
(a) a first polynucleotide comprising (i) a repressor binding element and (ii) an open reading frame encoding a polypeptide; and
(b) a second polynucleotide comprising (i) a sequence encoding a repressor that binds to the repressor binding element and (ii) a recognition site, wherein the cell expresses a microRNA or an endonuclease that binds to the recognition site and reduces translation of the repressor from the second polynucleotide.
   E18. The method of any one of embodiments 15-17, wherein the cell is a liver cell.
   E19. The method of any one of embodiments 15-17, wherein the first polynucleotide is DNA or RNA and the second polynucleotide is DNA or RNA.
   E20. A composition comprising:
(a) a first polynucleotide comprising (i) an open reading frame encoding a first polypeptide, (ii) an effector binding element, and optionally (iii) a recognition site, wherein the first polynucleotide is mRNA; and
(b) a second polynucleotide comprising a sequence encoding a second polypeptide, wherein the second polypeptide comprises an effector, wherein binding of the effector to the effector binding element enables and/or increases translation of the first polypeptide from the first polynucleotide.
   E21. The composition of embodiment 20, wherein the second polynucleotide is an mRNA and comprises a polyA tail or is a DNA.
   E22. The composition of embodiment 20 or 21, wherein the effector binding element comprises a sequence selected from the group consisting of MS2, PRE, PRE2, PP7, BoxB, U1A hairpin and 7SK.
   E23. The composition of any one of embodiments 20-22, wherein the second polypeptide comprises a sequence selected from the group consisting of Pumilio and FBF (PUF) protein, PUF2 protein, MBP-LacZ, 50S ribosomal L7Ae protein, PCP, Lambda N, U1A, 15.5 kd, LARP7, and an MBP polypeptide set forth in Table 4, wherein the second polypeptide binds to the respective effector binding element set forth in Table 1.
   E24. The composition of any one of embodiments 20-23, wherein the recognition site is (a) between the open reading frame encoding the first polypeptide and the effector binding element in the first polynucleotide; or
(b) located after the effector binding element in the first polynucleotide that
   (i) is linear and 5′-3′ oriented, or
   (ii) has a circular clockwise orientation.
      E25. The composition of any one of embodiments 20-24, wherein the first polynucleotide (a) has no polyA tail; (b) is circular; (c) has no 5′ cap; (d) has no 5′ cap and no polyA tail; or (e) has a 5′ and/or 3′ end that is unsuitable for canonical translation.
      E26. The composition of any one of embodiments 20-25, wherein the effector is the Eukaryotic translation initiation factor 4 G (eIF4G) or a fragment thereof.
      E27. The composition of any one of embodiments 20-25, wherein the effector is an internal ribosome entry site (IRES)-Trans activating factor (ITAF) protein or a variant thereof.
      E28. The composition of any one of embodiments 20-25, wherein the effector is La protein or a variant thereof.
      E29. The composition of any one of embodiments 20-28, wherein the recognition site is a microRNA target site or an endonuclease cleavage site.
      E30. The composition of any one of embodiment 29, wherein the microRNA target site is selected from a group consisting of SEQ ID NOs: 75-80.
      E31. The composition of any one of embodiments 20-30, wherein the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.
      E32. The composition of any one of embodiments 20-31, wherein (a) and (b) are in separate dosage forms packaged together.
      E33. The composition of any one of embodiments 20-31, wherein (a) and (b) are in a unit dosage form.
      E34. A method of expressing a polypeptide in a cell, the method comprising contacting the cell with the composition of any one of embodiments 20-29, wherein the cell expresses a microRNA or an endonuclease that binds to the recognition site of the first polynucleotide and enhances the ability of the effector to bind to the effector binding element and promote translation of the first polynucleotide.
      E35. A method of expressing a polypeptide in a cell, the method comprising contacting the cell with
(a) a first polynucleotide comprising (i) an open reading frame encoding a first polypeptide, and (ii) an effector binding element, and optionally (iii) a recognition site, wherein the first polynucleotide is mRNA; and
(b) a second polynucleotide comprising a sequence encoding a second polypeptide, wherein the second polypeptide comprises an effector, wherein binding of the effector to the effector binding element increases translation of the first polypeptide from the first polynucleotide,
   optionally wherein the cell expresses a microRNA or an endonuclease that binds to the recognition site of the first polynucleotide and enhances the ability of the effector to bind to the effector binding element and promote translation of the first polynucleotide.
      E36. A composition comprising:
(a) a first polynucleotide comprising (i) an open reading frame encoding a first polypeptide, and (ii) an effector binding element, wherein the first polynucleotide is mRNA which lacks a polyA tail; and
(b) a second polynucleotide comprising a sequence encoding a second polypeptide, wherein the second polypeptide comprises an effector, wherein binding of the effector to the effector binding element enables and/or increases translation of the first polypeptide from the first polynucleotide.
   E37. The composition of embodiment 36, wherein the second polynucleotide is an mRNA and comprises a polyA tail or is a DNA.
   E38. The composition of embodiment 37, wherein when the second polynucleotide is a DNA, the sequence encoding the effector is under the control of a tissue-specific promoter.
   E39. The composition of embodiment 36, wherein expression and/or recruitment of the effector is under the control of a trigger in a specific microenvironment or specific cell-type.
   E40. The composition of embodiment 39, wherein the trigger is microRNA, receptor-mediated activation, and/or a change in pH and/or hypoxia.
   E41. The composition of embodiment 35, wherein the effector is a stabilizer and effector binding element is a destabilizing sequence or structure.
   E42. A composition comprising:
(a) an RNA molecule comprising in order from the 5′ to 3′ end of the RNA (i) an open reading frame encoding a polypeptide, (ii) a polyA tail, (iii) a cleavage site, and (iv) a destabilizing sequence; and
(b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide.

### BRIEF DESCRIPTION OF THE DRAWINGS

FIGS. 1A-B provide a schematic of a 2 RNA system where one RNA encodes a repressor protein to suppress protein translation from the target RNA. FIG. 1A shows that in the absence of microRNA binding to the microRNA target site (miRts), the repressor protein is made and binds to the repressor binding site on the target RNA, thereby suppressing (turning OFF) target protein translation. FIG. 1B shows that microRNA binding to the microRNA target site (miRts) of repressor encoding RNA turns ON expression of target RNA, by reducing expression of the repressor and thereby preventing the repressor from binding to the repressor binding site on the target RNA.

FIGS. 2A-2B depict the level of green fluorescent protein (GFP) reporter gene expression (Total Integrated Green Intensity plotted as Total Area Under Curve; AUC) measured 48 h after transfection of Hela cells with the indicated RNA constructs. The RNA constructs are indicated on the y-axis. Experiments were done in 3× molar excess of target. FIG. 2A shows GFP protein output as measured on the Incucyte® Live-Cell Analysis System from HeLa cells transfected with control erythropoietin (EPO) RNA (SEQ ID NO: 147) and degGFP RNAs with 5′ UTR as depicted (SEQ ID NOs: 6 and 7); 5′v1.1-GFP RNA construct (5′v1.1; positive control), 5′kink-turn-GFP RNA construct (5′ kt; test), or no RNA (negative control). FIG. 2B shows GFP protein output from HeLa cells transfected with L7Ae repressor RNA construct (SEQ ID NO: 56) in combination with the 5′v1.1-GFP RNA construct or the 5′kt-GFP RNA construct.

FIGS. 3A-3B depict the level of green fluorescent protein (GFP) reporter gene expression (Total Integrated Green Intensity plotted as Total Area Under Curve; AUC) in miR142 negative (miR142 low) or miR142 positive (miR142 high) cells measured 48 h after transfection of indicated cells with the indicated RNA constructs. The species of RNA cotransfected with GFP RNA is indicated on the y-axis. Experiments were done in 3× molar excess of target. FIG. 3A shows GFP protein output from AMLL12 cells (miR142 low) transfected with the target RNA (5′kt-GFP RNA construct in combination with Control EPO RNA construct or L7AE repressor RNA construct, or L7AE_3x142ts repressor RNA construct). FIG. 3B shows GFP protein output from JAWS cells (miR142 high) transfected with the target (5′kt-GFP RNA construct in combination with Control EPO RNA construct or L7AE repressor RNA construct, or L7AE_3x142ts repressor RNA construct).

FIGS. 4A-4B depict the level of green fluorescent protein (GFP) reporter gene expression (Total Integrated Green Intensity plotted as Total Area Under Curve; AUC) in miR122+ cells measured 48 h after transfection of miR122 expressing cells with the indicated RNA constructs. The species of RNA cotransfected with GFP RNA is indicated on the y-axis. Experiments were done in 16× molar excess of target. FIG. 4A shows GFP protein output from HeLa cells (miR122 low) transfected with the target RNA (5′kt-GFP RNA construct) in combination with Control (EPO) RNA construct or L7AE repressor RNA construct, or L7AE_3x122ts repressor RNA construct. FIG. 4B shows GFP protein output from Huh7 cells (miR122 high) transfected with the target (5′kt-GFP RNA construct in combination with Control (EPO) RNA construct or L7AE repressor RNA construct, or L7AE_3x122ts repressor RNA construct).

FIGS. 5A-5C depict the level of luminescence from the luciferase (Luc) target gene. BALB/c mice were injected intravenously with 0.05 mg/kg the 5′ kink-turn containing Luc target mRNA (5′ kt target) in combination with a control (EPO) RNA, or L7Ae repressor RNA. The control/L7Ae repressor RNA was in molar excess (10×) of the Luc target RNA construct. FIG. 5A shows the RNA constructs used for the study. FIG. 5B shows luciferase expression (the level of luminescence) as determined by ex vivo imaging of spleen 6 h post IV injection. FIG. 5C shows luciferase expression (the level of luminescence) as determined by ex vivo imaging of liver 6 h post IV injection.

FIGS. 6A-6B depict the level of luminescence from the luciferase (Luc) target gene. FIG. 6A is a schematic of the 2 RNA construct system used for the study. BALB/c mice were injected intravenously with 0.05 mg/kg the Luc target RNA (5′ kt target) and L7Ae repressor RNA constructs (L7Ae repressor RNA with or without miRNA target sites) depicted in FIG. 6A. The L7Ae repressor RNA was in molar excess (10×) of the Luc target RNA construct (SEQ ID NO: 145). The spleen and liver of the mice were imaged by whole body luminescence 6 h post IV injection. FIG. 6B shows the level of luminescence in the spleen of mice injected with the Luc target RNA construct with Control EPO RNA or L7Ae repressor RNA or L7Ae repressor-miR122ts RNA or L7Ae repressor-miR142ts RNA. Luminescence observed for mice injected with phosphate-buffered saline (PBS) is also shown.

FIG. 7 depicts the level of green fluorescent protein (GFP) reporter gene expression (Total Integrated Green Intensity collected over 48 h, Total Area Under Curve; AUC plotted) measured after transfection of Hek293 cells with target GFP RNA constructs with Puf-Recognition Elements (PREs)—PRE_p2, p3 or p4, (SEQ ID NOs: 13-15), No PRE, or negative control as indicated on the y-axis. LUC RNA construct (SEQ ID NO: 145) or PUF repressor RNA constructs (SEQ ID NO: 66) were co-transfected with the target GFP RNA as indicated by +. Experiments were done in 1× molar excess of target.

FIG. 8 depicts the level of green fluorescent protein (GFP) reporter gene expression (Total Integrated Green Intensity collected over 48 h, Total Area Under Curve; AUC plotted) measured after transfection of Hek293 GFP cells with target GFP RNA constructs with Puf2-Recognition Elements (PRE2)—PRE2_p1, p2, 3, p4, p5, p6, (SEQ ID NOs: 12-17), No PRE, or negative control as indicated on the y-axis. LUC RNA construct (SEQ ID NO: 145) or PUF2 repressor RNA constructs (SEQ ID NO: 66) were co-transfected with the target GFP RNA as indicated by +. Experiments were done in 1× molar excess of target.

FIG. 9 depicts the level of green fluorescent protein (GFP) reporter gene expression (Total Integrated Green Intensity collected over 48 h, Total Area Under Curve; AUC plotted) after transfection of Hek293 cells with target GFP RNA constructs containing 6 MS2 hairpins in the 5′ UTR (SEQ ID NO: 8) cotransfected with LacZ (SEQ ID NO: 137) or MBP-LacZ repressor RNA (SEQ ID NO: 54) constructs or no RNA constructs as indicated on the y-axis. Experiments were done in 10× molar excess of target.

FIG. 10 provides a schematic of a 2 RNA system where one RNA encodes an effector protein and the other RNA encodes a target protein. The top panel shows that in an OFF target environment (no miRNA), the effector protein is unable to bind to its binding partner on the target RNA due to a steric block, thereby suppressing target protein translation. The bottom panel shows that in an ON target environment (with miRNA), the effector protein can bind to its binding partner on the target RNA, thereby removing the steric block, and permitting target protein translation.

FIG. 11 depicts the level of green fluorescent protein (GFP fluorescence, y-axis) measured at the indicated times (x-axis) after transfection of HEP3B cells with a no RNA control, or deg-GFP constructs lacking a poly A tail (A0) or having a poly A tail with 100 A residues (A100).

FIGS. 12A-12B provide schematics of two-RNA or RNA-DNA systems where one RNA encodes a target protein, and the other RNA/DNA encodes an effector protein. FIG. 12A provides a schematic of a two-RNA system to recruit potential effectors to target RNA with an MS2 binding protein (MBP)-MS2 tether. The target mRNA has MS2 loops in the 3′UTR and lacks a polyA tail (A0). The second mRNA encodes MS2-binding protein (MBP) fused to an effector/eIF4G-mid (a truncated fragment of the eIF4G protein). The RNA-binding protein, MBP tethers the effector, eIF4G to target RNA via the recognition of the MS2 hairpins. This system can be coupled to a miRNA-dependent switch gate to permit tethering in specific cells, thereby turning ON expression of the target protein. FIG. 12B provides a schematic of an RNA-DNA system to recruit potential effectors to target RNA with an MS2 binding protein (MBP)-MS2 tether. The target mRNA has MS2 loops in the 3′UTR and lacks a polyA tail (A0). The DNA encodes MS2-binding protein (MBP) fused to an effector/eIF4G-mid (a truncated fragment of the eIF4G protein). The RNA-binding protein, MBP tethers the effector, eIF4G to target RNA via the recognition of the MS2 hairpins. The effector expression is under the control of a tissue-specific promoter. Specific cells expressing that promoter would thus turn ON expression of the effector and permit tethering and rescue of expression of the target protein from the first polynucleotide. nt: nucleotide; aa: amino acids.

FIGS. 13A-13B provide schematics of a 1 RNA-1 DNA systems. FIG. 13A provides a 1 RNA-1 DNA system where the DNA encodes a stabilizer and the RNA encodes a target protein. The RNA is unstable due to a destabilizing structure (e.g., at the 3′ end of the RNA molecule). The unstable RNA can be specifically stabilized (enhanced half-life; and/or allowed to express protein) when the stabilizer (under the control of a tissue-specific promoter) binds to the destabilizing structure. FIG. 13B provides a 1 RNA-1 DNA system where the DNA encodes an effector and the RNA encodes a target protein. The RNA is inherently incapable of translation (i.e., it is dormant) due to a steric block (e.g., at the 3′ end of the RNA molecule). The dormant RNA can be specifically activated (allowed to express protein) when the effector (under the control of a tissue-specific promoter) binds to and removes the steric block.

FIGS. 14A-14B show that tailless target RNA shows detectable expression only when co-delivered with effector. FIG. 14A depicts the level of green fluorescent protein (GFP fluorescence, y-axis) measured at the indicated times (x-axis) after transfection of HEP3B cells with 3′v1.1_A100 and 3′v1.1_MS2_A0 (Control (A100) target RNA) deg-GFP constructs with Effector (t-eff, MBP-eIF4G (SEQ ID NO: 60) or Control (t-ctrl; MBP-LacZ (SEQ ID NO: 54). FIG. 14B depicts the level of total integrated green intensity plotted as total Area Under Curve; AUC).

FIGS. 15A-15B show that tailless target RNA shows detectable expression only when co-delivered with effector. FIG. 15A depicts the level of green fluorescent protein (GFP fluorescence, y-axis) measured at the indicated times (x-axis) after transfection of HeLa cells with 3′v1.1_A100 and 3′v1.1_MS2_A0 deg-GFP constructs with Effector (t-eff, MBP-eIF4G (SEQ ID NO: 60) or Control (t-ctrl; MBP-LacZ (SEQ ID NO: 54)) RNA. FIG. 15B depicts the level of total integrated green intensity plotted as total Area Under Curve; AUC).

FIG. 16 shows that tailless target RNA shows detectable expression only when co-delivered with effector, and overall expression is further improved when tailless RNA is ligated to idT. The figure depicts the level of green fluorescent protein (GFP fluorescence, y-axis) measured at the indicated times (x-axis) after transfection of Hep3b cells with 3′v1.1_MS2_A0 or 3′v1.1_MS2-A0-idT deg-GFP constructs with Effector (t-eff; MBP-eIF4G (SEQ ID NO: 38) or Control (t-ctrl; MBP-LacZ (SEQ ID NO: 54)) RNA.

FIG. 17 shows that capless target RNA shows detectable expression only when co-delivered with effector. The figure depicts the level of Relative Light Units (RLU, y-axis) measured at the indicated times (x-axis) after transfection of HeLa cells with 5′PPP-end NpiLUC constructs with Effector (t-eff, MBP-eIF4G-mid2 (SEQ ID NO: 38)) or Control (t-ctrl; MBP-eIF4G-mid2mut (SEQ ID NO: 60)) RNA.

FIG. 18 shows that capless-tailless target RNA shows detectable expression only when co-delivered with effector. The figure depicts the level of Relative Light Units (RLU, y-axis) measured at the indicated times (x-axis) after transfection of HeLa cells with 5′PPP-A0 ends NpiLUC constructs with Effector (t-eff, MBP-eIF4G-mid2 (SEQ ID NO: 38)) or Control (t-ctrl; MBP-eIF4G-mid2mut (SEQ ID NO: 60)) RNA.

FIG. 19 shows that shows that target circular RNA shows detectable expression only when co-delivered with effector-encoding RNA. The figure depicts the level of Relative Light Units (RLU, y-axis) measured at the indicated times (x-axis) after transfection of HeLa cells with circular RNA with NpiLUC ORF constructs with Effectors (t-eIF4G; MBP-mid2 (SEQ ID NO: 38)); t-La; (MBP-LA (SEQ ID NO: 152)); or t-LACZ (MBP-LacZ (SEQ ID NO: 54)). FIGS. 20A-20B provide a schematic of a 1 RNA-1 DNA system where the DNA encodes an endonuclease and the RNA encodes a target protein along with an endonuclease sensitive sequence between the polyA tail and a destabilizing sequence.

FIG. 20A shows that in an OFF target environment (e.g., in the absence of a tissue-specific promoter), the endonuclease is not expressed and is unable to cleave off the destabilizing sequence from the target RNA, thereby suppressing translation of the target protein. FIG. 20B shows that in an ON target environment (e.g., in the presence of a tissue-specific promoter), the endonuclease is expressed and can bind to the endonuclease sensitive sequence, thereby cleaving off the destabilizing sequence from the target RNA, and permitting target protein translation.

### DETAILED DESCRIPTION

Efforts to limit off-target effects of RNA-based therapeutics have focused on turning off expression of the therapeutic RNA in undesired cells and locations. The present disclosure is based on the discovery that RNA expression can be turned on in specific cells and/or locations, thereby permitting targeted and precise therapeutic and/or prophylactic action and preventing off-target effects.

Exemplary effects with the systems disclosed herein are provided in Examples 2-12. Example 2 shows that L7Ae represses expression of target RNA (5′ kink turn-RNA) in cells. Examples 3 and 4 show that L7Ae RNA with miR target sites lead to selective expression of target mRNA in cells expressing the particular miRNAs. Example 5 shows that L7Ae represses target RNA in vivo, and Example 6 shows that L7Ae_miRts RNA leads to selective expression of target RNA in vivo. Example 7 shows that RBP-RNA repressors PUF, PUF2 and MBP-LacZ repress target RNA expression in vitro. Example 8 shows that tail-less target RNAs do not show detectable expression in vitro. Example 9 shows that tail-less target RNAs can be rescued by tethering effector proteins using known RNA-RBP interactions such as MS2-MBP. Example 10 shows that tailless target RNAs with idT tails can be rescued by effectors recruited using RBP-RNA (MBP-MS2) tethers. Example 11 shows that capless target RNAs can be rescued by effectors recruited using RBP-RNA (MBP-MS2) tethers. Example 12 shows that capless target RNAs can be rescued by effectors recruited using RBP-RNA (MBP-MS2) tethers. Example 13 shows that dormant circular target RNAs can be rescued by effectors recruited using RBP-RNA (MBP-MS2) tethers.

Accordingly, disclosed herein are compositions or systems comprising polynucleotide constructs which are dependent on triggers in specific cells and/or microenvironments to express the polypeptide of interest, and minimize off-target expression. In some embodiments, the compositions comprise (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein modification of the recognition site reduces translation of the repressor from the second polynucleotide, wherein binding of the repressor to the repressor binding element reduces translation of the polypeptide from the first polynucleotide. In some embodiments, the compositions comprise (a) a first polynucleotide comprising (i) an open reading frame encoding a first polypeptide, (ii) an effector binding element, and (iii) a recognition site, wherein the first polynucleotide is mRNA; and (b) a second polynucleotide comprising a sequence encoding a second polypeptide, wherein the second polypeptide comprises an effector, wherein binding of the effector to the effector binding element enables and/or increases translation of the first polypeptide from the first polynucleotide. In some embodiments, the compositions comprise (a) an RNA molecule comprising in order from the 5′ to 3′ end of the RNA (i) an open reading frame encoding a polypeptide, (ii) a polyA tail, (iii) a cleavage site, and (iv) a destabilizing sequence; and (b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide. In some embodiments, the compositions comprise (a) a first polynucleotide comprising (i) an open reading frame encoding a first polypeptide, and (ii) an effector binding element, wherein the first polynucleotide is mRNA; and (b) a second polynucleotide comprising a sequence encoding a second polypeptide, wherein the second polypeptide comprises an effector, wherein the effector sequence is under the control of a tissue-specific promoter, wherein binding of the effector to the effector binding element enables and/or increases translation of the first polypeptide from the first polynucleotide.

In an embodiment, the first polynucleotide and the second polynucleotide are disposed in the same or different polynucleotides. In an embodiment, a system disclosed herein is formulated with a delivery agent selected from a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates. In an embodiment, the first polynucleotide is formulated with a delivery agent (e.g., a lipid nanoparticle). In some embodiments, the second polynucleotide is formulated with a delivery agent (e.g., a lipid nanoparticle). In an embodiment, the first polynucleotide and the second polynucleotide are formulated within the same delivery agent. In an embodiment, the first polynucleotide and the second polynucleotide are formulated in different delivery agents (e.g., different lipid nanoparticles). In an aspect, the compositions or systems of the present disclosure can increase the expression of a polypeptide of interest in specific cells/microenvironments. In an aspect, the compositions or systems can be used in a method of treating a disease or disorder, or for modulating an immune response in a subject.

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intravenous or subcutaneous.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean+/−5% of the recited value. For instance, an LNP including a lipid component having about 40% of a given compound may include 30-50% of the compound.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering one or more polynucleotides of this disclosure to a subject may involve administering a composition (e.g., an LNP including the one or more polynucleotides) to the subject (e.g., by an intravenous, intramuscular, intradermal, pulmonary or subcutaneous route). Administration of a composition (e.g., an LNP) to a mammal or mammalian cell may involve contacting one or more cells with the composition.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a polynucleotide that becomes part of a nanoparticle composition, relative to the initial total amount of the polynucleotide used in the preparation of a nanoparticle composition. For example, if 97 mg of polynucleotide are encapsulated in a nanoparticle composition out of a total 100 mg of polynucleotide initially provided to the composition, the encapsulation efficiency may be given as 97%. As used herein, “encapsulation” may refer to complete, substantial, or partial enclosure, confinement, surrounding, or encasement.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Modified: As used herein “modified” refers to a changed state or structure of a molecule of the disclosure, e.g., a change in a composition or structure of a polynucleotide (e.g., mRNA). Molecules, e.g., polynucleotides, may be modified in various ways including chemically, structurally, and/or functionally. For example, molecules, e.g., polynucleotides, may be structurally modified by the incorporation of one or more RNA elements, wherein the RNA element comprises a sequence and/or an RNA secondary structure(s) that provides one or more functions (e.g., translational regulatory activity). Accordingly, molecules, e.g., polynucleotides, of the disclosure may be comprised of one or more modifications (e.g., may include one or more chemical, structural, or functional modifications, including any combination thereof). In one embodiment, polynucleotides, e.g., mRNA molecules, of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence. In an embodiment, the mRNA is a circular mRNA.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a polynucleotide of the disclosure by a delivery agent (e.g., a nanoparticle) to a target cell of interest (e.g., mammalian target cell) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of a first and/or second polypeptide in a target cell versus non-target cell to the amount of total first and/or second polypeptide in said target cell versus non-target cell. It will be understood that the ability of a nanoparticle to specifically deliver to a target cell need not be determined in a subject being treated, it may be determined in a surrogate such as an animal model (e.g., a mouse or NHP model).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Effector Molecule: As used herein, the term “effector molecule” or “effector” refers to a molecule that can modulate a parameter of, e.g., level and/or activity of: an RNA (e.g., an mRNA); or a protein encoded by the RNA. In an embodiment, the parameter comprises one, two or all of: (1) mRNA level and/or activity and/or subcellular localization (e.g., half-life and/or expression); (2) protein level and/or activity (e.g., half-life and/or expression); (3) protein translation rate or (4) protein localization, e.g., location. In an embodiment, an effector molecule comprises a translation factor. An effector molecule comprises wildtype (e.g., naturally occurring, e.g., human), full length, a fragment (e.g., biologically active or functional fragment), or a variant thereof. In an embodiment, the effector molecule further comprises a tether molecule. In an embodiment the effector molecule polypeptide comprising a tether molecule comprises a first domain which modulates a parameter of, e.g., level and/or activity of: an RNA (e.g., an mRNA); or a protein encoded by the RNA, e.g., as described herein. In an embodiment the effector molecule polypeptide comprising a tether molecule comprises a second domain which binds to, e.g., recognizes, the binding element (a tether molecule). In an embodiment, the effector molecule comprises an RNA-binding protein or a fragment thereof. In an embodiment, an effector molecule comprises a translation factor, e.g., eIF4G. In an embodiment, an effector molecule comprises wildtype (e.g., naturally occurring, e.g., human), full length, a fragment (e.g., biologically active or functional fragment), or a variant of eIF4G. Exemplary eIF4G constructs are provided herein, e.g., in Table 4. In some embodiments, an effector is a repressor (e.g., L7Ae) or an activator (e.g., eIF4G).

Stabilizer: A “stabilizer” is just a subtype of an effector that enhances the stability of an unstable RNA molecule. In some embodiments, the stabilizer binds to a “destabilizing sequence” or “destabilizing structure” on an RNA molecule, thereby enhancing the stability of that RNA molecule.

Binding element: As used herein, the term “binding element” refers to a nucleic acid sequence, e.g., a DNA or RNA sequence, which is recognized by a tether molecule. In an embodiment, the binding element forms a structure, e.g., a three-dimensional structure, e.g., a kink-turn, a loop, a stem or other known structure. Exemplary binding elements are provided in Table 1.

Tether Molecule: As used herein, the term “tether molecule” refers to a molecule which binds to, e.g., recognizes, a binding element or a fragment thereof. In an embodiment, the tether molecule binds to, e.g., recognizes, a sequence, e.g., a DNA or RNA sequence, comprising the binding element, or fragment thereof. In an embodiment, the tether molecule binds to, e.g., recognizes, a structure comprising a sequence, e.g., a DNA or RNA sequence, comprising the binding element, or fragment thereof. In an embodiment, the effector molecule comprises an RNA-binding protein or a fragment thereof. Exemplary tether molecules are provided in Table 2.

Therapeutic Agent: The term “therapeutic agent” refers to any agent that, when administered to a subject, has a therapeutic, diagnostic, and/or prophylactic effect and/or elicits a desired biological and/or pharmacological effect. In some embodiments, the therapeutic agent comprises or is a therapeutic payload. In some embodiments, the therapeutic agent comprises or is a small molecule or a biologic (e.g., an antibody molecule).

First polypeptide: As used herein, the term “first polypeptide” refers to an agent which elicits a desired biological and/or pharmacological effect. In an embodiment, the first polypeptide has a therapeutic and/or prophylactic effect. In an embodiment, the first polypeptide comprises a protein, a polypeptide, a peptide or a fragment (e.g., a biologically active fragment) thereof. In an embodiment, the first polypeptide includes a sequence encoding a protein, e.g., a therapeutic protein. Some examples of first polypeptides include, but are not limited to a secreted protein, a membrane-bound protein, or an intracellular protein. In an embodiment, the first polypeptide includes a cytokine, an antibody, a vaccine (e.g., an antigen, or an immunogenic epitope), a receptor, an enzyme, a hormone, a transcription factor, a ligand, a membrane transporter, a structural protein, a nuclease, or a component, a variant or a fragment (e.g., a biologically active fragment) thereof. The terms protein, polypeptide and peptide are used interchangeably herein.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

Variant: As used herein, the term “variant” refers to a molecule having at least 50%, 60%, 70%, 80%, 85%, 90%, 95%, 96%, 97%, 98%, 99% or 100% activity of the wild type molecule, e.g., as measured by an art-recognized assay.

Target Site: As used herein, the term “target site”, “cleavage site”, and “binding site” are used interchangeably and refer to the recognition sequence (“recognition site”) within a polynucleotide molecule. A target site in the context of this disclosure can be a microRNA target site, or an endonuclease cleavage site. A target site can be engineered to be in the 5′ UTR or the 3′ UTR of the polynucleotide molecule.

Turning ON expression: As used herein, the term “turning ON expression” or “turning ON translation” refers to refers to increasing the degree of transcription or translation from a particular polynucleotide when the polynucleotide comes into contact with a trigger in a particular cell/microenvironment. The trigger can be a particular microRNA, a nuclease, an endonuclease, etc. The trigger can also be a promoter element, a miRNA target site recognition or endonuclease target site cleavage, and/or binding of a ligand and/or change of environment such as pH change or oxygen availability (e.g., hypoxia). In some embodiments, the trigger changes the target polynucleotide to allow effector binding (e.g., recognition or cleavage site on the target RNA). In other embodiments, the trigger is receptor-mediated activation of an effector, wherein the effector protein will change upon activation (e.g., an effector protein that cannot bind to the polynucleotide until there is receptor mediated activation of that effector), thereby allowing target RNA binding. In some embodiments, the trigger can cause partial cleavage of a polynucleotide disclosed herein (e.g., the first polynucleotide), such that the polynucleotide is capable of expressing the target protein it encodes for, after the trigger event. In other embodiments, the trigger can cause partial cleavage of a second polynucleotide disclosed herein (e.g., the repressor/effector polynucleotide). When the trigger causes partial cleavage of the second polynucleotide, it can turn ON expression or translation of the first polynucleotide. When the transcription or translation of the first polypeptide it turned ON with a trigger event (e.g, the presence of a miRNA in a specific cell/tissue), it can increase the level of transcription or translation of the first polypeptide in that specific cell/tissue by at least about 20%, 30%, 40%, 50%, 60%, 70%, 80%, 85%, 90%, 95%, 96%, 97%, 98%, 99% or 100% when compared to the level of transcription or translation in the absence of a trigger event.

Unsuitable for canonical translation: A polynucleotide (e.g., RNA) that is “unsuitable for canonical translation” is a polynucleotide with a nucleotide modification, sequence modification, and/or a structure that is not suitable for translation. In some embodiments, the modification(s) are at the 5′ end, and/or the 3′ end. In some embodiments, the modification(s) stabilize the polynucleotide. In some embodiments, a polynucleotide that is unsuitable for canonical translation (a) does not have a polyA tail; (b) is circular; (c) has no cap; and/or (d) has no cap and no tail.

### Dual Polynucleotide Systems

Disclosed herein, inter alia, are compositions and systems comprising two polynucleotides. In some embodiments, both the polynucleotides are RNA molecules. In some embodiments, both the polynucleotides are DNA molecules. In some embodiments, one polynucleotide is a DNA molecule and the other polynucleotide is an RNA molecule.

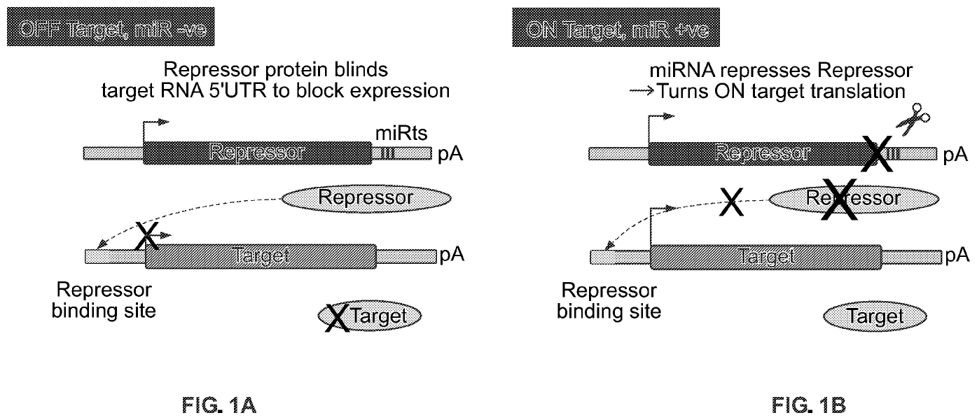

In one aspect, the composition comprises (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein modification of the recognition site reduces translation of the repressor from the second polynucleotide reduces translation of the repressor from the second polynucleotide, wherein binding of the repressor to the repressor binding element reduces translation of the polypeptide from the first polynucleotide. An example of such a system (a dual RNA system) is depicted in FIG. 1A. In this example, one RNA encodes a repressor protein to suppress protein translation from the target RNA. FIG. 1A shows that in the absence of microRNA binding to the microRNA target site (miRts), the repressor can bind to the repressor binding site on the target RNA, thereby suppressing (turning OFF) target protein translation. FIG. 1B shows that microRNA binding to the microRNA target site (miRts) turns ON expression of target RNA, by reducing expression of the repressor and thereby preventing the repressor from binding to the repressor binding site on the target RNA. Thus, the first polynucleotide that is turned OFF is under the control of a co-delivered second polynucleotide encoding a second protein (e.g., a repressor). For example, the first polynucleotide has a repressor binding site upstream of the target encoding region. The translation of the first protein is turned ON in the presence of a trigger in a specific cell and/or microenvironment that can either (a) repress the translation of the repressor or (b) eliminate the repressor binding site on the first polynucleotide.

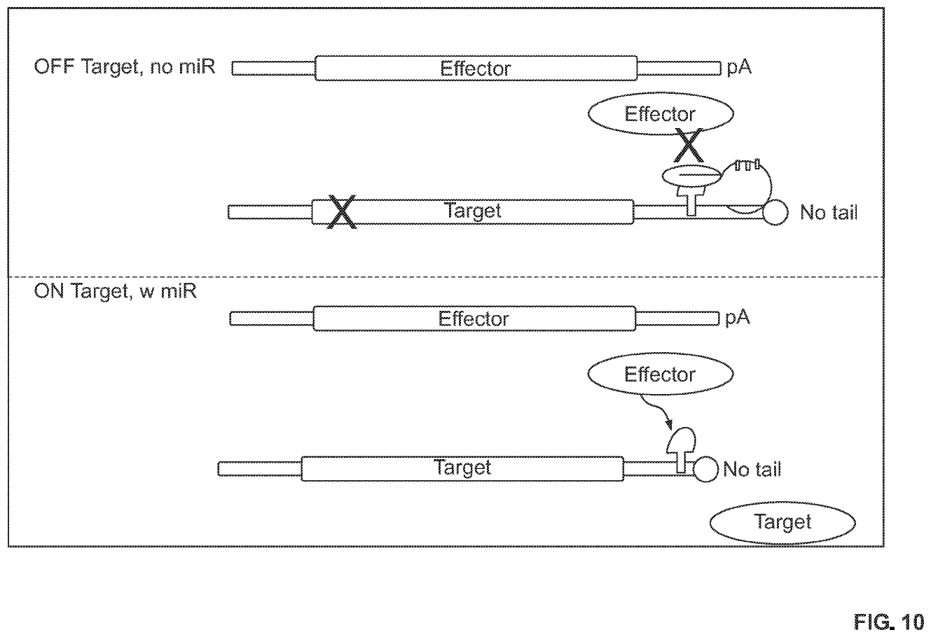

In another aspect, the composition comprises (a) a first polynucleotide comprising (i) an open reading frame encoding a first polypeptide, (ii) an effector binding element, and optionally (iii) a recognition site, wherein the first polynucleotide is mRNA; and (b) a second polynucleotide comprising a sequence encoding a second polypeptide, wherein the second polypeptide comprises an effector, wherein binding of the effector to the effector binding element enables and/or increases translation of the first polypeptide from the first polynucleotide. An example of such a system (a dual RNA system) is depicted in FIG. 10. In this example, one RNA encodes an effector protein and the other RNA encodes a target protein. The top panel shows that in an OFF target environment (no miRNA), the effector protein is unable to bind to its binding partner on the target RNA due to a steric block, thereby suppressing target protein translation. The bottom panel shows that in an ON target environment (with miRNA), the effector protein can bind to its binding partner on the target RNA, because of the removal of the steric block, and permit target protein translation. Thus, the first polynucleotide that is turned OFF because it is inherently incapable of translation (e.g., because it is circular or due to the absence of (a) a cap, (b) a tail, or (c) a cap and a tail), can only translate when the effector protein binds; but the effector protein binding is sterically occluded until the first polynucleotide is in the ON-target environment. Further, the first polynucleotide is co-delivered with a second polynucleotide encoding a second protein (e.g., an effector) that may be under the control of a tissue-specific promoter or miRNA. The translation of the first protein is turned ON when (a) the effector is expressed in that specific tissue, and (b) a trigger in a specific cell and/or microenvironment facilitates the removal of that steric occlusion from first polynucleotide, thereby enabling binding of the second protein t the first RNA, turning ON translation of the first polynucleotide. In some embodiments, the first polynucleotide encoding the target protein is circular RNA, capless RNA, tailless RNA, capless and tailless RNA, and/or RNA unsuitable for canonical translation due to unsuitable 5′ and/or 3′ ends.

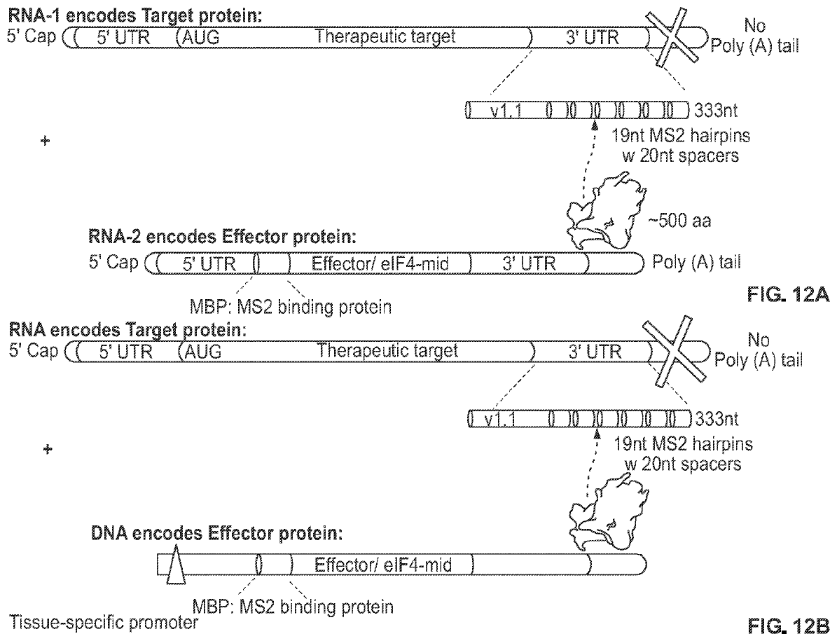

In another aspect, the composition comprises (a) a first polynucleotide comprising (i) an open reading frame encoding a first polypeptide, and (ii) an effector binding element, wherein the first polynucleotide is mRNA; and (b) a second polynucleotide comprising a sequence encoding a second polypeptide, wherein the second polypeptide encodes an effector, wherein the effector is under the control of a tissue-specific promoter, wherein binding of the effector protein to the effector binding element enables and/or increases translation of the first polypeptide from the first polynucleotide. In some embodiments, the first polynucleotide encoding a target protein is circular RNA, capless RNA, tailless RNA, capless and tailless RNA, and/or RNA unsuitable for canonical translation due to unsuitable 5′ and/or 3′ ends. FIG. 12A shows an example of a tailless first polynucleotide (RNA), and the second polynucleotide (RNA) encoding an effector under the control of a tissue-specific promoter. FIG. 12B shows an example of a tailless first polynucleotide, and the second polynucleotide is a DNA encoding an effector under the control of a tissue-specific promoter. Specific cells expressing that promoter would thus turn ON expression of the effector and permit tethering and rescue of expression of the target protein from the first polynucleotide.

In yet another aspect, the composition comprises (a) an RNA molecule comprising in order from the 5′ to 3′ end of the RNA (i) an open reading frame encoding a polypeptide, (ii) a polyA tail, (iii) a cleavage site, and (iv) a destabilizing sequence; and (b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide.

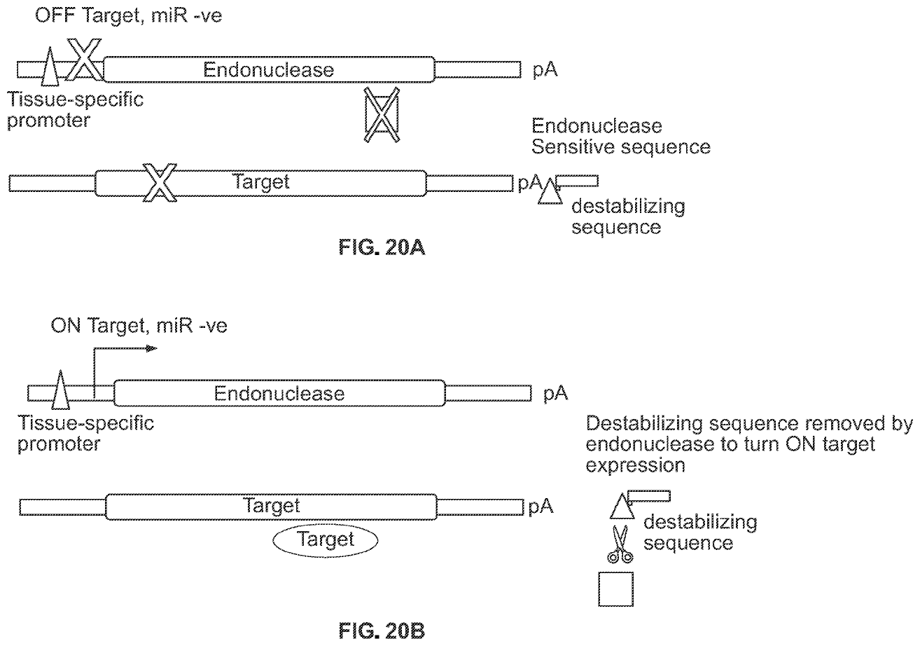

An example of such a system (a RNA/DNA system) is depicted in FIGS. 20A-20B. In this example, one DNA encodes an endonuclease and the other RNA encodes a target protein along with an endonuclease sensitive sequence between the polyA tail and a destabilizing sequence. FIG. 20A shows that in an OFF target environment, the endonuclease is not expressed and is unable to cleave off the destabilizing sequence from the target RNA, thereby suppressing translation of the target protein. FIG. 20B shows that in an ON target environment, the endonuclease is expressed and can bind to the endonuclease sensitive sequence, thereby cleaving off the destabilizing sequence from the target RNA, and permitting target protein translation. Thus, the first polynucleotide that is turned OFF is inherently unstable due to the presence of a destabilizing sequence in the first polynucleotide. Further, the first polynucleotide is co-delivered with a second polynucleotide encoding a second protein (e.g., an endonuclease) under the control of a tissue-specific promoter. The translation of the first protein is turned ON when (a) the endonuclease is expressed in that specific tissue, and (b) the endonuclease cleaves off the destabilizing sequence from the first polynucleotide.

In some embodiments, the destabilizing sequence is co-delivered with a destabilizer using RNA engineering methods known in the art. See e.g., Choudhary et al. Nature Comm, 2012; 3:1147.

### First Polypeptide

Disclosed herein, inter alia, is a composition or system comprising a first polynucleotide comprising an open reading frame encoding a polypeptide (also referred to as a first polypeptide). In some embodiments, the polypeptide encodes: a secreted protein; a membrane-bound protein; or an intercellular protein, or peptides, polypeptides or biologically active fragments thereof.

In some embodiments, the polypeptide is a secreted protein, or a peptide, a polypeptide or a biologically active fragment thereof. In some embodiments, the secreted protein comprises a cytokine, or a variant or fragment (e.g., a biologically active fragment) thereof. In some embodiments, the secreted protein comprises an antibody or a variant or fragment (e.g., a biologically active fragment) thereof. In some embodiments, the secreted protein comprises an enzyme or a variant or fragment (e.g., a biologically active fragment) thereof. In some embodiments, the secreted protein comprises a hormone or a variant or fragment (e.g., a biologically active fragment) thereof. In some embodiments, the secreted protein comprises a ligand, or a variant or fragment (e.g., a biologically active fragment) thereof. In some embodiments, the secreted protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the secreted protein comprises a growth factor or a component, variant or fragment (e.g., a biologically active fragment) thereof. In some embodiments, the secreted protein comprises an immune modulator, e.g., an immune checkpoint agonist or antagonist.

In some embodiments, the polypeptide is a membrane-bound protein, or a peptide, a polypeptide or a biologically active fragment thereof. In some embodiments, the membrane-bound protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the membrane-bound protein comprises a ligand, a variant or fragment (e.g., a biologically active fragment) thereof. In some embodiments, the membrane-bound protein comprises a membrane transporter, a variant or fragment (e.g., a biologically active fragment) thereof. In some embodiments, the membrane-bound protein comprises a structural protein, a variant or fragment (e.g., a biologically active fragment) thereof. In some embodiments, the membrane-bound protein comprises an immune modulator, e.g., an immune checkpoint agonist or antagonist.

In some embodiments, the polypeptide is an intracellular protein, or a peptide, a polypeptide or a biologically active fragment thereof. In some embodiments, the intracellular protein comprises an enzyme, or a variant or fragment (e.g., a biologically active fragment) thereof. In some embodiments, the intracellular protein comprises a hormone, or a variant or fragment (e.g., a biologically active fragment) thereof. In some embodiments, the intracellular protein comprises a cytokine, or a variant or fragment (e.g., a biologically active fragment) thereof. In some embodiments, the intracellular protein comprises a transcription factor, or a variant or fragment (e.g., a biologically active fragment) thereof. In some embodiments, the intracellular protein comprises a nuclease, or a variant or fragment (e.g., a biologically active fragment) thereof. In some embodiments, the intracellular protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the intracellular protein comprises a structural protein, or a variant or fragment (e.g., a biologically active fragment) thereof.

In some embodiments, the polypeptide is chosen from a cytokine, an antibody, a vaccine (e.g., an antigen, an immunogenic epitope), a receptor, an enzyme, a hormone, a transcription factor, a ligand, a membrane transporter, a structural protein, a nuclease, a growth factor, an immune modulator, or a component, variant or fragment (e.g., a biologically active fragment) thereof.

In some embodiments, the polypeptide comprises a cytokine, or a variant or fragment (e.g., a biologically active fragment) thereof.

In some embodiments, the polypeptide comprises an antibody or a variant or fragment (e.g., a biologically active fragment) thereof.

In some embodiments, the polypeptide comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine.

In some embodiments, the polypeptide comprises a receptor, or a variant or fragment (e.g., a biologically active fragment) thereof.

In some embodiments, the polypeptide comprises an enzyme, or a variant or fragment (e.g., a biologically active fragment) thereof.

In some embodiments, the polypeptide comprises a hormone, or a variant or fragment (e.g., a biologically active fragment) thereof.

In some embodiments, the polypeptide comprises a growth factor, or a variant or fragment (e.g., a biologically active fragment) thereof.

In some embodiments, the polypeptide comprises a nuclease, or a variant or fragment (e.g., a biologically active fragment) thereof.

In some embodiments, the polypeptide comprises a transcription factor, or a variant or fragment (e.g., a biologically active fragment) thereof.

In some embodiments, the polypeptide comprises a ligand, or a variant or fragment (e.g., a biologically active fragment) thereof.

In some embodiments, the polypeptide comprises a membrane transporter, or a variant or fragment (e.g., a biologically active fragment) thereof.

In some embodiments, the polypeptide comprises a structural protein, or a variant or fragment (e.g., a biologically active fragment) thereof.

In some embodiments, the polypeptide comprises an immune modulator, or a variant or fragment (e.g., a biologically active fragment) thereof. In some embodiments, the immune modulator comprises an immune checkpoint agonist or antagonist.

In some embodiments, the polypeptide comprises a protein or peptide.

In some embodiments, the first polynucleotide encoding a target protein is circular RNA. In some embodiments, the circular RNA has modified or unmodified nucleotides and no internal ribosome entry site (IRES) elements. In some embodiments, MS2 loops are present in single or multiple copies on the target circular RNA and can be anywhere in the non-coding region.

### Binding Element

Disclosed herein, inter alia, is a composition or system comprising a polynucleotide, e.g., a first polynucleotide, e.g, an mRNA, comprising a binding element. A binding element comprises a sequence, e.g., a DNA or RNA sequence, which is bound, e.g., recognized by, a second polypeptide or a fragment thereof, e.g., a tether molecule (e.g., an RNA binding protein), a repressor, an effector, an activator, or an endonuclease as described elsewhere in this disclosure. In some embodiments, the second polypeptide binds to a sequence comprising the binding element, or a fragment thereof. In some embodiments, the second polypeptide binds to a structure comprising the binding element, or a fragment thereof.

In some embodiments, the composition or system comprises a second polynucleotide encoding an RNA binding protein or a fragment thereof, e.g., a tether molecule, which binds to, e.g., recognizes, the binding element of the first polynucleotide.

In some embodiments, the binding element of the first polynucleotide is situated upstream (5′) or downstream (3′), or in the open reading frame of the sequence encoding the polypeptide.

In some embodiments, the binding element of the first polynucleotide is situated upstream (5′) or downstream (3′) of a 5′ UTR of the first polynucleotide. In some embodiments, the binding element of the first polynucleotide is situated upstream (5′) or downstream (3′) of a 3′ UTR of the first polynucleotide. In some embodiments, the binding element of the first polynucleotide is situated in the 5′ UTR of the first polynucleotide. In some embodiments, the binding element of the first polynucleotide is situated downstream of a 3′ UTR of the first polynucleotide. In some embodiments, the binding element of the first polynucleotide is situated adjacent, e.g., next to, a Poly A tail.

In some embodiments, the binding element of the first polynucleotide is bound by the tether molecule of the second polynucleotide, e.g., an effector molecule further comprising a tether molecule.

In some embodiments, the binding element comprises a sequence which is bound, e.g., recognized, by the tether molecule. In some embodiments, the binding element comprises a sequence comprising a structure that is bound, e.g., recognized, by the tether molecule.

In some embodiments, the binding element is MS2. In some embodiments, the binding element is PP7. In some embodiments, the binding element is BoxB. In some embodiments, the binding element is U1A hairpin. In some embodiments, the binding element is PRE. In some embodiments, the binding element is PRE2. In some embodiments, the binding element is a kink-turn forming sequence. In some embodiments, the binding element is 7SK. In some embodiments, the binding element is an RNA sequence/structure element that binds to a protein.

In some embodiments, when the binding element is MS2 (e.g., wildtype MS2, or a variant or fragment thereof) the tether molecule is MBP (e.g., wildtype MBP, a variant or fragment thereof).

In some embodiments, when the binding element is PP7 (e.g., wildtype PP7, or a variant or fragment thereof) the tether molecule is PCP (e.g., wildtype PCP, or a variant or fragment thereof). PP7 can comprise the sequence of any one of the PP7 and variants thereof described in Lim F, and Peabody D S. Nucleic Acids Res. 2002; 30(19):4138-4144, and U.S. Pat. No. 9,365,831, incorporated by reference herein in its entirety.

In some embodiments, when the binding element is BoxB (e.g., wildtype BoxB, or a variant or fragment thereof) the tether molecule is Lambda N (e.g., wildtype Lambda N, or a variant or fragment thereof).

In some embodiments, when the binding element is U1A hairpin (e.g., wildtype U1A hairpin, or a variant or fragment thereof) the tether molecule is U1A (e.g., wildtype U1A, or a variant or fragment thereof).

In some embodiments, when the binding element is PRE (e.g., wildtype PRE, or a variant or fragment thereof) the tether molecule is PUF (e.g., wildtype PUF, or a variant or fragment thereof).

In some embodiments, when the binding element is a kink-turn forming sequence the tether molecule is 15.5 kd (e.g., wildtype 15.5 kd, or a variant or fragment thereof).

In some embodiments, when the binding element is a 7SK sequence the tether molecule is LARP7 (e.g., wildtype LARP7, or a variant or fragment thereof).

In some embodiments, the binding element comprises a sequence comprising 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the binding element comprises a sequence comprising about 5-100, about 5-90, about 5-80, about 5-70, about 5-60, about 5-50, about 5-40, about 5-30, about 5-25, about 5-20, about 5-19, about 5-18, about 5-17, about 5-16, about 5-15, about 5-14, about 5-13, about 5-12, about 5-11, about 5-10, about 5-9, about 5-8, about 5-7 or about 5-6 nucleotides. In some embodiments, the binding element comprises a sequence comprising about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the binding element comprises a sequence comprising about 5-100, about 6-90, about 7-80, about 8-70, about 9-60, about 10-50, about 11-40, about 12-30, about 13-25, about 14-24, about 15-23, about 16-22, about 17-21, or about 18-20 nucleotides. In some embodiments, the binding element comprises a sequence comprising 19 nucleotides.

In some embodiments, the binding element comprises a binding element nucleotide sequence provided in Table 1 or a sequence with at least 80%, 85%, 90%, 95%, 96%, 97%, 98%, 99% or 100% identity thereof. In some embodiments, the binding element comprises a binding element sequence provided in SEQ ID NO: 1, SEQ ID NO: 2, SEQ ID NO: 3, or a sequence with at least 80%, 85%, 90%, 95%, 96%, 97%, 98%, 99% or 100% identity thereof.

In some embodiments of any of the compositions, systems, methods or uses disclosed herein, the binding element comprises at least 1, 2, 3, 4, 5, 6, 7, 8, 9, 10, 11, 12, 13, 14, 15, 20 or 30 repeats of the sequence bound by the second polypeptide. In some embodiments, the binding element comprises no more than 80, 70, 60, 50, 40 or 30 repeats of the sequence bound by the the second polypeptide. In some embodiments, n the binding element comprises about 1-30, about 1-20, about 1-10, about 1-9, about 1-8, about 1-7, about 1-6, about 1-5, about 1-4, about 1-3, or about 1-2 repeats of the sequence bound by the second polypeptide. In some embodiments, the binding element comprises about 1-30, about 2-30, about 3-30, about 4-30 about, 5-30 about, 6-30, about 7-30, about 8-30, about 9-30, about 10-30, about 11-30, about 12-30, about 13-30, about 14-30, about 15-30, or about 20-30 repeats of the sequence bound by the second polypeptide. In some embodiments, the binding element comprises about 1-30, about 2-20, about 3-15, about 4-14, about 5-13, about 6-12, about 7-11, or about 8-10 repeats of the sequence bound by the second polypeptide. In some embodiments, the binding element comprises 6 repeats of the sequence bound by the second polypeptide.

In some embodiments of any of the compositions, systems, methods or uses disclosed herein, each repeat is separated by a spacer sequence comprising 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the spacer sequence comprises about 1-100, about 1-90, about 1-80, about 1-70, about 1-60, about 1-50, about 1-40, about 1-30, about 1-25, about 1-20, about 1-19, about 1-18, about 1-17, about 1-16, about 1-15, about 1-14, about 1-13, about 1-12, about 1-11, about 1-10, about 1-9, about 1-8, about 1-7, about 1-6, about 1-5, about 1-4, about 1-3, or about 1-2 nucleotides. In some embodiments, the spacer sequence comprises about 1-100, about 2-100, about 3-100, about 4-100, about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the spacer sequence comprises about 1-100, about 2-90, about 3-80, about 4-70, about 5-60, about 6-50, about 7-40, about 8-40, about 9-30, about 10-25, about 11-24, about 12-23, about 13-22, about 14-21, about 15-20, about 16-19, about 17-18 nucleotides. In some embodiment, the spacer sequence comprises 20 nucleotides.

In some embodiments, the spacer sequence comprises a spacer sequence provided in Table 1 or a sequence with at least 80%, 85%, 90%, 95%, 96%, 97%, 98%, 99% or 100% identity thereof.

In some embodiments, the binding element is an MS2 dimer which comprises monomers linked by a linker sequence. The linker sequence could be any peptide sequence known to link two protein sequences, including but not limited to those known in the art (See, e.g., Chen et al., Adv Drug Deliv Rev. (2013) Oct. 15; 65(10): 1357-1369).

TABLE 1
Exemplary sequences of a binding element

| | | |
|---|---|---|
| SEQ | | |
| ID | Sequence | |
| NO | information | Sequence |

3′ UTR sequence

| | | |
|---|---|---|
| 1 | 3′v1.1_6xMS2 | TGATAATAGGCTGGAGCCTCGGTGGCCTAGCTTCTTGCCCCT |
| | | TGGGCCTCCCCCCAGCCCCTCCTCCCCTTCCTGCACCCGTACCCC |
| | | CGTGGTCTTTGAATAAAGTCTGAGTGGGCGGCCAGGTATGATTA |
| | | CAACCTGTGCTACTTTAGATGACTATTGAAAGACCATTAGGCTTT |
| | | CTAACGGACGCTCACGAGAGAGGGGCACCAATAGGGCCCCACG |
| | | GCACTTCAAAGGTTTGGCTTGGAGTAGTAACCCAAGCAGCAACA |
| | | GTTTTGACTTTCGGACCACCATCAGGGGTCCCACGTTGGGAACA |
| | | CGTAACTCTCCTACTAACAAGAGGAG |
| 2 | 3′6xMS2_3′v1.1 | TGATAATAGCAGGTATGATTACAACCTGTGCTACTTTAGATG |
| | | ACTATTGAAAGACCATTAGGCTTTCTAACGGACGCTCACGAGAG |
| | | AGGGGCACCAATAGGGCCCCACGGCACTTCAAAGGTTTGGCTTG |
| | | GAGTAGTAACCCAAGCAGCAACAGTTTTGACTTTCGGACCACCA |
| | | TCAGGGGTCCCACGTTGGGAACACGTAACTCTCCTACTAACAAG |
| | | AGGAGGCTGGAGCCTCGGTGGCCTAGCTTCTTGCCCCTTGGGCCT |
| | | CCCCCCAGCCCCTCCTCCCCTTCCTGCACCCGTACCCCCGTGGTC |
| | | TTTGAATAAAGTCTGAGTGGGCGGC |
| 3 | 3′_6xMS2 | TGATAATAGCAGGTATGATTACAACCTGTGCTACTTTAGAT |
| | (MS2 sequences | GACTATTGAAAGACCATTAGGCTTTCTAACGGACGCTCACGAG |
| | in bold and | AGAGGGGCACCAATAGGGCCCCACGGCACTTCAAAGGTTTGG |
| | underline) | CTTGGAGTAGTAACCCAAGCAGCAACAGTTTTGACTTTCGGAC |
| | | CACCATCAGGGGTCCCACGTTGGGAACACGTAACTCTCCTACT |
| | | AACAAGAGGAG |
| 4 | 3′v1.1 | TGATAATAGGCTGGAGCCTCGGTGGCCTAGCTTCTTGCCCCT |
| | | TGGGCCTCCCCCCAGCCCCTCCTCCCCTTCCTGCACCCGTACCCC |
| | | CGTGGTCTTTGAATAAAGTCTGAGTGGGCGGC |
| 5 | 3′v1.1 6xMS2 | TGATAATAGGCTGGAGCCTCGGTGGCCTAGCTTCTTGCCCCT |
| | | TGGGCCTCCCCCCAGCCCCTCCTCCCCTTCCTGCACCCGTACCCC |
| | | CGTGGTCTTTGAATAAAGTCTGAGTGGGCGGCCAGGTATGATTA |
| | | CAACCTGTGCTACTTTAGATGACTATTGAAAGACCATTAGGCTTT |
| | | CTAACGGACGCTCACGAGAGAGGGGCACCAATAGGGCCCCACG |
| | | GCACTTCAAAGGTTTGGCTTGGAGTAGTAACCCAAGCAGCAACA |
| | | GTTTTGACTTTCGGACCACCATCAGGGGTCCCACGTTGGGAACA |
| | | CGTAACTCTCCTACTAACAAGAGGAG |

5′ UTR sequence*

| | | |
|---|---|---|
| 6 | 5′ v1.1 | GGGAAATAAGAGAGAAAAGAAGAGTAAGAAGAAATATAAG |
| | | ACCCCGGCGCCGCCACC |
| 7 | 5′kink turn (5′kt) | GGAUCCGUGAUCGGAAACGUGAGAUCCACCUCAGAUCCGC |
| | | UAGGACACCCGCAGAUCGAGAAGAAGGCGAAUUAAGAGAGAA |
| | | AAGAAGAGUA AGAAGAAAUAUAAGACACCGGUCGC CACC |
| 8 | 5′_6xMS2 | GGGAAACGGCAAGCAGCAGCUGCCGCUGUUUUGUCUUAAC |
| | (underlined | CGGUUGAAAGACCAUUAGGCUUUCUAACGGACGCUCACGAGAG |
| | sequence indicates | AGGGGCACCAAUAGGGCCCCACGGCACUUCAAAGGUUUGGCUU |
| | the region MBP | GGAGUAGUAACCCAAGCAGCAACAGUUUUGACUUUCGGACCAC |
| | binds to) | CAUCAGGGGUCCCACGUUGGGAACACGUAACUCUCCUACUAAC |
| | | AAGAGGAGCCCCGGCGCCGCCACC |
| 9 | PRE_p2 | GGGAAAUAAGAGAGAAAAAGAGAGAGGGAGAGGAGAAGA |
| | | GUAAGAAGAAAUAUAAGACCCCGGCGCCGCCACC |
| 10 | PRE_p3 | GGGAAAUAAGAGAGAAAAGAAGAGUAAGAAAGAGAGAGG |
| | | GAGAGGAGAAAUAUAAGACCCCGGCGCCGCCACC |
| 11 | PRE_p4 | GGGAAAUAAGAGAGAAAAGAAGAGUAAGAAGAAAUAUAA |
| | | GACAGAGAGAGGGAGAGGACCCGGCGCCGCCACC |
| 12 | PRE2_p1_3x | GGGAAAUUUUUUUUUGAUAUUAUAAGAGUUUUUUUUUGA |
| | | UAUUAAGAAAAUUUUUUUUUGAUAUUAGAAGAGUAAGAAGAA |
| | | AUAUAAGACCCCGGCGCCGCCACC |
| 13 | PRE2_p2_3x | GGGAAAUAAGAGUUUUUUUUUGAUAUUAAGAAAAUUUUU |
| | | UUUUGAUAUUAGAAGAGUUUUUUUUUGAUAUUAUAAGAAGAA |
| | | AUAUAAGACCCCGGCGCCGCCACC |
| 14 | PRE2 p3_3x | GGGAAAUAAGAGAGAAAAUUUUUUUUUGAUAUUAGAAGA |
| | | GUUUUUUUUUGAUAUUAUAAGAAUUUUUUUUUGAUAUUAGAA |
| | | AUAUAAGACCCCGGCGCCGCCACC |
| 15 | PRE2_p4_3x | GGGAAAUAAGAGAGAAAAGAAGAGUUUUUUUUUGAUAUU |
| | | AUAAGAAUUUUUUUUUGAUAUUAGAAAUAUUUUUUUUUGAUA |
| | | UUAUAAGACCCCGGCGCCGCCACC |
| 16 | PRE2_p5_3x | GGGAAAUAAGAGAGAAAAGAAGAGUAAGAAUUUUUUUUU |
| | | GAUAUUAGAAAUAUUUUUUUUUGAUAUUAUAAGACUUUUUUU |
| | | UUGAUAUUACCCGGCGCCGCCACC |
| 17 | PRE2_p6_3x | GGGAAAUAAGAGAGAAAAGAAGAGUAAGAAGAAAUAUUU |
| | | UUUUUUGAUAUUAUAAGACUUUUUUUUUGAUAUUACCCGGCU |
| | | UUUUUUUUGAUAUUAGCCGCCACC |

Miscellaneous sequences

| | | |
|---|---|---|
| 18 | BoxB | GGGCCCUGAAGAAGGGCCC |
| 19 | U1A hairpin | AAUCCAUUGCACUCCGGAUUU |
| 20 | 7SK | GGATGTGTGGCCAGTTGGTGCTGTCTGTCTTCTTGGTGACCA |
| | | GGAGTAATTTGGACCATCTGGGAAACCAGACCTCTTTTCACCCA |
| | | GGCTGTTTCGTGTGCATCACTTCTGAGGCTGTGTGCTCAGTCAAG |
| | | ACCTTGCCAGAGAGTTGGGGGATTAGCCTTAGGTCGAGAATATT |
| | | CTGAGCAGTAGTAAGAAATTTAAAAATCATCCATAATTTCATCA |
| | | CTCTTATTTTAAGAGATTAGTTTAT |

*underlined sequence indicates the region the RBP binds to

### Second Polypeptide

Disclosed herein, inter alia, is a composition or system comprising a first and a second polynucleotide. The second polynucleotide encodes a second polypeptide. In some embodiments, the second polypeptide is: a tether molecule (including but not limited to a repressor), an effector, an activator, an endonuclease, or peptides, polypeptides or biologically active fragments thereof.

### Tether Molecule

In some embodiments, the second polypeptide is an RNA binding protein or a fragment thereof, e.g., a tether molecule, which binds to, e.g., recognizes, the binding element of the first polynucleotide. In some embodiments, a tether molecule of the second polynucleotide is an effector molecule further comprising a tether molecule.

In some embodiments, the second polypeptide is a tether molecule chosen from the molecules provided in Table 2, e.g., 50S ribosomal L7Ae protein, Pumilio and FBF (PUF) protein, PUF2 protein, MBP-LacZ, MBP, PCP, Lambda N, U1A, 15.5 kd, LARP7 or a variant or fragment thereof.

L7Ae is an archaeal ribosomal protein which regulates the translation of a designed mRNA in vitro and in human cells (see, e.g., Saito H, et al., Nat Chem Biol. 2010 January; 6(1):71-8); and Wroblewska L, et al. Nat Biotechnol. 2015; 33(8):839-841.

In some embodiments, when the tether molecule is 50S ribosomal L7Ae protein (e.g., wildtype MBP, a variant or fragment thereof) the binding element is a kink-turn forming sequence (e.g., SEQ ID NO: 7, or a variant or fragment thereof).

PUF is a family of proteins that bind RNA sequence stretches defined by their amino acid identities at specific positions. Some amino acids in the protein can be engineered to change binding to any other RNA sequence. PUF2 is such an engineered protein. Filipovska A, et al. Nat Chem Biol. 2011 May 15; 7(7):425-7.

In some embodiments, when the tether molecule is PUF (e.g., wildtype PUF, or a variant or fragment thereof) the binding element is PRE (e.g., wildtype PRE, or a variant or fragment thereof).

In some embodiments, when the tether molecule is PUF2 (e.g., wildtype PUF2, or a variant or fragment thereof) the binding element is PRE2 (e.g., wildtype PRE2, or a variant or fragment thereof).

In some embodiments, when the tether molecule is MBP (e.g., wildtype MBP, a variant or fragment thereof) the binding element is MS2 (e.g., wildtype MS2, or a variant or fragment thereof). In some instances, MBP predimerizes prior to binding to MS2 hairpins.

In some embodiments, when the tether molecule is MBP-LacZ or a variant or fragment thereof, the binding element is MS2 (e.g., wildtype MS2, or a variant or fragment thereof).

In some embodiments, when the tether molecule is PCP (e.g., wildtype PCP, or a variant or fragment thereof) the binding element is PP7 (e.g., wildtype PP7, or a variant or fragment thereof).

In some embodiments, when the tether molecule is Lambda N (e.g., wildtype Lambda N, or a variant or fragment thereof) the binding element is BoxB (e.g., wildtype BoxB, or a variant or fragment thereof).

In some embodiments, when the tether molecule is UIA (e.g., wildtype U1A, or a variant or fragment thereof) the binding element is U1A hairpin (e.g., wildtype U1A hairpin, or a variant or fragment thereof).

In some embodiments, when the tether molecule is 15.5 kd (e.g., wildtype 15.5 kd, or a variant or fragment thereof) the binding element is a kink-turn forming sequence (e.g., wildtype U1A hairpin, or a variant or fragment thereof).

In some embodiments, when the tether molecule is LARP7 (e.g., wildtype LARP7, or a variant or fragment thereof) the binding element is 7SK (e.g., wildtype 7SK, or a variant or fragment thereof).

In some embodiments, the second polypeptide is an effector molecule further comprising a tether molecule, which binds to a binding element in the first polynucleotide. In some embodiments, the tether molecule, e.g., effector molecule further comprising a tether molecule, binds to a sequence of the binding element or to a structure comprising the sequence of the binding element. In some embodiments, a tether molecule comprises an RNA-binding protein or a variant or a fragment thereof. Exemplary RNA-binding proteins are provided in Tables 2 and 3.

TABLE 2
Exemplary binding elements and tether molecules

| RNA binding | | Recognition |
|---|---|---|
| protein (RBP) | RNA element | basis |
| PUF | PRE | sequence |
| PUF2 | PRE2 | sequence |
| MBP | MS2 | structure |
| MBP-LacZ | MS2 | structure |
| PCP | PP7 | structure |
| Lambda N | BoxB | structure |
| U1A | U1A hairpin | structure |
| 15.5kd | Kink-turn forming | structure |
| | sequence | |
| 50S ribosomal | Kink-turn forming | structure |
| L7Ae protein | sequence | |
| LARP7 | 7SK | structure |

Additional exemplary RNA-binding proteins or RNA-binding domains which can be used as tether molecules are disclosed in Corley et al, Molecular Cell 78:1 pp. 9-29, the entire contents of which are hereby incorporated by reference. For example, Table 3 provides additional exemplary RNA-binding proteins or domains which can be used as tether molecules. In an embodiment, a tether molecule disclosed herein comprises a domain (or a variant, or a fragment thereof) or a protein (or a variant or a fragment thereof) listed in Table 3.

TABLE 3
Exemplary RNA-binding proteins and domains

| Domain name | Protein family containing domain |
|---|---|
| Cold shock domain | Cold shock proteins, Y-box proteins |
| Double stranded RNA binding | RNAses, ADARs, DICER |
| domain | |
| Helicase | DExH/D-box, Ski2-like, RIGI-like, |
| | NS3, UPF1-like RNA binding helicases |
| Intrinsically disordered | Most RBPs |
| regions (IDR) | |
| K homology | hnRNPs, translation regulation proteins, |
| La motif (LAM) | La proteins, La-related proteins (LARPs) |
| Piwi-Argonaute-Zwille (PAZ) | Argonaute proteins, Dicer |
| P-element Induced Wimpy | Argonaute proteins, |
| Testis (PIWI) | |
| Pentatricopeptide repeat | RNA editing proteins |
| (PPR) | |
| Pseudouridine synthase and | RNA modifying enzymes, metabolic |
| archaeosine transglycoslyase | enzymes |
| (PUA) | |
| Pumillo-like repeat (PUM) | PUF proteins |
| Ribosomal S1-like | Ribosomal proteins, Translation |
| | initiation factors, RNase II, PNPase |
| RNA recognition motif | hnRNPs, splicing factors |
| (RRM) | |
| Sm and Like-Sm (Sm/Lsm) | U1 spliceosomal proteins, Hfq |
| thiouridine synthases, RNA | tRNA modifying enzyme |
| methylases and pseudouridine | |
| synthases (THUMP) | |
| YT521-B homology (YTH) | YTH family m6A readers |
| Zn finger | Transcription factors, METTL |
| | enzymes, |

In some embodiments, the second polypeptide comprises MBP. In some embodiments, the second polypeptide comprises an amino acid sequence provided in Table 4 or a sequence with at least 80%, 85%, 90%, 95%, 96%, 97%, 98%, 99% or 100% identity thereof. In some embodiments, the second polypeptide comprises the amino acid sequence of SEQ ID NO: 21, or an amino acid sequence with at least 80%, 85%, 90%, 95%, 96%, 97%, 98%, 99% or 100% identity thereof.

In some embodiments, the second polypeptide is encoded by a nucleotide sequence provided in Table 4 or a sequence with at least 80%, 85%, 90%, 95%, 96%, 97%, 98%, 99% or 100% identity thereof. In some embodiments, the second polypeptide is encoded by the nucleotide sequence of SEQ ID NO: 22, or a sequence with at least 80%, 85%, 90%, 95%, 96%, 97%, 98%, 99% or 100% identity thereof.

### Effector Molecule

Disclosed herein, inter alia, is a composition or system comprising a polynucleotide, e.g., a second polynucleotide, e.g., mRNA, encoding an effector molecule. In some embodiments, the effector molecule is a translation factor, e.g., Eukaryotic translation initiation factor 4 G (eIF4G). In some embodiments, the effector molecule further comprises a polypeptide that binds to, e.g., recognizes, the binding element (a tether molecule).

In an embodiment, the effector molecule polypeptide comprising a tether molecule comprises a domain which modulates a parameter of, e.g., level and/or activity of: an RNA (e.g., an mRNA); or a protein encoded by the RNA. In an embodiment, the parameter comprises one, two, three or all of: (1) mRNA level and/or activity and/or subcellular localization (e.g., half-life and/or expression); (2) protein level and/or activity (e.g., half-life and/or expression); (3) protein translation rate or (4) protein localization, e.g., location. In an embodiment the effector molecule polypeptide comprising a tether molecule comprises a second domain which binds to, e.g., recognizes, the binding element (a tether molecule).

In an embodiment the effector molecule comprising the tether molecule comprises a polypeptide comprising the first domain and the second domain. In an embodiment, the first and second domains are operatively linked.

In an embodiment, in the second polynucleotide encoding the effector molecule which further comprises a tether molecule, the nucleotide sequence encoding the effector molecule is upstream of the nucleotide sequence encoding the tether molecule. In an embodiment, the nucleotide sequence encoding the effector molecule is downstream of the nucleotide sequence encoding the tether molecule. In an embodiment, the nucleotide sequence encoding the effector molecule is separated from the nucleotide sequence encoding the tether molecule by a protease cleavage site (e.g., a P2A or T2A site) or an internal ribosomal entry site.

In an embodiment, in the second polynucleotide encoding the effector molecule which further comprises a tether molecule, the nucleotide sequence encoding the effector molecule is adjacent to the nucleotide sequence encoding the tether molecule.

In some embodiments, the effector molecule is a translation factor which modulates, e.g., facilitates, ribosome binding, e.g., recruitment, pre-initiation complex formation, or RNA unwinding. In some embodiments, the effector molecule comprises eIF4G, e.g., wildtype eIF4G, a variant of eIF4G, or a fragment thereof (e.g., eIF4G-mid).

In some embodiments, the effector molecule comprises wildtype eIF4G. In some embodiments, wildtype eIF4G comprises a sequence of about 1600 amino acids.

In some embodiments, the effector molecule comprises a fragment of eIF4G, e.g., as disclosed herein. In some embodiments, the eIF4G fragment retains ribosome binding, e.g., recruitment.

In some embodiments, the eIF4G fragment is about 1,500-200 amino acids, about 1,400-300 amino acids, about 1,300-350 amino acids, about 1,200-400 amino acids, about 1,100-450 amino acids, about 1,000-500 amino acids, about 900-550 amino acids, about 800-600 amino acids, about 1,500-300 amino acids, 1,500-400 amino acids, 1,500-500 amino acids, about 1,500-600 amino acids, amino acids, about 1,500-700 amino acids, about 1,500-800 amino acids, about 1,500-900 amino acids, about 1,500-1000 amino acids, about 1,500-1,100 amino acids, about 1,500-1,200 amino acids, about 1,500-1,300 amino acids, about 1,500-1,400 amino acids, about 1,400-200 amino acids, about 1,300-200 amino acids, about 1,200-200 amino acids, about 1,100-200 amino acids, about 1,000-200 amino acids, about 900-200 amino acids, about 800-200 amino acids, about 700-200 amino acids, about 600-200 amino acids, or about 500-200 amino acids in length.

In some embodiments, the eIF4G fragment is about 500 amino acids in length. In some embodiments, the eIF4G fragment is about 600 amino acids in length. In some embodiments, the eIF4G fragment is about 700 amino acids in length. In some embodiments, the eIF4G fragment is about 800 amino acids in length. In some embodiments, the eIF4G fragment is about 900 amino acids in length. In some embodiments, the eIF4G fragment is about 1000 amino acids in length. In some embodiments, the eIF4G fragment is about 1100 amino acids in length. In some embodiments, the eIF4G fragment is about 1200 amino acids in length. In some embodiments, the eIF4G fragment is about 1300 amino acids in length. In some embodiments, the eIF4G fragment is about 1400 amino acids in length. In some embodiments, the eIF4G fragment is about 1500 amino acids in length.

In some embodiments, the effector molecule comprises a variant of eIF4G, e.g., as disclosed herein. In some embodiments, the eIF4G variant retains ribosome binding, e.g., recruitment. In some embodiments, the eIF4G variant comprises a mutation (e.g., substitution) in the eIF4G polypeptide sequence at any one, two, all or a combination of the following positions: amino acid 768, amino acid 771, or amino acid 776. In some embodiments, the eIF4G variant comprises a mutation, e.g., substitution, at position 768 of the eIF4G polypeptide sequence, e.g., a Leucine to Alanine substitution at position 768. In some embodiments, the eIF4G variant comprises a mutation, e.g., substitution, at position 771 of the eIF4G polypeptide sequence, e.g., a Leucine to Alanine substitution at position 771. In some embodiments, the eIF4G variant comprises a mutation, e.g., substitution, at position 776 of the eIF4G polypeptide sequence, e.g., a Phenylalanine to Alanine at position 776. In some embodiments, the eIF4G variant comprises a mutation, e.g., substitution, at position 768 of the eIF4G polypeptide sequence, e.g., an Alanine at position 768; and a mutation, e.g., substitution, at position 771 of the eIF4G polypeptide sequence, e.g., an Alanine at position 771. In some embodiments, the eIF4G variant comprises a mutation, e.g., substitution, at position 768 of the eIF4G polypeptide sequence, e.g., an Alanine at position 768; and a mutation, e.g., substitution, at position 776 of the eIF4G polypeptide sequence, e.g., an Alanine at position 776. In some embodiments, the eIF4G variant comprises a mutation, e.g., substitution, at position 771 of the eIF4G polypeptide sequence, e.g., an Alanine at position 771; and a mutation, e.g., substitution, at position 776 of the eIF4G polypeptide sequence, e.g., an Alanine at position 776. In some embodiments, the eIF4G variant comprises a mutation, e.g., substitution, at position 771 of the eIF4G polypeptide sequence, e.g., an Alanine at position 771; a mutation, e.g., substitution, at position 771 of the eIF4G polypeptide sequence, e.g., an Alanine at position 771; and a mutation, e.g., substitution, at position 776 of the eIF4G polypeptide sequence, e.g., an Alanine at position 776.

In some embodiments, the effector molecule, e.g., eIF4G, comprises an amino acid sequence provided in Table 4, or a sequence with at least 80%, 85%, 90%, 95%, 96%, 97%, 98%, 99% or 100% identity thereof. In some embodiments, the effector molecule, e.g., eIF4G, comprises SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, or SEQ ID NO: 51, or a sequence with at least 80%, 85%, 90%, 95%, 96%, 97%, 98%, 99% or 100% identity thereof.

In some embodiments, the effector molecule e.g., eIF4G, is encoded by a nucleotide sequence provided in Table 4, or a sequence with at least 80%, 85%, 90%, 95%, 96%, 97%, 98%, 99% or 100% identity thereof. In some embodiments, the effector molecule e.g., eIF4G, is encoded by the nucleotide sequence of SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, or SEQ ID NO: 52, or a sequence with at least 80%, 85%, 90%, 95%, 96%, 97%, 98%, 99% or 100% identity thereof.

In some embodiments, the effector molecule is an eIF4G mutant (mutant 798 in Marcotrigiano J. et al., Molecular Cell 7(1):193-203 (2001)) with wild-type levels of IRES activity in the presence of eIF4A and a reduction in activity without it. In some embodiments, the effector molecule is an eIF4G mutant (mutant 802 or 803 in Marcotrigiano J. et al., Molecular Cell 7(1):193-203 (2001)) with similar binding affinity to IRES as the wild-type eIF4G.

In some embodiments, the effector molecule comprises wildtype (e.g., naturally occurring, e.g., human), full length, a fragment (e.g., biologically active or functional fragment), or a variant of an IRES-Trans activating factor (ITAF) protein. In some embodiments, the ITAF protein variant comprises a mutation (e.g., substitution) or a deletion in the ITAF polypeptide sequence that makes it more biologically active. In some embodiments, an ITAF protein has the nuclear localization signal (NLS) and is predominantly expressed in the nucleus. In other embodiments, an ITAF protein is a variant that lacks the NLS and is predominantly expressed in the cytosol. In some embodiments, the effector molecule is an ITAF protein, such as the La protein (La) (e.g., SEQ ID NO: 157). In some embodiments, the La protein is a variant that lacks the NLS (e.g., SEQ ID NO: 158). In some embodiments, the NLS has the following sequence: GKKTKFASDDEHDEHDENGATGPVKRAREETDKEEPASKQQKTENGAGDQ (SEQ ID NO: 159).

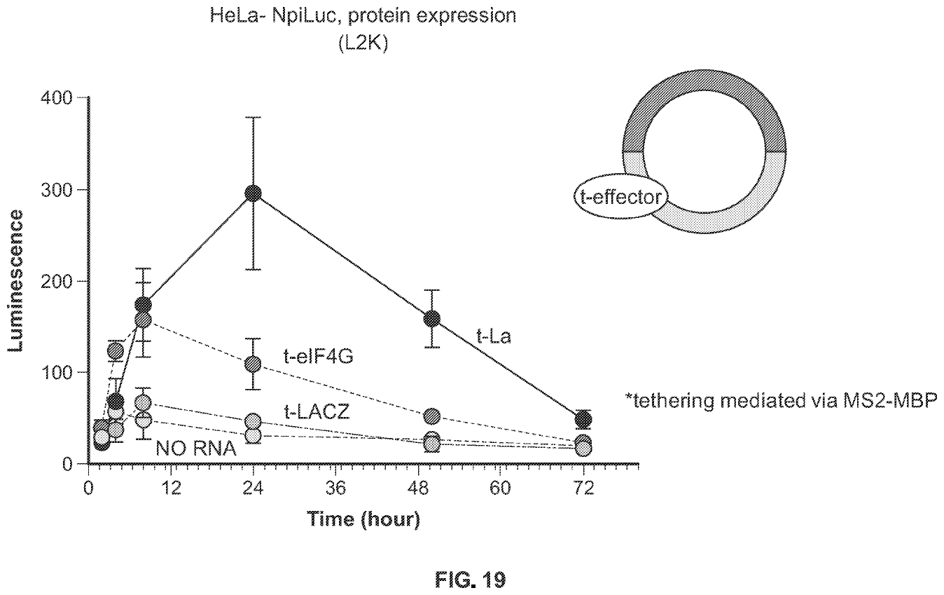

The effector molecule La can be the effector to mediate a translation ON switch, as demonstrated in FIG. 19. Alternatively, the effector molecule can be an ITAF protein known in the art, e.g., PTBP1, CSDE-1, hnrnp-k, DAP5, DDX3, etc, or a variant thereof. Examples of ITAF proteins are provided in King, H. A. et al, Biochem Soc Trans 1 Dec. 2010; 38 (6): 1581-1586; Lee K. M., et al., Trends Microbiol. 2017 July; 25(7):546-561; Godet A. C., et al. Int J Mol Sci. 2019 Feb. 20; 20(4); and Shih, J W., et al. Oncogene 27, 70D-714 (2008). Another example of an ITAF is a truncated form of DAP5, which was shown to be more active in stimulating IRES translation (Henis-Korenblit S., et al. Mol Cell Biol. 2000 January; 20(2):496-506.) Yet another example of an ITAF is hnRNP A1 with S4D/S6D mutations which was found to be more active (Barrera A, et al. Nucleic Acids Res. 2020 Oct. 9; 48(18):10479-10499).

TABLE 4
Exemplary sequences of tether and effector molecules

| | | |
|---|---|---|
| SEQ | | |
| ID | Sequence | |
| NO | information | Sequence |

Second polypeptide (Effector molecule or tether molecule) sequences

| | | |
|---|---|---|
| 21 | MBP (aa) | MASNFTQFVLVDNGGTGDVTVAPSNFANGIAEWISSNSRSQAYKVTCS |
| | | VRQSSAQNRKYTIKVEVPKGAWRSYLNMELTIPIFATNSDCELIVKAMQ |
| | | GLLKDGNPIPSAIAANSGIY |
| 22 | MBP (nt) | ATGGCCAGCAACTTCACCCAGTTCGTGCTGGTGGACAACGGCGGTA |
| | | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTAC |
| 23 | eIF4GdN | IFASMQKPEGLPHISDVVLDKANKTPLRPLDPTRLQGINCGPDFTPSFAN |
| | (aa) | LGRTTLSTRGPPRGGPGGELPRGPAGLGPRRSQQGPRKEPRKIIATVLMT |
| | | EDIKLNKAEKAWKPSSKRTAADKDRGEEDADGSKTQDLFRRVRSILNK |
| | | LTPQMFQQLMKQVTQLAIDTEERLKGVIDLIFEKAISEPNFSVAYANMC |
| | | RCLMALKVPTTEKPTVTVNFRKLLLNRCQKEFEKDKDDDEVFEKKQKE |
| | | MDEAATAEERGRLKEELEEARDIARRRSLGNIKFIGELFKLKMLTEAIM |
| | | HDCVVKLLKNHDEESLECLCRLLTTIGKDLDFEKAKPRMDQYFNQMEK |
| | | IIKEKKTSSRIRFMLQDVLDLRGSNWVPRRGDQGPKTIDQIHKEAEMEE |
| | | HREHIKVQQLMAKGSDKRRGGPPGPPISRGLPLVDDGGWNTVPISKGSR |
| | | PIDTSRLTKITKPGSIDSNNQLFAPGGRLSWGKGSSGGSGAKPSDAASEA |
| | | ARPATSTLNRFSALQQAVPTESTDNRRVVQRSSLSRERGEKAGDRGDR |
| | | LERSERGGDRGDRLDRARTPATKRSFSKEVEERSRERPSQPEGLRKAAS |
| | | LTEDRDRGRDAVKREAALPPVSPLKAALSEEELEKKSKAIIEEYLHLND |
| | | MKEAVQCVQELASPSLLFIFVRHGVESTLERSAIAREHMGQLLHQLLCA |
| | | GHLSTAQYYQGLYEILELAEDMEIDIPHVWLYLAELVTPILQEGGVPMG |
| | | ELFREITKPLRPLGKAASLLLEILGLLCKSMGPKKVGTLWREAGLSWKE |
| | | FLPEGQDIGAFVAEQKVEYTLGEESEAPGQRALPSEELNRQLEKLLKEG |
| | | SSNQRVFDWIEANLSEQQIVSNTLVRALMTAVCYSAIIFETPLRVDVAV |
| | | LKARAKLLQKYLCDEQKELQALYALQALVVTLEQPPNLLRMFFDALY |
| | | DEDVVKEDAFYSWESSKDPAEQQGKGVALKSVTAFFKWLREAEEESD |
| | | HN |
| 24 | eIF4GdN (nt) | ATATTCGCGAGCATGCAGAAGCCTGAAGGTCTGCCTCACATCAGCG |
| | | ACGTGGTGCTGGACAAGGCCAACAAGACCCCTCTTAGACCTCTGGA |
| | | CCCTACCAGACTGCAGGGCATCAACTGCGGCCCTGACTTCACTCCTT |
| | | CTTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTIGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCA |
| | | TCGAGGAGTACCTGCACCTGAACGACATGAAGGAGGCCGTGCAGTG |
| | | CGTGCAAGAGCTCGCGAGCCCATCACTGCTGTTCATCTTCGTCCGGC |
| | | ACGGCGTGGAGTCCACACTGGAAAGATCTGCCATTGCTAGGGAGCA |
| | | TATGGGCCAGTTGTTGCACCAATTGCTTTGCGCCGGCCACCTAAGTA |
| | | CTGCCCAGTACTATCAGGGTTTATACGAGATCCTCGAACTGGCCGAA |
| | | GACATGGAGATCGACATCCCTCACGTGTGGCTGTACCTCGCAGAACT |
| | | GGTGACCCCTATCCTGCAGGAGGGCGGCGTTCCAATGGGTGAGTTGT |
| | | TCAGAGAAATCACAAAGCCACTGCGCCCACTGGGCAAGGCGGCTAG |
| | | TCTCCTTCTGGAGATTCTCGGCCTGCTCTGTAAGAGCATGGGTCCAA |
| | | AGAAGGTGGGCACCCTGTGGAGGGAAGCTGGACTCTCCTGGAAGGA |
| | | ATTCCTCCCTGAGGGTCAGGACATCGGCGCCTTCGTGGCCGAACAGA |
| | | AGGTTGAGTACACCCTGGGAGAGGAATCGGAAGCGCCGGGTCAGCG |
| | | GGCTCTGCCGAGTGAGGAGCTCAACAGACAACTCGAGAAGCTCCTC |
| | | AAGGAGGGATCCTCAAATCAGAGAGTGTTCGACTGGATCGAGGCCA |
| | | ATCTCAGCGAGCAGCAAATCGTGAGCAACACGTTGGTTCGCGCTCTC |
| | | ATGACAGCCGTGTGCTACTCAGCCATTATCTTCGAGACGCCTCTCCG |
| | | CGTGGACGTGGCAGTGCTCAAGGCCCGCGCTAAGCTGTTACAGAAG |
| | | TACCTGTGCGACGAGCAGAAGGAACTGCAGGCCCTGTACGCCCTGC |
| | | AAGCTCTGGTCGTCACACTCGAGCAGCCTCCTAACCTGCTGAGAATG |
| | | TTCTTCGACGCATTGTACGACGAGGACGTTGTGAAGGAAGACGCTTT |
| | | CTACAGCTGGGAGTCTAGTAAGGATCCTGCGGAACAACAAGGCAAG |
| | | GGCGTTGCTCTAAAGAGCGTGACCGCCTTCTTCAAGTGGCTGCGAGA |
| | | GGCTGAGGAAGAGTCGGACCACAAC |
| 25 | MBP- | MASNFTQFVLVDNGGTGDVTVAPSNFANGIAEWISSNSRSQAYKVTCS |
| | eIF4GdN | VRQSSAQNRKYTIKVEVPKGAWRSYLNMELTIPIFATNSDCELIVKAMQ |
| | (aa) | GLLKDGNPIPSAIAANSGIYGGGGSIFASMQKPEGLPHISDVVLDKANKT |
| | | PLRPLDPTRLQGINCGPDFTPSFANLGRTTLSTRGPPRGGPGGELPRGPA |
| | | GLGPRRSQQGPRKEPRKIIATVLMTEDIKLNKAEKAWKPSSKRTAADK |
| | | DRGEEDADGSKTQDLFRRVRSILNKLTPQMFQQLMKQVTQLAIDTEER |
| | | LKGVIDLIFEKAISEPNFSVAYANMCRCLMALKVPTTEKPTVTVNFRKL |
| | | LLNRCQKEFEKDKDDDEVFEKKQKEMDEAATAEERGRLKEELEEARDI |
| | | ARRRSLGNIKFIGELFKLKMLTEAIMHDCVVKLLKNHDEESLECLCRLL |
| | | TTIGKDLDFEKAKPRMDQYFNQMEKIIKEKKTSSRIRFMLQDVLDLRGS |
| | | NWVPRRGDQGPKTIDQIHKEAEMEEHREHIKVQQLMAKGSDKRRGGP |
| | | PGPPISRGLPLVDDGGWNTVPISKGSRPIDTSRLTKITKPGSIDSNNQLFA |
| | | PGGRLSWGKGSSGGSGAKPSDAASEAARPATSTLNRFSALQQAVPTEST |
| | | DNRRVVQRSSLSRERGEKAGDRGDRLERSERGGDRGDRLDRARTPATK |
| | | RSFSKEVEERSRERPSQPEGLRKAASLTEDRDRGRDAVKREAALPPVSP |
| | | LKAALSEEELEKKSKAIIEEYLHLNDMKEAVQCVQELASPSLLFIFVRHG |
| | | VESTLERSAIAREHMGQLLHQLLCAGHLSTAQYYQGLYEILELAEDMEI |
| | | DIPHVWLYLAELVTPILQEGGVPMGELFREITKPLRPLGKAASLLLEILG |
| | | LLCKSMGPKKVGTLWREAGLSWKEFLPEGQDIGAFVAEQKVEYTLGEE |
| | | SEAPGQRALPSEELNRQLEKLLKEGSSNQRVFDWIEANLSEQQIVSNTL |
| | | VRALMTAVCYSAIIFETPLRVDVAVLKARAKLLQKYLCDEQKELQALY |
| | | ALQALVVTLEQPPNLLRMFFDALYDEDVVKEDAFYSWESSKDPAEQQ |
| | | GKGVALKSVTAFFKWLREAEEESDHN |
| 26 | MBP- | ATGGCCAGCAACTTCACCCAGTTCGTGCTGGTGGACAACGGCGGTA |
| | eIF4GdN (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCATA |
| | | TTCGCGAGCATGCAGAAGCCTGAAGGTCTGCCTCACATCAGCGACG |
| | | TGGTGCTGGACAAGGCCAACAAGACCCCTCTTAGACCTCTGGACCCT |
| | | ACCAGACTGCAGGGCATCAACTGCGGCCCTGACTTCACTCCTTCTTT |
| | | CGCAAACCTGGGCAGAACCACCCTGAGCACCAGAGGCCCTCCTAGA |
| | | GGTGGTCCCGGCGGAGAACTCCCCAGGGGTCCTGCCGGCCTGGGCC |
| | | CTAGACGCTCCCAGCAAGGTCCTAGAAAGGAGCCAAGGAAGATCAT |
| | | CGCCACCGTGCTGATGACCGAGGACATCAAGCTGAACAAAGCTGAG |
| | | AAGGCCTGGAAGCCTAGCAGCAAGAGAACCGCCGCCGACAAGGAC |
| | | AGAGGCGAGGAGGACGCCGACGGATCCAAGACCCAGGACCTGTTCA |
| | | GAAGAGTGAGAAGCATCCTCAACAAGCTGACCCCTCAGATGTTCCA |
| | | GCAGCTGATGAAGCAGGTGACGCAGCTCGCCATCGACACCGAGGAG |
| | | AGACTGAAGGGCGTGATCGACCTGATCTTTGAGAAGGCTATCTCAG |
| | | AGCCTAACTTCAGCGTGGCCTACGCCAACATGTGCCGTTGCCTGATG |
| | | GCATTGAAGGTGCCAACCACCGAGAAGCCTACTGTGACCGTCAATTT |
| | | CCGTAAACTGCTGCTGAACCGGTGCCAGAAAGAGTTCGAGAAGGAT |
| | | AAGGACGACGACGAGGTCTTCGAGAAGAAACAGAAAGAAATGGAC |
| | | GAGGCCGCCACCGCAGAGGAAAGGGGCCGATTAAAGGAGGAGCTG |
| | | GAGGAGGCCAGAGACATCGCCAGACGGCGTTCTCTGGGCAACATCA |
| | | AGTTCATAGGTGAGCTGTTCAAGCTAAAGATGCTCACCGAGGCCAT |
| | | AATGCACGACTGCGTGGTGAAGCTACTGAAGAACCACGACGAAGAA |
| | | AGCCTGGAGTGCCTGTGCAGACTGCTGACCACCATCGGCAAGGACC |
| | | TGGACTTCGAGAAGGCAAAGCCTCGAATGGACCAGTACTTCAACCA |
| | | GATGGAGAAGATTATCAAGGAGAAGAAGACCAGCAGCAGAATCAG |
| | | ATTCATGCTGCAGGACGTACTGGACCTGCGCGGAAGCAACTGGGTG |
| | | CCAAGGAGAGGGGACCAAGGACCAAAGACCATCGACCAGATCCAC |
| | | AAGGAAGCGGAGATGGAGGAGCACAGAGAGCACATAAAGGTGCAG |
| | | CAGCTTATGGCCAAGGGCAGCGACAAGCGAAGAGGCGGCCCGCCCG |
| | | GACCTCCTATCAGCAGAGGCCTTCCTCTGGTAGACGACGGCGGCTGG |
| | | AACACCGTGCCTATCTCTAAGGGCTCCAGACCTATCGACACTTCCCG |
| | | TCTTACCAAGATCACCAAGCCAGGATCTATTGACAGCAACAACCAG |
| | | CTGTTCGCCCCAGGAGGAAGACTTAGCTGGGGCAAGGGAAGTTCCG |
| | | GCGGATCCGGCGCCAAGCCTTCCGACGCCGCCAGCGAGGCTGCCAG |
| | | ACCTGCCACCAGCACCTTGAACCGCTTTTCCGCTCTGCAGCAAGCTG |
| | | TGCCGACCGAGAGCACCGACAACCGGCGAGTTGTGCAGAGAAGCAG |
| | | CCTGAGCAGAGAGAGGGGCGAGAAAGCCGGCGATAGAGGTGACCG |
| | | ACTGGAGAGAAGCGAGAGAGGAGGTGATAGAGGCGACCGGCTTGA |
| | | CAGAGCCAGAACCCCTGCCACAAAGCGATCGTTCAGCAAGGAGGTC |
| | | GAAGAGAGGTCCAGGGAGCGCCCTAGCCAGCCGGAAGGACTCAGA |
| | | AAGGCAGCCAGTCTAACAGAGGACCGCGACAGGGGAAGAGACGCC |
| | | GTGAAGAGGGAGGCCGCACTGCCTCCTGTGAGCCCTCTGAAGGCGG |
| | | CACTGAGCGAAGAAGAACTTGAGAAGAAGAGTAAGGCGATCATCG |
| | | AGGAGTACCTGCACCTGAACGACATGAAGGAGGCCGTGCAGTGCGT |
| | | GCAAGAGCTCGCGAGCCCATCACTGCTGTTCATCTTCGTCCGGCACG |
| | | GCGTGGAGTCCACACTGGAAAGATCTGCCATTGCTAGGGAGCATAT |
| | | GGGCCAGTTGTTGCACCAATTGCTTTGCGCCGGCCACCTAAGTACTG |
| | | CCCAGTACTATCAGGGTTTATACGAGATCCTCGAACTGGCCGAAGAC |
| | | ATGGAGATCGACATCCCTCACGTGTGGCTGTACCTCGCAGAACTGGT |
| | | GACCCCTATCCTGCAGGAGGGCGGCGTTCCAATGGGTGAGTTGTTCA |
| | | GAGAAATCACAAAGCCACTGCGCCCACTGGGCAAGGCGGCTAGTCT |
| | | CCTTCTGGAGATTCTCGGCCTGCTCTGTAAGAGCATGGGTCCAAAGA |
| | | AGGTGGGCACCCTGTGGAGGGAAGCTGGACTCTCCTGGAAGGAATT |
| | | CCTCCCTGAGGGTCAGGACATCGGCGCCTTCGTGGCCGAACAGAAG |
| | | GTTGAGTACACCCTGGGAGAGGAATCGGAAGCGCCGGGTCAGCGGG |
| | | CTCTGCCGAGTGAGGAGCTCAACAGACAACTCGAGAAGCTCCTCAA |
| | | GGAGGGATCCTCAAATCAGAGAGTGTTCGACTGGATCGAGGCCAAT |
| | | CTCAGCGAGCAGCAAATCGTGAGCAACACGTTGGTTCGCGCTCTCAT |
| | | GACAGCCGTGTGCTACTCAGCCATTATCTTCGAGACGCCTCTCCGCG |
| | | TGGACGTGGCAGTGCTCAAGGCCCGCGCTAAGCTGTTACAGAAGTA |
| | | CCTGTGCGACGAGCAGAAGGAACTGCAGGCCCTGTACGCCCTGCAA |
| | | GCTCTGGTCGTCACACTCGAGCAGCCTCCTAACCTGCTGAGAATGTT |
| | | CTTCGACGCATTGTACGACGAGGACGTTGTGAAGGAAGACGCTTTCT |
| | | ACAGCTGGGAGTCTAGTAAGGATCCTGCGGAACAACAAGGCAAGGG |
| | | CGTTGCTCTAAAGAGCGTGACCGCCTTCTTCAAGTGGCTGCGAGAGG |
| | | CTGAGGAAGAGTCGGACCACAAC |
| 27 | MBP eIF4G | MASNFTQFVLVDNGGTGDVTVAPSNFANGIAEWISSNSRSQAYKVTCS |
| | (fl) (aa) | VRQSSAQNRKYTIKVEVPKGAWRSYLNMELTIPIFATNSDCELIVKAMQ |
| | | GLLKDGNPIPSAIAANSGIYGGGGSNKAPQSTGPPPAPSPGLPQPAFPPG |
| | | QTAPVVFSTPQATQMNTPSQPRQHFYPSRAQPPSSAASRVQSAAPARPG |
| | | PAAHVYPAGSQVMMIPSQISYPASQGAYYIPGQGRSTYVVPTQQYPVQP |
| | | GAPGFYPGASPTEFGTYAGAYYPAQGVQQFPTGVAPTPVLMNQPPQIA |
| | | PKRERKTIRIRDPNQGGKDITEEIMSGARTASTPTPPQTGGGLEPQANGE |
| | | TPQVAVIVRPDDRSQGAIIADRPGLPGPEHSPSESQPSSPSPTPSPSPVLEP |
| | | GSEPNLAVLSIPGDTMTTIQMSVEESTPISRETGEPYRLSPEPTPLAEPILE |
| | | VEVTLSKPVPESEFSSSPLQAPTPLASHTVEIHEPNGMVPSEDLEPEVESS |
| | | PELAPPPACPSESPVPIAPTAQPEELLNGAPSPPAVDLSPVSEPEEQAKEV |
| | | TASMAPPTIPSATPATAPSATSPAQEEEMEEEEEEEEGEAGEAGEAESEK |
| | | GGEELLPPESTPIPANLSQNLEAAAATQVAVSVPKRRRKIKELNKKEAV |
| | | GDLLDAFKEANPAVPEVENQPPAGSNPGPESEGSGVPPRPEEADETWDS |
| | | KEDKIHNAENIQPGEQKYEYKSDQWKPLNLEEKKRYDREFLLGFQFIFA |
| | | SMQKPEGLPHISDVVLDKANKTPLRPLDPTRLQGINCGPDFTPSFANLG |
| | | RTTLSTRGPPRGGPGGELPRGPAGLGPRRSQQGPRKEPRKIIATVLMTED |
| | | IKLNKAEKAWKPSSKRTAADKDRGEEDADGSKTQDLFRRVRSILNKLT |
| | | PQMFQQLMKQVTQLAIDTEERLKGVIDLIFEKAISEPNFSVAYANMCRC |
| | | LMALKVPTTEKPTVTVNFRKLLLNRCQKEFEKDKDDDEVFEKKQKEM |
| | | DEAATAEERGRLKEELEEARDIARRRSLGNIKFIGELFKLKMLTEAIMH |
| | | DCVVKLLKNHDEESLECLCRLLTTIGKDLDFEKAKPRMDQYFNQMEKII |
| | | KEKKTSSRIRFMLQDVLDLRGSNWVPRRGDQGPKTIDQIHKEAEMEEH |
| | | REHIKVQQLMAKGSDKRRGGPPGPPISRGLPLVDDGGWNTVPISKGSRP |
| | | IDTSRLTKITKPGSIDSNNQLFAPGGRLSWGKGSSGGSGAKPSDAASEAA |
| | | RPATSTLNRFSALQQAVPTESTDNRRVVQRSSLSRERGEKAGDRGDRLE |
| | | RSERGGDRGDRLDRARTPATKRSFSKEVEERSRERPSQPEGLRKAASLT |
| | | EDRDRGRDAVKREAALPPVSPLKAALSEEELEKKSKAIIEEYLHLNDMK |
| | | EAVQCVQELASPSLLFIFVRHGVESTLERSAIAREHMGQLLHQLLCAGH |
| | | LSTAQYYQGLYEILELAEDMEIDIPHVWLYLAELVTPILQEGGVPMGEL |
| | | FREITKPLRPLGKAASLLLEILGLLCKSMGPKKVGTLWREAGLSWKEFL |
| | | PEGQDIGAFVAEQKVEYTLGEESEAPGQRALPSEELNRQLEKLLKEGSS |
| | | NQRVFDWIEANLSEQQIVSNTLVRALMTAVCYSAIIFETPLRVDVAVLK |
| | | ARAKLLQKYLCDEQKELQALYALQALVVTLEQPPNLLRMFFDALYDE |
| | | DVVKEDAFYSWESSKDPAEQQGKGVALKSVTAFFKWLREAEEESDHN |
| 28 | MBP eIF4G | ATGGCCAGCAACTTCACCCAGTTCGTGCTGGTGGACAACGGCGGTA |
| | (fl) (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCAAC |
| | | AAGGCCCCTCAGAGCACCGGCCCTCCTCCTGCCCCTAGCCCTGGCCT |
| | | GCCTCAGCCTGCCTTCCCTCCTGGCCAGACCGCCCCTGTGGTGTTCA |
| | | GCACCCCTCAGGCCACCCAGATGAACACACCTAGCCAGCCTAGACA |
| | | GCACTTCTACCCTAGCAGAGCCCAGCCTCCTAGCAGCGCCGCCAGCA |
| | | GAGTGCAGTCCGCTGCACCTGCCAGACCTGGCCCTGCCGCCCACGTG |
| | | TACCCTGCCGGCAGCCAGGTGATGATGATCCCGAGTCAAATCAGCT |
| | | ACCCTGCAAGCCAGGGCGCCTACTACATCCCGGGCCAGGGCAGAAG |
| | | CACCTACGTGGTGCCTACCCAGCAGTACCCTGTGCAGCCTGGCGCCC |
| | | CTGGTTTCTATCCTGGCGCAAGCCCTACCGAGTTCGGAACTTACGCC |
| | | GGCGCTTACTATCCAGCTCAGGGCGTGCAGCAGTTCCCTACCGGCGT |
| | | GGCCCCTACCCCTGTGCTGATGAATCAGCCACCTCAGATCGCCCCTA |
| | | AGCGCGAGCGCAAGACCATCAGAATCCGCGATCCTAACCAGGGCGG |
| | | CAAGGACATCACCGAGGAGATCATGAGCGGCGCCAGAACAGCAAGT |
| | | ACTCCAACCCCGCCACAAACCGGCGGCGGCCTGGAGCCTCAAGCCA |
| | | ACGGCGAGACGCCACAAGTGGCCGTGATCGTACGCCCTGACGACCG |
| | | GAGCCAAGGTGCAATCATCGCCGATAGGCCTGGCCTCCCAGGTCCG |
| | | GAGCACAGCCCTAGCGAGTCCCAGCCGTCTTCACCATCACCAACCCC |
| | | TAGTCCATCCCCTGTTCTCGAACCAGGCAGCGAGCCTAACCTGGCCG |
| | | TGCTGAGCATACCAGGTGACACCATGACCACCATCCAGATGAGCGT |
| | | GGAGGAGAGCACCCCAATCAGCAGAGAAACTGGAGAGCCTTACAG |
| | | ACTGTCCCCAGAGCCGACCCCACTGGCCGAGCCAATACTGGAGGTG |
| | | GAGGTGACCCTGAGCAAGCCTGTGCCTGAGAGCGAGTTCAGCTCCT |
| | | CTCCACTGCAGGCCCCAACTCCTCTCGCAAGCCACACCGTGGAGATC |
| | | CACGAACCTAACGGCATGGTACCAAGCGAAGATCTTGAGCCAGAGG |
| | | TCGAATCAAGCCCAGAACTGGCCCCTCCACCTGCCTGCCCGTCTGAA |
| | | TCTCCGGTCCCTATCGCTCCTACGGCACAGCCTGAGGAGCTGCTGAA |
| | | CGGTGCCCCGAGCCCTCCAGCAGTGGACTTATCCCCAGTATCAGAGC |
| | | CTGAAGAACAGGCCAAGGAGGTAACTGCCTCTATGGCGCCACCTAC |
| | | CATACCTTCGGCAACACCGGCTACAGCACCATCTGCGACTAGTCCGG |
| | | CTCAGGAGGAGGAGATGGAGGAAGAAGAAGAGGAAGAGGAGGGCG |
| | | AGGCCGGAGAGGCCGGTGAAGCCGAGTCCGAGAAGGGCGGCGAAG |
| | | AACTTCTCCCTCCAGAGTCAACTCCTATCCCTGCCAACCTTAGTCAG |
| | | AATCTGGAGGCCGCCGCCGCTACTCAGGTTGCAGTGAGCGTGCCAA |
| | | AGAGACGTCGCAAGATCAAGGAGCTGAACAAGAAGGAGGCCGTGG |
| | | GCGACCTGCTGGACGCCTTCAAGGAGGCAAACCCGGCGGTGCCTGA |
| | | AGTGGAGAATCAGCCTCCGGCCGGATCAAACCCTGGTCCTGAGAGT |
| | | GAAGGCAGCGGCGTCCCACCAAGACCTGAAGAGGCTGACGAAACTT |
| | | GGGACAGCAAGGAGGACAAGATCCACAACGCCGAGAACATCCAGC |
| | | CAGGCGAGCAGAAGTACGAGTACAAGAGCGACCAGTGGAAGCCTCT |
| | | TAACCTTGAAGAGAAGAAGAGATACGACAGAGAGTTCCTGCTGGGC |
| | | TTCCAGTTCATATTCGCGAGCATGCAGAAGCCTGAAGGTCTGCCTCA |
| | | CATCAGCGACGTGGTGCTGGACAAGGCCAACAAGACCCCTCTTAGA |
| | | CCTCTGGACCCTACCAGACTGCAGGGCATCAACTGCGGCCCTGACTT |
| | | CACTCCTTCTTTCGCAAACCTGGGCAGAACCACCCTGAGCACCAGAG |
| | | GCCCTCCTAGAGGTGGTCCCGGCGGAGAACTCCCCAGGGGTCCTGC |
| | | CGGCCTGGGCCCTAGACGCTCCCAGCAAGGTCCTAGAAAGGAGCCA |
| | | AGGAAGATCATCGCCACCGTGCTGATGACCGAGGACATCAAGCTGA |
| | | ACAAAGCTGAGAAGGCCTGGAAGCCTAGCAGCAAGAGAACCGCCG |
| | | CCGACAAGGACAGAGGCGAGGAGGACGCCGACGGATCCAAGACCC |
| | | AGGACCTGTTCAGAAGAGTGAGAAGCATCCTCAACAAGCTGACCCC |
| | | TCAGATGTTCCAGCAGCTGATGAAGCAGGTGACGCAGCTCGCCATC |
| | | GACACCGAGGAGAGACTGAAGGGCGTGATCGACCTGATCTTTGAGA |
| | | AGGCTATCTCAGAGCCTAACTTCAGCGTGGCCTACGCCAACATGTGC |
| | | CGTTGCCTGATGGCATTGAAGGTGCCAACCACCGAGAAGCCTACTGT |
| | | GACCGTCAATTTCCGTAAACTGCTGCTGAACCGGTGCCAGAAAGAG |
| | | TTCGAGAAGGATAAGGACGACGACGAGGTCTTCGAGAAGAAACAG |
| | | AAAGAAATGGACGAGGCCGCCACCGCAGAGGAAAGGGGCCGATTA |
| | | AAGGAGGAGCTGGAGGAGGCCAGAGACATCGCCAGACGGCGTTCTC |
| | | TGGGCAACATCAAGTTCATAGGTGAGCTGTTCAAGCTAAAGATGCTC |
| | | ACCGAGGCCATAATGCACGACTGCGTGGTGAAGCTACTGAAGAACC |
| | | ACGACGAAGAAAGCCTGGAGTGCCTGTGCAGACTGCTGACCACCAT |
| | | CGGCAAGGACCTGGACTTCGAGAAGGCAAAGCCTCGAATGGACCAG |
| | | TACTTCAACCAGATGGAGAAGATTATCAAGGAGAAGAAGACCAGCA |
| | | GCAGAATCAGATTCATGCTGCAGGACGTACTGGACCTGCGCGGAAG |
| | | CAACTGGGTGCCAAGGAGAGGGGACCAAGGACCAAAGACCATCGA |
| | | CCAGATCCACAAGGAAGCGGAGATGGAGGAGCACAGAGAGCACAT |
| | | AAAGGTGCAGCAGCTTATGGCCAAGGGCAGCGACAAGCGAAGAGG |
| | | CGGCCCGCCCGGACCTCCTATCAGCAGAGGCCTTCCTCTGGTAGACG |
| | | ACGGCGGCTGGAACACCGTGCCTATCTCTAAGGGCTCCAGACCTATC |
| | | GACACTTCCCGTCTTACCAAGATCACCAAGCCAGGATCTATTGACAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCTGTGCCGACCGAGAGCACCGACAACCGGCGAGTTGTGC |
| | | AGAGAAGCAGCCTGAGCAGAGAGAGGGGCGAGAAAGCCGGCGATA |
| | | GAGGTGACCGACTGGAGAGAAGCGAGAGAGGAGGTGATAGAGGCG |
| | | ACCGGCTTGACAGAGCCAGAACCCCTGCCACAAAGCGATCGTTCAG |
| | | CAAGGAGGTCGAAGAGAGGTCCAGGGAGCGCCCTAGCCAGCCGGA |
| | | AGGACTCAGAAAGGCAGCCAGTCTAACAGAGGACCGCGACAGGGG |
| | | AAGAGACGCCGTGAAGAGGGAGGCCGCACTGCCTCCTGTGAGCCCT |
| | | CTGAAGGCGGCACTGAGCGAAGAAGAACTTGAGAAGAAGAGTAAG |
| | | GCGATCATCGAGGAGTACCTGCACCTGAACGACATGAAGGAGGCCG |
| | | TGCAGTGCGTGCAAGAGCTCGCGAGCCCATCACTGCTGTTCATCTTC |
| | | GTCCGGCACGGCGTGGAGTCCACACTGGAAAGATCTGCCATTGCTA |
| | | GGGAGCATATGGGCCAGTTGTTGCACCAATTGCTTTGCGCCGGCCAC |
| | | CTAAGTACTGCCCAGTACTATCAGGGTTTATACGAGATCCTCGAACT |
| | | GGCCGAAGACATGGAGATCGACATCCCTCACGTGTGGCTGTACCTC |
| | | GCAGAACTGGTGACCCCTATCCTGCAGGAGGGCGGCGTTCCAATGG |
| | | GTGAGTTGTTCAGAGAAATCACAAAGCCACTGCGCCCACTGGGCAA |
| | | GGCGGCTAGTCTCCTTCTGGAGATTCTCGGCCTGCTCTGTAAGAGCA |
| | | TGGGTCCAAAGAAGGTGGGCACCCTGTGGAGGGAAGCTGGACTCTC |
| | | CTGGAAGGAATTCCTCCCTGAGGGTCAGGACATCGGCGCCTTCGTGG |
| | | CCGAACAGAAGGTTGAGTACACCCTGGGAGAGGAATCGGAAGCGCC |
| | | GGGTCAGCGGGCTCTGCCGAGTGAGGAGCTCAACAGACAACTCGAG |
| | | AAGCTCCTCAAGGAGGGATCCTCAAATCAGAGAGTGTTCGACTGGA |
| | | TCGAGGCCAATCTCAGCGAGCAGCAAATCGTGAGCAACACGTTGGT |
| | | TCGCGCTCTCATGACAGCCGTGTGCTACTCAGCCATTATCTTCGAGA |
| | | CGCCTCTCCGCGTGGACGTGGCAGTGCTCAAGGCCCGCGCTAAGCTG |
| | | TTACAGAAGTACCTGTGCGACGAGCAGAAGGAACTGCAGGCCCTGT |
| | | ACGCCCTGCAAGCTCTGGTCGTCACACTCGAGCAGCCTCCTAACCTG |
| | | CTGAGAATGTTCTTCGACGCATTGTACGACGAGGACGTTGTGAAGG |
| | | AAGACGCTTTCTACAGCTGGGAGTCTAGTAAGGATCCTGCGGAACA |
| | | ACAAGGCAAGGGCGTTGCTCTAAAGAGCGTGACCGCCTTCTTCAAG |
| | | TGGCTGCGAGAGGCTGAGGAAGAGTCGGACCACAAC |
| 29 | MBP eIF4G- | MASNFTQFVLVDNGGTGDVTVAPSNFANGIAEWISSNSRSQAYKVTCS |
| | dN (623- | VRQSSAQNRKYTIKVEVPKGAWRSYLNMELTIPIFATNSDCELIVKAMQ |
| | 1599, 3A) | GLLKDGNPIPSAIAANSGIYGGGGSIFASMQKPEGLPHISDVVLDKANKT |
| | (aa) | PLRPLDPTRLQGINCGPDFTPSFANLGRTTLSTRGPPRGGPGGELPRGPA |
| | | GLGPRRSQQGPRKEPRKIIATVLMTEDIKLNKAEKAWKPSSKRTAADK |
| | | DRGEEDADGSKTQDLFRRVRSIANKATPQMAQQLMKQVTQLAIDTEER |
| | | LKGVIDLIFEKAISEPNFSVAYANMCRCLMALKVPTTEKPTVTVNFRKL |
| | | LLNRCQKEFEKDKDDDEVFEKKQKEMDEAATAEERGRLKEELEEARDI |
| | | ARRRSLGNIKFIGELFKLKMLTEAIMHDCVVKLLKNHDEESLECLCRLL |
| | | TTIGKDLDFEKAKPRMDQYFNQMEKIIKEKKTSSRIRFMLQDVLDLRGS |
| | | NWVPRRGDQGPKTIDQIHKEAEMEEHREHIKVQQLMAKGSDKRRGGP |
| | | PGPPISRGLPLVDDGGWNTVPISKGSRPIDTSRLTKITKPGSIDSNNQLFA |
| | | PGGRLSWGKGSSGG |
| | | SGAKPSDAASEAARPATSTLNRFSALQQAVPTESTDNRRVVQRSSLSRE |
| | | RGEKAGDRGDRLERSERGGDRGDRLDRARTPATKRSFSKEVEERSRER |
| | | PSQPEGLRKAASLTEDRDRGRDAVKREAALPPVSPLKAALSEEELEKKS |
| | | KAIIEEYLHLNDMKEAVQCVQELASPSLLFIFVRHGVESTLERSAIAREH |
| | | MGQLLHQLLCAGHLSTAQYYQGLYEILELAEDMEIDIPHVWLYLAELV |
| | | TPILQEGGVPMGELFREITKPLRPLGKAASLLLEILGLLCKSMGPKKVGT |
| | | LWREAGLSWKEFLPEGQDIGAFVAEQKVEYTLGEESEAPGQRALPSEEL |
| | | NRQLEKLLKEGSSNQRVFDWIEANLSEQQIVSNTLVRALMTAVCYSAII |
| | | FETPLRVDVAVLKARAKLLQKYLCDEQKELQALYALQALVVTLEQPPN |
| | | LLRMFFDALYDEDVVKEDAFYSWESSKDPAEQQGKGVALK |
| | | SVTAFFKWLREAEEESDHN |
| 30 | MBP eIF4G- | ATGGCCAGCAACTTCACCCAGTTCGTGCTGGTGGACAACGGCGGTA |
| | dN (623- | CCGGAGACGTGACCGTGGCCCCTTCTAACTTCGCCAACGGCATCGCC |
| | 1599, 3A) | GAGTGGATCAGCAGCAACAGCAGAAGCCAGGCCTACAAGGTGACCT |
| | (nt) | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCATA |
| | | TTCGCGAGCATGCAGAAGCCTGAAGGTCTGCCTCACATCAGCGACG |
| | | TGGTGCTGGACAAGGCCAACAAGACCCCTCTTAGACCTCTGGACCCT |
| | | ACCAGACTGCAGGGCATCAACTGCGGCCCTGACTTCACTCCTTCTTT |
| | | CGCAAACCTGGGCAGAACCACCCTGAGCACCAGAGGCCCTCCTAGA |
| | | GGTGGTCCCGGCGGAGAACTCCCCAGGGGTCCTGCCGGCCTGGGCC |
| | | CTAGACGCTCCCAGCAAGGTCCTAGAAAGGAGCCAAGGAAGATCAT |
| | | CGCCACCGTGCTGATGACCGAGGACATCAAGCTGAACAAAGCTGAG |
| | | AAGGCCTGGAAGCCTAGCAGCAAGAGAACCGCCGCCGACAAGGAC |
| | | AGAGGCGAGGAGGACGCCGACGGATCCAAGACCCAGGACCTGTTCA |
| | | GAAGAGTGAGAAGCATCGCGAACAAGGCGACCCCTCAGATGGCGCA |
| | | GCAGCTGATGAAGCAGGTGACGCAGCTCGCCATCGACACCGAGGAG |
| | | AGACTGAAGGGCGTGATCGACCTGATCTTTGAGAAGGCTATCTCAG |
| | | AGCCTAACTTCAGCGTGGCCTACGCCAACATGTGCCGTTGCCTGATG |
| | | GCATTGAAGGTGCCAACCACCGAGAAGCCTACTGTGACCGTCAATTT |
| | | CCGTAAACTGCTGCTGAACCGGTGCCAGAAAGAGTTCGAGAAGGAT |
| | | AAGGACGACGACGAGGTCTTCGAGAAGAAACAGAAAGAAATGGAC |
| | | GAGGCCGCCACCGCAGAGGAAAGGGGCCGATTAAAGGAGGAGCTG |
| | | GAGGAGGCCAGAGACATCGCCAGACGGCGTTCTCTGGGCAACATCA |
| | | AGTTCATAGGTGAGCTGTTCAAGCTAAAGATGCTCACCGAGGCCAT |
| | | AATGCACGACTGCGTGGTGAAGCTACTGAAGAACCACGACGAAGAA |
| | | AGCCTGGAGTGCCTGTGCAGACTGCTGACCACCATCGGCAAGGACC |
| | | TGGACTTCGAGAAGGCAAAGCCTCGAATGGACCAGTACTTCAACCA |
| | | GATGGAGAAGATTATCAAGGAGAAGAAGACCAGCAGCAGAATCAG |
| | | ATTCATGCTGCAGGACGTACTGGACCTGCGCGGAAGCAACTGGGTG |
| | | CCAAGGAGAGGGGACCAAGGACCAAAGACCATCGACCAGATCCAC |
| | | AAGGAAGCGGAGATGGAGGAGCACAGAGAGCACATAAAGGTGCAG |
| | | CAGCTTATGGCCAAGGGCAGCGACAAGCGAAGAGGCGGCCCGCCCG |
| | | GACCTCCTATCAGCAGAGGCCTTCCTCTGGTAGACGACGGCGGCTGG |
| | | AACACCGTGCCTATCTCTAAGGGCTCCAGACCTATCGACACTTCCCG |
| | | TCTTACCAAGATCACCAAGCCAGGATCTATTGACAGCAACAACCAG |
| | | CTGTTCGCCCCAGGAGGAAGACTTAGCTGGGGCAAGGGAAGTTCCG |
| | | GCGGATCCGGCGCCAAGCCTTCCGACGCCGCCAGCGAGGCTGCCAG |
| | | ACCTGCCACCAGCACCTTGAACCGCTTTTCCGCTCTGCAGCAAGCTG |
| | | TGCCGACCGAGAGCACCGACAACCGGCGAGTTGTGCAGAGAAGCAG |
| | | CCTGAGCAGAGAGAGGGGCGAGAAAGCCGGCGATAGAGGTGACCG |
| | | ACTGGAGAGAAGCGAGAGAGGAGGTGATAGAGGCGACCGGCTTGA |
| | | CAGAGCCAGAACCCCTGCCACAAAGCGATCGTTCAGCAAGGAGGTC |
| | | GAAGAGAGGTCCAGGGAGCGCCCTAGCCAGCCGGAAGGACTCAGA |
| | | AAGGCAGCCAGTCTAACAGAGGACCGCGACAGGGGAAGAGACGCC |
| | | GTGAAGAGGGAGGCCGCACTGCCTCCTGTGAGCCCTCTGAAGGCGG |
| | | CACTGAGCGAAGAAGAACTTGAGAAGAAGAGTAAGGCGATCATCG |
| | | AGGAGTACCTGCACCTGAACGACATGAAGGAGGCCGTGCAGTGCGT |
| | | GCAAGAGCTCGCGAGCCCATCACTGCTGTTCATCTTCGTCCGGCACG |
| | | GCGTGGAGTCCACACTGGAAAGATCTGCCATTGCTAGGGAGCATAT |
| | | GGGCCAGTTGTTGCACCAATTGCTTTGCGCCGGCCACCTAAGTACTG |
| | | CCCAGTACTATCAGGGTTTATACGAGATCCTCGAACTGGCCGAAGAC |
| | | ATGGAGATCGACATCCCTCACGTGTGGCTGTACCTCGCAGAACTGGT |
| | | GACCCCTATCCTGCAGGAGGGCGGCGTTCCAATGGGTGAGTTGTTCA |
| | | GAGAAATCACAAAGCCACTGCGCCCACTGGGCAAGGCGGCTAGTCT |
| | | CCTTCTGGAGATTCTCGGCCTGCTCTGTAAGAGCATGGGTCCAAAGA |
| | | AGGTGGGCACCCTGTGGAGGGAAGCTGGACTCTCCTGGAAGGAATT |
| | | CCTCCCTGAGGGTCAGGACATCGGCGCCTTCGTGGCCGAACAGAAG |
| | | GTTGAGTACACCCTGGGAGAGGAATCGGAAGCGCCGGGTCAGCGGG |
| | | CTCTGCCGAGTGAGGAGCTCAACAGACAACTCGAGAAGCTCCTCAA |
| | | GGAGGGATCCTCAAATCAGAGAGTGTTCGACTGGATCGAGGCCAAT |
| | | CTCAGCGAGCAGCAAATCGTGAGCAACACGTTGGTTCGCGCTCTCAT |
| | | GACAGCCGTGTGCTACTCAGCCATTATCTTCGAGACGCCTCTCCGCG |
| | | TGGACGTGGCAGTGCTCAAGGCCCGCGCTAAGCTGTTACAGAAGTA |
| | | CCTGTGCGACGAGCAGAAGGAACTGCAGGCCCTGTACGCCCTGCAA |
| | | GCTCTGGTCGTCACACTCGAGCAGCCTCCTAACCTGCTGAGAATGTT |
| | | CTTCGACGCATTGTACGACGAGGACGTTGTGAAGGAAGACGCTTTCT |
| | | ACAGCTGGGAGTCTAGTAAGGATCCTGCGGAACAACAAGGCAAGGG |
| | | CGTTGCTCTAAAGAGCGTGACCGCCTTCTTCAAGTGGCTGCGAGAGG |
| | | CTGAGGAAGAGTCGGACCACAAC |
| 31 | MBP eIF4G- | MASNFTQFVLVDNGGTGDVTVAPSNFANGIAEWISSNSRSQAYKVTCS |
| | dN2 (654- | VRQSSAQNRKYTIKVEVPKGAWRSYLNMELTIPIFATNSDCELIVKAMQ |
| | 1599) (aa) | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKFIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAVPTESTDNRRVVQRSSLSRERGEKAGDRGDRLER |
| | | SERGGDRGDRLDRARTPATKRSFSKEVEERSRERPSQPEGLRKAASLTE |
| | | DRDRGRDAVKREAALPPVSPLKAALSEEELEKKSKAIIEEYLHLNDMKE |
| | | AVQCVQELASPSLLFIFVRHGVESTLERSAIAREHMGQLLHQLLCAGHL |
| | | STAQYYQGLYEILELAEDMEIDIPHVWLYLAELVTPILQEGGVPMGELF |
| | | REITKPLRPLGKAASLLLEILGLLCKSMGPKKVGTLWREAGLSWKEFLP |
| | | EGQDIGAFVAEQKVEYTLGEESEAPGQRALPSEELNRQLEKLLKEGSSN |
| | | QRVFDWIEANLSEQQIVSNTLVRALMTAVCYSAIIFETPLRVDVAVLKA |
| | | RAKLLQKYLCDEQKELQALYALQALVVTLEQPPNLLRMFFDALYDED |
| | | VVKEDAFYSWESSKDPAEQQGKGVALKSVTAFFKWLREAEEESDHN |
| 32 | MBP eIF4G- | ATGGCCAGCAACTTCACCCAGTTCGTGCTGGTGGACAACGGCGGTA |
| | dN2 (654- | CCGGAGACGTGACCGTGGCCCCTTCTAACTTCGCCAACGGCATCGCC |
| | 1599) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTIGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCA |
| | | TCGAGGAGTACCTGCACCTGAACGACATGAAGGAGGCCGTGCAGTG |
| | | CGTGCAAGAGCTCGCGAGCCCATCACTGCTGTTCATCTTCGTCCGGC |
| | | ACGGCGTGGAGTCCACACTGGAAAGATCTGCCATTGCTAGGGAGCA |
| | | TATGGGCCAGTTGTTGCACCAATTGCTTTGCGCCGGCCACCTAAGTA |
| | | CTGCCCAGTACTATCAGGGTTTATACGAGATCCTCGAACTGGCCGAA |
| | | GACATGGAGATCGACATCCCTCACGTGTGGCTGTACCTCGCAGAACT |
| | | GGTGACCCCTATCCTGCAGGAGGGCGGCGTTCCAATGGGTGAGTTGT |
| | | TCAGAGAAATCACAAAGCCACTGCGCCCACTGGGCAAGGCGGCTAG |
| | | TCTCCTTCTGGAGATTCTCGGCCTGCTCTGTAAGAGCATGGGTCCAA |
| | | AGAAGGTGGGCACCCTGTGGAGGGAAGCTGGACTCTCCTGGAAGGA |
| | | ATTCCTCCCTGAGGGTCAGGACATCGGCGCCTTCGTGGCCGAACAGA |
| | | AGGTTGAGTACACCCTGGGAGAGGAATCGGAAGCGCCGGGTCAGCG |
| | | GGCTCTGCCGAGTGAGGAGCTCAACAGACAACTCGAGAAGCTCCTC |
| | | AAGGAGGGATCCTCAAATCAGAGAGTGTTCGACTGGATCGAGGCCA |
| | | ATCTCAGCGAGCAGCAAATCGTGAGCAACACGTTGGTTCGCGCTCTC |
| | | ATGACAGCCGTGTGCTACTCAGCCATTATCTTCGAGACGCCTCTCCG |
| | | CGTGGACGTGGCAGTGCTCAAGGCCCGCGCTAAGCTGTTACAGAAG |
| | | TACCTGTGCGACGAGCAGAAGGAACTGCAGGCCCTGTACGCCCTGC |
| | | AAGCTCTGGTCGTCACACTCGAGCAGCCTCCTAACCTGCTGAGAATG |
| | | TTCTTCGACGCATTGTACGACGAGGACGTTGTGAAGGAAGACGCTTT |
| | | CTACAGCTGGGAGTCTAGTAAGGATCCTGCGGAACAACAAGGCAAG |
| | | GGCGTTGCTCTAAAGAGCGTGACCGCCTTCTTCAAGTGGCTGCGAGA |
| | | GGCTGAGGAAGAGTCGGACCACAAC |
| 33 | MBP eIF4G- | MASNFTQFVLVDNGGTGDVTVAPSNFANGIAEWISSNSRSQAYKVTCS |
| | dN3 (654- | VRQSSAQNRKYTIKVEVPKGAWRSYLNMELTIPIFATNSDCELIVKAMQ |
| | 1451) (aa) | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKFIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAVPTESTDNRRVVQRSSLSRERGEKAGDRGDRLER |
| | | SERGGDRGDRLDRARTPATKRSFSKEVEERSRERPSQPEGLRKAASLTE |
| | | DRDRGRDAVKREAALPPVSPLKAALSEEELEKKSKAIIEEYLHLNDMKE |
| | | AVQCVQELASPSLLFIFVRHGVESTLERSAIAREHMGQLLHQLLCAGHL |
| | | STAQYYQGLYEILELAEDMEIDIPHVWLYLAELVTPILQEGGVPMGELF |
| | | REITKPLRPLGKAASLLLEILGLLCKSMGPKKVGTLWREAGLSWKEFLP |
| | | EGQDIGAFVAEQKVEYTLGEESEAPGQRALPSEELNRQLEKLL |
| 34 | MBP eIF4G- | ATGGCCAGCAACTTCACCCAGTTCGTGCTGGTGGACAACGGCGGTA |
| | dN3 (654- | CCGGAGACGTGACCGTGGCCCCTTCTAACTTCGCCAACGGCATCGCC |
| | 1451) (nt) | GCAGCGTGAGACAGAGCAGCGCCCAGAACAGAAAGTACACCATCA |
| | | GAGTGGATCAGCAGCAACAGCAGAAGCCAGGCCTACAAGGTGACCT |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTIGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCA |
| | | TCGAGGAGTACCTGCACCTGAACGACATGAAGGAGGCCGTGCAGTG |
| | | CGTGCAAGAGCTCGCGAGCCCATCACTGCTGTTCATCTTCGTCCGGC |
| | | ACGGCGTGGAGTCCACACTGGAAAGATCTGCCATTGCTAGGGAGCA |
| | | TATGGGCCAGTTGTTGCACCAATTGCTTTGCGCCGGCCACCTAAGTA |
| | | CTGCCCAGTACTATCAGGGTTTATACGAGATCCTCGAACTGGCCGAA |
| | | GACATGGAGATCGACATCCCTCACGTGTGGCTGTACCTCGCAGAACT |
| | | GGTGACCCCTATCCTGCAGGAGGGCGGCGTTCCAATGGGTGAGTTGT |
| | | TCAGAGAAATCACAAAGCCACTGCGCCCACTGGGCAAGGCGGCTAG |
| | | TCTCCTTCTGGAGATTCTCGGCCTGCTCTGTAAGAGCATGGGTCCAA |
| | | AGAAGGTGGGCACCCTGTGGAGGGAAGCTGGACTCTCCTGGAAGGA |
| | | ATTCCTCCCTGAGGGTCAGGACATCGGCGCCTTCGTGGCCGAACAGA |
| | | AGGTTGAGTACACCCTGGGAGAGGAATCGGAAGCGCCGGGTCAGCG |
| | | GGCTCTGCCGAGTGAGGAGCTCAACAGACAACTCGAGAAGCTCCTC |
| 35 | MBP eIF4G- | MASNFTQFVLVDNGGTGDVTVAPSNFANGIAEWISSNSRSQAYKVTCS |
| | mid1 (674- | VRQSSAQNRKYTIKVEVPKGAWRSYLNMELTIPIFATNSDCELIVKAMQ |
| | 1079) (aa) | GLLKDGNPIPSAIAANSGIYGGGGSGRTTLSTRGPPRGGPGGELPRGPAG |
| | | LGPRRSQQGPRKEPRKIIATVLMTEDIKLNKAEKAWKPSSKRTAADKDR |
| | | GEEDADGSKTQDLFRRVRSILNKLTPQMFQQLMKQVTQLAIDTEERLK |
| | | GVIDLIFEKAISEPNFSVAYANMCRCLMALKVPTTEKPTVTVNFRKLLL |
| | | NRCQKEFEKDKDDDEVFEKKQKEMDEAATAEERGRLKEELEEARDIAR |
| | | RRSLGNIKFIGELFKLKMLTEAIMHDCVVKLLKNHDEESLECLCRLLTTI |
| | | GKDLDFEKAKPRMDQYFNQMEKIIKEKKTSSRIRFMLQDVLDLRGSNW |
| | | VPRRGDQGPKTIDQIHKEAEMEEHREHIKVQQLMAKGSDKRRGGPPGP |
| | | PISRGLPLVDDGGWNTVPISKGSRPIDTSRLTKITKPGSID |
| 36 | MBP eIF4G- | ATGGCCAGCAACTTCACCCAGTTCGTGCTGGTGGACAACGGCGGTA |
| | mid1 (674- | CCGGAGACGTGACCGTGGCCCCTTCTAACTTCGCCAACGGCATCGCC |
| | 1079) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGGC |
| | | AGAACCACCCTGAGCACCAGAGGCCCTCCTAGAGGTGGTCCCGGCG |
| | | GAGAACTCCCCAGGGGTCCTGCCGGCCTGGGCCCTAGACGCTCCCA |
| | | GCAAGGTCCTAGAAAGGAGCCAAGGAAGATCATCGCCACCGTGCTG |
| | | ATGACCGAGGACATCAAGCTGAACAAAGCTGAGAAGGCCTGGAAGC |
| | | CTAGCAGCAAGAGAACCGCCGCCGACAAGGACAGAGGCGAGGAGG |
| | | ACGCCGACGGATCCAAGACCCAGGACCTGTTCAGAAGAGTGAGAAG |
| | | CATCCTCAACAAGCTGACCCCTCAGATGTTCCAGCAGCTGATGAAGC |
| | | AGGTGACGCAGCTCGCCATCGACACCGAGGAGAGACTGAAGGGCGT |
| | | GATCGACCTGATCTTTGAGAAGGCTATCTCAGAGCCTAACTTCAGCG |
| | | TGGCCTACGCCAACATGTGCCGTTGCCTGATGGCATTGAAGGTGCCA |
| | | ACCACCGAGAAGCCTACTGTGACCGTCAATTTCCGTAAACTGCTGCT |
| | | GAACCGGTGCCAGAAAGAGTTCGAGAAGGATAAGGACGACGACGA |
| | | GGTCTTCGAGAAGAAACAGAAAGAAATGGACGAGGCCGCCACCGC |
| | | AGAGGAAAGGGGCCGATTAAAGGAGGAGCTGGAGGAGGCCAGAGA |
| | | CATCGCCAGACGGCGTTCTCTGGGCAACATCAAGTTCATAGGTGAGC |
| | | TGTTCAAGCTAAAGATGCTCACCGAGGCCATAATGCACGACTGCGT |
| | | GGTGAAGCTACTGAAGAACCACGACGAAGAAAGCCTGGAGTGCCTG |
| | | TGCAGACTGCTGACCACCATCGGCAAGGACCTGGACTTCGAGAAGG |
| | | CAAAGCCTCGAATGGACCAGTACTTCAACCAGATGGAGAAGATTAT |
| | | CAAGGAGAAGAAGACCAGCAGCAGAATCAGATTCATGCTGCAGGAC |
| | | GTACTGGACCTGCGCGGAAGCAACTGGGTGCCAAGGAGAGGGGACC |
| | | AAGGACCAAAGACCATCGACCAGATCCACAAGGAAGCGGAGATGG |
| | | AGGAGCACAGAGAGCACATAAAGGTGCAGCAGCTTATGGCCAAGG |
| | | GCAGCGACAAGCGAAGAGGCGGCCCGCCCGGACCTCCTATCAGCAG |
| | | AGGCCTTCCTCTGGTAGACGACGGCGGCTGGAACACCGTGCCTATCT |
| | | CTAAGGGCTCCAGACCTATCGACACTTCCCGTCTTACCAAGATCACC |
| | | AAGCCAGGATCTATTGAC |
| 37 | MBP eIF4G- | MASNFTQFVLVDNGGTGDVTVAPSNFANGIAEWISSNSRSQAYKVTCS |
| | mid2 (654- | VRQSSAQNRKYTIKVEVPKGAWRSYLNMELTIPIFATNSDCELIVKAMQ |
| | 1130) (aa) | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKFIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAV |
| 38 | MBP eIF4G- | ATGGCCAGCAACTTCACCCAGTTCGTGCTGGTGGACAACGGCGGTA |
| | mid2 (654- | CCGGAGACGTGACCGTGGCCCCTTCTAACTTCGCCAACGGCATCGCC |
| | 1130) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTG |
| 39 | MBP eIF4G- | MASNFTQFVLVDNGGTGDVTVAPSNFANGIAEWISSNSRSQAYKVTCS |
| | mid3 (712- | VRQSSAQNRKYTIKVEVPKGAWRSYLNMELTIPIFATNSDCELIVKAMQ |
| | 1130) (aa) | GLLKDGNPIPSAIAANSGIYGGGGSPRKIIATVLMTEDIKLNKAEKAWKP |
| | | SSKRTAADKDRGEEDADGSKTQDLFRRVRSILNKLTPQMFQQLMKQVT |
| | | QLAIDTEERLKGVIDLIFEKAISEPNFSVAYANMCRCLMALKVPTTEKPT |
| | | VTVNFRKLLLNRCQKEFEKDKDDDEVFEKKQKEMDEAATAEERGRLK |
| | | EELEEARDIARRRSLGNIKFIGELFKLKMLTEAIMHDCVVKLLKNHDEE |
| | | SLECLCRLLTTIGKDLDFEKAKPRMDQYFNQMEKIIKEKKTSSRIRFMLQ |
| | | DVLDLRGSNWVPRRGDQGPKTIDQIHKEAEMEEHREHIKVQQLMAKG |
| | | SDKRRGGPPGPPISRGLPLVDDGGWNTVPISKGSRPIDTSRLTKITKPGSI |
| | | DSNNQLFAPGGRLSWGKGSSGGSGAKPSDAASEAARPATSTLNRFSAL |
| | | QQAV |
| 40 | MBP eIF4G- | ATGGCCAGCAACTTCACCCAGTTCGTGCTGGTGGACAACGGCGGTA |
| | mid3 (712- | CCGGAGACGTGACCGTGGCCCCTTCTAACTTCGCCAACGGCATCGCC |
| | 1130) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCCCA |
| | | AGGAAGATCATCGCCACCGTGCTGATGACCGAGGACATCAAGCTGA |
| | | ACAAAGCTGAGAAGGCCTGGAAGCCTAGCAGCAAGAGAACCGCCG |
| | | CCGACAAGGACAGAGGCGAGGAGGACGCCGACGGATCCAAGACCC |
| | | AGGACCTGTTCAGAAGAGTGAGAAGCATCCTCAACAAGCTGACCCC |
| | | TCAGATGTTCCAGCAGCTGATGAAGCAGGTGACGCAGCTCGCCATC |
| | | GACACCGAGGAGAGACTGAAGGGCGTGATCGACCTGATCTTTGAGA |
| | | AGGCTATCTCAGAGCCTAACTTCAGCGTGGCCTACGCCAACATGTGC |
| | | CGTTGCCTGATGGCATTGAAGGTGCCAACCACCGAGAAGCCTACTGT |
| | | GACCGTCAATTTCCGTAAACTGCTGCTGAACCGGTGCCAGAAAGAG |
| | | TTCGAGAAGGATAAGGACGACGACGAGGTCTTCGAGAAGAAACAG |
| | | AAAGAAATGGACGAGGCCGCCACCGCAGAGGAAAGGGGCCGATTA |
| | | AAGGAGGAGCTGGAGGAGGCCAGAGACATCGCCAGACGGCGTTCTC |
| | | TGGGCAACATCAAGTTCATAGGTGAGCTGTTCAAGCTAAAGATGCTC |
| | | ACCGAGGCCATAATGCACGACTGCGTGGTGAAGCTACTGAAGAACC |
| | | ACGACGAAGAAAGCCTGGAGTGCCTGTGCAGACTGCTGACCACCAT |
| | | CGGCAAGGACCTGGACTTCGAGAAGGCAAAGCCTCGAATGGACCAG |
| | | TACTTCAACCAGATGGAGAAGATTATCAAGGAGAAGAAGACCAGCA |
| | | GCAGAATCAGATTCATGCTGCAGGACGTACTGGACCTGCGCGGAAG |
| | | CAACTGGGTGCCAAGGAGAGGGGACCAAGGACCAAAGACCATCGA |
| | | CCAGATCCACAAGGAAGCGGAGATGGAGGAGCACAGAGAGCACAT |
| | | AAAGGTGCAGCAGCTTATGGCCAAGGGCAGCGACAAGCGAAGAGG |
| | | CGGCCCGCCCGGACCTCCTATCAGCAGAGGCCTTCCTCTGGTAGACG |
| | | ACGGCGGCTGGAACACCGTGCCTATCTCTAAGGGCTCCAGACCTATC |
| | | GACACTTCCCGTCTTACCAAGATCACCAAGCCAGGATCTATTGACAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCTGTG |
| 41 | MBP eIF4G- | MASNFTQFVLVDNGGTGDVTVAPSNFANGIAEWISSNSRSQAYKVTCS |
| | C1 (1080- | VRQSSAQNRKYTIKVEVPKGAWRSYLNMELTIPIFATNSDCELIVKAMQ |
| | 1599) (aa) | GLLKDGNPIPSAIAANSGIYGGGGSSNNQLFAPGGRLSWGKGSSGGSGA |
| | | KPSDAASEAARPATSTLNRFSALQQAVPTESTDNRRVVQRSSLSRERGE |
| | | KAGDRGDRLERSERGGDRGDRLDRARTPATKRSFSKEVEERSRERPSQP |
| | | EGLRKAASLTEDRDRGRDAVKREAALPPVSPLKAALSEEELEKKSKAII |
| | | EEYLHLNDMKEAVQCVQELASPSLLFIFVRHGVESTLERSAIAREHMGQ |
| | | LLHQLLCAGHLSTAQYYQGLYEILELAEDMEIDIPHVWLYLAELVTPIL |
| | | QEGGVPMGELFREITKPLRPLGKAASLLLEILGLLCKSMGPKKVGTLWR |
| | | EAGLSWKEFLPEGQDIGAFVAEQKVEYTLGEESEAPGQRALPSEELNRQ |
| | | LEKLLKEGSSNQRVFDWIEANLSEQQIVSNTLVRALMTAVCYSAIIFETP |
| | | LRVDVAVLKARAKLLQKYLCDEQKELQALYALQALVVTLEQPPNLLR |
| | | MFFDALYDEDVVKEDAFYSWESSKDPAEQQGKGVALKSVTAFFKWLR |
| | | EAEEESDHN |
| 42 | MBP eIF4G- | ATGGCCAGCAACTTCACCCAGTTCGTGCTGGTGGACAACGGCGGTA |
| | C1 (1080- | CCGGAGACGTGACCGTGGCCCCTTCTAACTTCGCCAACGGCATCGCC |
| | 1599) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCAGC |
| | | AACAACCAGCTGTTCGCCCCAGGAGGAAGACTTAGCTGGGGCAAGG |
| | | GAAGTTCCGGCGGATCCGGCGCCAAGCCTTCCGACGCCGCCAGCGA |
| | | GGCTGCCAGACCTGCCACCAGCACCTTGAACCGCTTTTCCGCTCTGC |
| | | AGCAAGCTGTGCCGACCGAGAGCACCGACAACCGGCGAGTTGTGCA |
| | | GAGAAGCAGCCTGAGCAGAGAGAGGGGCGAGAAAGCCGGCGATAG |
| | | AGGTGACCGACTGGAGAGAAGCGAGAGAGGAGGTGATAGAGGCGA |
| | | CCGGCTTGACAGAGCCAGAACCCCTGCCACAAAGCGATCGTTCAGC |
| | | AAGGAGGTCGAAGAGAGGTCCAGGGAGCGCCCTAGCCAGCCGGAA |
| | | GGACTCAGAAAGGCAGCCAGTCTAACAGAGGACCGCGACAGGGGA |
| | | AGAGACGCCGTGAAGAGGGAGGCCGCACTGCCTCCTGTGAGCCCTC |
| | | TGAAGGCGGCACTGAGCGAAGAAGAACTTGAGAAGAAGAGTAAGG |
| | | CGATCATCGAGGAGTACCTGCACCTGAACGACATGAAGGAGGCCGT |
| | | GCAGTGCGTGCAAGAGCTCGCGAGCCCATCACTGCTGTTCATCTTCG |
| | | TCCGGCACGGCGTGGAGTCCACACTGGAAAGATCTGCCATTGCTAG |
| | | GGAGCATATGGGCCAGTTGTTGCACCAATTGCTTTGCGCCGGCCACC |
| | | TAAGTACTGCCCAGTACTATCAGGGTTTATACGAGATCCTCGAACTG |
| | | GCCGAAGACATGGAGATCGACATCCCTCACGTGTGGCTGTACCTCGC |
| | | AGAACTGGTGACCCCTATCCTGCAGGAGGGCGGCGTTCCAATGGGT |
| | | GAGTTGTTCAGAGAAATCACAAAGCCACTGCGCCCACTGGGCAAGG |
| | | CGGCTAGTCTCCTTCTGGAGATTCTCGGCCTGCTCTGTAAGAGCATG |
| | | GGTCCAAAGAAGGTGGGCACCCTGTGGAGGGAAGCTGGACTCTCCT |
| | | GGAAGGAATTCCTCCCTGAGGGTCAGGACATCGGCGCCTTCGTGGC |
| | | CGAACAGAAGGTTGAGTACACCCTGGGAGAGGAATCGGAAGCGCCG |
| | | GGTCAGCGGGCTCTGCCGAGTGAGGAGCTCAACAGACAACTCGAGA |
| | | AGCTCCTCAAGGAGGGATCCTCAAATCAGAGAGTGTTCGACTGGAT |
| | | CGAGGCCAATCTCAGCGAGCAGCAAATCGTGAGCAACACGTTGGTT |
| | | CGCGCTCTCATGACAGCCGTGTGCTACTCAGCCATTATCTTCGAGAC |
| | | GCCTCTCCGCGTGGACGTGGCAGTGCTCAAGGCCCGCGCTAAGCTGT |
| | | TACAGAAGTACCTGTGCGACGAGCAGAAGGAACTGCAGGCCCTGTA |
| | | CGCCCTGCAAGCTCTGGTCGTCACACTCGAGCAGCCTCCTAACCTGC |
| | | TGAGAATGTTCTTCGACGCATTGTACGACGAGGACGTTGTGAAGGA |
| | | AGACGCTTTCTACAGCTGGGAGTCTAGTAAGGATCCTGCGGAACAA |
| | | CAAGGCAAGGGCGTTGCTCTAAAGAGCGTGACCGCCTTCTTCAAGT |
| | | GGCTGCGAGAGGCTGAGGAAGAGTCGGACCACAAC |
| 43 | MBP eIF4G- | MASNFTQFVLVDNGGTGDVTVAPSNFANGIAEWISSNSRSQAYKVTCS |
| | C2 (1080- | VRQSSAQNRKYTIKVEVPKGAWRSYLNMELTIPIFATNSDCELIVKAMQ |
| | 1451) (aa) | GLLKDGNPIPSAIAANSGIYGGGGSSNNQLFAPGGRLSWGKGSSGGSGA |
| | | KPSDAASEAARPATSTLNRFSALQQAVPTESTDNRRVVQRSSLSRERGE |
| | | KAGDRGDRLERSERGGDRGDRLDRARTPATKRSFSKEVEERSRERPSQP |
| | | EGLRKAASLTEDRDRGRDAVKREAALPPVSPLKAALSEEELEKKSKAII |
| | | EEYLHLNDMKEAVQCVQELASPSLLFIFVRHGVESTLERSAIAREHMGQ |
| | | LLHQLLCAGHLSTAQYYQGLYEILELAEDMEIDIPHVWLYLAELVTPIL |
| | | QEGGVPMGELFREITKPLRPLGKAASLLLEILGLLCKSMGPKKVGTLWR |
| | | EAGLSWKEFLPEGQDIGAFVAEQKVEYTLGEESEAPGQRALPSEELNRQ |
| | | LEKLL |
| 44 | MBP eIF4G- | ATGGCCAGCAACTTCACCCAGTTCGTGCTGGTGGACAACGGCGGTA |
| | C2 (1080- | CCGGAGACGTGACCGTGGCCCCTTCTAACTTCGCCAACGGCATCGCC |
| | 1451) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCAGC |
| | | AACAACCAGCTGTTCGCCCCAGGAGGAAGACTTAGCTGGGGCAAGG |
| | | GAAGTTCCGGCGGATCCGGCGCCAAGCCTTCCGACGCCGCCAGCGA |
| | | GGCTGCCAGACCTGCCACCAGCACCTTGAACCGCTTTTCCGCTCTGC |
| | | AGCAAGCTGTGCCGACCGAGAGCACCGACAACCGGCGAGTTGTGCA |
| | | GAGAAGCAGCCTGAGCAGAGAGAGGGGCGAGAAAGCCGGCGATAG |
| | | AGGTGACCGACTGGAGAGAAGCGAGAGAGGAGGTGATAGAGGCGA |
| | | CCGGCTTGACAGAGCCAGAACCCCTGCCACAAAGCGATCGTTCAGC |
| | | AAGGAGGTCGAAGAGAGGTCCAGGGAGCGCCCTAGCCAGCCGGAA |
| | | GGACTCAGAAAGGCAGCCAGTCTAACAGAGGACCGCGACAGGGGA |
| | | AGAGACGCCGTGAAGAGGGAGGCCGCACTGCCTCCTGTGAGCCCTC |
| | | TGAAGGCGGCACTGAGCGAAGAAGAACTTGAGAAGAAGAGTAAGG |
| | | CGATCATCGAGGAGTACCTGCACCTGAACGACATGAAGGAGGCCGT |
| | | GCAGTGCGTGCAAGAGCTCGCGAGCCCATCACTGCTGTTCATCTTCG |
| | | TCCGGCACGGCGTGGAGTCCACACTGGAAAGATCTGCCATTGCTAG |
| | | GGAGCATATGGGCCAGTTGTTGCACCAATTGCTTTGCGCCGGCCACC |
| | | TAAGTACTGCCCAGTACTATCAGGGTTTATACGAGATCCTCGAACTG |
| | | GCCGAAGACATGGAGATCGACATCCCTCACGTGTGGCTGTACCTCGC |
| | | AGAACTGGTGACCCCTATCCTGCAGGAGGGCGGCGTTCCAATGGGT |
| | | GAGTTGTTCAGAGAAATCACAAAGCCACTGCGCCCACTGGGCAAGG |
| | | CGGCTAGTCTCCTTCTGGAGATTCTCGGCCTGCTCTGTAAGAGCATG |
| | | GGTCCAAAGAAGGTGGGCACCCTGTGGAGGGAAGCTGGACTCTCCT |
| | | GGAAGGAATTCCTCCCTGAGGGTCAGGACATCGGCGCCTTCGTGGC |
| | | CGAACAGAAGGTTGAGTACACCCTGGGAGAGGAATCGGAAGCGCCG |
| | | GGTCAGCGGGCTCTGCCGAGTGAGGAGCTCAACAGACAACTCGAGA |
| | | AGCTCCTC |
| 45 | MBP-CTIF | MASNFTQFVLVDNGGTGDVTVAPSNFANGIAEWISSNSRSQAYKVTCS |
| | (aa) | VRQSSAQNRKYTIKVEVPKGAWRSYLNMELTIPIFATNSDCELIVKAMQ |
| | | GLLKDGNPIPSAIAANSGIYGGGGSENSSAASASSEAGSSRSQEIEELERF |
| | | IDSYVLEYQVQGLLADKTEGDGESERTQSHISQWTADCSEPLDSSCSFS |
| | | RGRAPPQQNGSKDNSLDMLGTDIWAANTFDSFSGATWDLQPEKLDFTQ |
| | | FHRKVRHTPKQPLPHIDREGCGKGKLEDGDGINLNDIEKVLPAWQGYH |
| | | PMPHEVEIAHTKKLFRRRRNDRRRQQRPPGGNKPQQHGDHQPGSAKH |
| | | NRDHQKSYQGGSAPHPSGRPTHHGYSQNRRWHHGNMKHPPGDKGEA |
| | | GAHRNAKETMTIENPKLEDTAGDTGHSSLEAPRSPDTLAPVASERLPPQ |
| | | QSGGPEVETKRKDSILPERIGERPKITLLQSSKDRLRRRLKEKDEVAVET |
| | | TTPQQNKMDKLIEILNSMRNNSSDVDTKLTTFMEEAQNSTNSEEMLGEI |
| | | VRTIYQKAVSDRSFAFTAAKLCDKMALFMVEGTKFRSLLLNMLQKDFT |
| | | VREELQQQDVERWLGFITFLCEVFGTMRSSTGEPFRVLVCPIYTCLRELL |
| | | QSQDVKEDAVLCCSMELQSTGRLLEEQLPEMMTELLASARDKMLCPSE |
| | | SMLTRSLLLEVIELHANSWNPLTPPITQYYNRTIQKLTA |
| 46 | MBP-CTIF | ATGGCCAGCAACTTCACCCAGTTCGTGCTGGTGGACAACGGCGGTA |
| | (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAG |
| | | AACAGTAGCGCCGCTAGCGCGAGCAGCGAAGCCGGGAGCAGCCGGT |
| | | CTCAGGAGATCGAGGAGCTGGAGCGGTTCATCGACAGCTACGTGCT |
| | | GGAGTACCAGGTGCAGGGCCTGCTGGCCGACAAGACCGAGGGCGAC |
| | | GGCGAGAGCGAGCGGACCCAGAGCCACATCAGCCAGTGGACCGCCG |
| | | ACTGCAGCGAGCCCCTGGACTCTAGCTGCTCATTCAGTCGTGGACGG |
| | | GCCCCTCCACAGCAGAACGGCAGCAAGGACAACAGCCTGGACATGC |
| | | TGGGCACCGACATCTGGGCCGCCAACACCTTCGACAGCTTCAGCGG |
| | | CGCCACCTGGGATCTGCAGCCCGAGAAGCTGGACTTTACCCAGTTCC |
| | | ACCGGAAGGTGCGGCACACTCCCAAGCAGCCCCTGCCCCACATCGA |
| | | TCGGGAGGGCTGCGGCAAGGGCAAGCTGGAAGACGGCGACGGCAT |
| | | CAACCTGAACGACATCGAGAAGGTGCTGCCTGCCTGGCAGGGCTAC |
| | | CACCCCATGCCCCACGAGGTGGAGATCGCCCACACCAAGAAGCTGT |
| | | TCCGGCGACGACGCAACGACCGGCGTAGGCAGCAACGGCCGCCTGG |
| | | AGGGAACAAGCCCCAGCAGCACGGAGACCACCAGCCCGGTAGCGCC |
| | | AAGCACAACCGGGACCACCAGAAGAGCTACCAGGGCGGAAGCGCA |
| | | CCACACCCCTCGGGCAGACCCACCCACCACGGCTACAGCCAGAACC |
| | | GGCGGTGGCATCACGGTAACATGAAGCACCCACCCGGCGACAAAGG |
| | | AGAGGCCGGCGCTCACCGTAACGCCAAGGAGACCATGACCATCGAG |
| | | AACCCCAAGCTGGAGGATACCGCCGGCGATACGGGTCACAGCAGCC |
| | | TGGAGGCACCGCGGTCTCCCGACACCCTGGCACCCGTGGCCAGCGA |
| | | ACGGCTGCCACCCCAACAGAGCGGCGGCCCTGAGGTTGAGACCAAG |
| | | CGGAAGGACAGCATCCTGCCCGAACGGATCGGTGAGCGGCCCAAGA |
| | | TCACCTTACTGCAGAGTAGCAAGGACCGGCTGAGACGGCGGCTGAA |
| | | GGAGAAGGACGAGGTGGCCGTGGAGACAACCACTCCCCAGCAGAA |
| | | CAAGATGGACAAGCTGATCGAGATCCTGAACAGCATGCGGAACAAC |
| | | AGCAGCGACGTGGACACCAAGCTGACCACCTTCATGGAGGAGGCCC |
| | | AGAACAGCACCAACAGCGAGGAGATGCTGGGCGAGATCGTGCGGA |
| | | CCATCTACCAGAAGGCCGTGAGCGACCGGAGCTTCGCCTTCACCGCC |
| | | GCCAAGCTGTGCGACAAGATGGCCCTGTTCATGGTGGAGGGCACCA |
| | | AGTTCCGGAGCTTACTGCTGAATATGCTGCAGAAGGACTTCACCGTG |
| | | CGGGAGGAGCTGCAGCAGCAGGACGTGGAGCGGTGGCTGGGCTTCA |
| | | TCACCTTCCTGTGCGAGGTGTTCGGCACCATGCGGAGCAGCACCGGC |
| | | GAACCCTTCCGGGTGCTGGTGTGCCCCATCTACACCTGCCTGCGGGA |
| | | GTTGCTGCAGAGCCAGGACGTGAAGGAGGACGCCGTGCTGTGCTGC |
| | | AGCATGGAACTGCAGAGCACTGGCCGGCTGCTGGAGGAGCAGCTGC |
| | | CCGAGATGATGACCGAGCTGCTCGCTAGCGCCCGGGACAAGATGCT |
| | | GTGCCCCAGCGAGAGCATGCTGACCCGGAGCCTGCTTCTGGAGGTG |
| | | ATCGAGCTGCACGCCAACAGCTGGAATCCCCTGACCCCTCCCATCAC |
| | | CCAGTACTACAACCGGACCATCCAGAAGCTGACCGCC |
| 47 | MBP- | MASNFTQFVLVDNGGTGDVTVAPSNFANGIAEWISSNSRSQAYKVTCS |
| | CTIF(379- | VRQSSAQNRKYTIKVEVPKGAWRSYLNMELTIPIFATNSDCELIVKAMQ |
| | 579) (aa) | GLLKDGNPIPSAIAANSGIYGGGGSLNSMRNNSSDVDTKLTTFMEEAQN |
| | | STNSEEMLGEIVRTIYQKAVSDRSFAFTAAKLCDKMALFMVEGTKFRSL |
| | | LLNMLQKDFTVREELQQQDVERWLGFITFLCEVFGTMRSSTGEPFRVL |
| | | VCPIYTCLRELLQSQDVKEDAVLCCSMELQSTGRLLEEQLPEMMTELLA |
| | | SARDKMLCPSESMLTRSLLLEVIELHANSWN |
| 48 | MBP- | ATGGCCAGCAACTTCACCCAGTTCGTGCTGGTGGACAACGGCGGTA |
| | CTIF(379- | CCGGAGACGTGACCGTGGCCCCTTCTAACTTCGCCAACGGCATCGCC |
| | 579) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCCTG |
| | | AACAGCATGAGAAACAACAGCAGCGACGTGGACACCAAGCTGACC |
| | | ACCTTCATGGAGGAGGCCCAGAACAGCACCAACAGCGAGGAGATGC |
| | | TGGGCGAGATCGTGAGAACCATCTACCAGAAGGCCGTGAGCGACAG |
| | | AAGCTTCGCCTTCACCGCCGCCAAGCTGTGCGACAAGATGGCCCTGT |
| | | TCATGGTGGAGGGCACCAAGTTCAGAAGCCTGCTGCTGAACATGCT |
| | | GCAGAAGGACTTCACCGTGAGAGAGGAGCTGCAGCAGCAGGACGTG |
| | | GAGCGATGGCTGGGCTTCATCACCTTCCTGTGCGAGGTGTTCGGCAC |
| | | CATGAGAAGCAGCACCGGCGAGCCTTTCAGAGTGCTGGTGTGCCCT |
| | | ATCTACACCTGCCTGAGAGAGCTGCTGCAGAGCCAGGACGTGAAGG |
| | | AGGACGCCGTGCTGTGCTGCAGCATGGAGCTGCAGAGCACCGGCAG |
| | | ACTGCTGGAGGAGCAGCTGCCTGAGATGATGACCGAGCTGCTGGCC |
| | | AGCGCCAGAGACAAGATGCTGTGCCCTAGCGAGAGCATGCTGACTA |
| | | GATCCCTACTGTTGGAGGTGATCGAGCTGCACGCCAACAGCTGGAA |
| | | C |
| 49 | MBP- | MASNFTQFVLVDNGGTGDVTVAPSNFANGIAEWISSNSRSQAYKVTCS |
| | CTIF(365- | VRQSSAQNRKYTIKVEVPKGAWRSYLNMELTIPIFATNSDCELIVKAMQ |
| | 598) (aa) | GLLKDGNPIPSAIAANSGIYGGGGSTTPQQNKMDKLIEILNSMRNNSSD |
| | | VDTKLTTFMEEAQNSTNSEEMLGEIVRTIYQKAVSDRSFAFTAAKLCDK |
| | | MALFMVEGTKFRSLLLNMLQKDFTVREELQQQDVERWLGFITFLCEVF |
| | | GTMRSSTGEPFRVLVCPIYTCLRELLQSQDVKEDAVLCCSMELQSTGRL |
| | | LEEQLPEMMTELLASARDKMLCPSESMLTRSLLLEVIELHANSWNPLTP |
| | | PITQYYNRTIQKLTA |
| 50 | MBP- | ATGGCCAGCAACTTCACCCAGTTCGTGCTGGTGGACAACGGCGGTA |
| | CTIF(365- | CCGGAGACGTGACCGTGGCCCCTTCTAACTTCGCCAACGGCATCGCC |
| | 598) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCACC |
| | | ACCCCTCAGCAGAACAAGATGGACAAGCTGATCGAGATCCTGAACA |
| | | GCATGAGAAACAACAGCAGCGACGTGGACACCAAGCTGACCACCTT |
| | | CATGGAGGAGGCCCAGAACAGCACCAACAGCGAGGAGATGCTGGG |
| | | CGAGATCGTGAGAACCATCTACCAGAAGGCCGTGAGCGACAGAAGC |
| | | TTCGCCTTCACCGCCGCCAAGCTGTGCGACAAGATGGCCCTGTTCAT |
| | | GGTGGAGGGCACCAAGTTCAGAAGCCTGCTGCTGAACATGCTGCAG |
| | | AAGGACTTCACCGTGAGAGAGGAGCTGCAGCAGCAGGACGTGGAG |
| | | AGGTGGCTGGGCTTCATCACCTTCCTGTGCGAGGTGTTCGGCACCAT |
| | | GAGAAGCAGCACCGGCGAGCCTTTCAGAGTGCTGGTGTGCCCTATCT |
| | | ACACCTGCCTGAGAGAGCTGCTGCAGAGCCAGGACGTGAAGGAGGA |
| | | CGCCGTGCTGTGCTGCAGCATGGAGCTGCAGAGCACCGGCAGACTG |
| | | CTGGAGGAGCAGCTGCCTGAGATGATGACCGAGCTGCTGGCCAGCG |
| | | CCAGAGACAAGATGCTGTGCCCTAGCGAGAGCATGCTGACGCGCAG |
| | | CCTCCTGCTGGAGGTGATCGAGCTGCACGCCAACAGCTGGAACCCTC |
| | | TGACCCCTCCTATCACCCAGTACTACAACAGAACCATCCAGAAGCTG |
| | | ACCGCC |
| 51 | MBP-eIF4G- | MASNFTQFVLVDNGGTGDVTVAPSNFANGIAEWISSNSRSQAYKVTCS |
| | mid4 (752- | VRQSSAQNRKYTIKVEVPKGAWRSYLNMELTIPIFATNSDCELIVKAMQ |
| | 993) (aa) | GLLKDGNPIPSAIAANSGIYGGGGSADGSKTQDLFRRVRSILNKLTPQMF |
| | | QQLMKQVTQLAIDTEERLKGVIDLIFEKAISEPNFSVAYANMCRCLMAL |
| | | KVPTTEKPTVTVNFRKLLLNRCQKEFEKDKDDDEVFEKKQKEMDEAA |
| | | TAEERGRLKEELEEARDIARRRSLGNIKFIGELFKLKMLTEAIMHDCVV |
| | | KLLKNHDEESLECLCRLLTTIGKDLDFEKAKPRMDQYFNQMEKIIKEKK |
| | | TSSRIRFMLQDVLDLRGSNWVPR |
| 52 | MBP-eIF4G- | ATGGCCAGCAACTTCACCCAGTTCGTGCTGGTGGACAACGGCGGTA |
| | mid4 (752- | CCGGAGACGTGACCGTGGCCCCTTCTAACTTCGCCAACGGCATCGCC |
| | 993) (nt) | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGCC |
| | | GACGGCAGCAAGACCCAGGACCTGTTCAGAAGAGTGAGAAGCATCC |
| | | TGAACAAGCTGACCCCTCAGATGTTCCAGCAGCTGATGAAGCAGGT |
| | | GACCCAGCTGGCCATCGACACCGAGGAGAGACTGAAGGGCGTGATC |
| | | GACCTGATCTTCGAGAAGGCCATCAGCGAGCCTAACTTCAGCGTGG |
| | | CCTACGCCAACATGTGCCGGTGCCTGATGGCCCTGAAGGTGCCTACC |
| | | ACCGAGAAGCCTACCGTGACCGTGAACTTCAGAAAGCTGCTGCTGA |
| | | ACCGGTGCCAGAAGGAGTTCGAGAAGGACAAGGACGACGACGAGG |
| | | TGTTCGAGAAGAAGCAGAAGGAGATGGACGAGGCCGCCACCGCCG |
| | | AGGAGAGAGGCAGACTGAAGGAGGAGCTGGAGGAGGCCAGAGACA |
| | | TCGCCAGAAGAAGAAGCCTGGGCAACATCAAGTTCATCGGCGAGCT |
| | | GTTCAAGCTGAAGATGCTGACCGAGGCCATCATGCACGACTGCGTG |
| | | GTGAAGCTGCTGAAGAACCACGACGAGGAGAGCCTGGAGTGCCTGT |
| | | GCAGACTGCTGACCACCATCGGCAAGGACCTGGATTTCGAGAAGGC |
| | | GAAGCCTAGAATGGACCAGTACTTCAACCAGATGGAGAAGATCATC |
| | | AAGGAGAAGAAGACCAGCAGCAGAATCAGATTCATGCTGCAGGAC |
| | | GTGCTGGACCTGAGAGGCAGCAACTGGGTGCCTAGA |
| 53 | MBP-LacZ | MASNFTQFVLVDNGGTGDVTVAPSNFANGIAEWISSNSRSQAYKVTCS |
| | (aa) | VRQSSAQNRKYTIKVEVPKGAWRSYLNMELTIPIFATNSDCELIVKAMQ |
| | | GLLKDGNPIPSAIAANSGIYGGGGSSFTLTNKNVIFVAGLGGIGLDTSKE |
| | | LLKRDPVVLQRRDWENPGVTQLNRLAAHPPFASWRNSEEARTDRPSQQ |
| | | LRSLNGEWRFAWFPAPEAVPESWLECDLPEADTVVVPSNWQMHGYDA |
| | | PIYTNVTYPITVNPPFVPTENPTGCYSLTFNVDESWLQEGQTRIIFDGVNS |
| | | AFHLWCNGRWVGYGQDSRLPSEFDLSAFLRAGENRLAVMVLRWSDGS |
| | | YLEDQDMWRMSGIFRDVSLLHKPTTQISDFHVATRENDDFSRAVLEAE |
| | | VQMCGELRDYLRVTVSLWQGETQVASGTAPFGGEIIDERGGYADRVTL |
| | | RLNVENPKLWSAEIPNLYRAVVELHTADGTLIEAEACDVGFREVRIENG |
| | | LLLLNGKPLLIRGVNRHEHHPLHGQVMDEQTMVQDILLMKQNNFNAV |
| | | RCSHYPNHPLWYTLCDRYGLYVVDEANIETHGMVPMNRLTDDPRWLP |
| | | AMSERVTRMVQRDRNHPSVIIWSLGNESGHGANHDALYRWIKSVDPSR |
| | | PVQYEGGGADTTATDIICPMYARVDEDQPFPAVPKWSIKKWLSLPGET |
| | | RPLILCEYAHAMGNSLGGFAKYWQAFRQYPRLQGGFVWDWVDQSLIK |
| | | YDENGNPWSAYGGDFGDTPNDRQFCMNGLVFADRTPHPALTEAKHQQ |
| | | QFFQFRLSGQTIEVTSEYLFRHSDNELLHWMVALDGKPLASGEVPLDV |
| | | APQGKQLIELPELPQPESAGQLWLTVRVVQPNATAWSEAGHISAWQQ |
| | | WRLAENLSVTLPAASHAIPHLTTSEMDFCIELGNKRWQFNRQSGFLSQ |
| | | MWIGDKKQLLTPLRDQFTRAPLDNDIGVSEATRIDPNAWVERWKAAG |
| | | HYQAEAALLQCTADTLADAVLITTAHAWQHQGKTLFISRKTYRIDGSG |
| | | QMAITVDVEVASDTPHPARIGLNCQLAQVAERVNWLGLGPQENYPDRL |
| | | TAACFDRWDLPLSDMYTPYVFPSENGLRCGTRELNYGPHQWRGDFQF |
| | | NISRYSQQQLMETSHRHLLHAEEGTWLNIDGFHMGIGGDDSWSPSVSA |
| | | ELQLSAGRYHYQLVWCQK |
| 54 | MBP-LacZ | ATGGCCAGCAACTTCACCCAGTTCGTGCTGGTGGACAACGGCGGTA |
| | (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCTCG |
| | | TTCACGCTAACGAACAAGAACGTCATCTTCGTAGCGGGACTTGGCG |
| | | GTATCGGCCTAGACACGTCGAAGGAACTACTAAAGCGTGACCCGGT |
| | | AGTCCTCCAACGTCGCGATTGGGAGAACCCGGGCGTAACGCAACTA |
| | | AACCGTCTTGCGGCGCACCCGCCGTTTGCGTCGTGGCGTAACTCGGA |
| | | GGAGGCGCGAACGGATCGTCCGTCGCAACAACTACGTTCGCTCAAC |
| | | GGGGAGTGGCGCTTCGCGTGGTTCCCGGCGCCGGAGGCGGTACCGG |
| | | AGTCGTGGCTCGAGTGCGATCTACCGGAGGCGGACACGGTCGTCGT |
| | | ACCGTCGAACTGGCAAATGCACGGTTACGACGCGCCGATATACACG |
| | | AACGTCACGTACCCGATAACGGTAAACCCGCCGTTCGTCCCGACGG |
| | | AGAACCCGACGGGGTGCTACTCGCTAACGTTCAACGTTGACGAGTC |
| | | GTGGTTGCAAGAGGGTCAAACGCGTATCATATTCGACGGTGTAAAC |
| | | TCGGCGTTCCACCTGTGGTGCAACGGGCGCTGGGTAGGGTACGGCC |
| | | AAGACTCGCGTCTACCGTCGGAGTTCGACCTATCGGCGTTCCTACGA |
| | | GCGGGTGAGAACCGGCTAGCGGTCATGGTCCTACGTTGGTCGGACG |
| | | GTTCGTACCTCGAGGACCAAGACATGTGGCGAATGTCGGGTATCTTC |
| | | CGCGACGTATCGCTCCTACACAAGCCGACGACGCAAATCTCGGACTT |
| | | CCACGTCGCGACGCGTTTCAACGACGATTTCTCGCGGGCAGTCCTAG |
| | | AGGCGGAGGTCCAAATGTGCGGGGAGCTACGTGACTACCTCCGTGT |
| | | CACGGTATCGCTCTGGCAAGGTGAGACGCAAGTAGCGTCGGGTACG |
| | | GCGCCGTTCGGCGGTGAGATCATCGACGAGCGTGGTGGGTACGCGG |
| | | ACCGTGTAACGCTACGTCTAAACGTCGAGAACCCGAAGCTCTGGTC |
| | | GGCGGAGATCCCGAACCTATACCGTGCGGTCGTCGAGCTACATACG |
| | | GCGGACGGGACGCTAATAGAGGCGGAAGCGTGCGACGTCGGGTTTC |
| | | GAGAGGTTCGTATAGAGAACGGGCTGCTACTTCTAAACGGGAAGCC |
| | | GTTGCTCATACGTGGTGTCAACCGTCACGAGCACCACCCGCTACACG |
| | | GTCAAGTAATGGACGAGCAAACGATGGTACAAGACATCCTACTAAT |
| | | GAAGCAGAACAACTTCAACGCGGTACGCTGTTCGCATTACCCGAAC |
| | | CATCCGTTGTGGTACACGCTTTGCGACCGATACGGTCTATACGTCGT |
| | | AGACGAGGCGAACATAGAGACGCACGGGATGGTACCGATGAATCGC |
| | | CTAACGGACGACCCGCGTTGGCTACCGGCGATGTCGGAGCGAGTCA |
| | | CGCGTATGGTCCAACGGGACCGTAACCACCCGTCGGTAATAATCTG |
| | | GTCGCTAGGCAACGAATCGGGGCACGGGGCGAACCACGACGCGCTA |
| | | TACCGTTGGATCAAGTCGGTAGACCCGTCGCGTCCGGTACAATACGA |
| | | AGGTGGCGGTGCGGACACGACGGCGACGGACATCATCTGCCCGATG |
| | | TACGCGCGCGTCGACGAAGACCAACCGTTCCCGGCGGTACCGAAGT |
| | | GGTCGATCAAGAAGTGGCTCTCGTTGCCGGGTGAAACGCGTCCGTTG |
| | | ATACTTTGCGAGTACGCGCACGCGATGGGCAACTCGTTGGGTGGGTT |
| | | CGCGAAGTACTGGCAGGCGTTCCGTCAATACCCGCGTCTACAGGGT |
| | | GGGTTCGTCTGGGACTGGGTAGACCAATCGCTAATCAAGTACGACG |
| | | AGAACGGCAACCCGTGGTCGGCGTACGGTGGGGACTTCGGGGACAC |
| | | GCCGAACGACCGCCAATTCTGTATGAACGGCCTAGTCTTCGCGGACC |
| | | GAACGCCGCACCCGGCGTTGACGGAGGCGAAGCATCAACAACAATT |
| | | CTTCCAATTCCGTCTATCGGGGCAAACGATCGAGGTAACGTCGGAGT |
| | | ACTTGTTCCGGCACTCGGACAACGAGCTACTACACTGGATGGTAGCA |
| | | CTAGACGGCAAGCCGCTAGCGTCGGGAGAAGTCCCTTTGGACGTCG |
| | | CGCCGCAAGGTAAGCAACTAATCGAGCTACCGGAGCTACCGCAACC |
| | | GGAGTCGGCGGGTCAACTGTGGTTGACGGTCCGTGTCGTTCAACCGA |
| | | ACGCGACGGCGTGGTCGGAGGCGGGTCACATCTCGGCGTGGCAGCA |
| | | GTGGCGTCTAGCGGAGAACCTCTCGGTCACGCTACCGGCGGCGTCG |
| | | CACGCGATACCGCATCTAACGACGTCGGAGATGGACTTCTGCATCG |
| | | AGTTGGGGAACAAGAGGTGGCAGTTCAACCGTCAATCGGGATTCCT |
| | | ATCGCAAATGTGGATAGGTGACAAGAAGCAACTACTAACGCCGCTA |
| | | CGTGATCAGTTCACGCGTGCTCCGCTAGACAACGACATAGGTGTTTC |
| | | GGAGGCGACGCGTATAGACCCGAACGCGTGGGTGGAGCGGTGGAA |
| | | GGCGGCGGGGCACTACCAAGCGGAGGCGGCGCTACTACAGTGCACG |
| | | GCGGACACGCTAGCGGACGCGGTATTGATCACGACGGCGCACGCGT |
| | | GGCAACACCAGGGGAAGACGCTATTCATCTCGCGTAAGACGTACCG |
| | | TATCGACGGTTCGGGCCAAATGGCGATCACGGTCGACGTAGAGGTA |
| | | GCGTCGGACACGCCGCATCCGGCGCGCATCGGTCTAAACTGCCAAC |
| | | TAGCGCAAGTAGCGGAGCGTGTAAACTGGCTAGGGCTAGGGCCGCA |
| | | AGAGAACTATCCGGACCGCCTAACGGCGGCGTGCTTCGACCGTTGG |
| | | GACCTACCGCTTTCGGACATGTATACCCCGTACGTCTTCCCGTCGGA |
| | | GAACGGGTTGAGGTGCGGGACGCGCGAGCTAAACTACGGGCCGCAC |
| | | CAGTGGCGAGGGGACTTCCAATTCAACATATCGCGTTACTCGCAACA |
| | | ACAACTAATGGAGACGTCGCACCGTCACCTACTACACGCGGAGGAG |
| | | GGGACGTGGCTAAACATCGACGGGTTCCACATGGGCATAGGTGGGG |
| | | ACGACTCGTGGTCGCCGTCGGTCTCGGCGGAGCTCCAACTCTCGGCG |
| | | GGTCGTTACCATTACCAACTAGTTTGGTGCCAGAAG |
| 151 | MBP-LA | MASNFTQFVLVDNGGTGDVTVAPSNFANGIAEWISSNSRSQAYKVTCS |
| | (aa) | VRQSSAQNRKYTIKVEVPKGAWRSYLNMELTIPIFATNSDCELIVKAMQ |
| | | GLLKDGNPIPSAIAANSGIYGGGGSAENGDNEKMAALEAKICHQIEYYF |
| | | GDFNLPRDKFLKEQIKLDEGWVPLEIMIKFNRLNRLTTDFNVIVEALSKS |
| | | KAELMEISEDKTKIRRSPSKPLPEVTDEYKNDVKNRSVYIKGFPTDATLD |
| | | DIKEWLEDKGQVLNIQMRRTLHKAFKGSIFVVFDSIESAKKFVETPGQK |
| | | YKETDLLILFKDDYFAKKNEERKQNKVEAKLRAKQEQEAKQKLEEDA |
| | | EMKSLEEKIGCLLKFSGDLDDQTCREDLHILFSNHGEIKWIDFVRGAKE |
| | | GIILFKEKAKEALGKAKDANNGNLQLRNKEVTWEVLEGEVEKEALKKI |
| | | IEDQQESLNKWKSKGRRFKGKGKGNKAAQPGSGKGKVQFQ |
| 152 | MBP-LA (nt) | ATGGCCAGCAACTTCACCCAGTTCGTGCTGGTGGACAACGGCGGTA |
| | | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGAGGGAGCGCA |
| | | GAGAACGGCGACAACGAGAAGATGGCCGCCCTGGAGGCCAAGATCT |
| | | GCCACCAGATCGAGTACTACTTCGGCGACTTTAACCTGCCCCGGGAC |
| | | AAGTTCCTGAAGGAGCAGATCAAGCTGGACGAGGGCTGGGTGCCCC |
| | | TGGAGATTATGATCAAGTTCAACCGGCTGAATCGGCTGACCACCGA |
| | | CTTCAACGTGATCGTGGAGGCTCTGAGCAAGAGCAAGGCCGAGCTG |
| | | ATGGAGATCAGCGAGGACAAGACAAAGATCCGGCGGAGCCCATCCA |
| | | AGCCCCTGCCCGAGGTTACCGACGAGTACAAGAACGACGTGAAGAA |
| | | CCGGAGCGTGTACATCAAGGGCTTCCCCACCGACGCCACCCTGGAC |
| | | GATATCAAGGAGTGGCTGGAAGACAAGGGCCAGGTGCTGAACATCC |
| | | AGATGCGGCGGACCCTGCACAAGGCCTTCAAGGGCAGCATCTTCGT |
| | | GGTGTTCGACAGCATCGAGAGCGCCAAGAAATTCGTGGAGACACCC |
| | | GGCCAGAAGTACAAAGAGACCGACCTGCTGATCCTGTTCAAGGACG |
| | | ACTACTTTGCCAAGAAGAACGAGGAGCGGAAGCAGAACAAGGTCG |
| | | AAGCCAAGCTGCGGGCCAAGCAGGAGCAGGAGGCTAAGCAGAAGC |
| | | TGGAGGAGGACGCCGAGATGAAGAGCTTGGAGGAGAAGATCGGCT |
| | | GCCTGCTGAAGTTCAGCGGCGATCTGGACGACCAGACCTGCCGGGA |
| | | GGACCTGCACATCCTTTTCAGCAACCACGGCGAGATCAAGTGGATC |
| | | GACTTCGTGCGGGGCGCCAAGGAGGGCATCATCCTGTTTAAGGAGA |
| | | AGGCCAAAGAAGCCCTGGGCAAGGCCAAGGACGCCAACAACGGCA |
| | | ACCTGCAGCTGCGGAACAAGGAGGTGACCTGGGAGGTGCTGGAGGG |
| | | CGAGGTGGAGAAGGAGGCCCTGAAGAAGATCATCGAGGACCAGCA |
| | | GGAGAGCCTGAACAAGTGGAAGTCCAAGGGCCGGCGGTTCAAGGG |
| | | GAAGGGTAAGGGCAACAAGGCAGCCCAGCCCGGCTCTGGGAAGGG |
| | | CAAGGTGCAGTTCCAG |
| 55 | 50S | MYVRFEVPEDMQNEALSLLEKVRESGKVKKGTNETTKAVERGLAKLV |
| | ribosomal | YIAEDVDPPEIVAHLPLLCEEKNVPYIYVKSKNDLGRAVGIEVPCASAAI |
| | L7Ae protein | INEGELRKELGSLVEKIKGLQK |
| | (aa) | |
| 56 | 50S | ATGTACGTGAGATTTGAGGTTCCTGAGGACATGCAGAACGAAGCTC |
| | ribosomal | TGAGTCTGCTGGAGAAGGTTAGGGAGAGCGGTAAGGTAAAGAAAG |
| | L7Ae protein | GTACCAACGAGACGACAAAGGCTGTGGAGAGGGGACTGGCAAAGC |
| | (nt) | TCGTTTACATCGCAGAGGATGTTGACCCGCCTGAGATCGTTGCTCAT |
| | | CTGCCCCTCCTCTGCGAGGAGAAGAATGTGCCGTACATTTACGTTAA |
| | | AAGCAAGAACGACCTTGGAAGGGCTGTGGGCATTGAGGTGCCATGC |
| | | GCTTCGGCAGCGATAATCAACGAGGGAGAGCTGAGAAAGGAGCTTG |
| | | GAAGCCTTGTGGAGAAGATTAAAGGCCTTCAGAAGAGATCTCATAT |
| | | GCATCTCGAG |
| 57 | Pre- | MASNFTQFVLVDNGGTGDVTVAPSNFANGVAEWISSNSRSQAYKVTCS |
| | dimerized | VRQSSAQNRKYTIKVEVPKVATQTVGGVELPVAAWRSYLNMELTIPIF |
| | MBP (aa) | ATNSDCELIVKAMQGLLKDGNPIPSAIAANSGIYGGGGSASNFTQFVLV |
| | (linker | DNGGTGDVTVAPSNFANGVAEWISSNSRSQAYKVTCSVRQSSAQNRK |
| | sequence | YTIKVEVPKVATQTVGGVELPVAAWRSYLNMELTIPIFATNSDCELIVK |
| | underlined) | AMQGLLKDGNPIPSAIAANSGIY |
| 58 | Pre- | ATGGCCAGCAATTTTACCCAGTTCGTGCTGGTCGACAACGGCGG |
| | dimerized | GACCGGTGACGTTACCGTGGCCCCTAGCAATTTCGCTAACGGCGTGG |
| | MBP (nt) | CTGAGTGGATCTCCAGCAACAGCCGGAGCCAAGCTTACAAGGTGAC |
| | (linker | CTGTAGCGTGCGGCAGAGCAGCGCCCAGAACCGGAAGTACACCATC |
| | sequence | AAGGTAGAAGTGCCTAAGGTGGCCACACAGACTGTGGGCGGTGTCG |
| | underlined) | AGCTGCCCGTGGCAGCTTGGAGGAGCTATCTGAATATGGAACTGAC |
| | | CATCCCCATCTTCGCCACTAACAGCGATTGTGAGCTGATTGTTAAGG |
| | | CCATGCAAGGCCTGCTAAAGGACGGGAACCCCATTCCCAGCGCCAT |
| | | CGCTGCCAACAGCGGCATCTACGGAGGAGGCGGAAGCGCTAGCAAC |
| | | TTCACCCAGTTTGTGCTGGTGGACAACGGTGGCACCGGCGACGTGAC |
| | | CGTGGCTCCCAGCAACTTTGCCAACGGTGTGGCCGAGTGGATCAGC |
| | | AGCAATTCTCGGAGCCAGGCCTACAAGGTCACGTGCAGCGTGCGCC |
| | | AAAGCAGCGCTCAGAATCGGAAGTATACTATCAAGGTGGAGGTGCC |
| | | CAAAGTGGCCACCCAGACCGTGGGTGGCGTGGAGCTGCCTGTGGCC |
| | | GCTTGGCGATCATACCTGAACATGGAGCTCACCATCCCTATCTTCGC |
| | | AACCAACAGCGACTGCGAGCTGATCGTGAAAGCCATGCAGGGCCTG |
| | | CTGAAGGACGGCAATCCCATCCCCAGCGCTATCGCCGCCAATAGCG |
| | | GAATCTAC |
| 59 | MBP-mid2- | MASNFTQFVLVDNGGTGDVTVAPSNFANGIAEWISSNSRSQAYKVTCS |
| | mut (aa) | VRQSSAQNRKYTIKVEVPKGAWRSYLNMELTIPIFATNSDCELIVKAMQ |
| | | GLLKDGNPIPSAIAANSGIYGGGGSDPTRLQGINCGPDFTPSFANLGRTT |
| | | LSTRGPPRGGPGGELPRGPAGLGPRRSQQGPRKEPRKIIATVLMTEDIKL |
| | | NKAEKAWKPSSKRTAADKDRGEEDADGSKTQDLFRRVRSILNKLTPQ |
| | | MFQQLMKQVTQLAIDTEERLKGVIDLIFEKAISEPNFSVAYANMCRCLM |
| | | ALKVPTTEKPTVTVNFRKLLLNRCQKEFEKDKDDDEVFEKKQKEMDE |
| | | AATAEERGRLKEELEEARDIARRRSLGNIKAIGELFKLKMLTEAIMHDC |
| | | VVKLLKNHDEESLECLCRLLTTIGKDLDFEKAKPRMDQYFNQMEKIIKE |
| | | KKTSSRIRFMLQDVLDLRGSNWVPRRGDQGPKTIDQIHKEAEMEEHRE |
| | | HIKVQQLMAKGSDKRRGGPPGPPISRGLPLVDDGGWNTVPISKGSRPID |
| | | TSRLTKITKPGSIDSNNQLFAPGGRLSWGKGSSGGSGAKPSDAASEAAR |
| | | PATSTLNRFSALQQAV |
| 60 | MBP-mid2- | ATGGCCAGCAACTTCACCCAGTTCGTGCTGGTGGACAACGGCGGTA |
| | mut (nt) | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCA |
| | | AGGTGGAGGTGCCTAAGGGCGCCTGGAGAAGCTACCTGAACATGGA |
| | | GCTGACCATCCCTATCTTCGCCACCAACAGCGACTGCGAGCTGATCG |
| | | TGAAGGCCATGCAGGGCCTGCTGAAGGACGGCAACCCTATCCCTAG |
| | | CGCCATCGCCGCCAATTCAGGCATCTACGGAGGCGGTGGAAGCGAC |
| | | CCTACCAGACTGCAGGGCATCAACTGCGGCCCTGACTTCACTCCTTC |
| | | TTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTG |
| | | TTCAGAAGAGTGAGAAGCATCCTCAACAAGCTGACCCCTCAGATGT |
| | | TCCAGCAGCTGATGAAGCAGGTGACGCAGCTCGCCATCGACACCGA |
| | | GGAGAGACTGAAGGGCGTGATCGACCTGATCTTTGAGAAGGCTATC |
| | | TCAGAGCCTAACTTCAGCGTGGCCTACGCCAACATGTGCCGTTGCCT |
| | | GATGGCATTGAAGGTGCCAACCACCGAGAAGCCTACTGTGACCGTC |
| | | AATTTCCGTAAACTGCTGCTGAACCGGTGCCAGAAAGAGTTCGAGA |
| | | AGGATAAGGACGACGACGAGGTCTTCGAGAAGAAACAGAAAGAAA |
| | | TGGACGAGGCCGCCACCGCAGAGGAAAGGGGCCGATTAAAGGAGG |
| | | AGCTGGAGGAGGCCAGAGACATCGCCAGACGGCGTTCTCTGGGCAA |
| | | CATCAAGGCGATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTG |
| 61 | PCP (aa) | MSKTIVLSVGEATRTLTEIQSTADRQIFEEKVGPLVGRLRLTASLRQNGA |
| | | KTAYRVNLKLDQADVVDCSTSVCGELPKVRYTQVWSHDVTIVANSTE |
| | | ASRKSLYDLTKSLVVQATSEDLVVNLVPLGR |
| 62 | PCP (nt) | ATGAGCAAGACCATCGTGCTGAGCGTGGGCGAGGCCACCAGAACCC |
| | | TGACCGAGATCCAGAGCACCGCCGACAGACAGATCTTCGAGGAGAA |
| | | GGTGGGCCCTCTGGTGGGCAGACTGAGACTGACCGCCAGCCTGAGA |
| | | CAGAACGGCGCCAAGACCGCCTACAGAGTGAACCTGAAGCTGGACC |
| | | AGGCCGACGTGGTGGACTGCAGCACCAGCGTGTGCGGCGAGCTGCC |
| | | TAAGGTGAGATACACCCAGGTGTGGAGCCACGACGTGACCATCGTG |
| | | GCCAACAGCACCGAGGCCAGCAGAAAGAGCCTGTACGACCTGACCA |
| | | AGAGCCTGGTGGTGCAGGCCACCAGCGAGGACCTGGTGGTGAACCT |
| | | GGTGCCTCTGGGCAGA |
| 63 | PUF (aa) | MGSSHHHHHHSQDLEVLFQGPHMGRSRLLEDFRNNRYPNLQLREIAGH |
| | | IMEFSQDQHGSRFIQLKLERATPAERQLVFNEILQAAYQLMVDVFGSYV |
| | | IEKFFEFGSLEQKLALAERIRGHVLSLALQMYGSRVIEKALEFIPSDQQN |
| | | EMVRELDGHVLKCVKDQNGCHVVQKCIECVQPQSLQFIIDAFKGQVFA |
| | | LSTHPYGSRVIERILEHCLPDQTLPILEELHQHTEQLVQDQYGCYVIQHV |
| | | LEHGRPEDKSKIVAEIRGNVLVLSQHKFASNVVEKCVTHASRTERAVLI |
| | | DEVCTMNDGPHSALYTMMKDQYASYVVEKMIDVAEPGQRKIVMHKIR |
| | | PHIMEFSQDQHGSRFIELKLERATPAERQLVFNEILQAAYQLMVDVFGC |
| | | YVIQKFFEFGSLEQKLALAERIRGHVLSLALQMYGSRVIEKALEFIPSDQ |
| | | QNEMVRELDGHVLKCVKDQNGCHVVQKCIECVQPQSLQFIIDAFKGQV |
| | | FALSTHPYGSRVIERILEHCLPDQTLPILEELHQHTEQLVQDQYGCYVIQ |
| | | HVLEHGRPEDKSKIVAEIRGNVLVLSQHKFASNVVEKCVTHASRTERA |
| | | VLIDEVCTMNDGPHSALYTMMKDQYACYVVQKMIDVAEPGQRKIVM |
| | | HKIRPHIATLRKYTYGKHILAKLEKYYMKNGVDLG |
| 64 | PUF (nt) | ATGGGCAGCAGCCACCATCACCACCACCACAGCCAGGACCTGGAGG |
| | | TGCTGTTCCAGGGCCCACACATGGGCCGTAGCCGGCTGCTGGAGGA |
| | | CTTCCGGAACAACCGGTACCCCAACCTGCAGCTGCGGGAGATCGCC |
| | | GGCCACATCATGGAGTTCAGCCAGGACCAGCACGGCAGCCGCTTCA |
| | | TCCAGCTGAAGTTGGAGCGGGCTACACCCGCTGAGCGGCAGCTGGT |
| | | GTTCAACGAGATCCTGCAGGCCGCCTATCAACTGATGGTAGACGTGT |
| | | TCGGCAGCTACGTGATCGAGAAGTTCTTTGAGTTCGGCAGTCTGGAG |
| | | CAGAAGCTCGCCCTGGCCGAACGGATACGGGGCCACGTGCTTAGCC |
| | | TGGCCCTACAGATGTACGGCAGCAGGGTGATCGAGAAAGCCCTGGA |
| | | GTTCATCCCCAGCGACCAACAGAACGAAATGGTGCGGGAGCTCGAC |
| | | GGCCACGTCCTGAAGTGCGTGAAGGACCAGAACGGTTGCCACGTGG |
| | | TGCAGAAGTGCATCGAGTGCGTCCAGCCCCAGTCTCTGCAGTTCATT |
| | | ATCGACGCCTTCAAGGGCCAAGTGTTCGCCCTGAGTACCCACCCCTA |
| | | CGGAAGCCGGGTGATCGAACGGATTCTGGAGCATTGCCTGCCCGAT |
| | | CAGACCCTGCCCATCCTGGAGGAGCTGCATCAACACACCGAACAGC |
| | | TTGTGCAAGACCAGTACGGCTGCTACGTGATCCAGCACGTGTTGGAG |
| | | CACGGACGGCCCGAGGACAAGAGCAAGATTGTGGCCGAGATCAGG |
| | | GGTAACGTGCTGGTGCTGTCTCAGCACAAATTCGCCAGCAACGTGGT |
| | | AGAGAAGTGCGTGACCCACGCCAGCCGGACTGAACGGGCCGTGCTG |
| | | ATCGACGAGGTGTGCACTATGAACGACGGGCCCCACTCTGCCCTGTA |
| | | CACAATGATGAAAGACCAATACGCCAGCTACGTGGTGGAGAAGATG |
| | | ATCGACGTGGCCGAGCCAGGCCAACGGAAGATCGTGATGCATAAGA |
| | | TCCGGCCTCATATCATGGAGTTTAGCCAGGATCAACACGGCAGCCG |
| | | GTTCATCGAGCTGAAGCTGGAACGGGCCACCCCTGCCGAACGGCAG |
| | | CTGGTCTTTAACGAGATACTGCAAGCCGCCTACCAGCTGATGGTGGA |
| | | CGTGTTTGGTTGCTACGTTATCCAGAAGTTCTTCGAGTTCGGAAGCC |
| | | TGGAGCAGAAACTGGCCCTGGCCGAGCGTATCCGGGGCCACGTTCT |
| | | GAGCTTAGCCCTGCAGATGTACGGGAGCCGGGTTATCGAGAAGGCC |
| | | CTGGAATTCATCCCTAGCGATCAGCAGAACGAGATGGTCAGAGAGC |
| | | TGGACGGCCACGTGCTCAAGTGCGTGAAAGACCAGAACGGCTGCCA |
| | | CGTGGTACAGAAGTGTATCGAGTGTGTGCAGCCCCAGAGCCTCCAG |
| | | TTTATCATTGACGCCTTTAAGGGCCAGGTGTTTGCCCTGAGCACACA |
| | | CCCCTACGGCAGCCGGGTAATCGAGCGGATCCTGGAGCACTGCCTG |
| | | CCCGACCAGACCTTGCCCATCCTCGAGGAACTGCACCAGCACACCG |
| | | AGCAGCTGGTGCAGGACCAATACGGCTGTTACGTCATCCAGCACGTT |
| | | CTGGAGCACGGCCGGCCTGAGGACAAGTCTAAGATCGTGGCCGAAA |
| | | TCCGGGGCAACGTGTTGGTGCTGAGCCAGCACAAGTTCGCCTCAAA |
| | | CGTCGTGGAGAAGTGTGTGACTCACGCCTCTCGGACCGAGCGGGCA |
| | | GTGCTGATTGACGAGGTGTGTACCATGAACGACGGCCCACACAGCG |
| | | CCCTGTACACCATGATGAAGGACCAGTACGCCTGCTACGTTGTGCAG |
| | | AAGATGATTGACGTGGCCGAACCCGGTCAGCGCAAGATTGTGATGC |
| | | ACAAGATCCGGCCCCACATCGCCACCCTGCGGAAGTACACCTACGG |
| | | CAAGCACATCCTGGCCAAGCTGGAGAAGTACTACATGAAGAACGGC |
| | | GTGGACCTGGGC |
| 65 | PUF2 (aa) | MGSSHHHHHHSQDLEVLFQGPHMGRSRLLEDFRNNRYPNLQLREIAGH |
| | | IMEFSQDQHGSRFIQLKLERATPAERQLVFNEILQAAYQLMVDVFGNYV |
| | | IQKFFEFGSLEQKLALAERIRGHVLSLALQMYGNRVIQKALEFIPSDQQN |
| | | EMVRELDGHVLKCVKDQNGCHVVQKCIECVQPQSLQFIIDAFKGQVFA |
| | | LSTHPYGNRVIQRILEHCLPDQTLPILEELHQHTEQLVQDQYGCYVIQHV |
| | | LEHGRPEDKSKIVAEIRGNVLVLSQHKFASNVVEKCVTHASRTERAVLI |
| | | DEVCTMNDGPHSALYTMMKDQYANYVVQKMIDVAEPGQRKIVMHKI |
| | | RPHIMEFSQDQHGNRFIQLKLERATPAERQLVFNEILQAAYQLMVDVFG |
| | | NYVIQKFFEFGSLEQKLALAERIRGHVLSLALQMYGNRVIQKALEFIPSD |
| | | QQNEMVRELDGHVLKCVKDQNGNHVVQKCIECVQPQSLQFIIDAFKGQ |
| | | VFALSTHPYGNRVIQRILEHCLPDQTLPILEELHQHTEQLVQDQYGNYVI |
| | | QHVLEHGRPEDKSKIVAEIRGNVLVLSQHKFANNVVQKCVTHASRTER |
| | | AVLIDEVCTMNDGPHSALYTMMKDQYANYVVQKMIDVAEPGQRKIV |
| | | MHKIRPHIATLRKYTYGKHILAKLEKYYMKNGVDLG |
| 66 | PUF2 (nt) | ATGGGCAGCTCCCATCACCACCACCATCATTCGCAGGACCTTGAAGT |
| | | CCTGTTCCAAGGTCCTCATATGGGTAGATCAAGGCTTTTGGAAGATT |
| | | TTCGAAATAACCGGTATCCTAACCTGCAGCTCAGGGAGATAGCAGG |
| | | ACACATCATGGAGTTCAGCCAGGACCAACACGGAAGCAGGTTTATC |
| | | CAACTGAAACTCGAGAGAGCAACGCCCGCAGAGCGTCAGCTGGTGT |
| | | TTAACGAGATCCTGCAGGCAGCCTACCAGCTGATGGTTGACGTGTTC |
| | | GGTAACTACGTAATTCAGAAGTTCTTTGAGTTCGGTTCTCTGGAACA |
| | | GAAACTCGCTCTGGCTGAGCGAATTCGGGGTCACGTGCTCTCACTGG |
| | | CGCTCCAGATGTACGGCAATAGAGTGATTCAGAAGGCTCTGGAATTT |
| | | ATTCCATCGGACCAGCAGAACGAAATGGTACGAGAACTGGACGGTC |
| | | ACGTCCTTAAGTGTGTCAAAGATCAGAACGGCTGTCACGTAGTACA |
| | | GAAGTGCATCGAGTGTGTGCAGCCGCAGAGCCTCCAGTTTATCATCG |
| | | ACGCCTTTAAAGGGCAGGTCTTCGCCTTGTCCACCCATCCGTACGGA |
| | | AACCGAGTCATTCAGCGCATCCTGGAACATTGCCTCCCGGATCAGAC |
| | | CCTGCCGATTCTGGAGGAGCTTCACCAGCACACCGAGCAACTAGTG |
| | | CAGGATCAGTACGGCTGTTACGTGATACAGCACGTCCTGGAGCACG |
| | | GTAGACCAGAGGACAAATCGAAGATTGTGGCCGAAATCAGAGGAA |
| | | ACGTGCTGGTCCTTTCCCAGCACAAGTTCGCTTCCAACGTGGTGGAG |
| | | AAGTGCGTGACACACGCAAGCCGCACCGAGAGGGCAGTCTTAATCG |
| | | ACGAAGTGTGCACAATGAACGACGGTCCACACTCTGCCTTATACAC |
| | | AATGATGAAGGACCAGTACGCAAACTACGTGGTGCAGAAGATGATT |
| | | GACGTAGCTGAGCCTGGCCAGCGAAAGATTGTCATGCATAAGATCA |
| | | GGCCTCATATTATGGAGTTTTCTCAGGATCAACACGGCAATCGGTTC |
| | | ATCCAATTAAAGCTGGAGAGGGCCACACCTGCGGAGAGGCAGTTGG |
| | | TCTTTAACGAAATTCTACAAGCCGCTTACCAATTAATGGTGGACGTC |
| | | TTTGGCAACTACGTGATCCAGAAATTCTTCGAGTTTGGAAGTCTTGA |
| | | ACAGAAGCTGGCGCTGGCTGAAAGAATCAGAGGTCACGTCCTCAGC |
| | | CTGGCATTGCAAATGTACGGAAATCGAGTCATCCAGAAAGCCCTTG |
| | | AGTTTATCCCCAGCGATCAACAGAACGAGATGGTTCGCGAATTGGA |
| | | CGGGCACGTGCTGAAGTGTGTTAAGGACCAGAACGGGAACCACGTC |
| | | GTGCAGAAGTGCATAGAGTGCGTCCAGCCACAGTCCCTGCAGTTTAT |
| | | TATTGACGCATTTAAGGGACAAGTGTTCGCCTTATCTACCCACCCTT |
| | | ACGGCAATAGGGTCATTCAGAGGATCTTGGAACACTGTCTTCCCGAC |
| | | CAGACACTGCCAATATTGGAAGAACTGCATCAACATACCGAACAGC |
| | | TCGTACAAGATCAGTACGGTAATTACGTCATTCAACACGTTCTCGAA |
| | | CACGGAAGGCCCGAAGACAAGTCTAAGATCGTCGCAGAGATACGTG |
| | | GGAACGTCCTCGTCTTAAGTCAGCATAAGTTCGCAAACAACGTTGTG |
| | | CAGAAGTGTGTAACACACGCGAGCAGAACAGAGAGAGCCGTGCTGA |
| | | TCGACGAGGTGTGTACCATGAACGACGGGCCACACAGCGCCCTCTA |
| | | CACCATGATGAAAGACCAATACGCTAACTACGTGGTTCAGAAGATG |
| | | ATCGACGTCGCCGAACCAGGTCAACGGAAGATCGTGATGCACAAGA |
| | | TCAGGCCACACATAGCCACCCTGAGAAAGTATACGTACGGTAAACA |
| | | TATCCTGGCAAAGTTAGAGAAGTACTATATGAAGAACGGCGTGGAC |
| | | TTAGGA |
| 67 | Lambda (aa) | MGNARTRRRERRAEKQAQWKAAN |
| 68 | Lambda (nt) | ATGGGCAACGCCCGGACCAGGAGAAGAGAGCGGCGGGCCGAGAAG |
| | | CAGGCCCAGTGGAAGGCCGCCAAC |
| 69 | U1A (aa) | MAVPETRPNHTIYINNLNEKIKKDELKKSLYAIFSQFGQILDILVSRSLK |
| | | MRGQAFVIFKEVSSATNALRSMQGFPFYDKPMRIQYAKTDSDIIAKMK |
| | | GTF |
| 70 | U1A (nt) | ATGGCCGTGCCTGAGACCAGACCTAACCACACCATCTACATCAACA |
| | | ACCTGAACGAGAAGATCAAGAAGGACGAGCTGAAGAAGAGCCTGT |
| | | ACGCCATCTTCAGCCAGTTCGGCCAGATCCTGGACATCCTGGTGAGC |
| | | AGAAGCCTGAAGATGAGAGGCCAGGCCTTCGTGATCTTCAAGGAGG |
| | | TGAGCAGCGCCACCAACGCCCTGAGAAGCATGCAGGGCTTCCCTTTC |
| | | TACGACAAGCCTATGAGAATCCAGTACGCCAAGACCGACAGCGACA |
| | | TCATCGCCAAGATGAAGGGCACCTTC |
| 71 | 15.5 kd (aa) | MTEADVNPKAYPLADAHLTKKLLDLVQQSCNYKQLRKGANEATKTLN |
| | | RGISEFIVMAADAEPLEIILHLPLLCEDKNVPYVFVRSKQALGRACGVSR |
| | | PVIACSVTIKEGSQLKQQIQSIQQSIERLLV |
| 72 | 15.5 kd (nt) | ATGACCGAGGCCGACGTGAACCCTAAGGCCTACCCTCTGGCCGACG |
| | | CCCACCTGACCAAGAAGCTGCTGGACCTGGTGCAGCAGAGCTGCAA |
| | | CTACAAGCAGCTGAGAAAGGGCGCCAACGAGGCCACCAAGACCCTG |
| | | AACAGAGGCATCAGCGAGTTCATCGTGATGGCCGCCGACGCCGAGC |
| | | CTCTGGAGATCATCCTGCACCTGCCTCTGCTGTGCGAGGACAAGAAC |
| | | GTGCCTTACGTGTTCGTGAGAAGCAAGCAGGCCCTGGGCAGAGCCT |
| | | GCGGCGTGAGCAGACCTGTGATCGCCTGCAGCGTGACCATCAAGGA |
| | | GGGCAGCCAGCTGAAGCAGCAGATCCAGAGCATCCAGCAGAGCATC |
| | | GAGAGACTGCTGGTG |
| 73 | LARP7 (aa) | METESGNQEKVMEEESTEKKKEVEKKKRSRVKQVLADIAKQVDFWFG |
| | | DANLHKDRFLREQIEKSRDGYVDISLLVSFNKMKKLTTDGKLIARALRS |
| | | SAVVELDLEGTRIRRKKPLGERPKDEDERTVYVELLPKNVNHSWIERVF |
| | | GKCGNVVYISIPHYKSTGDPKGFAFVEFETKEQAAKAIEFLNNPPEEAPR |
| | | KPGIFPKTVKNKPIPALRVVEEKKKKKKKKGRMKKEDNIQAKEENMDT |
| | | SNTSISKMKRSRPTSEGSDIESTEPQKQCSKKKKKRDRVEASSLPEVRTG |
| | | KRKRSSSEDAESLAPRSKVKKIIQKDIIKEASEASKENRDIEISTEEEKDT |
| | | GDLKDSSLLKTKRKHKKKHKERHKMGEEVIPLRVLSKSEWMDLKKEY |
| | | LALQKASMASLKKTISQIKSESEMETDSGVPQNTGMKNEKTANREECR |
| | | TQEKVNATGPQFVSGVIVKIISTEPLPGRKQVRDTLAAISEVLYVDLLEG |
| | | DTECHARFKTPEDAQAVINAYTEINKKHCWKLEILSGDHEQRYWQKIL |
| | | VDRQAKLNQPREKKRGTEKLITKAEKIRLAKTQQASKHIRFSEYD |
| 74 | LARP7 (nt) | ATGGAGACCGAGAGCGGCAACCAGGAGAAGGTGATGGAGGAGGAG |
| | | AGCACCGAGAAGAAGAAGGAGGTGGAGAAGAAGAAGAGAAGCAG |
| | | AGTGAAGCAGGTGCTGGCCGACATCGCCAAGCAGGTGGACTTCTGG |
| | | TTCGGCGACGCCAACCTGCACAAGGACAGATTCCTGAGAGAGCAGA |
| | | TCGAGAAGAGCAGAGACGGCTACGTGGACATCAGCCTGCTGGTGAG |
| | | CTTCAACAAGATGAAGAAGCTGACCACCGACGGCAAGCTGATCGCC |
| | | AGAGCCCTGAGAAGCAGCGCCGTGGTGGAGCTGGACCTGGAGGGCA |
| | | CCAGAATCAGAAGAAAGAAGCCTCTGGGCGAGAGACCTAAGGACG |
| | | AGGACGAGAGAACCGTGTACGTGGAGCTGCTGCCTAAGAACGTGAA |
| | | CCACAGCTGGATCGAGAGAGTGTTCGGCAAGTGCGGCAACGTGGTG |
| | | TACATCAGCATCCCTCACTACAAGAGCACCGGCGACCCTAAGGGCTT |
| | | CGCCTTCGTGGAGTTCGAGACCAAGGAGCAGGCCGCCAAGGCCATC |
| | | GAGTTCCTGAACAACCCTCCTGAGGAGGCCCCTAGAAAGCCTGGCA |
| | | TCTTCCCTAAGACCGTGAAGAACAAGCCTATCCCTGCCCTGAGAGTG |
| | | GTGGAGGAGAAGAAGAAGAAGAAGAAGAAGAAGGGCAGAATGAA |
| | | GAAGGAGGACAACATCCAGGCCAAGGAGGAGAACATGGACACCAG |
| | | CAACACCAGCATCAGCAAGATGAAGAGAAGCAGACCTACCAGCGA |
| | | GGGCAGCGACATCGAGAGCACCGAGCCTCAGAAGCAGTGCAGCAA |
| | | GAAGAAGAAGAAGAGAGACAGAGTGGAGGCCAGCAGCCTGCCTGA |
| | | GGTGAGAACCGGCAAGAGAAAGAGAAGCAGCAGCGAGGACGCCGA |
| | | GAGCCTGGCCCCTAGAAGCAAGGTGAAGAAGATCATCCAGAAGGAC |
| | | ATCATCAAGGAGGCCAGCGAGGCCAGCAAGGAGAACAGAGACATC |
| | | GAGATCAGCACCGAGGAGGAGAAGGACACCGGCGACCTGAAGGAC |
| | | AGCAGCCTGCTGAAGACCAAGAGAAAGCACAAGAAGAAGCACAAG |
| | | GAGAGACACAAGATGGGCGAGGAGGTGATCCCTCTGAGAGTGCTGA |
| | | GCAAGAGCGAGTGGATGGACCTGAAGAAGGAGTACCTGGCCCTGCA |
| | | GAAGGCCAGCATGGCCAGCCTGAAGAAGACCATCAGCCAGATCAAG |
| | | AGCGAGAGCGAGATGGAGACCGACAGCGGCGTGCCTCAGAACACC |
| | | GGCATGAAGAACGAGAAGACCGCCAACAGAGAGGAGTGCAGAACC |
| | | CAGGAGAAGGTGAACGCCACCGGCCCTCAGTTCGTGAGCGGCGTGA |
| | | TCGTGAAGATCATCAGCACCGAGCCTCTGCCTGGCAGAAAGCAGGT |
| | | GAGAGACACCCTGGCCGCCATCAGCGAGGTGCTGTACGTGGACCTG |
| | | CTGGAGGGCGACACCGAGTGCCACGCCAGATTCAAGACCCCTGAGG |
| | | ACGCCCAGGCCGTGATCAACGCCTACACCGAGATCAACAAGAAGCA |
| | | CTGCTGGAAGCTGGAGATCCTGAGCGGCGACCACGAGCAGAGATAC |
| | | TGGCAGAAGATCCTGGTGGACAGACAGGCCAAGCTGAACCAGCCTA |
| | | GAGAGAAGAAGAGAGGCACCGAGAAGCTGATCACCAAGGCCGAGA |
| | | AGATCAGACTGGCCAAGACCCAGCAGGCCAGCAAGCACATCAGATT |
| | | CAGCGAGTACGAC |

### Cleavage Site

Disclosed herein, inter alia, is a composition or system comprising a first polynucleotide. In some embodiments, the first polynucleotide has a cleavage site. In some embodiments, the composition or system further comprises a second polynucleotide. In some embodiments, the second polynucleotide has a cleavage site. In some embodiments, the cleavage site is on the first polynucleotide but not on the second polynucleotide. In some embodiments, the cleavage site is on the second polynucleotide but not on the first polynucleotide.

In some embodiments, the cleavage site situated on the first or second polynucleotide is selected from a microRNA target site, or an endonuclease recognition site.

In some embodiments, the cleavage site is situated on the first polypeptide upstream (5′) or downstream (3′) of the open reading frame encoding the first polypeptide. In some embodiments, the cleavage site is situated on the first polypeptide downstream of the open reading frame encoding the first polypeptide. In some embodiments, the cleavage site is situated on the first polypeptide between the repressor binding site and upstream of the open reading frame encoding the first polypeptide. In some embodiments, the cleavage site is situated on the first polypeptide downstream of the poly A tail. In some embodiments, the cleavage site is situated on the first polypeptide between the poly A tail and upstream of a destabilizing sequence or degradation tag.

In some embodiments, the cleavage site is situated on the second polypeptide downstream (3′) of the open reading frame encoding the second polypeptide. In some embodiments, the cleavage site is situated on the second polynucleotide between the open reading frame encoding the second polypeptide and upstream of the poly A tail.

In some embodiments, the cleavage site is within a microRNA (miRNA) target sequence selected from, but not limited to, the sequences in Table 5 below:

TABLE 5
miRNA target sequences

| | Sequence | |
|---|---|---|
| SEQ ID NO | information | Sequence |
| 75 | miR122-ts | CAAACACCATTGTCACACTCCA |
| 76 | miR142-ts | TCCATAAAGTAGGAAACACTACA |
| 77 | miR126-ts | CGCATTATTACTCACGGTACGA |
| 78 | miR150-ts | CACTGGTACAAGGGTTGGGAGA |
| 79 | miR155-ts | ACCCCTATCACGATTAGCATTAA |
| 80 | miR223-ts | TGGGGTATTTGACAAACTGACA |

For example, the microRNA target site is a miR122 target site or a miR142 target site.

In some embodiments, miRNAs of interest can be determined from miRNA databases as referenced in sources known in the art, e.g., Kozomara A, et al., Nucleic Acids Res 2019 47:D155-D162; Kozomara A, Griffiths-Jones S., Nucleic Acids Res 2014 42:D68-D73; Kozomara A, Griffiths-Jones S. Nucleic Acids Res 2011 39:D152-D157; Griffiths-Jones S, Saini H K, van Dongen S, Enright A J. Nucleic Acids Res 2008 36:D154-D158; Griffiths-Jones S, Grocock R J, van Dongen S, Bateman A, Enright A J. Nucleic Acids Res 2006 34:D140-D144; Griffiths-Jones S. Nucleic Acids Res 2004 32:D109-D111; Ambros V, et al., RNA 2003 9(3):277-279; and Meyers B C, et al., Plant Cell. 2008 20(12):3186-3190. A nucleotide sequence that is a reverse complement of a miRNA sequence selected from such a database would be the miRNA target-site. In some embodiments, the cleavage site is, e.g., an endonuclease site exemplified in Tomecki R and Dziembowski A., RNA 2010. 16: 1692-1724; and Schoenberg D R., Wiley Interdiscip Rev RNA. 2011; 2(4): 582-600.

### Tissue-Specific Promoters

Disclosed herein, inter alia, is a composition or system comprising a polynucleotide encoding an endonuclease sequence that is under the control of a tissue-specific promoter. Further described is a composition or system comprising a polynucleotide encoding an effector sequence that is under the control of a tissue-specific promoter. A tissue-specific promoter may be any promoter known in the art that is expressed in some but not all tissues. See, e.g., Weeratna R D et al., Gene Therapy (2001) 8, 1872-1878; van Dijk M A, et al. Nucleic Acids Res. (1992); 20(12):3099-3104; and Chuah M K, et al. Mol Ther. (2014) September; 22(9):1605-13.

### Methods of Using the Systems or Compositions

The present disclosure provides compositions, which can be delivered to cells, e.g., target cells, e.g., in vitro or in vivo. For in vitro protein expression, the cell is contacted with the composition by incubating the composition and the cell ex vivo. Such cells may subsequently be introduced in vivo. For in vivo protein expression, the cell is contacted with the composition by administering the composition to a subject to thereby induce protein expression in or on the desired cells within the subject. For example, in one embodiment, the composition is administered intravenously. In another embodiment, the composition is administered intramuscularly. In yet other embodiment, the composition is administered by a route selected from the group consisting of subcutaneously, intranodally and intratumorally.

For in vitro delivery, in one embodiment the cell is contacted with the composition by incubating the composition and the target cell ex vivo. In one embodiment, the cell is a human cell. Various types of cells have been demonstrated to be transfectable by the composition (e.g., the LNP).

In another embodiment, the cell is contacted with the composition for, e.g., at least 30 minutes, at least 1 hour, at least 2 hours, at least 3 hours, at least 4 hours, at least 5 hours, at least 6 hours, at least 12 hours or at least 24 hours.

In one embodiment, the cell is contacted with the composition for a single treatment/transfection. In another embodiment, the cell is contacted with the composition for multiple treatments/transfections (e.g., two, three, four or more treatments/transfections of the same cells).

In another embodiment, for in vivo delivery, the cell is contacted with the composition by administering the composition to a subject to thereby deliver the polynucleotide(s) to cells within the subject. For example, in one embodiment, the composition is administered intravenously. In another embodiment, the composition is administered intramuscularly. In yet other embodiment, the composition is administered by a route selected from the group consisting of subcutaneously, intranodally and intratumorally.

In an aspect, provided herein is a method of expressing a polypeptide in a cell, comprising administering to the cell a composition disclosed herein.

In a related aspect, provided herein is a composition or system for use in a method of expressing a polypeptide in a cell in a cell.

In another aspect, the disclosure provides a method of expressing a polypeptide in a cell in a subject, comprising administering to the subject an effective amount of a composition disclosed herein.

In a related aspect, provided herein is a composition or system for use in a method of expressing a polypeptide in a cell in a subject.

In yet another aspect, provided herein is a method of delivering a composition disclosed herein.

In a related aspect, provided herein is a composition or system for use in a method of delivering the composition to a cell.

In an embodiment, the method or use, comprises contacting the cell in vitro, in vivo or ex vivo with the composition or system.

In an embodiment, the composition or system formulated as an LNP, a liposome composition, a lipoplex composition, or a polyplex composition of the present disclosure is contacted with cells, e.g., ex vivo or in vivo and can be used to deliver a secreted polypeptide, an intracellular polypeptide, a transmembrane polypeptide, or peptides, polypeptides or biologically active fragments thereof to a subject.

In an aspect, the disclosure provides a method of delivering a composition or system disclosed herein to a subject having a disease or disorder, e.g., as described herein.

In a related aspect, provided herein is a composition or system for use in a method of delivering the composition or system to a subject having a disease or disorder, e.g., as described herein.

In another aspect, provided herein is a method of modulating an immune response in a subject, comprising administering to the subject in need thereof an effective amount of a composition or system disclosed herein.

In a related aspect, provided herein is a composition or system for use in a method of modulating an immune response in a subject, comprising administering to the subject an effective amount of the composition or system.

In another aspect, provided herein is a method of delivering a secreted polypeptide, an intracellular polypeptide, a transmembrane polypeptide, or peptides, polypeptides or biologically active fragments thereof to a subject.

In an aspect, provided herein is a method of treating, preventing, or preventing a symptom of, a disease or disorder comprising administering to a subject in need thereof an effective amount of a composition or system disclosed herein.

In a related aspect, provided herein is a composition or system for use in a method of treating, preventing, or preventing a symptom of, a disease or disorder in a subject, comprising administering to the subject in need thereof an effective amount of the composition or system.

In an embodiment, the first polynucleotide and/or the second polynucleotide of the system is formulated as an LNP. In an embodiment, the first polynucleotide of the system is formulated as an LNP. In an embodiment, the second polynucleotide of the system is formulated as an LNP. In an embodiment, both the first and the second polynucleotides of the system are formulated as LNPs.

In an embodiment, the LNP comprising the first polynucleotide is the same as the LNP comprising the second polynucleotide. In an embodiment, the LNP comprising the first polynucleotide is different from the LNP comprising the second polynucleotide.

In an embodiment, the LNP comprising the first polynucleotide is in a composition. In an embodiment, the LNP comprising the second polynucleotide is in a separate composition. In an embodiment, the LNP comprising the first polynucleotide and the LNP comprising the second polynucleotide are in the same composition. In some embodiments, the first and second polynucleotides are in separate dosage forms packaged together. In some embodiments, the first and second polynucleotides are in a unit dosage form.

In an embodiment, the LNP comprising the first polynucleotide and the LNP comprising the second polynucleotide are administered simultaneously, e.g., substantially simultaneously. In some embodiments, the LNP comprising the first polynucleotide and the LNP comprising the second polynucleotide are co-delivered.

In an embodiment, the LNP comprising the first polynucleotide and the LNP comprising the second polynucleotide are administered sequentially.

In an embodiment, the LNP comprising the first polynucleotide is administered first.

In an embodiment, the LNP comprising the first polynucleotide is administered first followed by administration of the LNP comprising the second polynucleotide.

In an embodiment, the LNP comprising the second polynucleotide is administered first.

In an embodiment, the LNP comprising the second polynucleotide is administered first followed by administration of the LNP comprising the first polynucleotide.

In some embodiments of the disclosed methods, the method comprises contacting the cell with a composition of the disclosure (for example a composition according to FIGS. 1A-1B), wherein the cell expresses a microRNA or endonuclease that binds to the recognition site or cleavage site and reduces translation of the repressor from the second polynucleotide. In some embodiments, the method comprising contacting the cell with a composition of the disclosure, wherein the cell expresses a microRNA or endonuclease that binds to the recognition site or cleavage site, cleaves the repressor binding element from the first polynucleotide, and enhances translation of the first polypeptide from the first polynucleotide.

In some embodiments of the disclosed methods, the method comprises contacting the cell with (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site or cleavage site, wherein the cell expresses a microRNA or an endonuclease that binds to the recognition site or cleavage site and reduces translation of the repressor from the second polynucleotide.

In some embodiments of the disclosed methods, the method comprises expressing a polypeptide in a cell, the method comprising contacting the cell with (a) a first polynucleotide comprising (i) a repressor binding element, (ii) a recognition site or cleavage site, and (iii) an open reading frame encoding a polypeptide; and (b) a second polynucleotide comprising a sequence encoding a repressor that binds to the repressor binding element, wherein the cell expresses a microRNA or endonuclease that binds to the recognition site or cleavage site, cleaves the repressor binding element from the first polynucleotide, and enhances translation of the polypeptide from the first polynucleotide.

In some embodiments of the disclosed methods, the method comprises expressing a polypeptide in a cell in a subject, the method comprising administering to the subject: (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site or cleavage site, wherein the cell expresses a microRNA or an endonuclease that binds to the recognition site or cleavage site and reduces translation of the repressor from the second polynucleotide.

In some embodiments of the disclosed methods, the method comprises expressing a polypeptide in a cell in a subject, the method comprising administering to the subject: (a) a first polynucleotide comprising (i) a repressor binding element, (ii) a recognition site or cleavage site, and (iii) an open reading frame encoding a polypeptide; and (b) a second polynucleotide comprising a sequence encoding a repressor that binds to the repressor binding element, wherein the cell expresses a microRNA or endonuclease that binds to the recognition site or cleavage site, cleaves the repressor binding element from the first polynucleotide, and enhances translation of the polypeptide from the first polynucleotide.

### Sequence Optimization and Methods Thereof

In some embodiments, a polynucleotide of the disclosure comprises a sequence-optimized nucleotide sequence encoding a polypeptide disclosed herein, e.g., a polynucleotide encoding a first polypeptide (e.g., a therapeutic or prophylactic protein), an effector molecule and/or a tether molecule. In some embodiments, the polynucleotide of the disclosure comprises an open reading frame (ORF) encoding a first polypeptide, an effector molecule, a tether molecule, etc, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics. In some embodiments, the percentage of uracil or thymine nucleobases in a sequence-optimized nucleotide sequence (e.g., encoding a first polypeptide, an effector molecule and/or a tether molecule, a functional fragment, or a variant thereof) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response in desired cells and/or microenvironments when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (% UTM or % TTM), relative to the wild-type (% UWT or % TWT), and relative to the total nucleotide content (% UTL or % TTL). For DNA it is recognized that thymine (T) is present instead of uracil (U), and one would substitute T where U appears. For RNA it is recognized that uracil (U) is present instead of thymine (T). One of skill in the art could readily obtain an RNA sequence when the DNA sequence is provided by substituting thymine in the DNA sequence to uracil. Thus, all the disclosures related to, e.g., % UTM, % UWT, or % UTL, with respect to RNA are equally applicable to % TTM, % TWT, or % TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % UTM or % TTM.

In some embodiments, a uracil-modified sequence encoding a polypeptide, an effector molecule, or a tether molecule of the disclosure has a reduced number of consecutive uracils with respect to the corresponding wild-type nucleic acid sequence. For example, two consecutive leucines can be encoded by the sequence CUUUUG, which includes a four uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence encoding a polypeptide, an effector molecule or a tether molecule of the disclosure has a reduced number of uracil triplets (UUU) with respect to the wild-type nucleic acid sequence. In some embodiments, a uracil-modified sequence encoding a polypeptide, an effector molecule or a tether molecule has a reduced number of uracil pairs (UU) with respect to the number of uracil pairs (UU) in the wild-type nucleic acid sequence. In some embodiments, a uracil-modified sequence encoding a polypeptide, an effector molecule or a tether molecule of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence”, refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UUwt. In some embodiments, a uracil-modified sequence encoding a polypeptide, an effector molecule or a tether molecule has a % UUwt between below 100%.

In some embodiments, the polynucleotide of the disclosure comprises a uracil-modified sequence encoding an encoding a polypeptide, an effector molecule or a tether molecule disclosed herein. In some embodiments, the uracil-modified sequence encoding a polypeptide, an effector molecule or a tether molecule comprises at least one chemically modified nucleobase, e.g., 5-methoxyuracil. In some embodiments, at least 95% of a nucleobase (e.g., uracil) in a uracil-modified sequence encoding a polypeptide, an effector molecule or a tether molecule of the disclosure are modified nucleobases. In some embodiments, at least 95% of uracil in a uracil-modified sequence encoding a polypeptide, an effector molecule or a tether molecule is 5-methoxyuracil.

In some embodiments, a polynucleotide of the disclosure (e.g., a polynucleotide comprising a nucleotide sequence encoding a polypeptide, an effector molecule or a tether molecule (e.g., the wild-type sequence, functional fragment, or variant thereof) is sequence optimized.

A sequence optimized nucleotide sequence (nucleotide sequence is also referred to as “nucleic acid” herein) comprises at least one codon modification with respect to a reference sequence (e.g., a wild-type sequence encoding a polypeptide, an effector molecule or a tether molecule). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence optimized nucleic acids (e.g., a RNA, e.g., an mRNA) can be administered to a subject in need thereof to facilitate in vivo expression of functionally active encoding a polypeptide, an effector molecule or a tether molecule.

Additional and exemplary methods of sequence optimization are disclosed in International PCT application WO 2017/201325, filed on 18 May 2017, the entire contents of which are hereby incorporated by reference.

Micro RNA (miRNA) and Endonuclease Recognition Sites

Nucleic acid molecules (e.g., RNA, e.g., mRNA) of the disclosure include regulatory elements, for example, microRNA (miRNA) binding sites, endonuclease cleavage sites, structured mRNA sequences and/or motifs, artificial binding sites engineered to act as pseudo-receptors for endogenous nucleic acid binding molecules, and combinations thereof. A regulatory element on an RNA molecule of the disclosure regulates translation of the RNA molecule. In some embodiments, binding of the miRNA or endonuclease to the recognition site within the regulatory element results in cleavage of the RNA molecule at the site of recognition, thereby enhancing or suppressing translation. In other embodiments, binding of the miRNA to the regulatory element on an RNA molecule suppressing translation of the RNA molecule without cleavage. The recognition site may be bound by an miRNA or an endonuclease in a cell-type-specific manner. In some embodiments, the term “modification of the recognition site” refers to the binding of miRNA or endonuclease to the recognition site within the regulatory element, which results in cleavage or non-cleavage based translation repression. In some embodiments, a nucleic acid molecule (e.g., RNA, e.g., mRNA) of the disclosure comprises an open reading frame (ORF) encoding a first polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific, cell-type specific, and/or microenvironment specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site”, “miR target site” (miRts) or “miR recognition site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) of the disclosure comprises an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion of the full length of a naturally-occurring miRNA sequence that is at least 15 nucleotides in length. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs may still be capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is only intended to be delivered to a specific tissue or cell, then a miRNA abundant in that tissue or cell can inhibit the repression of the expression of the gene of interest on a first polynucleotide if one or multiple binding sites of the miRNA are engineered into the 5′UTR and/or 3′UTR of a second polynucleotide (e.g., an mRNA encoding a repressor). The expression of the gene of interest (e.g., a gene encoding a first polypeptide) would be suppressed in tissues/cells that do not express the miRNA.

For example, one of skill in the art would understand that one or more miR binding sites can be included in a nucleic acid molecule (e.g., an RNA, e.g., mRNA) to minimize expression of the gene of interest in cell types other than liver cells (e.g., cells in the lymphoid, myeloid, endothelial, epithelial, or hematopoietic lineages). In one embodiment, a miR122 binding site can be used. In another embodiment, a miR126 binding site can be used. In another embodiment, a miR142 binding site can be used. In still another embodiment, multiple copies of these miR binding sites or combinations may be used.

Regulation of expression of the gene of interest in specific tissues can be accomplished through introduction one or more miRNA binding sites, e.g., one or more distinct miRNA binding sites. The decision on which miRNA binding site to insert can be made based on miRNA expression patterns and/or their profiling in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), spleen (miR142), lymphoid cells (miR150) and lung epithelial cells (let-7, miR-133, miR-126).

Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

In one example, a nucleic acid molecule (e.g., RNA, e.g., mRNA) of the disclosure can comprise a repressor, such as L7Ae under the control of miR142 or miR223 which are selectively abundant in immune cells such as APCs. This would lead to target RNA expression being selectively turned on in APCs (possibly other immune cells such as T cells as well), while target expression would be suppressed in cells (e.g., hepatocytes) that do not express these miRNAs or express them at a negligible level.

In another example, the methods of this disclosure can be used to turn ON expression of a target gene only in APCs. For example, the expression of a target gene may be desired specifically in APCs post vaccination. This could help minimize any unintended events in bystander cells after e.g., intramuscular dosing. In some embodiments, the methods of this disclosure can be used in other immune applications where ON switches could be enabling/increase safety. For example, for Chimeric antigen receptor (CAR) T-cell therapy, it is detrimental to have the CAR expressed in tumor samples since it can mask the target epitope. It can also be detrimental to express the CAR in regulatory T cells (Treg cells). The methods of this disclosure can be used to turn ON expression only in specific T cells.

In some embodiments, the methods of this disclosure can be used to turn ON expression in certain differentiated immune cells for use in immune-oncology applications. In some embodiments, the methods of this disclosure can be used to turn ON expression in particular cell lineages, e.g., hematopoietic progenitor cells in which gene editing is desired, thereby increasing safety of gene-editing technologies.

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA encoding a repressor) of the disclosure, the degree of expression of the gene of interest in specific cell types (e.g., hepatocytes, myeloid cells, endothelial cells, cancer cells, etc.) can be enhanced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can comprise at least one miRNA binding site in the 3′UTR in order to selectively inhibit repression of mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure that is encoding the repressor to target RNA, more unstable in antigen presenting cells. Non-limiting examples of these miRNAs include mir-142-5p, mir-142-3p, mir-146a-5p, and mir-146-3p.

An endonuclease for use in the present disclosure could be effectively any known RNA endonuclease that is sequence or structure specific. See e.g., Tomecki R and Dziembowski A., RNA 2010. 16: 1692-1724; and Schoenberg D R., Wiley Interdiscip Rev RNA. 2011; 2(4): 582-600. In some embodiments, an endonuclease could be an engineered nuclease where a non-specific endonuclease is fused to a specific RNA recognition element (See eg. Choudhary et al. Nature Comm, 2012; 3:1147.) An endonuclease cleavage site can be any site known in the art. See e.g., Mendez A S et al, Nucleic Acids Research, (2018), 46(22): 11968-11979; Zhou W et al Proceedings of the National Academy of Sciences February 2017, 114 (8) E1554-E1563; Floyd-Smith G et al., Science (1981) 212: 4498: 1030-1032.

### IVT Polynucleotide Architecture

In some embodiments, the polynucleotide of the present disclosure comprising an mRNA encoding a therapeutic payload or prophylactic payload, an effector molecule and/or a tether molecule is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded therapeutic payload or prophylactic payload, an effector molecule and/or a tether molecule. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding a therapeutic payload or prophylactic payload, an effector molecule and/or a tether molecule can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of a therapeutic payload or prophylactic payload, an effector molecule and/or a tether molecule or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture are disclosed in International PCT application WO 2017/201325, filed on 18 May 2017, the entire contents of which are hereby incorporated by reference.

### 5′UTR and 3′ UTR

A UTR can be homologous or heterologous to the coding region in a polynucleotide. In some embodiments, the UTR is homologous to the ORF encoding a first polypeptide, an effector molecule or a tether molecule. In some embodiments, the UTR is heterologous to the ORF encoding a first polypeptide, an effector molecule or a tether molecule.

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO: 148), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IEl)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 al (EEF1A1)); a manganese superoxide dismutase (MnSOD); a myocyte enhancer factor 2A (MEF2A); a 3-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nntl); calreticulin (Calr); a procollagen-lysine, 2-oxoglutarate 5-dioxygenase 1 (Plodl); and a nucleobindin (e.g., Nucb1).

In some embodiments, the 5′ UTR is selected from the group consisting of a β-globin 5′ UTR; a 5′UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 al (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a R-F1-ATPase 3′ UTR; functional fragments thereof and combinations thereof.

Wild-type UTRs derived from any gene or mRNA can be incorporated into the polynucleotides of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the disclosure comprise a 5′ UTR and/or a 3′ UTR selected from any of the UTRs disclosed herein, e.g., in Table 6.

TABLE 6
Exemplary 5′ UTR and 3′ UTR sequences

| | |
|---|---|
| SEQ ID NO: | Sequence |

3′ UTR sequences

| | |
|---|---|
| 81 | GCUGGAGCCUCGGUGGCCAUGCUUCUUGCCCCUUGGGCCUCCCCCCAGC |
| | CCCUCCUCCCCUUCCUGCACCCGUACCCCCUCCAUAAAGUAGGAAACAC |
| | UACAGUGGUCUUUGAAUAAAGUCUGAGUGGGCGGC |
| | (3′ UTR with miR 142-3p binding site) |
| 82 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCC [symbol] |
| | [symbol] GUGGUCUUUGAAUAAAGUCUGAGUGGGGGC |
| | (3′ UTR with miR 126-3p binding site) |
| 83 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCGUGGUCUUU |
| | GAAUAAAGUCUGAGUGGGCGGC |
| | (3′ UTR, no miR binding sites) |
| 84 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCUCCAUAAAG |
| | UAGGAAACACUACAGUGGUCUUUGAAUAAAGUCUGAGUGGGCGGC |
| | (3′ UTR with miR 142-3p binding site) |
| 85 | UGAUAAUAGUCCAUAAAGUAGGAAACACUACAGCUGGAGCCUCGGUGG |
| | CCAUGCUUCUUGCCCCUUGGGCCUCCCCCCAGCCCCUCCUCCCCUUCCU |
| | GCACCCGUACCCCC [symbol] [symbol] GUGGUCUUUGAA |
| | UAAAGUCUGAGUGGGCGGC |
| | (3′ UTR with miR 142-3p and miR 126-3p binding sites variant 1) |
| 86 | UGAUAAUAGUCCAUAAAGUAGGAAACACUACAGCUGGAGCCUCGGUGG |
| | CCAUGCUUCUUGCCCCUUGGGCCUCCAUAAAGUAGGAAACACUACAUC |
| | CCCCCAGCCCCUCCUCCCCUUCCUGCACCCGUACCCCCUCCAUAAAGUA |
| | GGAAACACUACAGUGGUCUUUGAAUAAAGUCUGAGUGGGCGGC |
| | (3′ UTR with 3 miR 142-3p binding sites) |
| 87 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 142-5p binding site) |
| 88 | UGAUAAUAGAG [symbol] [symbol] GCUGGAGCCUCGGUGGCC |
| | AUGCUUCUUGCCCCUUGGGCC [symbol] [symbol] UCCCCCC |
| | AGCCCCUCCUCCCCUUCCUGCACCCGUACCCCC [symbol] |
| | [symbol] GUGGUCUUUGAAUAAAGUCUGAGUGGGCGGC |
| | (3′ UTR with 3 miR 142-5p binding sites) |
| 89 | UGAUAAUAG [symbol] GCUGGAGCCUCGGUGGCC |
| | AUGCUUCUUGCCCCUUGGGCCUCCAUAAAGUAGGAAACACUACAUCCC |
| | CCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with 2 miR 142-5p binding sites and 1 miR 142-3p binding site) |
| 90 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCACCCCUAUCA |
| | CAAUUAGCAUUAAGUGGUCUUUGAAUAAAGUCUGAGUGGGCGGC |
| | (3′ UTR with miR 155-5p binding site) |
| 91 | UGAUAAUAGACCCCUAUCACAAUUAGCAUUAAGCUGGAGCCUCGGUGGC |
| | CAUGCUUCUUGCCCCUUGGGCCACCCCUAUCACAAUUAGCAUUAAUCCCC |
| | CCAGCCCCUCCUCCCCUUCCUGCACCCGUACCCCCACCCCUAUCACAAUU |
| | AGCAUUAAGUGGUCUUUGAAUAAAGUCUGAGUGGGCGGC |
| | (3′ UTR with 3 miR 155-5p binding sites) |
| 92 | UGAUAAUAGACCCCUAUCACAAUUAGCAUUAAGCUGGAGCCUCGGUGGC |
| | CAUGCUUCUUGCCCCUUGGGCCUCCAUAAAGUAGGAAACACUACAUCC |
| | CCCCAGCCCCUCCUCCCCUUCCUGCACCCGUACCCCCACCCCUAUCACAA |
| | UUAGCAUUAAGUGGUCUUUGAAUAAAGUCUGAGUGGGCGGC |
| | (3′ UTR with 2 miR 155-5p binding sites and 1 miR 142-3p binding site) |
| 93 | UGAUAAUAGUCCAUAAAGUAGGAAACACUACAGCUGGAGCCUCGGUGG |
| | CCAUGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P1 insertion) |
| 94 | UGAUAAUAGGCUGGAGCCUCGGUGGCUCCAUAAAGUAGGAAACACUAC |
| | ACAUGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P2 insertion) |
| 95 | UGAUAAUAGGCUGGAGCCUCGGUGGCCAUGCUUCUUGCCCCUUGGGCC |
| | UCCAUAAAGUAGGAAACACUACAUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P3 insertion) |
| 96 | UGAUAAUAG [symbol] [symbol] GCUGGAGCCUCGGUGGCC |
| | AUGCUUCUUGCCCCUUGGGCC [symbol] [symbol] UCCCCCC |
| | AGCCCCUCUCCCCUUCCUGCACCCGUACCCCC [symbol] |
| | [symbol] GUGGUCUUUGAAUAAAGUCUGAGUGGGCGGC |
| | (3′ UTR with 3 miR 142-5p binding sites) |
| 97 | UGAUAAUAGGCUGGAGCCUCGGUGGCCAUGCUUCUUGCCCCUUCCAUA |
| | AAGUAGGAAACACUACAUGGGCCUCCCCCCAGCCCCUCCUCCCCUUCCU |
| | GCACCCGUACCCCCGUGGUCUUUGAAUAAAGUCUGAGUGGGCGGC |
| | (3′UTR including miR142-3p binding site) |
| 98 | UGAUAAUAGGCUGGAGCCUCGGUGGCCAUGCUUCUUGCCCCUUGGGCC |
| | UCCCCCCAGUCCAUAAAGUAGGAAACACUACACCCCUCCUCCCCUUCCU |
| | GCACCCGUACCCCCGUGGUCUUUGAAUAAAGUCUGAGUGGGCGGC |
| | (3′UTR including miR142-3p binding site) |
| 99 | UGAUAAUAGGCUGGAGCCUCGGUGGCCAUGCUUCUUGCCCCUUGGGCC |
| | UCCCCCCAGCCCCUCCUCCCCUUCUCCAUAAAGUAGGAAACACUACACU |
| | GCACCCGUACCCCCGUGGUCUUUGAAUAAAGUCUGAGUGGGCGGC |
| | (3′UTR including miR142-3p binding site) |
| 100 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCGUGGUCUUU |
| | GAAUAAAGUUCCAUAAAGUAGGAAACACUACACUGAGUGGGCGGC |
| | (3′UTR including miR142-3p binding site) |
| 101 | UGAUAAUAGUCCAUAAAGUAGGAAACACUACAGCUGGAGCCUCGGUGG |
| | CCUAGCUUCUUGCCCCUUGGGCCUCCCCCCAGCCCCUCCUCCCCUUCCU |
| | GCACCCGUACCCCC [symbol] [symbol] GUGGUCUUUGAA |
| | UAAAGUCUGAGUGGGCGGC |
| | (3′ UTR with miR 142-3p and miR 126-3p binding sites variant 2) |
| 102 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCGUGGUCUUU |
| | GAAUAAAGUCUGAGUGGGCGGC (3′ UTR, no miR binding sites variant 2) |
| 103 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCUCCAUAAAG |
| | UAGGAAACACUACAGUGGUCUUUGAAUAAAGUCUGAGUGGGCGGC |
| | (3′ UTR with miR 142-3p binding site variant 3) |
| 104 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 126-3p binding site variant 3) |
| 105 | UGAUAAUAGUCCAUAAAGUAGGAAACACUACAGCUGGAGCCUCGGUGG |
| | CCUAGCUUCUUGCCCCUUGGGCCUCCAUAAAGUAGGAAACACUACAUC |
| | CCCCCAGCCCCUCCUCCCCUUCCUGCACCCGUACCCCCUCCAUAAAGUA |
| | GGAAACACUACAGUGGUCUUUGAAUAAAGUCUGAGUGGGCGGC |
| | (3′ UTR with 3 miR 142-3p binding sites variant 2) |
| 106 | UGAUAAUAGUCCAUAAAGUAGGAAACACUACAGCUGGAGCCUCGGUGG |
| | CCUAGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′UTR with miR 142-3p binding site, Pl insertion variant 2) |
| 107 | UGAUAAUAGGCUGGAGCCUCGGUGGCUCCAUAAAGUAGGAAACACUAC |
| | ACUAGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′UTR with miR 142-3p binding site, P2 insertion variant 2) |
| 108 | UGAUAAUAGGCUGGAGCCUCGGUGGCCUAGCUUCUUGCCCCUUGGGCC |
| | UCCAUAAAGUAGGAAACACUACAUCCCCCCAGCCCCUCCUCCCCUUCCU |
| | GCACCCGUACCCCCGUGGUCUUUGAAUAAAGUCUGAGUGGGCGGC |
| | (3′UTR with miR 142-3p binding site, P3 insertion variant 2) |
| 109 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCACCCCUAUCA |
| | CAAUUAGCAUUAAGUGGUCUUUGAAUAAAGUCUGAGUGGGCGGC |
| | (3′UTR with miR 155-5p binding site variant 2) |
| 110 | UGAUAAUAGACCCCUAUCACAAUUAGCAUUAAGCUGGAGCCUCGGUGGC |
| | CUAGCUUCUUGCCCCUUGGGCCACCCCUAUCACAAUUAGCAUUAAUCCCC |
| | CCAGCCCCUCCUCCCCUUCCUGCACCCGUACCCCCACCCCUAUCACAAUU |
| | AGCAUUAAGUGGUCUUUGAAUAAAGUCUGAGUGGGCGGC |
| | (3′ UTR with 3 miR 155-5p binding sites variant 2) |
| 111 | UGAUAAUAGACCCCUAUCACAAUUAGCAUUAAGCUGGAGCCUCGGUGGC |
| | CUAGCUUCUUGCCCCUUGGGCCUCCAUAAAGUAGGAAACACUACAUCC |
| | CCCCAGCCCCUCCUCCCCUUCCUGCACCCGUACCCCCACCCCUAUCACAA |
| | UUAGCAUUAAGUGGUCUUUGAAUAAAGUCUGAGUGGGCGGC |
| | (3′UTR with 2 miR 155-5p binding sites and 1 miR 142-3p |
| | binding site variant 2) |
| 112 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCCAAACACCAU |
| | UGUCACACUCCAGUGGUCUUUGAAUAAAGUCUGAGUGGGCGGC |
| | (3′UTR (MIR122)) |
| 113 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCCAAACACCAU |
| | UGUCACACUCCAGUGGUCUUUGAAUAAAGUCUGAGUGGGCGGC |
| | (3′ UTR (MIR122-TAG) |
| 114 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCCGCAUUAUU |
| | ACUCACGGUACGAGUGGUCUUUGAAUAAAGUCUGAGUGGGCGGC |
| | (3′UTR V1.1 (MIR126-3P)) |

5′ UTR SEQUENCES

| | |
|---|---|
| 115 | GGGAAAUAAGAGAGAAAAGAAGAGUAAGAAGAAAUAUAAGAGCCACC |
| | (5′ UTR) |
| 116 | GGGAAAUAAGAGUCCAUAAAGUAGGAAACACUACAAGAAAAGAAGAGU |
| | AAGAAGAAAUAUAAGAGCCACC |
| | (5′ UTR with miR142-3p binding site at position p1) |
| 117 | GGGAAAUAAGAGAGAAAAGAAGAGUAAUCCAUAAAGUAGGAAACACU |
| | ACAGAAGAAAUAUAAGAGCCACC |
| | (5′ UTR with miR142-3p binding site at position p2) |
| 118 | GGGAAAUAAGAGAGAAAAGAAGAGUAAGAAGAAAUAUAAUCCAUAAA |
| | GUAGGAAACACUACAGAGCCACC |
| | (5′ UTR with miR142-3p binding site at position p3) |
| 119 | AGGAAAUAAGAGAGAAAAGAAGAGUAAGAAGAAAUAUAAGAGCCACC |
| | (5′-UTR (v1 A-start)) |
| 120 | GGGAAAUAAGAGAGAAAAGAAGAGUAAGAAGAAAUAUAAGACCCCGGC |
| | GCCGCCACC |
| | (5′ UTR v1.1) |
| 121 | AGGAAAUAAGAGAGAAAAGAAGAGUAAGAAGAAAUAUAAGACCCCGGC |
| | GCCGCCACC |
| | (5′UTR v1.1 A-start) |
| 122 | GGGAGAUCAGAGAGAAAAGAAGAGUAAGAAGAAAUAUAAGAGCCACC |
| | 5′ UTR 002 (upstream UTR) |
| 123 | GGGAGACAAGCUUGGCAUUCCGGUACUGUUGGUAAAGCCACC |
| | 5′ UTR-004 (Upstream UTR) |
| 124 | GGGAAUUAACAGAGAAAAGAAGAGUAAGAAGAAAUAUAAGAGCCACC |
| | 5′ UTR-008 (Upstream UTR) |
| 125 | GGGAAAUUAGACAGAAAAGAAGAGUAAGAAGAAAUAUAAGAGCCACC |
| | 5′ UTR-009 (Upstream UTR) |
| 126 | GGGAAAUAAGAGAGUAAAGAACAGUAAGAAGAAAUAUAAGAGCCACC |
| | 5′ UTR-010, Upstream |
| 127 | GGGAAAAAAGAGAGAAAAGAAGACUAAGAAGAAAUAUAAGAGCCACC |
| | 5′ UTR-011 (Upstream UTR) |
| 128 | GGGAAAUAAGAGAGAAAAGAAGAGUAAGAAGAUAUAUAAGAGCCACC |
| | 5′ UTR-012 (Upstream UTR) |
| 129 | GGGAAAUAAGAGACAAAACAAGAGUAAGAAGAAAUAUAAGAGCCACC |
| | 5′ UTR-013 (Upstream UTR) |
| 130 | GGGAAAUUAGAGAGUAAAGAACAGUAAGUAGAAUUAAAAGAGCCACC |
| | 5′ UTR-014 (Upstream UTR) |
| 131 | GGGAAAUAAGAGAGAAUAGAAGAGUAAGAAGAAAUAUAAGAGCCACC |
| | 5′ UTR-015 (Upstream UTR) |
| 132 | GGGAAAUAAGAGAGAAAAGAAGAGUAAGAAGAAAAUUAAGAGCCACC |
| | 5′ UTR-016 (Upstream UTR) |
| 133 | GGGAAAUAAGAGAGAAAAGAAGAGUAAGAAGAAAUUUAAGAGCCACC |
| | 5′ UTR-017 (Upstream UTR) |
| 134 | UCAAGCUUUUGGACCCUCGUACAGAAGCUAAUACGACUCACUAUAGGG |
| | AAAUAAGAGAGAAAAGAAGAGUAAGAAGAAAUAUAAGAGCCACC |
| | 5′ UTR-018 (Upstream UTR) |
| 135 | GGGAAACGGCAAGCAGCAGCUGCCGCUGUUUUGUCUUAACCGGUUGAA |
| | AGACCAUUAGGCUUUCUAACGGACGCUCACGAGAGAGGGGCACCAAUA |
| | GGGCCCCACGGCACUUCAAAGGUUUGGCUUGGAGUAGUAACCCAAGCA |
| | GCAACAGUUUUGACUUUCGGACCACCAUCAGGGGUCCCACGUUGGGAA |
| | CACGUAACUCUCCUACUAACAAGAGGAGCCCCGGCGCCGCCACC |
| | 5′UTR with 6XMS2 sites |
| 153 | GGGAAAUCGCAAAAUUUGCUCUUCGCGUUAGAUUUCUUUUAGUUUUCU |
| | CGCAACUAGCAAGCUUUUUGUUCUCGCC |
| | 5′UTR for MBP-LA |

Stop codon = bold
miR 142-3p binding site = underline
miR 126-3p binding site = bold underline
miR 155-5p binding site = italicized
miR 142-5p binding site = italicized and bold underline

In certain embodiments, the 5′ UTR and/or 3′ UTR sequence of the disclosure comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in Table 6.

In some embodiments, the 5′ UTR comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in Table 6.

In some embodiments, the 3′ UTR comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in Table 6.

In some embodiments, the polynucleotide disclosed herein, e.g., the polynucleotide encoding a first polypeptide, an effector molecule or a tether molecule, comprises a 5′ UTR having the sequence of a 5′ UTR provided in Table 6, or a sequence with at least about 60%, at least about 70%, at least about 80%, at least about 90%, at least about 95%, at least about 96%, at least about 97%, at least about 98%, at least about 99%, or about 100% identity thereto. In some embodiments, the polynucleotide comprises a 5′ UTR comprising the sequence of any one of SEQ ID NOs: 115-135, or a sequence with at least about 60%, at least about 70%, at least about 80%, at least about 90%, at least about 95%, at least about 96%, at least about 97%, at least about 98%, at least about 99%, or about 100% identity thereto.

In some embodiments, the polynucleotide disclosed herein, e.g., the polynucleotide encoding a first polypeptide, an effector molecule or a tether molecule, comprises a 3′ UTR having the sequence of a 3′ UTR provided in Table 6, or a sequence with at least about 60%, at least about 70%, at least about 80%, at least about 90%, at least about 95%, at least about 96%, at least about 97%, at least about 98%, at least about 99%, or about 100% identity thereto. In some embodiments, the polynucleotide comprises a 3′ UTR comprising the sequence of any one of SEQ ID NOs: 81-114, or a sequence with at least about 60%, at least about 70%, at least about 80%, at least about 90%, at least about 95%, at least about 96%, at least about 97%, at least about 98%, at least about 99%, or about 100% identity thereto.

The polynucleotides of the disclosure can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the disclosure. For example, introns or portions of intron sequences can be incorporated into the polynucleotides of the disclosure. Incorporation of intronic sequences can increase protein production as well as polynucleotide expression levels. In some embodiments, the polynucleotide of the disclosure comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation.

### Regions Having a 5′ Cap

The disclosure also includes a polynucleotide that comprises both a 5′ Cap and a polynucleotide of the present disclosure (e.g., a polynucleotide comprising a nucleotide sequence encoding a first polypeptide, an effector molecule or a tether molecule).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present disclosure (e.g., a polynucleotide comprising a nucleotide sequence encoding a first polypeptide, an effector molecule or a tether molecule) incorporate a cap moiety.

In some embodiments, polynucleotides of the present disclosure (e.g., a polynucleotide comprising a nucleotide sequence encoding a first polypeptide, an effector molecule or a tether molecule) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the disclosure (e.g., a polynucleotide comprising a nucleotide sequence encoding a first polypeptide, an effector molecule or a tether molecule), can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

As a non-limiting example, capping chimeric polynucleotides post-manufacture can be more efficient as nearly 100% of the chimeric polynucleotides can be capped. This is in contrast to ˜80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly A Tails

In some embodiments, the polynucleotides of the present disclosure (e.g., a polynucleotide comprising a nucleotide sequence encoding a first polypeptide, an effector molecule or a tether molecule) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO: 149).

(SEQ ID NO: 149)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa

PolyA tails can also be added after the construct is exported from the nucleus.

According to the present disclosure, terminal groups on the poly A tail can be incorporated for stabilization. Polynucleotides of the present disclosure can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present disclosure can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs (Norbury, Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present disclosure. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO: 150).

(SEQ ID NO: 150)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa aaaaaaaaaa aaaaaaaaaa

### Start Codon Region

The disclosure also includes a polynucleotide that comprises both a start codon region and the polynucleotide described herein (e.g., a polynucleotide comprising a nucleotide sequence encoding a first polypeptide, an effector molecule or a tether molecule). In some embodiments, the polynucleotides of the present disclosure can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The disclosure also includes a polynucleotide that comprises both a stop codon region and the polynucleotide described herein (e.g., a polynucleotide comprising a nucleotide sequence encoding a first polypeptide, an effector molecule or a tether molecule). In some embodiments, the polynucleotides of the present disclosure can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present disclosure include the stop codon TGA in the case or DNA, or the stop codon UGA in the case of RNA, and one additional stop codon. In a further embodiment the addition stop codon can be TAA or UAA. In another embodiment, the polynucleotides of the present disclosure include three consecutive stop codons, four stop codons, or more.

### Chemical Modifications of Polynucleotides

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises N1-methyl-pseudouridine (m1ψ) substitutions at one or more or all uridine positions of the nucleic acid.

In some embodiments, a RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Pharmaceutical Compositions

The present disclosure provides pharmaceutical formulations comprising any of the systems, or compositions disclosed herein. In some embodiments, the pharmaceutical formulation comprises (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein modification of the recognition site reduces translation of the repressor from the second polynucleotide, wherein binding of the repressor to the repressor binding element reduces translation of the polypeptide from the first polynucleotide.

In some embodiments, the pharmaceutical formulation comprises (a) a first polynucleotide comprising (i) an open reading frame encoding a first polypeptide, (ii) an effector binding element, and (iii) a recognition site, wherein the first polynucleotide is mRNA; and (b) a second polynucleotide comprising a sequence encoding a second polypeptide, wherein the second polypeptide comprises an effector, wherein binding of the effector to the effector binding element increases translation of the first polypeptide from the first polynucleotide.

In some embodiments, the pharmaceutical formulation comprises (a) an RNA molecule comprising in order from the 5′ to 3′ end of the RNA (i) an open reading frame encoding a polypeptide, (ii) a polyA tail, (iii) a cleavage site, and (iv) a destabilizing sequence; and
(b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide.

In some embodiments of the disclosure, the polynucleotide are formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intra-synovial, intrasternal, intrathecal, intrahepatic, intralesional, intracranial, intraventricular, oral, inhalation spray, pulmonary, topical, rectal, nasal, buccal, vaginal, or implanted reservoir intramuscular, subcutaneous, or intradermal delivery. In other embodiments, the polynucleotide is formulated for subcutaneous or intravenous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations and Delivery

The polynucleotide comprising an mRNA of the disclosure can be formulated using one or more excipients.

The function of the one or more excipients is, e.g., to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotides is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core.

A polynucleotide comprising an mRNA of the disclosure can be delivered to a cell using any method known in the art. For example, the polynucleotide comprising an mRNA of the disclosure can be delivered to a cell by a lipid-based delivery, e.g., transfection, or by electroporation.

### Delivery Agents

The compositions and systems disclosed herein further comprises a delivery agent. The delivery agent of the present disclosure can include, without limitation, liposomes, lipid nanoparticles, lipidoids, polymers, lipoplexes, polyplexes, microvesicles, exosomes, peptides, proteins, cells transfected with polynucleotides, hyaluronidase, nanoparticle mimics, nanotubes, conjugates, and combinations thereof.

a. Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and one or more polynucleotides disclosed herein, e.g., mRNA, has an increased therapeutic index as compared to a corresponding formulation which comprises a reference lipid (e.g., MC3, KC2, or DLinDMA) and the same one or more polynucleotides.

In certain embodiments, the present application provides pharmaceutical compositions comprising: (a) a first polynucleotide comprising (i) a repressor binding element and (ii) an open reading frame encoding a polypeptide; (b) a second polynucleotide comprising (i) a sequence encoding a repressor that binds to the repressor binding element and (ii) a recognition site reduces translation of the repressor from the second polynucleotide, wherein binding of the repressor to the repressor binding element reduces translation of the polypeptide from the first polynucleotide; and (c) a delivery agent.

In some embodiments, the pharmaceutical formulation comprises (a) a first polynucleotide comprising (i) an open reading frame encoding a first polypeptide, (ii) an effector binding element, and (iii) a recognition site, wherein the first polynucleotide is mRNA; (b) a second polynucleotide comprising a sequence encoding a second polypeptide, wherein the second polypeptide comprises an effector, wherein binding of the effector to the effector binding element increases translation of the first polypeptide from the first polynucleotide; and (c) a delivery agent.

In some embodiments, the pharmaceutical formulation comprises (a) an RNA molecule comprising in order from the 5′ to 3′ end of the RNA (i) an open reading frame encoding a polypeptide, (ii) a polyA tail, (iii) a cleavage site, and (iv) a destabilizing sequence;
(b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide; and (c) a delivery agent.

### (I) Lipid Nanoparticle Compositions

In some embodiments, the nucleic acids of the disclosure are formulated as lipid nanoparticle (LNP) compositions. The LNPs disclosed herein comprise an (i) ionizable lipid; (ii) sterol or other structural lipid; (iii) a non-cationic helper lipid or phospholipid; and, optionally a (iv) PEG lipid. These categories of lipids are set forth in more detail below. Lipid nanoparticles typically comprise amino lipid, phospholipid, structural lipid and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entireties.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

### (I)(a) Amino Lipids

In some aspects, the amino lipids of a lipid nanoparticle composition disclosed herein may be one or more of compounds of Formula (I):

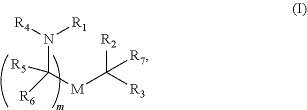

or their N-oxides, or salts or isomers thereof, wherein:
R1 is selected from the group consisting of C5-30 alkyl, C5-20 alkenyl, —R*YR″, —YR″, and —R″M′R′;
R2 and R3 are independently selected from the group consisting of H, C1-14 alkyl, C2-14 alkenyl, —R*YR″, —YR″, and —R*OR″, or R2 and R3, together with the atom to which they are attached, form a heterocycle or carbocycle;
R4 is selected from the group consisting of hydrogen, a C3-6 carbocycle, —(CH2)nQ, —(CH2)nCHQR,
—CHQR, —CQ(R)2, and unsubstituted C1-6 alkyl, where Q is selected from a carbocycle, heterocycle, —OR, —O(CH2)nN(R)2, —C(O)OR, —OC(O)R, —CX3, —CX2H, —CXH2, —CN, —N(R)2, —C(O)N(R)2, —N(R)C(O)R, —N(R)S(O)2R, —N(R)C(O)N(R)2, —N(R)C(S)N(R)2, —N(R)R8, —N(R)S(O)2R8, —O(CH2)nOR, —N(R)C(═NR9)N(R)2, —N(R)C(═CHR9)N(R)2, —OC(O)N(R)2,
—N(R)C(O)OR, —N(OR)C(O)R, —N(OR)S(O)2R, —N(OR)C(O)OR, —N(OR)C(O)N(R)2, —N(OR)C(S)N(R)2, —N(OR)C(═NR9)N(R)2, —N(OR)C(═CHR9)N(R)2, —C(═NR9)N(R)2, —C(═NR9)R, —C(O)N(R)OR, and —C(R)N(R)2C(O)OR, and each n is independently selected from 1, 2, 3, 4, and 5;
each R5 is independently selected from the group consisting of C1-3 alkyl, C2-3 alkenyl, and H;
each R6 is independently selected from the group consisting of C1-3 alkyl, C2-3 alkenyl, and H;
M and M′ are independently selected
from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)2-, —S—S—, an aryl group, and a heteroaryl group, in which M″ is a bond, C1-13 alkyl or C2-13 alkenyl;
R7 is selected from the group consisting of C1-3 alkyl, C2-3 alkenyl, and H;
R8 is selected from the group consisting of C3-6 carbocycle and heterocycle;
R9 is selected from the group consisting of H, CN, NO2, C1-6 alkyl, —OR, —S(O)2R, —S(O)2N(R)2, C2-6 alkenyl, C3-6 carbocycle and heterocycle;
each R is independently selected from the group consisting of C1-3 alkyl, C2-3 alkenyl, and H;
each R′ is independently selected from the group consisting of C1-18 alkyl, C2-18 alkenyl, —R*YR″, —YR″, and H;
each R″ is independently selected from the group consisting of C3-15 alkyl and C3-15 alkenyl;
each R* is independently selected from the group consisting of C1-12 alkyl and C2-12 alkenyl;
each Y is independently a C3-6 carbocycle;
each X is independently selected from the group consisting of F, Cl, Br, and I; and m is selected from 5, 6, 7, 8, 9, 10, 11, 12, and 13; and wherein when R4
is —(CH2)nQ, —(CH2)nCHQR, —CHQR, or —CQ(R)2, then (i) Q is not —N(R)2 when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

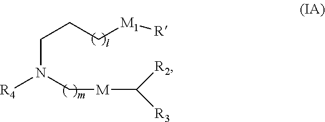

or its N-oxide, or a salt or isomer thereof, wherein 1 is selected from 1, 2, 3, 4, and 5; m is selected from 5, 6, 7, 8, and 9; M1 is a bond or M′; R4 is hydrogen, unsubstituted C1-3 alkyl, or —(CH2)nQ, in which Q is
OH, —NHC(S)N(R)2, —NHC(O)N(R)2, —N(R)C(O)R, —N(R)S(O)2R, —N(R)R8, —NHC(═NR9)N(R)2, —NHC(═CHR9)N(R)2, —OC(O)N(R)2, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl. For example, m is 5, 7, or 9. For example, Q is OH, —NHC(S)N(R)2, or —NHC(O)N(R)2. For example, Q is —N(R)C(O)R, or —N(R)S(O)2R.

In certain embodiments, a subset of compounds of Formula (I) includes those of Formula (IB):

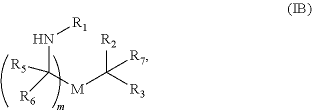

or its N-oxide, or a salt or isomer thereof in which all variables are as defined herein. For example, m is selected from 5, 6, 7, 8, and 9; R4 is hydrogen, unsubstituted C1-3 alkyl, or —(CH2)nQ, in which Q is OH, —NHC(S)N(R)2, —NHC(O)N(R)2, —N(R)C(O)R, —N(R)S(O)2R, —N(R)R8, —NHC(═NR9)N(R)2, —NHC(═CHR9)N(R)2, —OC(O)N(R)2, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl. For example, m is 5, 7, or 9. For example, Q is OH, —NHC(S)N(R)2, or —NHC(O)N(R)2. For example, Q is —N(R)C(O)R, or —N(R)S(O)2R.

In certain embodiments, a subset of compounds of Formula (I) includes those of Formula (II):

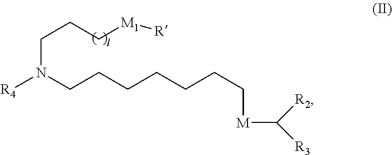

or its N-oxide, or a salt or isomer thereof, wherein 1 is selected from 1, 2, 3, 4, and 5; M1 is a bond or M′; R4 is hydrogen, unsubstituted C1-3 alkyl, or —(CH2)nQ, in which n is 2, 3, or 4, and Q is OH, —NHC(S)N(R)2, —NHC(O)N(R)2, —N(R)C(O)R, —N(R)S(O)2R, —N(R)R8, —NHC(═NR9)N(R)2, —NHC(═CHR9)N(R)2, —OC(O)N(R)2, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl.

In one embodiment, the compounds of Formula (I) are of Formula (IIa),

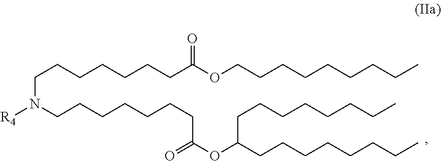

or their N-oxides, or salts or isomers thereof, wherein R4 is as described herein.

In another embodiment, the compounds of Formula (I) are of Formula (IIb),

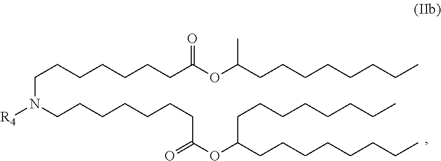

or their N-oxides, or salts or isomers thereof, wherein R4 is as described herein.

In another embodiment, the compounds of Formula (I) are of Formula (IIc) or (IIe):

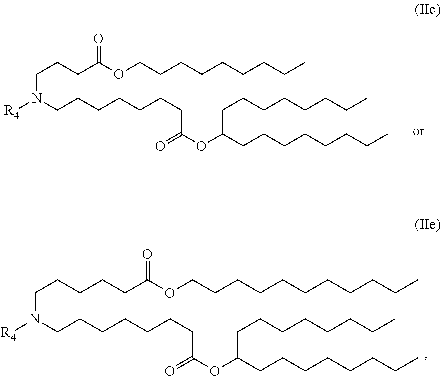

or their N-oxides, or salts or isomers thereof, wherein R4 is as described herein.

In another embodiment, the compounds of Formula (I) are of Formula (IIf):

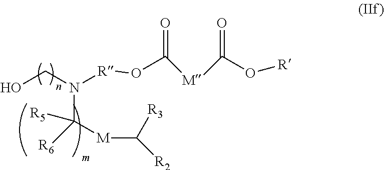

or their N-oxides, or salts or isomers thereof,
wherein M is —C(O)O— or —OC(O)—, M″ is C1-6 alkyl or C2-6 alkenyl, R2 and R3 are independently selected from the group consisting of C5-14 alkyl and C5-14 alkenyl, and n is selected from 2, 3, and 4.

In a further embodiment, the compounds of Formula (I) are of Formula (IId),

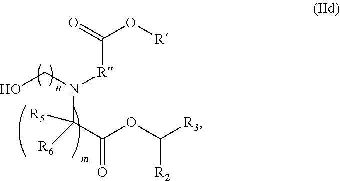

or their N-oxides, or salts or isomers thereof, wherein n is 2, 3, or 4; and m, R′, R″, and R2 through R6 are as described herein. For example, each of R2 and R3 may be independently selected from the group consisting of C5-14 alkyl and C5-14 alkenyl.

In a further embodiment, the compounds of Formula (I) are of Formula (IIg),

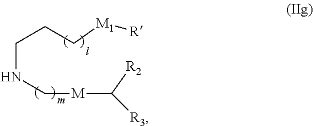

or their N-oxides, or salts or isomers thereof, wherein 1 is selected from 1, 2, 3, 4, and 5; m is selected from 5, 6, 7, 8, and 9; M1 is a bond or M′; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R2 and R3 are independently selected from the group consisting of H, C1-14 alkyl, and C2-14 alkenyl. For example, M″ is C1-6 alkyl (e.g., C1-4 alkyl) or C2-6 alkenyl (e.g. C2-4 alkenyl). For example, R2 and R3 are independently selected from the group consisting of C5-14 alkyl and C5-14 alkenyl.

In some embodiments, the amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the amino lipid is

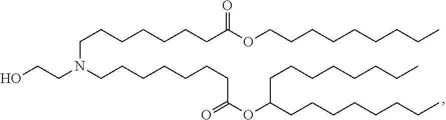 [SMILES: CCCCCCCCCOC(=O)CCCCCCCN(CCO)CCCCCCCC(=O)OC(CCCCCCCC)CCCCCCCC]

or a salt thereof.

In some embodiments, the amino lipid is

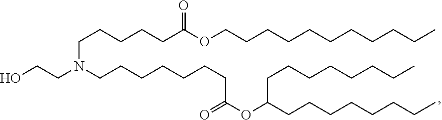 [SMILES: CCCCCCCCCCCOC(=O)CCCCCN(CCO)CCCCCCCC(=O)OC(CCCCCCCC)CCCCCCCC]

or a salt thereof.

The central amine moiety of a lipid according to Formula (I), (IA), (IB), (II), (IIa), (IIb), (IIc), (IId), (IIe), (IIf), or (IIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some aspects, the amino lipids of the present disclosure may be one or more of compounds of formula (III),

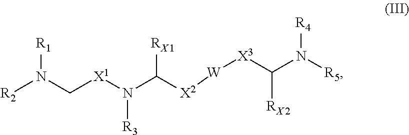

or salts or isomers thereof, wherein

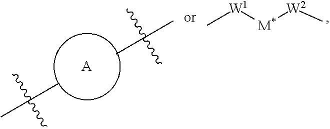

W is or M
ring A is

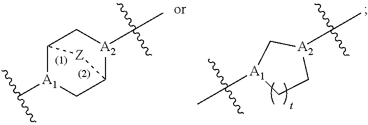

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C1-3 alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —S C(S)—,
   —CH(OH)—, —P(O)(OR′)O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and C₁₋₅ alkyl; X¹, X², and X³ are independently selected from the group consisting of a bond, —CH₂—,
   —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

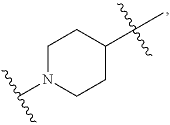 [SMILES: *C1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (IIIa1)-(IIIa8):

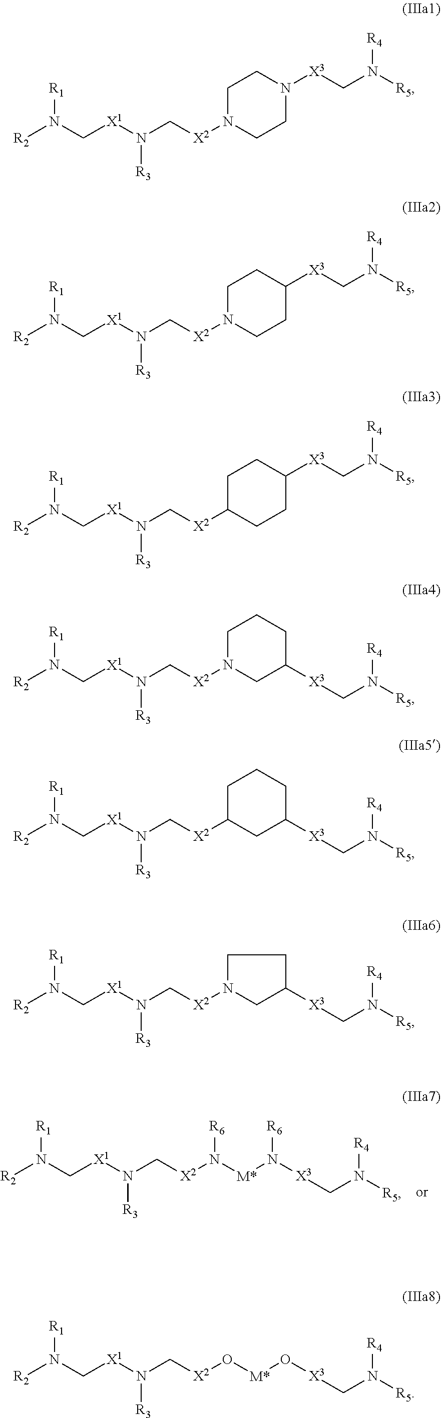

In some embodiments, the amino lipid is

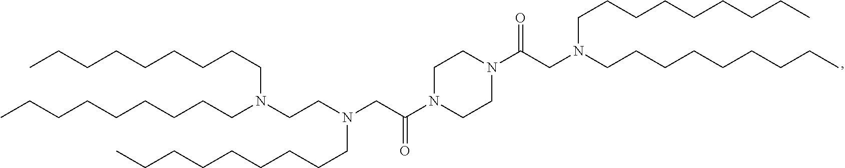 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH.

Thus, a lipid may have a positive or partial positive charge at physiological pH.

### (I)(b) Phospholipids

The lipid composition of a lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present disclosure is a compound of Formula (IV):

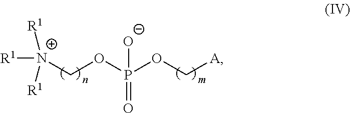

or a salt thereof, wherein:
each R1 is independently optionally substituted alkyl; or optionally two R1 are joined together with the intervening atoms to form optionally substituted monocyclic carbocyclyl or optionally substituted monocyclic heterocyclyl; or optionally three R1 are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;

A is of the formula:

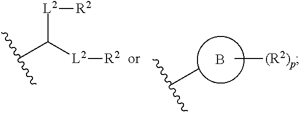

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NRNC(O) C(O)O, OC(O), —OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O)—NR^{N}C(O)N(R^{N}), C(O)O, OC(O), OC(O)O, OC(O)N(R^{N}), NR^{N}C(O)O C(O)S, SC(O), —C(═NR^{N}), C(═NR^{N})N(R^{N}), NR^{C}((═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

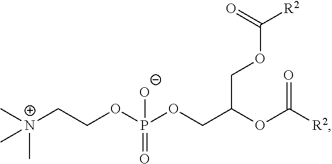

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530, or in International Application PCT/US2018/037922 filed on 15 Jun. 2018, the entire contents of each of which is hereby incorporated by reference in its entirety.

### i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following formulae:

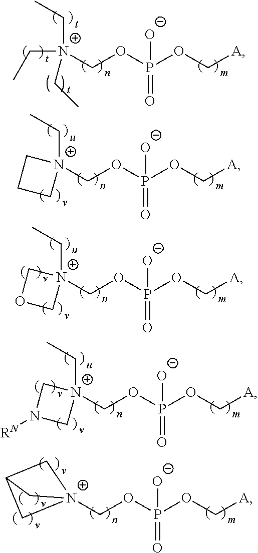

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

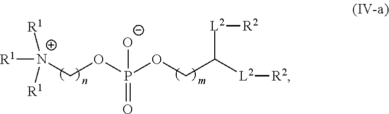

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

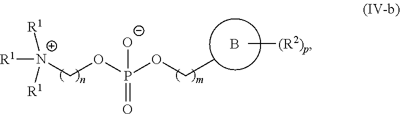

or a salt thereof.

### (ii) Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C1-30 alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O)—NR^{N}C(O)N(R^{N}), C(O)O, OC(O), OC(O)O, OC(O)N(R^{N}), NR^{N}C(O)O C(O)S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O.

In certain embodiments, the compound of Formula (IV) is of Formula (IV-c):

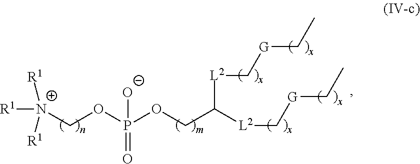

or a salt thereof, wherein:
each x is independently an integer between 0-30, inclusive; and each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O) NR^{N}C(O)N(R^{N}), C(O)O, OC(O), OC(O)O, OC(O)N(R^{N}), NR^{N}C(O)O C(O)S, SC(O), —C(═NR^{N}), C(═NR^{N})N(R^{N}), NRC((═NR^{N}) NR^{N}C(═N^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following formulae:

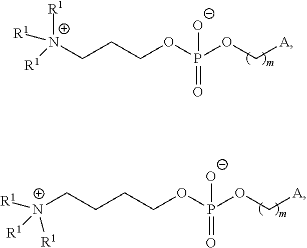

or a salt thereof.
   (iii) Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, an alternative lipid is useful.

In certain embodiments, an alternative lipid is used in place of a phospholipid of the present disclosure. In certain embodiments, an alternative lipid of the invention is oleic acid. In certain embodiments, the alternative lipid is one of the following:

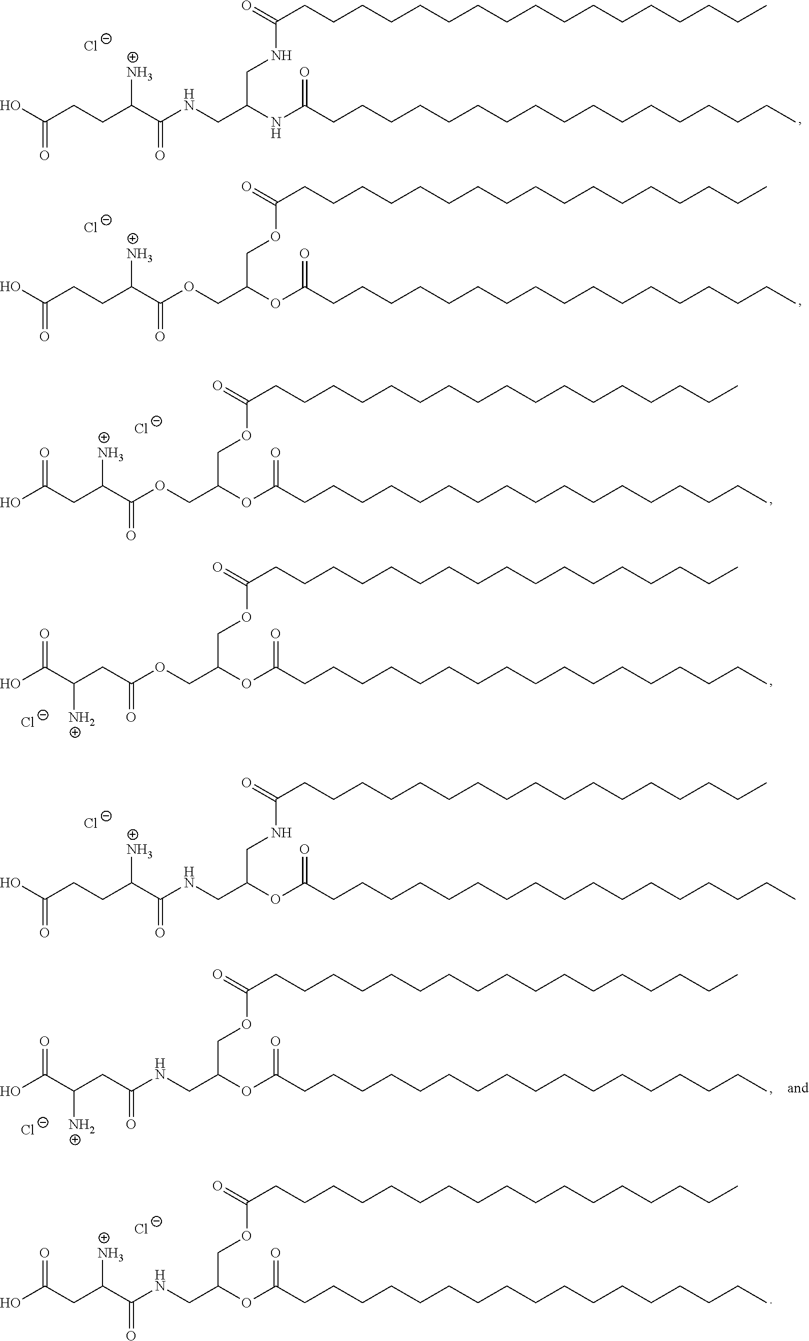

### (I)(c) Structural Lipids

The lipid composition of a lipid nanoparticle composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

### (I)(d) Polyethylene Glycol (PEG)-Lipids

The lipid composition of a lipid nanoparticle composition disclosed herein disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C14 to about C22, preferably from about C14 to about C16. In some embodiments, a PEG moiety, for example an mPEG-NH2, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

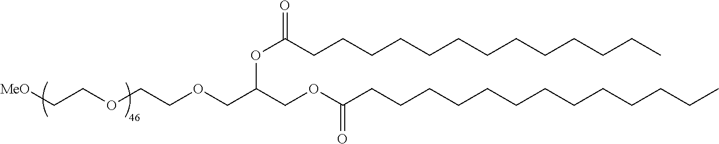

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

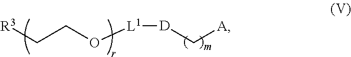

or salts thereof, wherein:
R3 is —ORO;
RO is hydrogen, optionally substituted alkyl, or an oxygen protecting group;
r is an integer between 1 and 100, inclusive;
L1 is optionally substituted C1-10 alkylene, wherein at least one methylene of the optionally substituted C1-10 alkylene is independently replaced with optionally substituted carbocyclylene, optionally substituted heterocyclylene, optionally substituted arylene, optionally substituted heteroarylene, O, N(RN), S, C(O), C(O)N(RN), —NRNC(O), C(O)O, OC(O), OC(O)O, OC(O)N(RN), NRNC(O)O, or NRNC(O)N(RN);
D is a moiety obtained by click chemistry or a moiety cleavable under physiological conditions;
m is 0, 1, 2, 3, 4, 5, 6, 7, 8, 9, or 10;
A is of the formula:

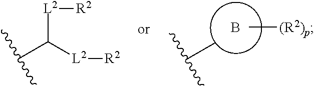

each instance of L2 is independently a bond or optionally substituted C1-6 alkylene, wherein one methylene unit of the optionally substituted C1-6 alkylene is optionally replaced with O, N(RN), S, C(O), C(O)N(RN), NRNC(O), C(O)O, OC(O), OC(O)O, —OC(O)N(RN), NRNC(O)O, or NRNC(O)N(RN);
each instance of R2 is independently optionally substituted C1-30 alkyl, optionally substituted C1-30 alkenyl, or optionally substituted C1-30 alkynyl; optionally wherein one or more methylene units of R2 are independently replaced with optionally substituted carbocyclylene, optionally substituted heterocyclylene, optionally substituted arylene, optionally substituted heteroarylene, N(RN), O, S, C(O), C(O)N(RN), —NRNC(O), NRNC(O)N(RN), C(O)O, OC(O), OC(O)O, OC(O)N(RN), NRNC(O)O, —C(O)S, SC(O), C(═NRN), C(═NRN)N(RN), NRNC(═NRN), NRNC(═NRN)N(RN), —C(S), C(S)N(RN), NRNC(S), NRNC(S)N(RN), S(O), OS(O), S(O)O, OS(O)O, OS(O)2, S(O)2O, OS(O)2O, N(RN)S(O), S(O)N(RN), N(RN)S(O)N(RN), OS(O)N(RN), —N(RN)S(O)O, S(O)2, N(RN)S(O)2, S(O)2N(RN), N(RN)S(O)2N(RN), OS(O)2N(RN), or N(RN)S(O)2O;
each instance of RN is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R3 is —ORO, and RO is hydrogen). In certain embodiments, the compound of Formula (V) is of Formula (V—OH):

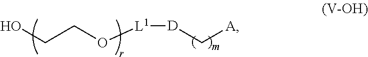

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI-A):

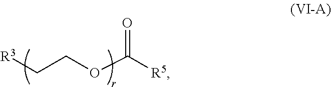

or a salts thereof, wherein:
R3 is-ORO;
RO is hydrogen, optionally substituted alkyl or an oxygen protecting group;
r is an integer between 1 and 100, inclusive;
R5 is optionally substituted C10-40 alkyl, optionally substituted C10-40 alkenyl, or optionally substituted C10-40 alkynyl; and optionally one or more methylene groups of R5 are replaced with optionally substituted carbocyclylene, optionally substituted heterocyclylene, optionally substituted arylene, optionally substituted heteroarylene, —N(RN), O, S, C(O), C(O)N(RN), NRNC(O), NRNC(O)N(RN), C(O)O, OC(O), OC(O)O, OC(O)N(RN), NRNC(O)O, C(O)S, SC(O), C(═NRN), C(═NRN)N(RN), NRNC(═NRN), NRNC(═NRN)N(RN), C(S), C(S)N(RN), NRNC(S), NRNC(S)N(RN), S(O), OS(O), —S(O)O, OS(O)O, OS(O)2, S(O)2O, OS(O)2O, N(RN)S(O), S(O)N(RN), —N(RN)S(O)N(RN), OS(O)N(RN), N(RN)S(O)O, S(O)2, N(RN)S(O)2, S(O)2N(RN), —N(RN)S(O)2N(RN), OS(O)2N(RN), or N(RN)S(O)2O; and
each instance of RN is independently hydrogen, optionally substituted alkyl, or a nitrogen protecting group.

In certain embodiments, the compound of Formula (VI) is of Formula (VI—OH):

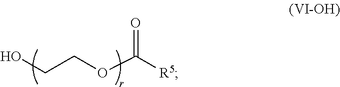

also referred to as (VI-B),
or a salt thereof. In some embodiments, r is 40-50.

In yet other embodiments the compound of Formula (VI-C) is:

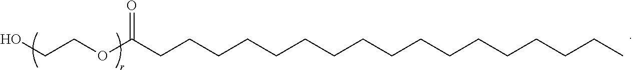

or a salt thereof.

In one embodiment, the compound of Formula (VI-D) is

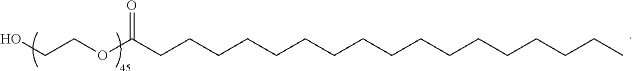

In some aspects, the lipid composition of the pharmaceutical compositions disclosed herein does not comprise a PEG-lipid. In some embodiments, the PEG-lipids may be one or more of the PEG lipids described in U.S. Application No. U.S. Ser. No. 15/674,872.

In some embodiments, a LNP of the disclosure comprises an amino lipid of any of Formula I, II or III, a phospholipid comprising DSPC, a structural lipid, and a PEG lipid comprising PEG-DMG. In some embodiments, a LNP of the disclosure comprises an amino lipid of any of Formula I, II or III, a phospholipid comprising DSPC, a structural lipid, and a PEG lipid comprising a compound having Formula VI. In some embodiments, a LNP of the disclosure comprises an amino lipid of Formula I, II or III, a phospholipid comprising a compound having Formula IV, a structural lipid, and the PEG lipid comprising a compound having Formula V or VI. In some embodiments, a LNP of the disclosure comprises an amino lipid of Formula I, II or III, a phospholipid comprising a compound having Formula IV, a structural lipid, and the PEG lipid comprising a compound having Formula V or VI. In some embodiments, a LNP of the disclosure comprises an amino lipid of Formula I, II or III, a phospholipid having Formula IV, a structural lipid, and a PEG lipid comprising a compound having Formula VI.

In some embodiments, a LNP of the disclosure comprises an N:P ratio of from about 2:1 to about 30:1. In some embodiments, a LNP of the disclosure comprises an N:P ratio of about 6:1. In some embodiments, a LNP of the disclosure comprises an N:P ratio of about 3:1, 4:1, or 5:1. In some embodiments, a LNP of the disclosure comprises a wt/wt ratio of the amino lipid component to the RNA of from about 10:1 to about 100:1. In some embodiments, a LNP of the disclosure comprises a wt/wt ratio of the amino lipid component to the RNA of about 20:1. In some embodiments, a LNP of the disclosure comprises a wt/wt ratio of the amino lipid component to the RNA of about 10:1.

In some embodiments, a LNP of the disclosure has a mean diameter from about 30 nm to about 150 nm. In some embodiments, a LNP of the disclosure has a mean diameter from about 60 nm to about 120 nm.

b. Nanoparticle Compositions

In some embodiments, the pharmaceutical compositions disclosed herein are formulated as lipid nanoparticles (LNP). Accordingly, the present disclosure also provides nanoparticle compositions comprising (I) a lipid composition comprising a delivery agent such as compound as described herein, and (II) (a) a first polynucleotide comprising (i) a repressor binding element and (ii) an open reading frame encoding a polypeptide; and (b) a second polynucleotide comprising (i) a sequence encoding a repressor that binds to the repressor binding element and (ii) a recognition site, wherein modification of the recognition site reduces translation of the repressor from the second polynucleotide, wherein binding of the repressor to the repressor binding element reduces translation of the polypeptide from the first polynucleotide.

In some embodiments, the present disclosure also provides nanoparticle compositions comprising (I) a lipid composition comprising a delivery agent such as compound as described herein, and (II)(a) a first polynucleotide comprising (i) an open reading frame encoding a first polypeptide, (ii) an effector binding element, and (iii) a recognition site, wherein the first polynucleotide is mRNA; and (b) a second polynucleotide comprising a sequence encoding a second polypeptide, wherein the second polypeptide comprises an effector, wherein binding of the effector to the effector binding element increases translation of the first polypeptide from the first polynucleotide.

In some embodiments, the present disclosure also provides nanoparticle compositions comprising (I) a lipid composition comprising a delivery agent such as compound as described herein, and (II) (a) an RNA molecule comprising in order from the 5′ to 3′ end of the RNA (i) an open reading frame encoding a polypeptide, (ii) a polyA tail, (iii) a cleavage site, and (iv) a destabilizing sequence; and (b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide.

In such nanoparticle compositions described above, the lipid composition disclosed herein can encapsulate the polynucleotide encoding a first polypeptide and, when present, the polynucleotide encoding the second polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a sterol and a structural lipid. In some embodiments, the LNP has a molar ratio of about 20-60% ionizable lipid: about 5-25% structural lipid: about 25-55% sterol; and about 0.5-15% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

In addition to these, an ionizable lipid may also be a lipid including a cyclic amine group.

In one embodiment, the ionizable lipid may be selected from, but not limited to, an ionizable lipid described in International Publication Nos. WO2013086354 and WO2013116126; the contents of each of which are herein incorporated by reference in their entirety.

In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a first polypeptide, and optionally in combination with the polynucleotide encoding a second polypeptide, are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

c. Liposomes, Lipoplexes, and Lipid Nanoparticles

In some embodiments, the nucleic acids of the disclosure are formulated as liposome compositions, lipoplex compositions, and/or polyplex compositions. Such compositions, and methods are generally known in the art, see for example Itziar Gómez-Aguado I. et al., Nanomaterials 2020, 10, 364; Kowalski P. S. et al., Mol Ther. 2019 Apr. 10; 27(4): 710-728; ur Rehman Z, et al. ACS Nano. 2013 May 28; 7(5):3767-77; and U.S. Patent Application Publication US20160367702, incorporated by reference herein in its entirety.

In some embodiments, the compositions or formulations of the present disclosure comprise a delivery agent, e.g., a liposome, a lipoplexes, a lipid nanoparticle, or any combination thereof. The polynucleotides described herein (e.g., a polynucleotide comprising first polypeptide) can be formulated using one or more liposomes, lipoplexes, or lipid nanoparticles. Liposomes, lipoplexes, or lipid nanoparticles can be used to improve the efficacy of the polynucleotides directed protein production as these formulations can increase cell transfection by the polynucleotide; and/or increase the translation of encoded protein. The liposomes, lipoplexes, or lipid nanoparticles can also be used to increase the stability of the polynucleotides.

Liposomes are artificially-prepared vesicles that can primarily be composed of a lipid bilayer and can be used as a delivery vehicle for the administration of pharmaceutical formulations. Liposomes can be of different sizes. A multilamellar vesicle (MLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos.

US20130177638, US20130177637, US20130177636, US20130177635, US20130177634, US20130177633, US20130183375, US20130183373, and US20130183372. In some embodiments, the polynucleotides described herein can be encapsulated by the liposome and/or it can be contained in an aqueous core that can then be encapsulated by the liposome as described in, e.g., Intl. Pub. Nos. WO2012031046, WO2012031043, WO2012030901, WO2012006378, and WO2013086526; and U.S. Pub. Nos. US20130189351, US20130195969 and US20130202684. Each of the references in herein incorporated by reference in its entirety.

In some embodiments, the polynucleotides described herein can be formulated in a cationic oil-in-water emulsion where the emulsion particle comprises an oil core and a cationic lipid that can interact with the polynucleotide anchoring the molecule to the emulsion particle. In some embodiments, the polynucleotides described herein can be formulated in a water-in-oil emulsion comprising a continuous hydrophobic phase in which the hydrophilic phase is dispersed. Exemplary emulsions can be made by the methods described in Intl. Pub. Nos. WO2012006380 and WO201087791, each of which is herein incorporated by reference in its entirety.

In some embodiments, the polynucleotides described herein can be formulated in a lipid-polycation complex. The formation of the lipid-polycation complex can be accomplished by methods as described in, e.g., U.S. Pub. No. US20120178702. As a non-limiting example, the polycation can include a cationic peptide or a polypeptide such as, but not limited to, polylysine, polyornithine and/or polyarginine and the cationic peptides described in Intl. Pub. No. WO2012013326 or U.S. Pub. No. US20130142818. Each of the references is herein incorporated by reference in its entirety.

In some embodiments, the polynucleotides described herein can be formulated in a lipid nanoparticle (LNP) such as those described in Intl. Pub. Nos. WO2013123523, WO2012170930, WO2011127255 and WO2008103276; and U.S. Pub. No. US20130171646, each of which is herein incorporated by reference in its entirety.

Lipid nanoparticle formulations typically comprise one or more lipids. In some embodiments, the lipid is an ionizable lipid (e.g., an ionizable amino lipid), sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable lipids include, but not limited to, any one of Compounds 1-342 disclosed herein, DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)—N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z, 16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)—N,N-dimethylnonacosa-11,20,2-trien-10-amine, and any combination thereof. Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some embodiments, the amount of PEG-lipid in the lipid composition ranges from about 0 mol % to about 5 mol %.

In some embodiments, the LNP formulations described herein can additionally comprise a permeability enhancer molecule. Non-limiting permeability enhancer molecules are described in U.S. Pub. No. US20050222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP formulation can also contain a polymer conjugate (e.g., a water soluble conjugate) as described in, e.g., U.S. Pub. Nos. US20130059360, US20130196948, and US20130072709. Each of the references is herein incorporated by reference in its entirety.

The LNP formulations can comprise a conjugate to enhance the delivery of nanoparticles of the present invention in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975, herein incorporated by reference in its entirety). As shown by Rodriguez et al. the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013110028, each of which is herein incorporated by reference in its entirety.

The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin j4 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028, herein incorporated by reference in its entirety.

In some embodiments, the polynucleotide described herein is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293 Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In some embodiments, the polynucleotides described herein are formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in Intl. Pub. No. WO2013105101, herein incorporated by reference in its entirety.

In some embodiments, the polynucleotides described herein can be formulated for controlled release and/or targeted delivery. As used herein, “controlled release” refers to a pharmaceutical composition or compound release profile that conforms to a particular pattern of release to effect a therapeutic outcome. In one embodiment, the polynucleotides can be encapsulated into a delivery agent described herein and/or known in the art for controlled release and/or targeted delivery. As used herein, the term “encapsulate” means to enclose, surround or encase. As it relates to the formulation of the compounds of the invention, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent.

Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, or greater than 99% of the pharmaceutical composition or compound of the invention are encapsulated in the delivery agent.

In some embodiments, the polynucleotides described herein can be encapsulated in a therapeutic nanoparticle, referred to herein as “therapeutic nanoparticle polynucleotides.” Therapeutic nanoparticles can be formulated by methods described in, e.g., Intl. Pub. Nos. WO2010005740, WO2010030763, WO2010005721, WO2010005723, and WO2012054923; and U.S. Pub. Nos. US20110262491, US20100104645, US20100087337, US20100068285, US20110274759, US20100068286, US20120288541, US20120140790, US20130123351 and US20130230567; and U.S. Pat. Nos. 8,206,747, 8,293,276, 8,318,208 and 8,318,211, each of which is herein incorporated by reference in its entirety.

In some embodiments, the therapeutic nanoparticle polynucleotide can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the polynucleotides described herein can be formulated as disclosed in Intl. Pub. No. WO2010075072 and U.S. Pub. Nos. US20100216804, US20110217377, US20120201859 and US20130150295, each of which is herein incorporated by reference in their entirety.

In some embodiments, the therapeutic nanoparticle polynucleotide can be formulated to be target specific, such as those described in Intl. Pub. Nos. WO2008121949, WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsev et al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM,) from the Institut für Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the polynucleotides described herein can be formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002); each of which is herein incorporated by reference in its entirety). In some embodiments, the polynucleotides can be formulated in lipid nanoparticles using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

In some embodiments, the polynucleotides described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the polynucleotides can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

The nanoparticles and microparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., Intl. Pub. No. WO2013082111, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

d. Lipidoids

In some embodiments, the compositions or formulations of the present disclosure comprise a delivery agent, e.g., a lipidoid. The polynucleotides described herein (e.g., a polynucleotide comprising a nucleotide sequence encoding a first polypeptide) can be formulated with lipidoids. Complexes, micelles, liposomes or particles can be prepared containing these lipidoids and therefore to achieve an effective delivery of the polynucleotide, as judged by the production of an encoded protein, following the injection of a lipidoid formulation via localized and/or systemic routes of administration. Lipidoid complexes of polynucleotides can be administered by various means including, but not limited to, intravenous, intramuscular, or subcutaneous routes.

The synthesis of lipidoids is described in literature (see Mahon et al., Bioconjug. Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

Formulations with the different lipidoids, including, but not limited to penta[3-(1-laurylaminopropionyl)]-triethylenetetramine hydrochloride (TETA-5LAP; also known as 98N12-5, see Murugaiah et al., Analytical Biochemistry, 401:61 (2010)), C12-200 (including derivatives and variants), and MD1, can be tested for in vivo activity. The lipidoid “98N12-5” is disclosed by Akinc et al., Mol Ther. 2009 17:872-879. The lipidoid “C12-200” is disclosed by Love et al., Proc Natl Acad Sci USA. 2010 107:1864-1869 and Liu and Huang, Molecular Therapy. 2010 669-670. Each of the references is herein incorporated by reference in its entirety.

In one embodiment, the polynucleotides described herein can be formulated in an aminoalcohol lipidoid. Aminoalcohol lipidoids can be prepared by the methods described in U.S. Pat. No. 8,450,298 (herein incorporated by reference in its entirety).

The lipidoid formulations can include particles comprising either 3 or 4 or more components in addition to polynucleotides. Lipidoids and polynucleotide formulations comprising lipidoids are described in Intl. Pub. No. WO 2015051214 (herein incorporated by reference in its entirety.

e. Hyaluronidase

In some embodiments, the polynucleotides described herein (e.g., a polynucleotide comprising a nucleotide sequence encoding a first polypeptide) and hyaluronidase for injection (e.g., intramuscular or subcutaneous injection). Hyaluronidase catalyzes the hydrolysis of hyaluronan, which is a constituent of the interstitial barrier. Hyaluronidase lowers the viscosity of hyaluronan, thereby increases tissue permeability (Frost, Expert Opin. Drug Deliv. (2007) 4:427-440). Alternatively, the hyaluronidase can be used to increase the number of cells exposed to the polynucleotides administered intramuscularly, or subcutaneously.

f. Nanoparticle Mimics

In some embodiments, the polynucleotides described herein (e.g., a polynucleotide comprising a nucleotide sequence encoding a first polypeptide) is encapsulated within and/or absorbed to a nanoparticle mimic. A nanoparticle mimic can mimic the delivery function organisms or particles such as, but not limited to, pathogens, viruses, bacteria, fungus, parasites, prions and cells. As a non-limiting example, the polynucleotides described herein can be encapsulated in a non-viron particle that can mimic the delivery function of a virus (see e.g., Intl. Pub. No. WO2012006376 and U.S. Pub. Nos. US20130171241 and US20130195968, each of which is herein incorporated by reference in its entirety).

g. Self-Assembled Nanoparticles, or Self-Assembled Macromolecules

In some embodiments, the compositions or formulations of the present disclosure comprise the polynucleotides described herein (e.g., a polynucleotide comprising a nucleotide sequence encoding a first polypeptide) in self-assembled nanoparticles, or amphiphilic macromolecules (AMs) for delivery. AMs comprise biocompatible amphiphilic polymers that have an alkylated sugar backbone covalently linked to poly(ethylene glycol). In aqueous solution, the AMs self-assemble to form micelles. Nucleic acid self-assembled nanoparticles are described in Intl. Appl. No. PCT/US2014/027077, and AMs and methods of forming AMs are described in U.S. Pub. No. US20130217753, each of which is herein incorporated by reference in its entirety.

h. Cations and Anions

In some embodiments, the compositions or formulations of the present disclosure comprise the polynucleotides described herein (e.g., a polynucleotide comprising a nucleotide sequence encoding a first polypeptide) and a cation or anion, such as Zn2+, Ca2+, Cu2+, Mg2+ and combinations thereof. Exemplary formulations can include polymers and a polynucleotide complexed with a metal cation as described in, e.g., U.S. Pat. Nos. 6,265,389 and 6,555,525, each of which is herein incorporated by reference in its entirety. In some embodiments, cationic nanoparticles can contain a combination of divalent and monovalent cations. The delivery of polynucleotides in cationic nanoparticles or in one or more depot comprising cationic nanoparticles can improve polynucleotide bioavailability by acting as a long-acting depot and/or reducing the rate of degradation by nucleases.

i. Amino Acid Lipids

In some embodiments, the compositions or formulations of the present disclosure comprise the polynucleotides described herein (e.g., a polynucleotide comprising a nucleotide sequence encoding a first polypeptide) that is formulation with an amino acid lipid. Amino acid lipids are lipophilic compounds comprising an amino acid residue and one or more lipophilic tails. Non-limiting examples of amino acid lipids and methods of making amino acid lipids are described in U.S. Pat. No. 8,501,824. The amino acid lipid formulations can deliver a polynucleotide in releasable form that comprises an amino acid lipid that binds and releases the polynucleotides. As a non-limiting example, the release of the polynucleotides described herein can be provided by an acid-labile linker as described in, e.g., U.S. Pat. Nos. 7,098,032, 6,897,196, 6,426,086, 7,138,382, 5,563,250, and 5,505,931, each of which is herein incorporated by reference in its entirety.

i. Interpolyelectrolyte Complexes

In some embodiments, the compositions or formulations of the present disclosure comprise the polynucleotides described herein (e.g., a polynucleotide comprising a nucleotide sequence encoding a first polypeptide) in an interpolyelectrolyte complex. Interpolyelectrolyte complexes are formed when charge-dynamic polymers are complexed with one or more anionic molecules. Non-limiting examples of charge-dynamic polymers and interpolyelectrolyte complexes and methods of making interpolyelectrolyte complexes are described in U.S. Pat. No. 8,524,368, herein incorporated by reference in its entirety.

k. Crystalline Polymeric Systems

In some embodiments, the compositions or formulations of the present disclosure comprise the polynucleotides described herein (e.g., a polynucleotide comprising a nucleotide sequence encoding a first polypeptide) in crystalline polymeric systems. Crystalline polymeric systems are polymers with crystalline moieties and/or terminal units comprising crystalline moieties. Exemplary polymers are described in U.S. Pat. No. 8,524,259 (herein incorporated by reference in its entirety).

l. Polymers, Biodegradable Nanoparticles, and Core-Shell Nanoparticles

In some embodiments, the compositions or formulations of the present disclosure comprise the polynucleotides described herein (e.g., a polynucleotide comprising a nucleotide sequence encoding a first polypeptide) and a natural and/or synthetic polymer. The polymers include, but not limited to, polyethenes, polyethylene glycol (PEG), poly(1-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, elastic biodegradable polymer, biodegradable copolymer, biodegradable polyester copolymer, biodegradable polyester copolymer, multiblock copolymers, poly[α-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyureas, polystyrenes, polyamines, polylysine, poly(ethylene imine), poly(serine ester), poly(L-lactide-co-L-lysine), poly(4-hydroxy-L-proline ester), amine-containing polymers, dextran polymers, dextran polymer derivatives or combinations thereof.

Exemplary polymers include, DYNAMIC POLYCONJUGATE® (Arrowhead Research Corp., Pasadena, CA) formulations from MIRUS® Bio (Madison, WI) and Roche Madison (Madison, WI), PHASERX™ polymer formulations such as, without limitation, SMARTT POLYMER TECHNOLOGY™ (PHASERX®, Seattle, WA), DMRI/DOPE, poloxamer, VAXFECTIN® adjuvant from Vical (San Diego, CA), chitosan, cyclodextrin from Calando Pharmaceuticals (Pasadena, CA), dendrimers and poly(lactic-co-glycolic acid) (PLGA) polymers. RONDEL™ (RNAi/Oligonucleotide Nanoparticle Delivery) polymers (Arrowhead Research Corporation, Pasadena, CA) and pH responsive co-block polymers such as PHASERX® (Seattle, WA).

The polymer formulations allow a sustained or delayed release of the polynucleotide (e.g., following intramuscular or subcutaneous injection). The altered release profile for the polynucleotide can result in, for example, translation of an encoded protein over an extended period of time. The polymer formulation can also be used to increase the stability of the polynucleotide. Sustained release formulations can include, but are not limited to, PLGA microspheres, ethylene vinyl acetate (EVAc), poloxamer, GELSITE® (Nanotherapeutics, Inc. Alachua, FL), HYLENEX® (Halozyme Therapeutics, San Diego CA), surgical sealants such as fibrinogen polymers (Ethicon Inc. Cornelia, GA), TISSELL® (Baxter International, Inc. Deerfield, IL), PEG-based sealants, and COSEAL® (Baxter International, Inc. Deerfield, IL).

As a non-limiting example modified mRNA can be formulated in PLGA microspheres by preparing the PLGA microspheres with tunable release rates (e.g., days and weeks) and encapsulating the modified mRNA in the PLGA microspheres while maintaining the integrity of the modified mRNA during the encapsulation process. EVAc are non-biodegradable, biocompatible polymers that are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C.

As a non-limiting example, the polynucleotides described herein can be formulated with the polymeric compound of PEG grafted with PLL as described in U.S. Pat. No. 6,177,274. As another non-limiting example, the polynucleotides described herein can be formulated with a block copolymer such as a PLGA-PEG block copolymer (see e.g., U.S. Pub. No. US20120004293 and U.S. Pat. Nos. 8,236,330 and 8,246,968), or a PLGA-PEG-PLGA block copolymer (see e.g., U.S. Pat. No. 6,004,573). Each of the references is herein incorporated by reference in its entirety.

In some embodiments, the polynucleotides described herein can be formulated with at least one amine-containing polymer such as, but not limited to polylysine, polyethylene imine, poly(amidoamine) dendrimers, poly(amine-co-esters) or combinations thereof. Exemplary polyamine polymers and their use as delivery agents are described in, e.g., U.S. Pat. Nos. 8,460,696, 8,236,280, each of which is herein incorporated by reference in its entirety.

In some embodiments, the polynucleotides described herein can be formulated in a biodegradable cationic lipopolymer, a biodegradable polymer, or a biodegradable copolymer, a biodegradable polyester copolymer, a biodegradable polyester polymer, a linear biodegradable copolymer, PAGA, a biodegradable cross-linked cationic multi-block copolymer or combinations thereof as described in, e.g., U.S. Pat. Nos. 6,696,038, 6,517,869, 6,267,987, 6,217,912, 6,652,886, 8,057,821, and 8,444,992; U.S. Pub. Nos. US20030073619, US20040142474, US20100004315, US2012009145 and US20130195920; and Intl Pub. Nos. WO2006063249 and WO2013086322, each of which is herein incorporated by reference in its entirety.

In some embodiments, the polynucleotides described herein can be formulated in or with at least one cyclodextrin polymer as described in U.S. Pub. No. US20130184453. In some embodiments, the polynucleotides described herein can be formulated in or with at least one crosslinked cation-binding polymers as described in Intl. Pub. Nos. WO2013106072, WO2013106073 and WO2013106086. In some embodiments, the polynucleotides described herein can be formulated in or with at least PEGylated albumin polymer as described in U.S. Pub. No. US20130231287. Each of the references is herein incorporated by reference in its entirety.

In some embodiments, the polynucleotides disclosed herein can be formulated as a nanoparticle using a combination of polymers, lipids, and/or other biodegradable agents, such as, but not limited to, calcium phosphate. Components can be combined in a core-shell, hybrid, and/or layer-by-layer architecture, to allow for fine-tuning of the nanoparticle for delivery (Wang et al., Nat Mater. 2006 5:791-796; Fuller et al., Biomaterials. 2008 29:1526-1532; DeKoker et al., Adv Drug Deliv Rev. 2011 63:748-761; Endres et al., Biomaterials. 2011 32:7721-7731; Su et al., Mol Pharm. 2011 Jun. 6; 8(3):774-87; herein incorporated by reference in their entireties). As a non-limiting example, the nanoparticle can comprise a plurality of polymers such as, but not limited to hydrophilic-hydrophobic polymers (e.g., PEG-PLGA), hydrophobic polymers (e.g., PEG) and/or hydrophilic polymers (Intl. Pub. No. WO20120225129, herein incorporated by reference in its entirety).

The use of core-shell nanoparticles has additionally focused on a high-throughput approach to synthesize cationic cross-linked nanogel cores and various shells (Siegwart et al., Proc Natl Acad Sci USA. 2011 108:12996-13001; herein incorporated by reference in its entirety). The complexation, delivery, and internalization of the polymeric nanoparticles can be precisely controlled by altering the chemical composition in both the core and shell components of the nanoparticle. For example, the core-shell nanoparticles can efficiently deliver siRNA to mouse hepatocytes after they covalently attach cholesterol to the nanoparticle.

In some embodiments, a hollow lipid core comprising a middle PLGA layer and an outer neutral lipid layer containing PEG can be used to delivery of the polynucleotides as described herein. In some embodiments, the lipid nanoparticles can comprise a core of the polynucleotides disclosed herein and a polymer shell, which is used to protect the polynucleotides in the core. The polymer shell can be any of the polymers described herein and are known in the art. The polymer shell can be used to protect the polynucleotides in the core.

Core-shell nanoparticles for use with the polynucleotides described herein are described in U.S. Pat. No. 8,313,777 or Intl. Pub. No. WO2013124867, each of which is herein incorporated by reference in their entirety.

m. Peptides and Proteins

In some embodiments, the compositions or formulations of the present disclosure comprise the polynucleotides described herein (e.g., a polynucleotide comprising a nucleotide sequence encoding a first polypeptide) that is formulated with peptides and/or proteins to increase transfection of cells by the polynucleotide, and/or to alter the biodistribution of the polynucleotide (e.g., by targeting specific tissues or cell types), and/or increase the translation of encoded protein (e.g., Intl. Pub. Nos. WO2012110636 and WO2013123298. In some embodiments, the peptides can be those described in U.S. Pub. Nos. US20130129726, US20130137644 and US20130164219. Each of the references is herein incorporated by reference in its entirety.

n. Conjugates

In some embodiments, the compositions or formulations of the present disclosure comprise the polynucleotides described herein (e.g., a polynucleotide comprising a nucleotide sequence encoding a first polypeptide) that is covalently linked to a carrier or targeting group, or including two encoding regions that together produce a fusion protein (e.g., bearing a targeting group and therapeutic protein or peptide) as a conjugate. The conjugate can be a peptide that selectively directs the nanoparticle to neurons in a tissue or organism, or assists in crossing the blood-brain barrier.

The conjugates include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid, an oligonucleotide (e.g., an aptamer). Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

In some embodiments, the conjugate can function as a carrier for the polynucleotide disclosed herein. The conjugate can comprise a cationic polymer such as, but not limited to, polyamine, polylysine, polyalkylenimine, and polyethylenimine that can be grafted to with poly(ethylene glycol). Exemplary conjugates and their preparations are described in U.S. Pat. No. 6,586,524 and U.S. Pub. No. US20130211249, each of which herein is incorporated by reference in its entirety.

The conjugates can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Targeting groups can be proteins, e.g., glycoproteins, or peptides, e.g., molecules having a specific affinity for a co-ligand, or antibodies e.g., an antibody, that binds to a specified cell type such as an endothelial cell or bone cell. Targeting groups can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent frucose, or aptamers. The ligand can be, for example, a lipopolysaccharide, or an activator of p38 MAP kinase.

The targeting group can be any ligand that is capable of targeting a specific receptor. Examples include, without limitation, folate, GalNAc, galactose, mannose, mannose-6P, apatamers, integrin receptor ligands, chemokine receptor ligands, transferrin, biotin, serotonin receptor ligands, PSMA, endothelin, GCPII, somatostatin, LDL, and HDL ligands. In particular embodiments, the targeting group is an aptamer. The aptamer can be unmodified or have any combination of modifications disclosed herein. As a non-limiting example, the targeting group can be a glutathione receptor (GR)-binding conjugate for targeted delivery across the blood-central nervous system barrier as described in, e.g., U.S. Pub. No. US2013021661012 (herein incorporated by reference in its entirety).

In some embodiments, the conjugate can be a synergistic biomolecule-polymer conjugate, which comprises a long-acting continuous-release system to provide a greater therapeutic efficacy. The synergistic biomolecule-polymer conjugate can be those described in U.S. Pub. No. US20130195799. In some embodiments, the conjugate can be an aptamer conjugate as described in Intl. Pat. Pub. No. WO2012040524. In some embodiments, the conjugate can be an amine containing polymer conjugate as described in U.S. Pat. No. 8,507,653. Each of the references is herein incorporated by reference in its entirety. In some embodiments, the polynucleotides can be conjugated to SMARTT POLYMER TECHNOLOGY® (PHASERX®, Inc. Seattle, WA).

In some embodiments, the polynucleotides described herein are covalently conjugated to a cell penetrating polypeptide, which can also include a signal sequence or a targeting sequence. The conjugates can be designed to have increased stability, and/or increased cell transfection; and/or altered the biodistribution (e.g., targeted to specific tissues or cell types).

In some embodiments, the polynucleotides described herein can be conjugated to an agent to enhance delivery. In some embodiments, the agent can be a monomer or polymer such as a targeting monomer or a polymer having targeting blocks as described in Intl. Pub. No. WO2011062965. In some embodiments, the agent can be a transport agent covalently coupled to a polynucleotide as described in, e.g., U.S. Pat. Nos. 6,835,393 and 7,374,778. In some embodiments, the agent can be a membrane barrier transport enhancing agent such as those described in U.S. Pat. Nos. 7,737,108 and 8,003,129. Each of the references is herein incorporated by reference in its entirety.

### Exemplary Additional Components of Compositions

### Surfactants

In certain embodiments, a composition of the disclosure optionally includes one or more surfactants.

In certain embodiments, the surfactant is an amphiphilic polymer. As used herein, an amphiphilic “polymer” is an amphiphilic compound that comprises an oligomer or a polymer. For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10-4 M in water at about 30° C. and atmospheric pressure.

For example, amphiphilic polymer has a critical micelle concentration (CMC) ranging between about 0.1×10-4 M and about 1.3×10-4 M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

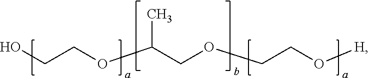

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

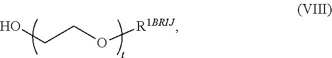

or a salt or isomer thereof, wherein:
t is an integer between 1 and 100;
R1BRIJ independently is C10-40 alkyl, C10-40 alkenyl, or C10-40 alkynyl; and optionally one or more methylene groups of R5PEG are independently replaced with C3-10 carbocyclylene, 4 to 10 membered heterocyclylene, C6-10 arylene, 4 to 10 membered heteroarylene, —N(RN)—, —O—, —S—, —C(O)—, —C(O)N(RN)—, —NRNC(O)—, —NRNC(O)N(RN)—, —C(O)O—, —OC(O)—, —OC(O)O—, —OC(O)N(RN)—, —NRNC(O)O—, —C(O)S—, —SC(O)—, —C(═NRN)—, —C(═NRN)N(RN)—, —NRNC(═NRN)—, —NRNC(═NRN)N(RN)—, —C(S)—, —C(S)N(RN)—, —NRNC(S)—, —NRNC(S)N(RN)—, —S(O)—, —OS(O)—, —S(O)O—, —OS(O)O—, —OS(O)2-, —S(O)2O—, —OS(O)2O—, —N(RN)S(O)—, —S(O)N(RN)—, —N(RN)S(O)N(RN)—, —OS(O)N(RN)—, —N(RN)S(O)O—, —S(O)2-, —N(RN)S(O)2-, —S(O)2N(RN)—, —N(RN)S(O)2N(RN)—, —OS(O)2N(RN)—, or —N(RN)S(O)2O—; and
each instance of RN is independently hydrogen, C1-6 alkyl, or a nitrogen protecting group

In some embodiment, R1BRIJ is C18 alkyl. For example, the polyethylene glycol ether is a compound of Formula (VIII-a):

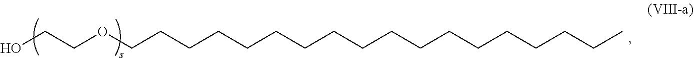

or a salt or isomer thereof.

In some embodiments, R1BRIJ is C18 alkenyl. For example, the polyethylene glycol ether is a compound of Formula (VIII-b):

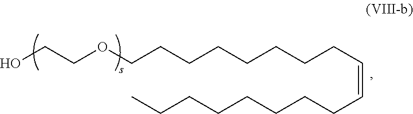

or a salt or isomer thereof

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt % to about 1 wt %, e.g., from about 0.000002 wt % to about 0.8 wt %, or from about 0.000005 wt % to about 0.5 wt %.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

### Adjuvants

In some embodiments, a lipid nanoparticle composition of the disclosure optionally includes one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

### Other Components

In some embodiments, a lipid nanoparticle composition of the disclosure may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

### Pharmaceutically Acceptable Excipients

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro; Lippincott, Williams & Wilkins, Baltimore, MD, 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN® 20], polyoxyethylene sorbitan [TWEEN® 60], polyoxyethylene sorbitan monooleate [TWEEN® 80], sorbitan monopalmitate [SPAN® 40], sorbitan monostearate [SPAN® 60], sorbitan tristearate [SPAN® 65], glyceryl monooleate, sorbitan monooleate [SPAN® 80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC® F 68, POLOXAMER® 188, cetrimonium bromide, cetylpyridinium chloride, benzalkonium chloride, docusate sodium, and/or combinations thereof. A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN® II, NEOLONE™, KATHON™, and/or EUXYL®. Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Production of LNP Compositions

### A. Production of Nanoparticle Compositions

In order to investigate safe and efficacious nanoparticle compositions for use in the delivery of polynucleotides of the disclosure to cells, a range of formulations are prepared and tested. Specifically, the particular elements and ratios thereof in the lipid component of nanoparticle compositions are optimized.

Nanoparticles can be made with mixing processes such as microfluidics and T-junction mixing of two fluid streams, one of which contains the polynucleotides of the disclosure and the other has the lipid components.

Lipid compositions are prepared by combining a lipid according to Formulae (I), (IA), (IB), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8) or a non-cationic helper lipid (such as DOPE, or DSPC obtainable from Avanti Polar Lipids, Alabaster, AL), a PEG lipid (such as 1,2 dimyristoyl sn glycerol methoxypolyethylene glycol, also known as PEG-DMG, obtainable from Avanti Polar Lipids, Alabaster, AL), and a phytosterol (optionally including a structural lipid such as cholesterol) at concentrations of about, e.g., 50 mM in a solvent, e.g., ethanol. Solutions should be refrigerated for storage at, for example, −20° C. Lipids are combined to yield desired molar ratios (see, for example, Table 7 below) and diluted with water and ethanol to a final lipid concentration of e.g., between about 5.5 mM and about 25 mM. Phytosterol* in Table 7 refers to phytosterol or optionally a combination of phytosterol and structural lipid such as beta-phytosterol and cholesterol.

TABLE 7
Exemplary formulations of LNP compositions

| Composition | |
|---|---|
| (mol %) | Components |
| 40:20:38.5:1.5 | Ionizable lipid:Phospholipid:Phytosterol*:PEG-DMG |
| 45:15:38.5:1.5 | Ionizable lipid:Phospholipid:Phytosterol*:PEG-DMG |
| 50:10:38.5:1.5 | Ionizable lipid:Phospholipid:Phytosterol*:PEG-DMG |
| 55:5:38.5:1.5 | Ionizable lipid:Phospholipid:Phytosterol*:PEG-DMG |
| 60:5:33.5:1.5 | Ionizable lipid:Phospholipid:Phytosterol*:PEG-DMG |
| 45:20:33.5:1.5 | Ionizable lipid:Phospholipid:Phytosterol*:PEG-DMG |
| 50:20:28.5:1.5 | Ionizable lipid:Phospholipid:Phytosterol*:PEG-DMG |
| 55:20:23.5:1.5 | Ionizable lipid:Phospholipid:Phytosterol*:PEG-DMG |
| 60:20:18.5:1.5 | Ionizable lipidd:Phospholipid:Phytosterol*:PEG-DMG |
| 40:15:43.5:1.5 | Ionizable lipid:Phospholipid:Phytosterol*:PEG-DMG |
| 50:15:33.5:1.5 | Ionizable lipid:Phospholipid:Phytosterol*:PEG-DMG |
| 55:15:28.5:1.5 | Ionizable lipid:Phospholipid:Phytosterol*:PEG-DMG |
| 60:15:23.5:1.5 | Ionizable lipid:Phospholipid:Phytosterol*:PEG-DMG |
| 40:10:48.5:1.5 | Ionizable lipid:Phospholipid:Phytosterol*:PEG-DMG |
| 45:10:43.5:1.5 | Ionizable lipid:Phospholipid:Phytosterol*:PEG-DMG |
| 55:10:33.5:1.5 | Ionizable lipid:Phospholipid:Phytosterol*:PEG-DMG |
| 60:10:28.5:1.5 | Ionizable lipidd:Phospholipid:Phytosterol*:PEG-DMG |
| 40:5:53.5:1.5 | Ionizable lipid:Phospholipid:Phytosterol*:PEG-DMG |
| 45:5:48.5:1.5 | Ionizable lipid:Phospholipid:Phytosterol*:PEG-DMG |
| 50:5:43.5:1.5 | Ionizable lipid:Phospholipid:Phytosterol*:PEG-DMG |
| 40:20:40:0 | Ionizable lipid:Phospholipid:Phytosterol*:PEG-DMG |
| 45:20:35:0 | Ionizable lipid:Phospholipid:Phytosterol*:PEG-DMG |
| 50:20:30:0 | Ionizable lipidd:Phospholipid:Phytosterol*:PEG-DMG |
| 55:20:25:0 | Ionizable lipid:Phospholipid:Phytosterol*:PEG-DMG |
| 60:20:20:0 | Ionizable lipid:Phospholipid:Phytosterol*:PEG-DMG |
| 40:15:45:0 | Ionizable lipid:Phospholipid:Phytosterol*:PEG-DMG |
| 45:15:40:0 | Ionizable lipid:Phospholipid:Phytosterol*:PEG-DMG |
| 50:15:35:0 | Ionizable lipid:Phospholipid:Phytosterol*:PEG-DMG |
| 55:15:30:0 | Ionizable lipidPhospholipid:Phytosterol*:PEG-DMG |
| 60:15:25:0 | Ionizable lipid:Phospholipid:Phytosterol*:PEG-DMG |
| 40:10:50:0 | Ionizable lipid:Phospholipid:Phytosterol*:PEG-DMG |
| 45:10:45:0 | Ionizable lipidPhospholipid:Phytosterol*:PEG-DMG |
| 50:0:48.5:1.5 | Ionizable lipid:Phospholipid:Phytosterol*:PEG-DMG |
| 50:10:40:0 | Ionizable lipid:Phospholipid:Phytosterol*:PEG-DMG |
| 55:10:35:0 | Ionizable lipid:Phospholipid:Phytosterol*:PEG-DMG |
| 60:10:30:0 | Ionizable lipidPhospholipid:Phytosterol*:PEG-DMG |

Nanoparticle compositions including polynucleotide(s) of the disclosure and a lipid component are prepared by combining the lipid solution with a solution including the polynucleotides of the disclosure at lipid component to polynucleotides wt:wt ratios between about 5:1 and about 50:1. The lipid solution is rapidly injected using a NanoAssemblr microfluidic based system at flow rates between about 10 ml/min and about 18 ml/min into the polynucleotides solution to produce a suspension with a water to ethanol ratio between about 1:1 and about 4:1.

For nanoparticle compositions including an RNA, solutions of the RNA at concentrations of 0.1 mg/ml in deionized water are diluted in a buffer, e.g., 50 mM sodium citrate buffer at a pH between 3 and 4 to form a stock solution.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kDa. The first dialysis is carried out at room temperature for 3 hours. The formulations are then dialyzed overnight at 4° C. The resulting nanoparticle suspension is filtered through 0.2 m sterile filters (Sarstedt, Numbrecht, Germany) into glass vials and sealed with crimp closures. Nanoparticle composition solutions of 0.01 mg/ml to 0.10 mg/ml are generally obtained.

The method described above induces nano-precipitation and particle formation. Alternative processes including, but not limited to, T-junction and direct injection, may be used to achieve the same nano-precipitation.

### B. Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of a polynucleotide (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of the polynucleotides of the disclosure in the nanoparticle composition can be calculated based on the extinction coefficient of the polynucleotides used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, CA) can be used to evaluate the encapsulation of an RNA by the nanoparticle composition. The samples are diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### C. In Vivo Formulation Studies

In order to monitor how effectively various nanoparticle compositions deliver polynucleotides of the disclosure to targeted cells, different nanoparticle compositions including a particular polynucleotide of the disclosure (for example, an mRNA) are prepared and administered to rodent populations. Mice are intravenously, intramuscularly, subcutaneously, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a polynucleotide of the disclosure in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

Nanoparticle compositions including mRNA are useful in the evaluation of the efficacy and usefulness of various formulations for the delivery of polynucleotides. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the polynucleotides by a given nanoparticle composition relative to other nanoparticle compositions or the absence thereof.

### Example 2: L7Ae Represses Expression of Target RNA (5′ Kink Turn-RNA) In Vitro

Examples 2-6 describe how to achieve ON target expression of a target mRNA in specific cells when co-delivered with an RNA encoding a repressor protein, e.g., L7Ae. The source of all cell lines is ATCC and the cells are plated at 10,000-20,000 cells per well in a 96 well plate.

The system used in this Example (depicted in FIGS. 1A-1B) is a 2 RNA system where one RNA encodes a repressor protein to suppress protein translation from the target RNA.

In one embodiment of the system, in the absence of microRNA binding to the microRNA target site (miRts), the repressor (e.g., L7Ae) can bind to the repressor binding site (e.g., kink-turn forming sequence) on the target RNA, thereby suppressing (turning OFF) target protein translation (FIG. 1A). When a specific microRNA binds to the miRts, it turns ON expression of target RNA, by reducing expression of the repressor and thereby preventing the repressor from binding to the repressor binding site on the target RNA (FIG. 1B).

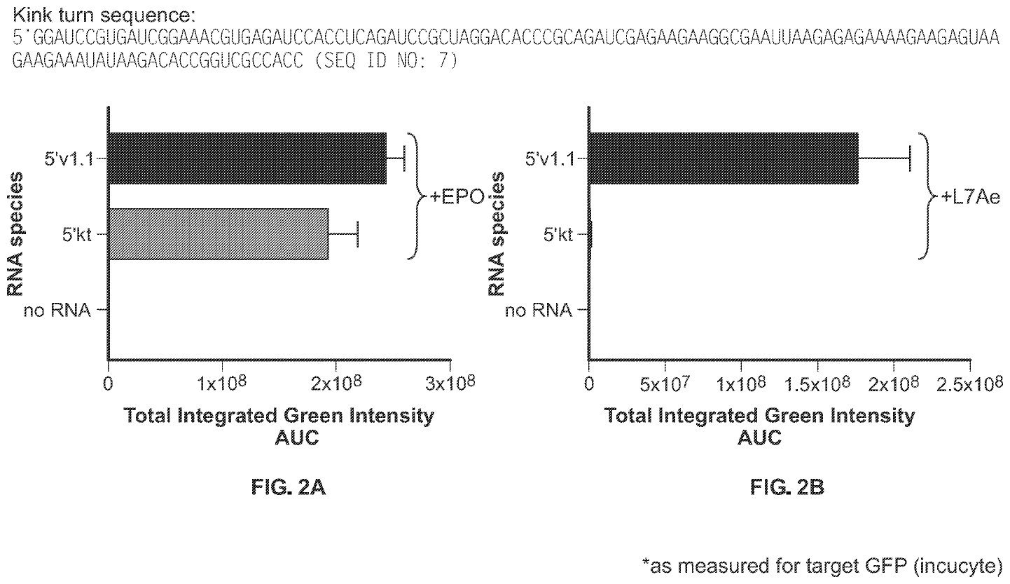

The initial experiments were conducted to establish that a repressor in the 2 RNA system can repress the expression of the target in a cell line. HeLa cells were transfected with target RNA (5′v1.1-GFP or 5′kt-GFP RNA constructs. GFP protein output was measured every hour with Live cell imaging using Incucyte®. Total AUC in 48 hrs post-transfection is plotted. As shown in FIG. 2A, HeLa cells transfected with the target RNA constructs, co-delivered with Control (Erythropoietin; EPO) encoding RNA have similar levels of GFP fluorescence (plotted as Total Area Under Curve of the total integrated green intensity; AUC). HeLa cells were also transfected with target RNA (5′v1.1-GFP or 5′kt-GFP RNA constructs in combination with repressor RNA (L7Ae RNA construct in 3× molar excess of the target RNA. As shown in FIG. 2B, GFP protein output is significantly reduced in the cells that were transfected with 5′kt-GFP along with L7Ae repressor construct. This indicates that the L7Ae repressor protein can bind to the kink-turn sequence in 5′UTR of target RNA and block target RNA (GFP) expression.

### Example 3: L7Ae_3x142ts Leads to Selective Expression of Target mRNA in miR142+ Cells

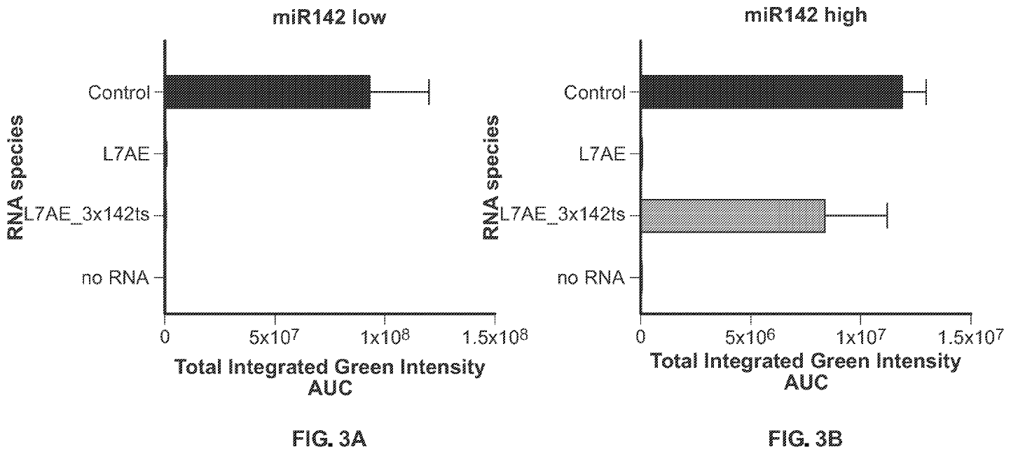

miR142 low (AML12) or miR142 high (JAWS) cells were transfected with target RNA (5′kt-GFP RNA, in combination with control (EPO) RNA, or L7Ae repressor RNA, or L7Ae_3x142ts repressor RNA. The target was in 3× molar excess of the control or repressor RNA. GFP protein output was measured on the Incucyte® Live-Cell Analysis System. Area under the curve after 48 h is plotted. As shown in FIGS. 3A-3B, GFP expression from the target RNA alone (control) is high. In the presence of the L7Ae repressor construct, GFP expression from the target RNA is suppressed in the miR142 low (AML12) cells as well as the miR142 high (JAWS) cells. In the presence of the L7Ae_3x142ts construct containing 3 miR142 target sites between the repressor sequence and the polyA tail, target GFP is selectively expressed in miR142 high (JAWS) cells but not in cells lacking miR142 (AML12 cells). These experiments confirm that miRNA (e.g., miR142) rescues translation of target GFP in cells that express that miRNA (e.g., miR142 in JAWS cells). miR142 in those cells may cleave and destabilize the repressor RNA with miRts (e.g., L7Ae_3x142ts) and prevent expression or directly repress L7AE expression. Thus, L7Ae is unable to bind to the 5′ repressor binding site on the target RNA, thereby permitting target GFP expression.

### Example 4: L7Ae_3x122ts Leads to Selective Expression of Target mRNA in miR122+ Cells

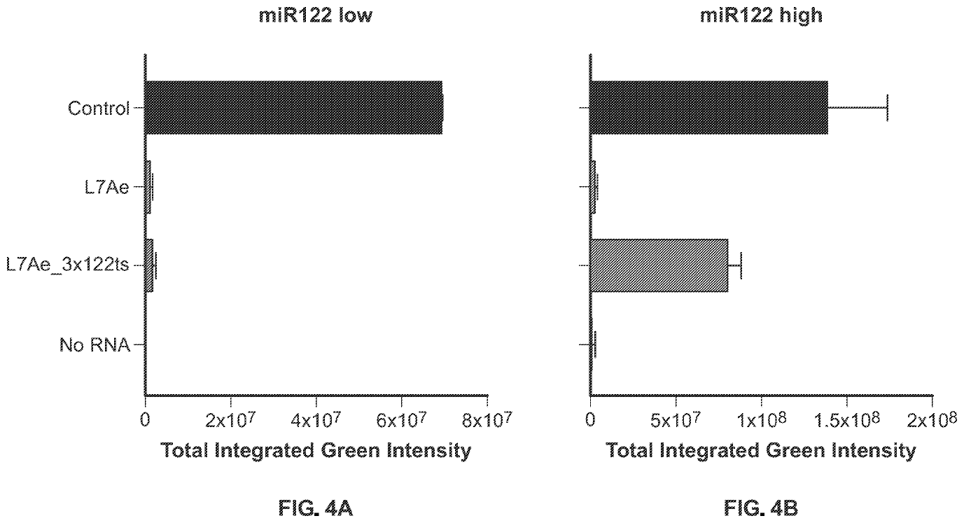

miR122 low (HeLa) or miR122 high (Huh7) cells were transfected with target RNA (5′kt-GFP RNA, in combination with Control EPO RNA or L7Ae repressor RNA, or L7Ae_3x122ts repressor RNA. The target RNA was in 16× molar excess of control or Repressor RNA. GFP protein output was measured using Incucyte® Live-Cell Analysis System. AUC after 48 h is plotted. As shown in FIGS. 4A-4B, GFP expression from the target RNA with control EPO is high. In the presence of the L7Ae repressor construct, GFP expression from the target RNA is suppressed in the miR122 low (HeLa) cells as well as the miR122 high (Huh7) cells. In the presence of the L7Ae_3x122ts construct containing 3 miR122 target sites between the repressor sequence and the polyA tail, target GFP is selectively expressed in miR122 high (Huh7) cells but not in cells with low miR122 (HeLa cells). These experiments confirm that miRNA (e.g., miR122) rescues translation of target GFP in cells that express that miRNA (e.g., miR122 in Huh7 cells). miRNA122 in those cells may cleave and destabilize the repressor RNA with miRts (e.g., L7Ae_3x122ts) and prevent expression or directly repress L7AE expression. Thus, L7Ae is unable to bind to the 5′ repressor binding site on the target RNA, thereby permitting target GFP expression.

### Example 5: L7Ae Represses Target RNA In Vivo

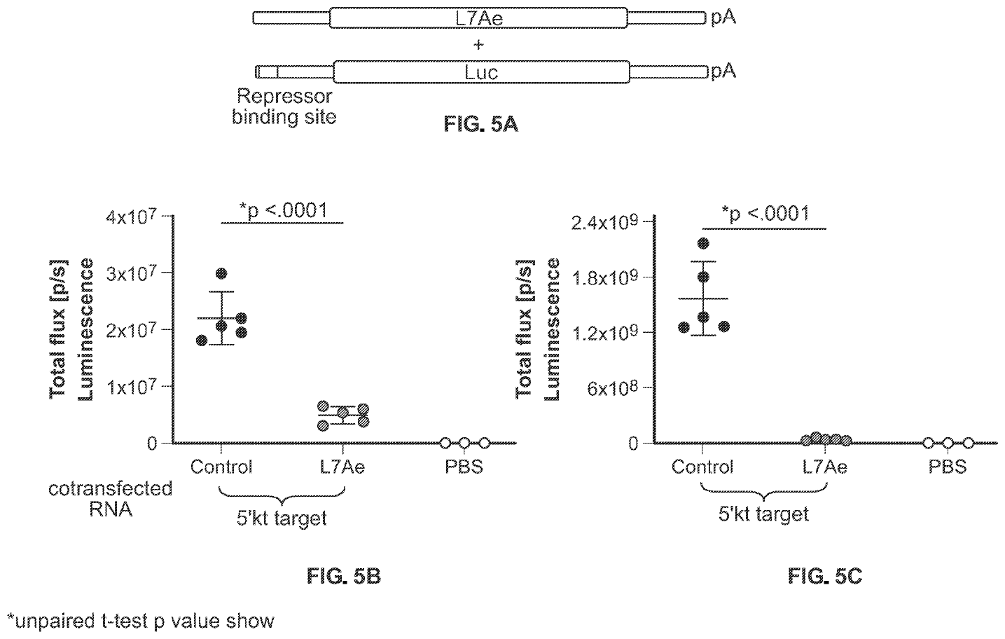

BALB/c mice (n=5 per group) were injected intravenously (IV) with 0.05 mg/kg of the 5′ kink-turn containing Luc target mRNA (5′ kt target) in combination with a control (EPO), RNA or L7Ae repressor RNA construct. The L7Ae repressor RNA was in molar excess (10×) of the Luc target RNA construct. FIG. 5A shows the RNA constructs used for the study. Luciferase expression was determined by ex vivo imaging 6 hrs post IV injection. FIG. 5B shows the level of luminescence in the spleen FIG. 5C shows the level of luminescence in the liver measured using IVIS instrument. The level of luminescence in the spleen and liver of mice injected with the 5′ kt Luc target construct in combination with the L7Ae repressor construct was significantly lower compared to cells transfected with the 5′ kt Luc target construct in combination with the control construct. These experiments indicate that L7Ae represses target RNA expression in vivo.

### Example 6: L7Ae_miRts RNA Leads to Selective Expression of Target RNA In Vivo

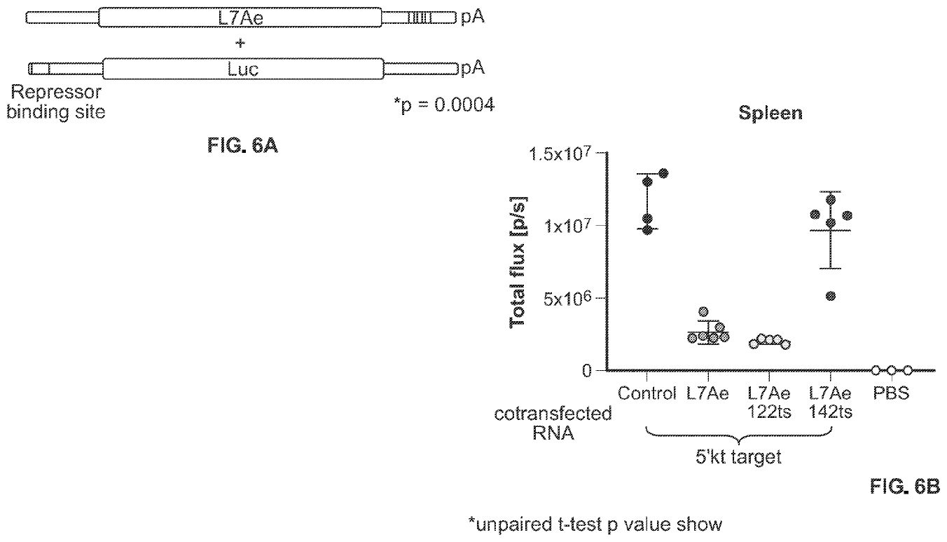

BALB/c mice (n=5 per group) were injected intravenously (IV) with 0.05 mg/kg of the 5′ kink-turn containing Luc target mRNA (5′ kt target) in combination with a control (EPO), RNA or L7Ae repressor RNA construct with miRNA target sites (miR122ts or miR142ts). The control EPO or L7Ae repressor_miRts RNA constructs were in molar excess (10×) of the Luc target RNA construct. FIG. 6A shows the RNA constructs used for the study. Luciferase expression was determined by ex vivo imaging 6 hrs post IV injection. FIG. 6B shows the level of luminescence in the spleen measured using IVIS imaging

The level of luminescence in the spleen of mice (FIG. 6B) injected with the 5′ kt Luc target construct in combination with the L7Ae_142ts repressor construct was significantly higher compared to that from the spleen of mice injected with 5′ kt Luc target construct in combination with the L7Ae construct or the L7Ae_122ts repressor construct, and comparable to that of mice injected with 5′ kt Luc target construct in combination with the control. The spleen expresses miR142 at a high level. These experiments indicate that L7Ae repression of target RNA expression in vivo can be reversed when the repressor expression is tied to a particular miRNA (e.g., miR142) in a cell-type where expression of the target is desired. miRNA (e.g., miR142) rescues translation of target GFP in vivo in tissues that express that miRNA (e.g., miR142 in spleen).

### Example 7: RBP-RNA Repressors PUF, PUF2 and MBP-LacZ Repress Target RNA Expression In Vitro

Alternative RNA binding protein (RBP)-RNA repressors were tested for suitability for use in the system of FIG. 1.

### PUF-PRE Interaction and Impact on Target RNA Expression

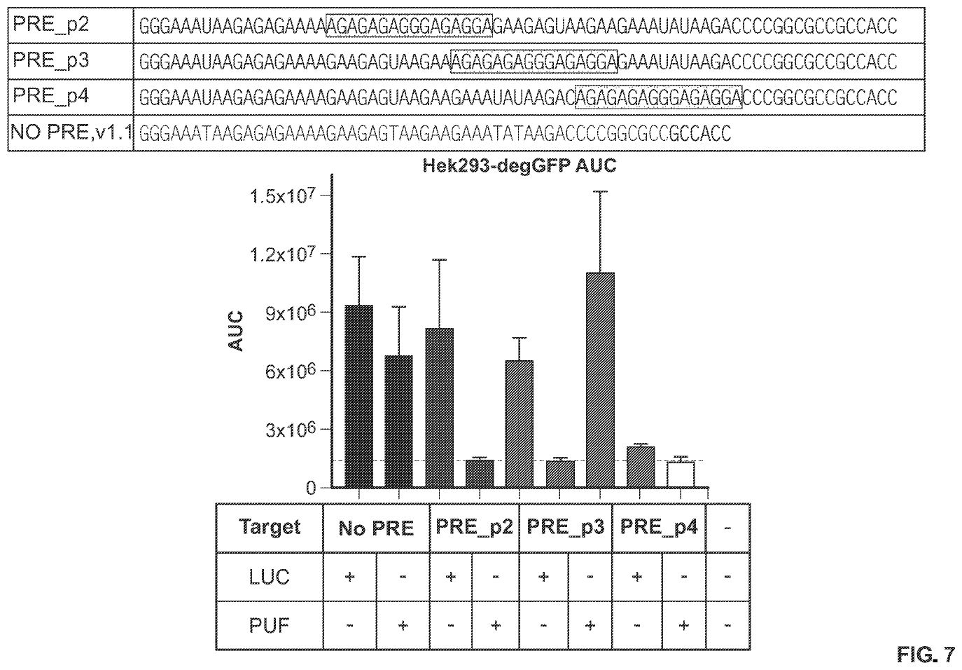

Hek293 cells were transfected with target RNA (degGFP) containing no PUF recognition elements, no PRE, or with PRE_p2, PRE_p3 or PRE_p4 in the 5′ UTR v1.1) (see FIG. 7). GFP protein output was measured using Incucyte® Live-Cell Analysis System live cell imaging. AUC after 48 hrs is plotted. As shown in FIG. 7, Hek293 cells co-transfected with the target RNA constructs with LUC RNA constructs (control) have high GFP target gene expression. LUC does not suppress GFP expression. However, Hek293 cells co-transfected with the target RNA constructs with PUF repressor RNA constructs (test) have significantly lower GFP target gene expression. LUC or PUF were delivered at equivalent moles to target RNA. These results indicate that the PUF-PRE interaction suppress target gene expression from target RNA.

### PUF2-PRE2 Interaction and Impact on Target RNA Expression

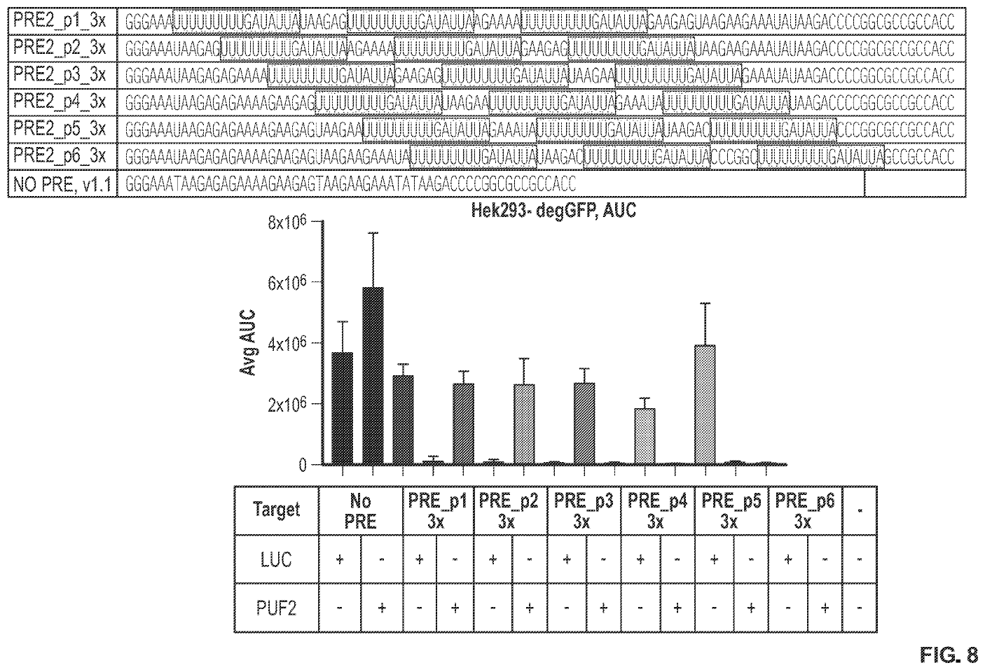

Hek293 cells were transfected with target RNA (degGFP) containing no PUF recognition elements, no PRE, or with PRE2_p1, PRE2_p2, PRE2_p3, PRE2_p4 or PRE2_p5 or PRE2_p6 in the 5′ UTR v1.1) (see FIG. 8). GFP protein output was measured using Incucyte® live cell imaging. AUC after 48 hrs is plotted. As shown in FIG. 8, Hek293 cells co-transfected with the target RNA constructs with LUC RNA constructs (control) have high GFP target gene expression. LUC does not suppress GFP expression. However, Hek293 cells co-transfected with the target RNA constructs with PUF2 repressor RNA constructs (test) have significantly lower GFP target gene expression. These results indicate that the PUF2-PRE2 interaction suppress target gene expression from target RNA.

### MBP-LacZ-MS2 Interaction and Impact on Target RNA Expression

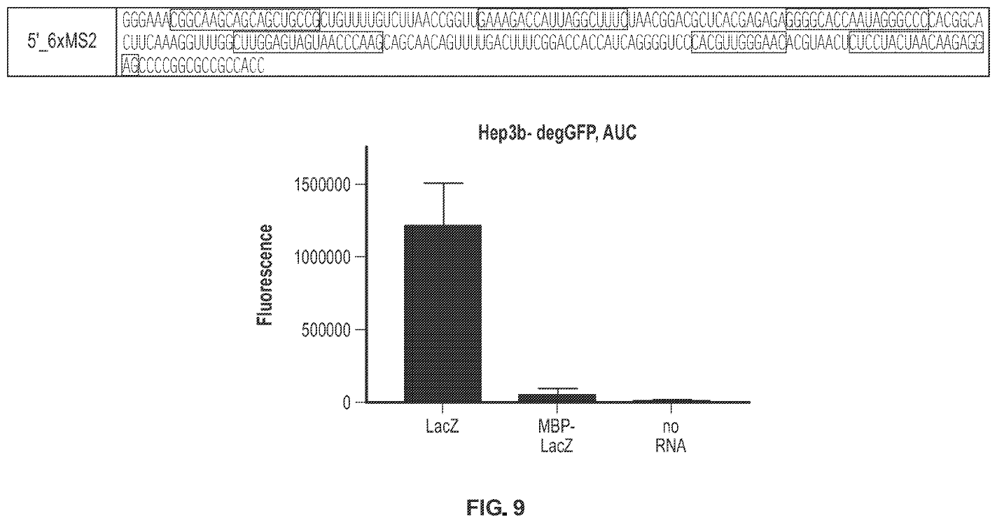

Hep3b cells were transfected with target RNA containing MS2 recognition elements in the 5′ UTR (5′v1.1-GFP constructs with 6×MS2 in the 5′ UTR (see FIG. 9)). GFP protein output was measured using Incucyte® live cell imaging. AUC after 48 hrs is plotted. As shown in FIG. 9, Hep3b cells co-transfected with the target RNA constructs with LacZ RNA constructs (control) have high GFP target gene expression. LacZ does not suppress GFP expression. However, Hep3b cells co-transfected with the target RNA constructs with MBP-LacZ repressor RNA construct (test) has significantly lower GFP target gene expression. These results indicate that the MBP-LacZ-MS2 interaction suppresses target gene expression from target RNA.

Collectively, these experiments indicate that proteins bound to the 5′UTR of target RNA can suppress translation of the target gene.

### Example 8: Tailless Target RNAs do not Show Detectable Expression In Vitro

Examples 7-8 describe achieving ON target expression of a target mRNA in specific cells when co-delivered with an RNA encoding an effector/activator protein, e.g., based on MBP-MS2 hairpin loop interactions.

The system used in this Example (depicted in FIG. 10) is a 2 RNA system where one RNA encodes an effector protein and the other RNA encodes a target protein. The top panel shows that in an OFF target environment (e.g., when a particular miRNA level is absent/low), the effector protein is unable to bind to its binding partner on the target RNA due to a steric block coupled to a cleavage site (e.g., a miRts specific to that particular miRNA) at the 3′ UTR end of the tailless target RNA, thereby suppressing target protein translation. The bottom panel shows that in an ON target environment (e.g., when a particular miRNA level is high), the effector protein can bind to its binding partner on the target RNA due to removal of the steric block (e.g., when the miRts is cleaved off by the miRNA), thereby removing the steric block, and permitting target protein translation. Instead of using miRNAs, one can also use a tissue-specific promoter to control the expression of the effector. In this case the effector is only made in on-target cells which can recognize the relevant tissue-specific promoter.

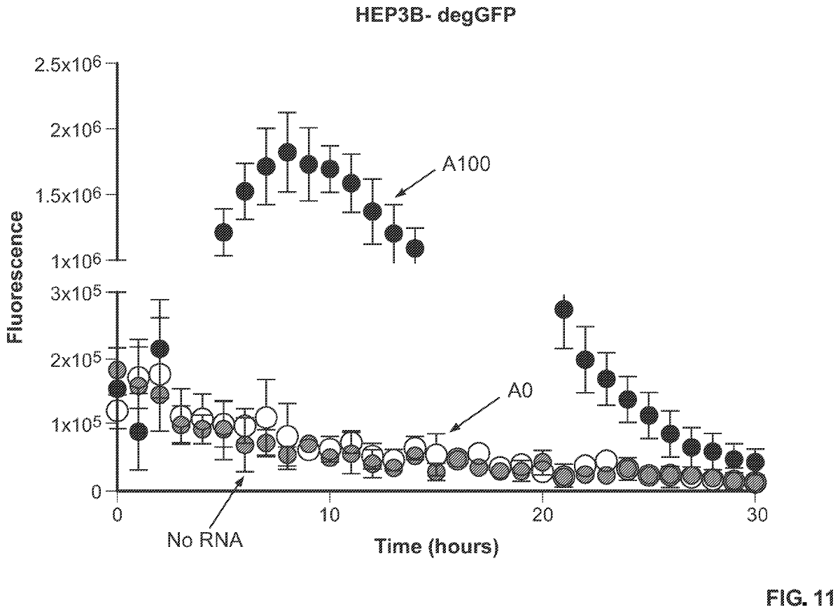

To establish that tailless RNAs can be used in the system of FIG. 10, the initial experiments were conducted to establish that tailless RNA does not show detectable expression. HEP3B cells were transfected with degGFP target RNA lacking a poly A tail (A0) or control GFP target RNA with a poly A tail (A100). GFP protein output was measured over time on the Incucyte® Live-Cell Analysis System. As shown in FIG. 11, HEP3B cells transfected with the tailless (A0) target RNA construct had significantly lesser GFP fluorescence compared to HeLa cells transfected with control target RNA with A100 tails over the course of 30 hours. These results establish that tailless target RNAs do not show detectable expression of the target when transfected into cells.

### Example 9: Tailless Target RNAs can be Rescued by Effectors Recruited Using RBP-RNA (MBP-MS2) Tethers

FIG. 12 provides a schematic of a system to recruit potential effectors to target RNA with an MS2 binding protein (MBP)-MS2 tether to rescue tailless target RNA. The target mRNA has MS2 loops in the 3′UTR and lacks a polyA tail (A0). The second mRNA encodes MS2-binding protein (MBP) fused to an effector/eIF4G-mid. The MBP-MS2 binding interaction recruits the effector protein to the target RNA. This system can be coupled to a miRNA-dependent switch gate to permit tethering in specific cells, thereby turning ON expression of the target protein. Alternatively, the system can be coupled to a miRNA-independent switch gate to permit tethering in specific cells by delivering the effector as a DNA molecule under the control of a tissue-specific promoter. Specific cells expressing that promoter would thus turn ON expression of the effector and permit tethering and rescue of target expression.

The eIF4G mid-to-C-terminal domain can support ribosome complex formation, support or enhance cap-dependent translation when recruited to mRNA 3′ UTR via cap-independent translation enhancers (CITEs), and stimulate translation of a capless mRNA when recruited via IRES elements (Kraft J J, et al. (2013) NAR. 41(5):3398-413; Paek K Y, et al. (2015) PNAS 112(4):1041-6; Pestova T V, et al. (1996) Mol Cell Biol. 16(12):6870-8). Thus, this domain (herein named eIF4GAN) can be used as the effector. The domain eIF4-mid can also be used as the effector. With no eIF4E and PABP binding abilities, these domains may not bind other mRNAs in the cell, thereby reducing probable off-target effects. As a tether, the MS2-MBP system can be used since it has been extensively validated over the last few decades. Notably the MS2-MBP interaction is expected to be very strong (K_{d}˜1-10 nM) and stable in a cell (Tutucci E, et al. (2018) Nat Methods. 15(1):81-89). Six MS2 loops were inserted in the 3′UTR of the target mRNA.

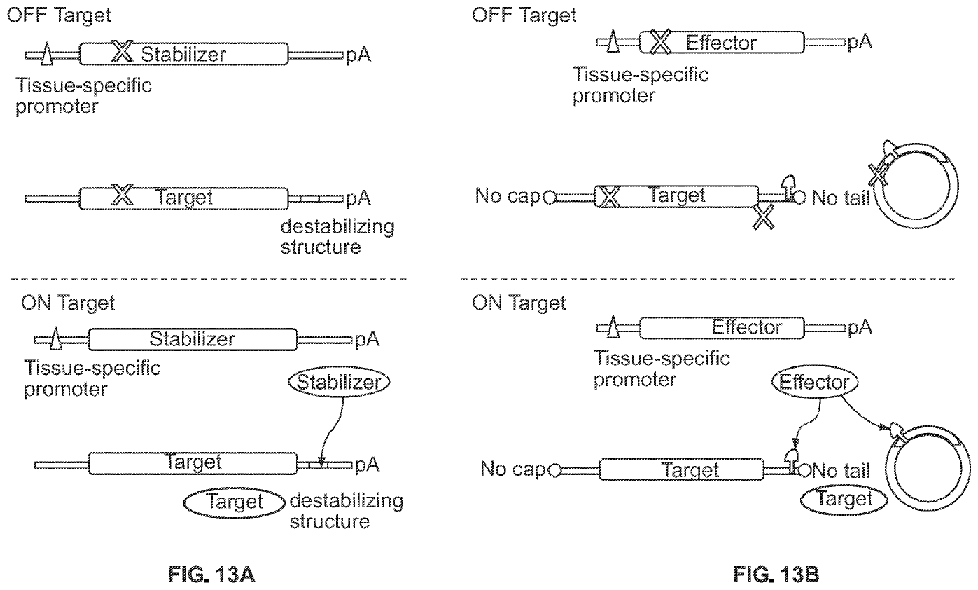

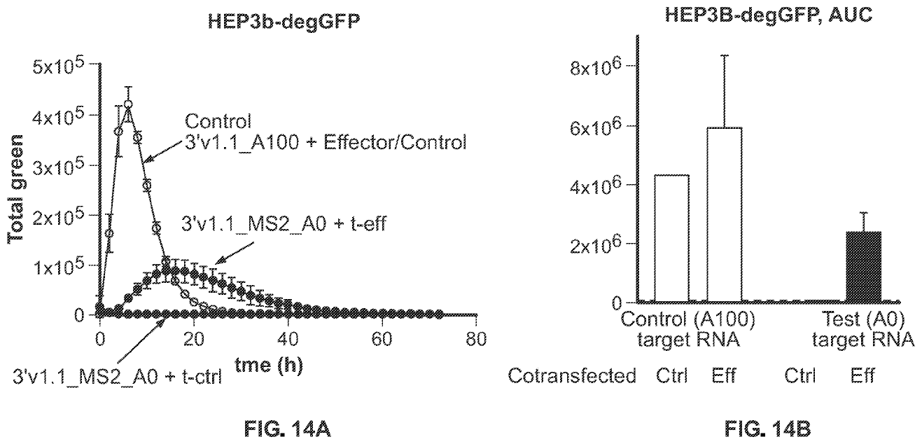

To illustrate that the system of FIGS. 12A-12B can be used to rescue tailless target RNA, HEP3B cells were transfected with 3′v1.1_A100+Effector/Control deg-GFP construct, and 3′v1.1_MS2_A0+Control or Effector (FIG. 13A). Control RNA here encodes for MBP-LacZ and effector RNA encodes for MBP-eIF4G-mid. Target RNA was in 10× molar excess of effector RNA. FIG. 14B depicts the level of total integrated green intensity plotted as total Area Under Curve; AUC). As seen in FIG. 14A, the 3′v1.1 RNA constructs with A100 tails expressed GFP at high levels for 20 hours post transfection in presence of effector or control. However, tailless 3′v1.1 RNA constructs with MS2 hairpin loops at the 3′ end expressed GFP in the presence of effector for an extended duration, but not detectable expression is seen in the absence of effector. These results show that tailless target RNAs can be rescued by an MS2-MBP tether system when fused to an effector protein.

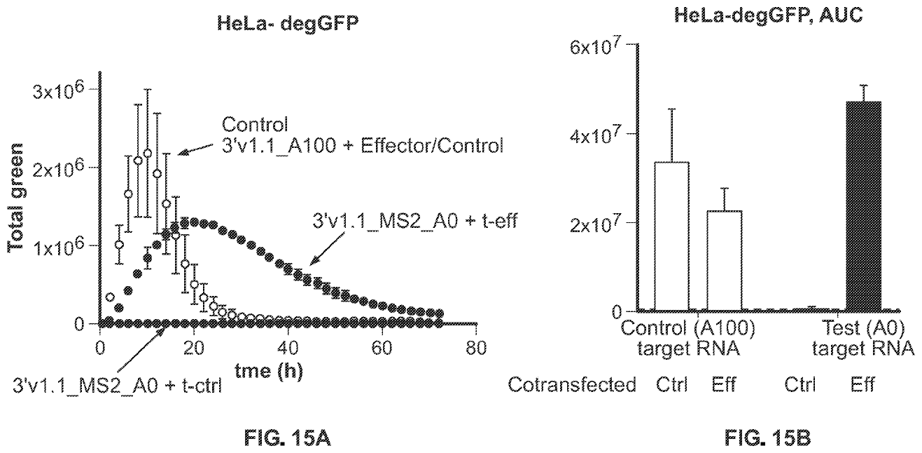

Similarly, FIG. 15A shows that tailless target RNA can be rescued only when co-delivered with an effector that can bind the target RNA. HEP3B cells were transfected with 3′v1.1_A100+Effector/Control deg-GFP construct, and 3′v1.1_MS2_tail-less+Control or Effector (FIG. 15A). Control RNA here encodes for MBP-LacZ and effector RNA encodes for MBP-eIF4G-mid. Target RNA was in 10× molar excess of effector RNA. FIG. 15B depicts the level of total integrated green intensity plotted as total Area Under Curve; AUC). As seen in FIG. 15A, the 3′v1.1 RNA constructs with A100 tails expressed GFP at high levels for 20 hours post transfection in presence of effector or control. However, tailless 3′v1.1 RNA constructs with MS2 hairpin loops at the 3′ end expressed GFP in the presence of effector for an extended duration, but not at all in the absence of activator. These results show that tailless target RNAs can be rescued by an MS2-MBP tether system.

### Example 10: Tailless Target RNAs with idT Tails can be Rescued by Effectors Recruited Using RBP-RNA (MBP-MS2) Tethers

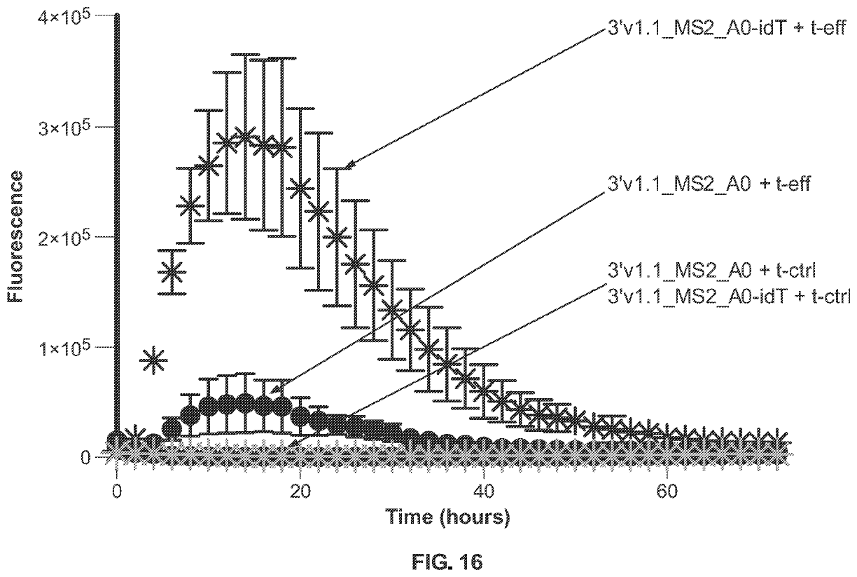

To illustrate that the system of FIGS. 12A-12B can be used to rescue modified tailless target RNA expression, HEP3B cells were transfected with 3′v1.1_MS2_A0 or 3′v1.1_MS2_A0-idT+Effector/Control deg-GFP constructs. In this experiment, tailless target RNA was modified with an inverted deoxythymidine (idT) tail, i.e., A20 oligo followed by an inverted idT. Control RNA encodes for MBP-LacZ and effector RNA encodes for MBP-eIF4G-mid. Target RNA was in 10× molar excess of effector RNA. GFP protein output was measured using Incucyte® Live-Cell Analysis System. As seen in FIG. 16, A0 or A0-idT constructs showed no detectable expression when co-transfected with control RNA. Tethering effector increased expression from A0 RNA. Tethering in the presence of idT rescued expression to a much higher level. These results demonstrate that translation from an A0 RNA can improve in the presence of idT, along with a tether.

### Example 11: Capless Target RNAs can be Rescued by Effectors Recruited Using RBP-RNA (MBP-MS2) Tethers

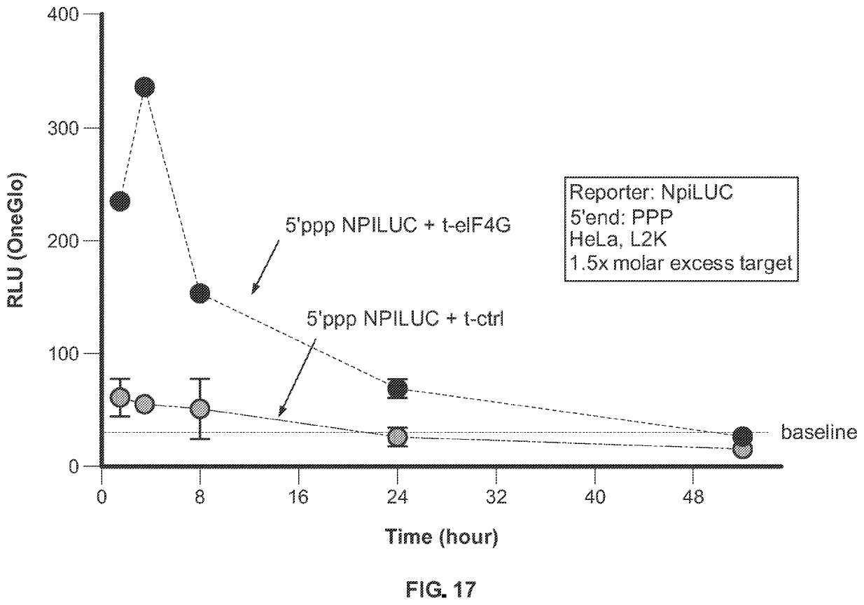

To illustrate that the system of FIGS. 12A-12B can be used to rescue capless target RNA, HeLa cells were transfected with 5′ triphosphate (5′ppp) ended NpiLuc-_3′v1.1_MS2_A100 target RNAs+Effector/Control NpiLuc constructs. Target RNA was in 1.5× molar excess of effector RNA. The ONE-Glo™ luciferase assay system was used to measure luminescence in cell lysate according to the manufacturer's protocol (Promega). As seen in the FIG. 17, while the capless RNA by itself showed no appreciable signal above background with a control tether RNA (MBP-mid2-Mut, encodes for a mutant mid domain), appreciable expression is restored on this RNA when tethered with effector (MBP-mid2)+Effector/Control deg-GFP construct, and 3′v1.1_MS2_A0+Control or Effector.

### Example 12: Capless Target RNAs can be Rescued by Effectors Recruited Using RBP-RNA (MBP-MS2) Tethers

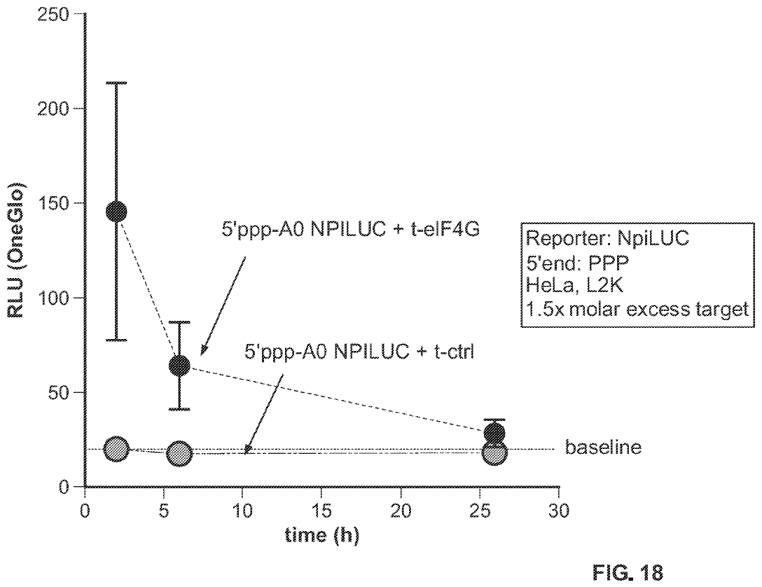

To illustrate that the system of FIG. 12 can be used to rescue capless-tailless target RNA, HeLa cells were transfected with 5′ppp ended NpiLuc_3′v1.1_MS2_A0 target RNAs+Effector/Control NpiLuc constructs. Target RNA was in 1.5× molar excess of effector RNA. The ONE-Glo™ luciferase assay system was used to measure luminescence in cell lysate according to the manufacturer's protocol (Promega). As seen in the FIG. 18, while the capless-tailless RNA by itself showed no appreciable signal above background with a control tether RNA (MBP-mid2-Mut, encodes for a mutant mid domain), appreciable expression was restored on this RNA when delivered with the tethered-effector construct (MBP-mid2).

### Example 13: Dormant Circular Target RNAs can be Rescued by Effectors Recruited Using RBP-RNA (MBP-MS2) Tethers

To illustrate that the system of FIG. 12 can be used to rescue dormant circular target RNA, HeLa cells were transfected with circular RNA, 5′MS2_NpiLuc_3′v1.1 target RNAs+different Effectors encoding RNAs as annotated. Target RNA was in 2× molar excess of effector RNA. The ONE-Glo™ luciferase assay system was used to measure luminescence in cell lysate according to the manufacturer's protocol (Promega). As seen in FIG. 19, while the dormant circular RINA by itself or with t-LACzs showed no appreciable signal abcve background, appreciable expression was restored on this RNA when delivered with the tethered-effector constructs t-eF4G or t-La.

TABLE 8
Miscellaneous sequences:

| | | |
|---|---|---|
| SEQ | Sequence | |
| ID NO | information | Sequence |

Second polypeptide (Effector molecule or tether molecule) sequences

| | | |
|---|---|---|
| 136 | LacZ (AA) | MALAVVLQRRDWENPGVTQLNRLAAHPPFASWRNSEEARTDRPSQ |
| | | QLRSLNGEWRFAWFPAPEAVPESWLECDLPEADTVVVPSNWQMH |
| | | GYDAPIYTNVTYPITVNPPFVPTENPTGCYSLTFNVDESWLQEGQTR |
| | | IIFDGVNSAFHLWCNGRWVGYGQDSRLPSEFDLSAFLRAGENRLAV |
| | | MVLRWSDGSYLEDQDMWRMSGIFRDVSLLHKPTTQISDFHVATRF |
| | | NDDFSRAVLEAEVQMCGELRDYLRVTVSLWQGETQVASGTAPFGG |
| | | EIIDERGGYADRVTLRLNVENPKLWSAEIPNLYRAVVELHTADGTLI |
| | | EAEACDVGFREVRIENGLLLLNGKPLLIRGVNRHEHHPLHGQVMDE |
| | | QTMVQDILLMKQNNFNAVRCSHYPNHPLWYTLCDRYGLYVVDEA |
| | | NIETHGMVPMNRLTDDPRWLPAMSERVTRMVQRDRNHPSVIIWSL |
| | | GNESGHGANHDALYRWIKSVDPSRPVQYEGGGADTTATDIICPMY |
| | | ARVDEDQPFPAVPKWSIKKWLSLPGETRPLILCEYAHAMGNSLGGF |
| | | AKYWQAFRQYPRLQGGFVWDWVDQSLIKYDENGNPWSAYGGDF |
| | | GDTPNDRQFCMNGLVFADRTPHPALTEAKHQQQFFQFRLSGQTIEV |
| | | TSEYLFRHSDNELLHWMVALDGKPLASGEVPLDVAPQGKQLIELPE |
| | | LPQPESAGQLWLTVRVVQPNATAWSEAGHISAWQQWRLAENLSVT |
| | | LPAASHAIPHLTTSEMDFCIELGNKRWQFNRQSGFLSQMWIGDKKQ |
| | | LLTPLRDQFTRAPLDNDIGVSEATRIDPNAWVERWKAAGHYQAEA |
| | | ALLQCTADTLADAVLITTAHAWQHQGKTLFISRKTYRIDGSGQMAI |
| | | TVDVEVASDTPHPARIGLNCQLAQVAERVNWLGLGPQENYPDRLT |
| | | AACFDRWDLPLSDMYTPYVFPSENGLRCGTRELNYGPHQWRGDFQ |
| | | FNISRYSQQQLMETSHRHLLHAEEGTWLNIDGFHMGIGGDDSWSPS |
| | | VSAELQLSAGRYHYQLVWCQK |
| 137 | LacZ (NT) | ATGGCCTTGGCTGTCGTCCTGCAAAGAAGAGATTGGGAAAATCC |
| | | TGGAGTTACGCAACTGAATAGACTCGCCGCACATCCACCGTTCG |
| | | CGTCCTGGCGAAATAGCGAAGAAGCGCGGACCGACAGACCTTCG |
| | | CAGCAGCTGCGCTCTCTCAACGGGGAATGGCGGTTCGCATGGTT |
| | | TCCGGCTCCTGAGGCAGTCCCGGAAAGCTGGCTCGAGTGCGACC |
| | | TCCCGGAAGCCGATACGGTGGTGGTGCCGTCAAATTGGCAAATG |
| | | CATGGATACGACGCCCCCATCTACACCAACGTCACTTACCCTATC |
| | | ACCGTGAATCCCCCATTCGTCCCGACTGAGAACCCGACTGGATG |
| | | CTACAGCCTGACCTTTAACGTGGACGAGTCGTGGCTGCAAGAAG |
| | | GGCAGACTCGCATCATTTTCGACGGAGTCAACTCCGCGTTCCATC |
| | | TTTGGTGTAACGGACGGTGGGTGGGATACGGGCAGGACTCCAGG |
| | | CTGCCGAGCGAATTCGACTTGTCAGCCTTCCTGCGCGCCGGCGA |
| | | AAACCGCCTGGCTGTCATGGTCCTTAGATGGTCGGATGGCTCGT |
| | | ACCTGGAGGATCAGGACATGTGGAGGATGTCAGGGATCTTCCGG |
| | | GATGTCTCGCTGCTCCACAAGCCAACTACCCAGATCTCCGACTTT |
| | | CATGTGGCCACCCGCTTCAACGATGACTTCAGCAGGGCGGTTCT |
| | | GGAAGCCGAGGTGCAAATGTGCGGAGAACTGAGGGACTACCTC |
| | | CGCGTGACTGTCTCGCTCTGGCAGGGTGAAACCCAAGTGGCTTC |
| | | AGGCACTGCACCGTTCGGAGGAGAAATCATCGACGAACGGGGA |
| | | GGATACGCCGATCGCGTCACCCTGCGCCTCAATGTGGAAAATCC |
| | | GAAACTGTGGTCGGCAGAAATCCCTAATTTGTACCGGGCCGTGG |
| | | TGGAGCTGCACACCGCCGACGGAACTCTGATCGAGGCCGAGGCA |
| | | TGCGATGTGGGATTCCGCGAGGTCCGCATCGAAAATGGACTGCT |
| | | TCTGCTTAATGGCAAACCGCTGCTCATCCGCGGAGTGAACAGAC |
| | | ACGAGCATCACCCGCTGCACGGTCAGGTCATGGATGAACAGACT |
| | | ATGGTGCAAGACATCCTGCTGATGAAACAAAACAACTTCAACGC |
| | | CGTTCGGTGCTCCCATTACCCTAATCACCCGTTGTGGTATACCCT |
| | | TTGCGATCGGTACGGCCTCTACGTGGTGGACGAAGCGAACATCG |
| | | AGACTCACGGAATGGTCCCTATGAACCGCCTCACTGACGACCCG |
| | | AGGTGGCTCCCGGCAATGTCGGAACGAGTGACTCGGATGGTGCA |
| | | GAGGGACCGCAACCATCCGTCGGTGATAATCTGGTCGCTGGGGA |
| | | ACGAATCTGGCCACGGAGCTAACCACGATGCGCTGTACCGCTGG |
| | | ATTAAGTCCGTGGACCCAAGCCGGCCCGTCCAGTACGAAGGAGG |
| | | TGGTGCTGATACCACTGCAACCGACATCATCTGCCCAATGTATGC |
| | | GCGGGTGGATGAGGACCAACCTTTCCCGGCGGTGCCAAAGTGGT |
| | | CCATCAAGAAATGGCTCTCGCTGCCCGGAGAAACGCGCCCGCTG |
| | | ATCCTGTGCGAATATGCGCACGCTATGGGAAATTCACTGGGGGG |
| | | ATTTGCGAAGTACTGGCAGGCTTTTCGACAGTACCCGAGACTCC |
| | | AGGGTGGCTTCGTGTGGGACTGGGTTGACCAGAGCCTCATCAAA |
| | | TACGATGAAAACGGCAACCCATGGTCCGCGTACGGCGGAGACTT |
| | | TGGAGACACCCCTAACGATCGCCAGTTCTGCATGAACGGCCTGG |
| | | TGTTCGCCGACAGAACTCCGCATCCAGCCCTTACTGAGGCTAAG |
| | | CACCAACAACAGTTCTTCCAGTTCAGACTGTCGGGGCAAACGAT |
| | | CGAAGTGACTTCCGAATACCTCTTCCGGCATTCGGACAACGAGT |
| | | TGCTGCACTGGATGGTCGCCCTGGATGGAAAGCCCCTCGCCTCC |
| | | GGAGAAGTGCCGCTCGACGTGGCGCCGCAGGGAAAGCAGTTGA |
| | | TCGAGTTGCCGGAACTGCCACAGCCCGAGTCAGCAGGACAGCTC |
| | | TGGCTTACCGTCAGAGTCGTGCAGCCAAATGCCACCGCCTGGTC |
| | | GGAGGCAGGACATATTTCAGCCTGGCAGCAGTGGCGCCTCGCCG |
| | | AGAATCTGAGCGTGACCTTGCCAGCAGCCTCACACGCCATTCCG |
| | | CATCTGACCACGTCCGAAATGGACTTTTGTATCGAACTGGGGAA |
| | | TAAGCGCTGGCAGTTCAATCGGCAATCAGGCTTCCTGTCCCAGA |
| | | TGTGGATTGGTGACAAGAAGCAGCTCCTGACCCCGCTGCGCGAT |
| | | CAGTTCACTCGCGCCCCACTTGACAACGACATTGGCGTGAGCGA |
| | | GGCCACGCGTATCGATCCAAACGCTTGGGTGGAGCGCTGGAAGG |
| | | CGGCTGGCCACTATCAGGCGGAGGCCGCGCTCCTGCAGTGTACC |
| | | GCGGATACCCTCGCAGACGCCGTTCTGATTACCACGGCGCATGC |
| | | CTGGCAACACCAGGGAAAGACCCTGTTTATCAGCCGCAAAACTT |
| | | ACCGGATCGATGGCAGCGGCCAAATGGCGATCACTGTGGACGTC |
| | | GAGGTGGCATCAGACACTCCACACCCAGCACGGATCGGACTCAA |
| | | TTGCCAACTGGCTCAAGTGGCTGAGAGAGTCAATTGGCTGGGCC |
| | | TCGGCCCCCAAGAGAACTACCCTGATCGGCTTACTGCCGCATGC |
| | | TTTGACCGGTGGGATCTGCCTCTGTCGGATATGTACACCCCCTAC |
| | | GTGTTCCCATCCGAGAACGGTCTGAGATGCGGTACTAGGGAGTT |
| | | GAACTACGGACCGCACCAATGGAGGGGGGACTTTCAGTTCAACA |
| | | TCTCAAGATACAGCCAGCAGCAATTGATGGAAACCTCGCACCGG |
| | | CATCTCTTGCATGCAGAGGAAGGGACCTGGCTGAACATCGATGG |
| | | ATTCCACATGGGAATTGGTGGGGATGACTCCTGGTCCCCTAGCG |
| | | TGTCCGCGGAACTTCAGCTGTCCGCCGGCCGGTACCACTACCAG |
| | | CTCGTGTGGTGTCAAAAG |
| 138 | GFP (AA) | MVSKGEELFTGVVPILVELDGDVNGHKFSVSGEGEGDATYGKLTL |
| | | KFICTTGKLPVPWPTLVTTLTYGVQCFSRYPDHMKQHDFFKSAMPE |
| | | GYVQERTIFFKDDGNYKTRAEVKFEGDTLVNRIELKGIDFKEDGNIL |
| | | GHKLEYNYNSHNVYIMADKQKNGIKVNFKIRHNIEDGSVQLADHY |
| | | QQNTPIGDGPVLLPDNHYLSTQSALSKDPNEKRDHMVLLEFVTAAG |
| | | ITLGMDELYK |
| 139 | GFP (NT) | ATGGTGTCCAAGGGTGAGGAATTGTTTACCGGGGTGGTGCCTAT |
| | | TCTCGTCGAACTTGACGGGGATGTGAATGGACACAAGTTTTCGG |
| | | TATCCGGAGAAGGAGAGGGTGACGCCACATACGGAAAGCTTAC |
| | | ACTCAAATTCATCTGTACGACGGGGAAACTGCCCGTACCCTGGC |
| | | CTACGCTCGTAACCACGCTGACTTATGGAGTGCAGTGCTTTAGCA |
| | | GATACCCCGACCATATGAAGCAGCACGACTTCTTCAAGTCGGCG |
| | | ATGCCCGAGGGGTACGTGCAAGAGAGGACCATTTTCTTCAAAGA |
| | | CGATGGCAATTACAAAACACGCGCAGAAGTCAAGTTTGAGGGCG |
| | | ATACTCTGGTCAATCGGATCGAATTGAAGGGAATCGATTTCAAA |
| | | GAAGATGGAAACATCCTTGGCCATAAGCTCGAGTACAACTATAA |
| | | CTCGCATAATGTCTATATCATGGCTGACAAGCAGAAAAACGGTA |
| | | TCAAAGTCAACTTTAAGATCCGACACAATATTGAGGACGGTTCG |
| | | GTGCAGCTTGCGGACCACTATCAACAGAATACGCCGATTGGGGA |
| | | TGGTCCGGTCCTTTTGCCGGATAACCATTATCTCTCAACCCAGTC |
| | | AGCCCTGAGCAAAGATCCAAACGAGAAGAGGGACCACATGGTC |
| | | TTGCTCGAATTCGTGACAGCGGCAGGGATCACTCTGGGAATGGA |
| | | CGAGTTGTACAAG |
| 140 | NpiLuc (AA) | MGKPIPNPLLGLDSTQGQEQKLISEEDLAGKPIPNPLLGLDSTQGQE |
| | | QKLISEEDLAGKPIPNPLLGLDSTQGQEQKLISEEDLAGKPIPNPLLG |
| | | LDSTQGQEQKLISEEDLAGKPIPNPLLGLDSTQGQEQKLISEEDLAPA |
| | | AKRVKLDEDAKNIKKGPAPFYPLEDGTAGEQLHKAMKRYALVPGT |
| | | IAFTDAHIEVDITYAEYFEMSVRLAEAMKRYGLNTNHRIVVCSENSL |
| | | QFFMPVLGALFIGVAVAPANDIYNERELLNSMGISQPTVVFVSKKGL |
| | | QKILNVQKKLPIIQKIIIMDSKTDYQGFQSMYTFVTSHLPPGFNEYDF |
| | | VPESFDRDKTIALIMNSSGSTGLPKGVALPHRTACVRFSHARDPIFG |
| | | NQIIPDTAILSVVPFHHGFGMFTTLGYLICGFRVVLMYRFEEELFLRS |
| | | LQDYKIQSALLVPTLFSFFAKSTLIDKYDLSNLHEIASGGAPLSKEVG |
| | | EAVAKRFHLPGIRQGYGLTETTSAILITPEGDDKPGAVGKVVPFFEA |
| | | KVVDLDTGKTLGVNQRGELCVRGPMIMSGYVNNPEATNALIDKDG |
| | | WLHSGDIAYWDEDEHFFIVDRLKSLIKYKGYQVAPAELESILLQHPN |
| | | IFDAGVAGLPDDDAGELPAAVVVLEHGKTMTEKEIVDYVASQVTT |
| | | AKKLRGGVVFVDEVPKGLTGKLDARKIREILIKAKKGGKIAV |
| 141 | NpiLuc (NT) | ATGGGGAAACCTATCCCAAACCCCTTGCTCGGGCTCGATTCAAC |
| | | ACAAGGGCAGGAACAGAAGCTTATTTCCGAGGAGGACTTAGCA |
| | | GGAAAGCCCATTCCTAATCCATTGCTGGGTTTGGACAGTACGCA |
| | | GGGGCAAGAGCAGAAATTAATTTCAGAGGAGGATCTCGCCGGA |
| | | AAACCCATACCCAACCCCCTTCTTGGCCTAGACAGTACTCAGGG |
| | | ACAAGAACAGAAGTTAATCTCAGAAGAGGACCTAGCAGGGAAG |
| | | CCTATCCCCAACCCCCTTCTAGGGCTAGATTCCACCCAGGGGCA |
| | | GGAGCAGAAACTGATTAGCGAGGAGGATTTGGCAGGCAAACCT |
| | | ATCCCTAACCCCCTGCTGGGACTCGATAGCACTCAAGGGCAGGA |
| | | ACAGAAGCTGATCTCCGAGGAAGACCTCGCCCCGGCTGCGAAGC |
| | | GCGTGAAGCTGGATGAAGATGCGAAGAACATCAAGAAGGGACC |
| | | TGCCCCGTTTTACCCTTTGGAGGACGGTACAGCAGGAGAACAGC |
| | | TCCACAAGGCGATGAAACGCTACGCCCTGGTCCCCGGAACGATT |
| | | GCGTTTACCGATGCACATATTGAGGTAGACATCACATACGCAGA |
| | | ATACTTCGAAATGTCGGTGAGGCTGGCGGAAGCGATGAAGAGAT |
| | | ATGGTCTTAACACTAATCACCGCATCGTGGTGTGTTCGGAGAACT |
| | | CATTGCAGTTTTTCATGCCGGTCCTTGGAGCACTTTTCATCGGGG |
| | | TCGCAGTCGCGCCAGCGAACGACATCTACAATGAGCGGGAACTC |
| | | TTGAATAGCATGGGAATCTCCCAGCCGACGGTCGTGTTTGTCTCC |
| | | AAAAAGGGGCTGCAGAAAATCCTCAACGTGCAGAAGAAGCTCC |
| | | CCATTATTCAAAAGATCATCATTATGGATAGCAAGACAGATTAC |
| | | CAAGGGTTCCAGTCGATGTATACCTTTGTGACATCGCATTTGCCG |
| | | CCAGGGTTTAACGAGTATGACTTCGTCCCCGAGTCATTTGACAG |
| | | AGATAAAACCATCGCGCTGATTATGAATTCCTCGGGTAGCACCG |
| | | GTTTGCCAAAGGGGGTGGCGTTGCCCCACCGCACTGCTTGTGTG |
| | | CGGTTCTCGCACGCTAGGGATCCTATCTTTGGTAATCAGATCATT |
| | | CCCGACACAGCAATCCTGTCCGTGGTACCTTTTCATCACGGTTTT |
| | | GGCATGTTCACGACTCTCGGCTATTTGATTTGCGGTTTCAGGGTC |
| | | GTACTTATGTATCGGTTCGAGGAAGAACTGTTTTTGAGATCCTTG |
| | | CAAGATTACAAGATCCAGTCGGCCCTCCTTGTGCCAACGCTTTTC |
| | | TCATTCTTTGCGAAATCGACACTTATTGATAAGTATGACCTTTCC |
| | | AATCTGCATGAGATTGCCTCAGGGGGAGCGCCGCTTAGCAAGGA |
| | | AGTCGGGGAGGCAGTGGCCAAGCGCTTCCACCTTCCCGGAATTC |
| | | GGCAGGGATACGGGCTCACGGAGACAACATCCGCGATCCTTATC |
| | | ACGCCCGAGGGTGACGATAAGCCGGGAGCCGTCGGAAAAGTGG |
| | | TCCCCTTCTTTGAAGCCAAGGTCGTAGACCTCGACACGGGAAAA |
| | | ACCCTCGGAGTGAACCAGAGGGGCGAGCTCTGCGTGAGAGGGC |
| | | CGATGATCATGTCAGGTTACGTGAATAACCCTGAAGCGACGAAT |
| | | GCGCTGATCGACAAGGATGGGTGGTTGCATTCGGGAGACATTGC |
| | | CTATTGGGATGAGGATGAGCACTTCTTTATCGTAGATCGACTTAA |
| | | GAGCTTGATCAAATACAAAGGCTATCAGGTAGCGCCTGCCGAGC |
| | | TCGAGTCAATCCTGCTCCAGCACCCCAACATTTTCGACGCCGGA |
| | | GTGGCCGGGTTGCCCGATGACGACGCGGGTGAGCTGCCAGCGGC |
| | | CGTGGTAGTCCTCGAACATGGGAAAACAATGACCGAAAAGGAG |
| | | ATCGTGGACTACGTAGCATCACAAGTGACGACTGCGAAGAAACT |
| | | GAGGGGAGGGGTAGTCTTTGTGGACGAGGTCCCGAAAGGCTTGA |
| | | CTGGGAAGCTTGACGCTCGCAAAATCCGGGAAATCCTGATTAAG |
| | | GCAAAGAAAGGCGGGAAAATCGCTGTC |
| 142 | degGFP (AA) | MVSKGEELFTGVVPILVELDGDVNGHKFSVSGEGEGDATYGKLTL |
| | | KFICTTGKLPVPWPTLVTTLTYGVQCFSRYPDHMKQHDFFKSAMPE |
| | | GYVQERTIFFKDDGNYKTRAEVKFEGDTLVNRIELKGIDFKEDGNIL |
| | | GHKLEYNYNSHNVYIMADKQKNGIKVNFKIRHNIEDGSVQLADHY |
| | | QQNTPIGDGPVLLPDNHYLSTQSALSKDPNEKRDHMVLLEFVTAAG |
| | | ITLGMDELYKRSRDISHGFPPAVAAQDDGTLPMSCAQESGMDRHPA |
| | | ACASARINV |
| 143 | degGFP (NT) | ATGGTGTCCAAGGGTGAGGAATTGTTTACCGGGGTGGTGCCTAT |
| | | TCTCGTCGAACTTGACGGGGATGTGAATGGACACAAGTTTTCGG |
| | | TATCCGGAGAAGGAGAGGGTGACGCCACATACGGAAAGCTTAC |
| | | ACTCAAATTCATCTGTACGACGGGGAAACTGCCCGTACCCTGGC |
| | | CTACGCTCGTAACCACGCTGACTTATGGAGTGCAGTGCTTTAGCA |
| | | GATACCCCGACCATATGAAGCAGCACGACTTCTTCAAGTCGGCG |
| | | ATGCCCGAGGGGTACGTGCAAGAGAGGACCATTTTCTTCAAAGA |
| | | CGATGGCAATTACAAAACACGCGCAGAAGTCAAGTTTGAGGGCG |
| | | ATACTCTGGTCAATCGGATCGAATTGAAGGGAATCGATTTCAAA |
| | | GAAGATGGAAACATCCTTGGCCATAAGCTCGAGTACAACTATAA |
| | | CTCGCATAATGTCTATATCATGGCTGACAAGCAGAAAAACGGTA |
| | | TCAAAGTCAACTTTAAGATCCGACACAATATTGAGGACGGTTCG |
| | | GTGCAGCTTGCGGACCACTATCAACAGAATACGCCGATTGGGGA |
| | | TGGTCCGGTCCTTTTGCCGGATAACCATTATCTCTCAACCCAGTC |
| | | AGCCCTGAGCAAAGATCCAAACGAGAAGAGGGACCACATGGTC |
| | | TTGCTCGAATTCGTGACAGCGGCAGGGATCACTCTGGGAATGGA |
| | | CGAGTTGTACAAGAGATCTCGAGATATCAGCCATGGCTTCCCGC |
| | | CGGCGGTGGCGGCGCAGGATGATGGCACGCTGCCCATGTCTTGT |
| | | GCCCAGGAGAGCGGGATGGACCGTCACCCTGCAGCCTGTGCTTC |
| | | TGCTAGGATCAATGTG |
| 144 | Luc (AA) | MEDAKNIKKGPAPFYPLEDGTAGEQLHKAMKRYALVPGTIAFTDA |
| | | HIEVDITYAEYFEMSVRLAEAMKRYGLNTNHRIVVCSENSLQFFMP |
| | | VLGALFIGVAVAPANDIYNERELLNSMGISQPTVVFVSKKGLQKILN |
| | | VQKKLPIIQKIIIMDSKTDYQGFQSMYTFVTSHLPPGFNEYDFVPESF |
| | | DRDKTIALIMNSSGSTGLPKGVALPHRTACVRFSHARDPIFGNQIIPD |
| | | TAILSVVPFHHGFGMFTTLGYLICGFRVVLMYRFEEELFLRSLQDYK |
| | | IQSALLVPTLFSFFAKSTLIDKYDLSNLHEIASGGAPLSKEVGEAVAK |
| | | RFHLPGIRQGYGLTETTSAILITPEGDDKPGAVGKVVPFFEAKVVDL |
| | | DTGKTLGVNQRGELCVRGPMIMSGYVNNPEATNALIDKDGWLHSG |
| | | DIAYWDEDEHFFIVDRLKSLIKYKGYQVAPAELESILLQHPNIFDAG |
| | | VAGLPDDDAGELPAAVVVLEHGKTMTEKEIVDYVASQVTTAKKLR |
| | | GGVVFVDEVPKGLTGKLDARKIREILIKAKKGGKIAV |
| 145 | Luc (NT) | ATGGAAGATGCGAAGAACATCAAGAAGGGACCTGCCCCGTTTTA |
| | | CCCTTTGGAGGACGGTACAGCAGGAGAACAGCTCCACAAGGCG |
| | | ATGAAACGCTACGCCCTGGTCCCCGGAACGATTGCGTTTACCGA |
| | | TGCACATATTGAGGTAGACATCACATACGCAGAATACTTCGAAA |
| | | TGTCGGTGAGGCTGGCGGAAGCGATGAAGAGATATGGTCTTAAC |
| | | ACTAATCACCGCATCGTGGTGTGTTCGGAGAACTCATTGCAGTTT |
| | | TTCATGCCGGTCCTTGGAGCACTTTTCATCGGGGTCGCAGTCGCG |
| | | CCAGCGAACGACATCTACAATGAGCGGGAACTCTTGAATAGCAT |
| | | GGGAATCTCCCAGCCGACGGTCGTGTTTGTCTCCAAAAAGGGGC |
| | | TGCAGAAAATCCTCAACGTGCAGAAGAAGCTCCCCATTATTCAA |
| | | AAGATCATCATTATGGATAGCAAGACAGATTACCAAGGGTTCCA |
| | | GTCGATGTATACCTTTGTGACATCGCATTTGCCGCCAGGGTTTAA |
| | | CGAGTATGACTTCGTCCCCGAGTCATTTGACAGAGATAAAACCA |
| | | TCGCGCTGATTATGAATTCCTCGGGTAGCACCGGTTTGCCAAAG |
| | | GGGGTGGCGTTGCCCCACCGCACTGCTTGTGTGCGGTTCTCGCAC |
| | | GCTAGGGATCCTATCTTTGGTAATCAGATCATTCCCGACACAGCA |
| | | ATCCTGTCCGTGGTACCTTTTCATCACGGTTTTGGCATGTTCACG |
| | | ACTCTCGGCTATTTGATTTGCGGTTTCAGGGTCGTACTTATGTAT |
| | | CGGTTCGAGGAAGAACTGTTTTTGAGATCCTTGCAAGATTACAA |
| | | GATCCAGTCGGCCCTCCTTGTGCCAACGCTTTTCTCATTCTTTGC |
| | | GAAATCGACACTTATTGATAAGTATGACCTTTCCAATCTGCATGA |
| | | GATTGCCTCAGGGGGAGCGCCGCTTAGCAAGGAAGTCGGGGAG |
| | | GCAGTGGCCAAGCGCTTCCACCTTCCCGGAATTCGGCAGGGATA |
| | | CGGGCTCACGGAGACAACATCCGCGATCCTTATCACGCCCGAGG |
| | | GTGACGATAAGCCGGGAGCCGTCGGAAAAGTGGTCCCCTTCTTT |
| | | GAAGCCAAGGTCGTAGACCTCGACACGGGAAAAACCCTCGGAG |
| | | TGAACCAGAGGGGCGAGCTCTGCGTGAGAGGGCCGATGATCATG |
| | | TCAGGTTACGTGAATAACCCTGAAGCGACGAATGCGCTGATCGA |
| | | CAAGGATGGGTGGTTGCATTCGGGAGACATTGCCTATTGGGATG |
| | | AGGATGAGCACTTCTTTATCGTAGATCGACTTAAGAGCTTGATCA |
| | | AATACAAAGGCTATCAGGTAGCGCCTGCCGAGCTCGAGTCAATC |
| | | CTGCTCCAGCACCCCAACATTTTCGACGCCGGAGTGGCCGGGTT |
| | | GCCCGATGACGACGCGGGTGAGCTGCCAGCGGCCGTGGTAGTCC |
| | | TCGAACATGGGAAAACAATGACCGAAAAGGAGATCGTGGACTA |
| | | CGTAGCATCACAAGTGACGACTGCGAAGAAACTGAGGGGAGGG |
| | | GTAGTCTTTGTGGACGAGGTCCCGAAAGGCTTGACTGGGAAGCT |
| | | TGACGCTCGCAAAATCCGGGAAATCCTGATTAAGGCAAAGAAAG |
| | | GCGGGAAAATCGCTGTC |
| 146 | EPO (AA) | MGVHECPAWLWLLLSLLSLPLGLPVLGAPPRLICDSRVLERYLLEA |
| | | KEAENITTGCAEHCSLNENITVPDTKVNFYAWKRMEVGQQAVEVW |
| | | QGLALLSEAVLRGQALLVNSSQPWEPLQLHVDKAVSGLRSLTTLLR |
| | | ALGAQKEAISPPDAASAAPLRTITADTFRKLFRVYSNFLRGKLKLYT |
| | | GEACRTGDR |
| 147 | EPO (NT) | ATGGGAGTGCACGAGTGTCCCGCGTGGTTGTGGTTGCTGCTGTC |
| | | GCTCTTGAGCCTCCCACTGGGACTGCCTGTGCTGGGGGCACCAC |
| | | CCAGATTGATCTGCGACTCACGGGTACTTGAGAGGTACCTTCTTG |
| | | AAGCCAAAGAAGCCGAAAACATCACAACCGGATGCGCCGAGCA |
| | | CTGCTCCCTCAATGAGAACATTACTGTACCGGATACAAAGGTCA |
| | | ATTTCTATGCATGGAAGAGAATGGAAGTAGGACAGCAGGCCGTC |
| | | GAAGTGTGGCAGGGGCTCGCGCTTTTGTCGGAGGCGGTGTTGCG |
| | | GGGTCAGGCCCTCCTCGTCAACTCATCACAGCCGTGGGAGCCCC |
| | | TCCAACTTCATGTCGATAAAGCGGTGTCGGGGCTCCGCAGCTTG |
| | | ACGACGTTGCTTCGGGCTCTGGGCGCACAAAAGGAGGCTATTTC |
| | | GCCGCCTGACGCGGCCTCCGCGGCACCCCTCCGAACGATCACCG |
| | | CGGACACGTTTAGGAAGCTTTTTAGAGTGTACAGCAATTTCCTCC |
| | | GCGGAAAGCTGAAATTGTATACTGGTGAAGCGTGTAGGACAGGG |
| | | GATCGC |
| 154 | NpiLuc (aa) in | MGKPIPNPLLGLDSTQGQEQKLISEEDLAGKPIPNPLLGLDSTQGQE |
| | circular construct | QKLISEEDLAGKPIPNPLLGLDSTQGQEQKLISEEDLAGKPIPNPLLG |
| | | LDSTQGQEQKLISEEDLAGKPIPNPLLGLDSTQGQEQKLISEEDLAPA |
| | | AKRVKLDEDAKNIKKGPAPFYPLEDGTAGEQLHKAMKRYALVPGT |
| | | IAFTDAHIEVDITYAEYFEMSVRLAEAMKRYGLNTNHRIVVCSENSL |
| | | QFFMPVLGALFIGVAVAPANDIYNERELLNSMGISQPTVVFVSKKGL |
| | | QKILNVQKKLPIIQKIIIMDSKTDYQGFQSMYTFVTSHLPPGFNEYDF |
| | | VPESFDRDKTIALIMNSSGSTGLPKGVALPHRTACVRFSHARDPIFG |
| | | NQIIPDTAILSVVPFHHGFGMFTTLGYLICGFRVVLMYRFEEELFLRS |
| | | LQDYKIQSALLVPTLFSFFAKSTLIDKYDLSNLHEIASGGAPLSKEVG |
| | | EAVAKRFHLPGIRQGYGLTETTSAILITPEGDDKPGAVGKVVPFFEA |
| | | KVVDLDTGKTLGVNQRGELCVRGPMIMSGYVNNPEATNALIDKDG |
| | | WLHSGDIAYWDEDEHFFIVDRLKSLIKYKGYQVAPAELESILLQHPN |
| | | IFDAGVAGLPDDDAGELPAAVVVLEHGKTMTEKEIVDYVASQVTT |
| | | AKKLRGGVVFVDEVPKGLTGKLDARKIREILIKAKKGGKIAV |
| 155 | Npi Luc (nt) in | ATGGGCAAGCCTATTCCCAACCCGCTGCTGGGCTTAGACAGCAC |
| | circular construct | CCAAGGCCAAGAGCAGAAGCTCATAAGCGAAGAGGATCTCGCC |
| | | GGCAAGCCGATCCCCAACCCACTGCTGGGCCTTGACAGTACCCA |
| | | GGGCCAGGAACAGAAACTGATCAGCGAAGAGGACTTAGCCGGG |
| | | AAGCCCATCCCCAACCCTCTGCTGGGTCTGGACTCCACCCAGGG |
| | | ACAAGAACAGAAGCTTATATCCGAGGAAGACCTGGCCGGGAAG |
| | | CCTATCCCCAATCCTCTGCTGGGCCTGGATAGCACCCAGGGTCA |
| | | GGAGCAGAAGCTGATCTCCGAAGAGGACCTTGCGGGCAAGCCC |
| | | ATCCCAAATCCCCTGTTAGGCCTGGACAGCACCCAGGGGCAGGA |
| | | ACAGAAGCTGATAAGCGAGGAAGATCTGGCTCCCGCTGCCAAAC |
| | | GGGTGAAGCTGGACGAGGACGCCAAGAACATCAAGAAGGGGCC |
| | | CGCTCCCTTCTACCCGCTGGAGGACGGCACCGCTGGCGAGCAGC |
| | | TGCACAAGGCCATGAAGAGGTACGCACTGGTGCCCGGCACAATC |
| | | GCCTTCACCGACGCCCACATCGAGGTGGACATCACCTACGCCGA |
| | | GTACTTCGAGATGAGCGTGCGGCTGGCCGAAGCCATGAAGCGGT |
| | | ACGGCCTGAACACCAACCACCGCATCGTGGTGTGCTCCGAGAAC |
| | | TCCCTGCAGTTCTTCATGCCCGTTCTGGGCGCCCTGTTCATTGGG |
| | | GTGGCCGTGGCACCAGCCAACGACATCTACAACGAGAGGGAGCT |
| | | GCTGAACAGCATGGGCATCAGCCAGCCCACCGTGGTGTTCGTGT |
| | | CCAAGAAGGGCCTGCAGAAGATCCTGAACGTGCAGAAGAAGCT |
| | | GCCCATCATCCAGAAGATCATCATCATGGACAGCAAGACCGACT |
| | | ACCAGGGCTTCCAGTCCATGTACACCTTCGTGACCAGCCACCTGC |
| | | CTCCCGGCTTCAACGAGTACGACTTCGTGCCCGAGTCCTTCGACA |
| | | GGGACAAGACCATCGCCCTGATCATGAACTCCAGCGGAAGCACC |
| | | GGCCTGCCCAAGGGTGTGGCTCTACCCCACCGGACCGCCTGTGT |
| | | ACGGTTCAGCCACGCCCGTGACCCCATCTTCGGGAACCAGATCA |
| | | TCCCCGACACCGCCATCCTGAGCGTGGTGCCCTTCCACCACGGCT |
| | | TCGGCATGTTCACCACCCTGGGCTACCTGATCTGCGGCTTCCGGG |
| | | TGGTGCTGATGTACCGGTTCGAGGAGGAGCTGTTCCTGCGGAGC |
| | | CTGCAGGACTACAAGATCCAGTCCGCCCTGCTGGTGCCCACCCT |
| | | GTTCTCCTTCTTCGCCAAGTCCACCCTGATCGACAAGTACGACCT |
| | | GAGCAACCTGCACGAGATTGCCTCTGGCGGCGCACCTCTGTCCA |
| | | AGGAGGTCGGGGAGGCCGTAGCCAAGCGGTTCCACCTGCCCGGC |
| | | ATCCGGCAAGGCTACGGCCTGACCGAGACCACCAGCGCCATCCT |
| | | GATCACCCCTGAGGGCGACGACAAGCCCGGCGCAGTGGGGAAG |
| | | GTGGTGCCATTCTTCGAGGCCAAGGTGGTGGACCTGGACACCGG |
| | | CAAGACCCTGGGCGTGAACCAGCGGGGCGAGCTGTGCGTGCGG |
| | | GGACCCATGATCATGTCCGGCTACGTGAACAACCCCGAGGCCAC |
| | | CAACGCCCTGATTGACAAGGACGGGTGGCTGCACAGCGGCGACA |
| | | TCGCCTACTGGGACGAGGACGAGCACTTCTTCATCGTGGACCGC |
| | | CTGAAGTCCCTGATCAAGTACAAGGGGTACCAGGTGGCTCCCGC |
| | | CGAGCTGGAGTCCATCCTGCTGCAGCACCCCAACATCTTTGACG |
| | | CCGGCGTTGCCGGACTGCCTGACGACGACGCTGGGGAACTCCCC |
| | | GCAGCTGTGGTGGTGCTGGAGCACGGCAAGACCATGACCGAGA |
| | | AGGAGATCGTGGACTACGTCGCCTCCCAGGTGACCACCGCCAAG |
| | | AAGTTACGCGGCGGCGTCGTGTTCGTGGACGAGGTGCCCAAGGG |
| | | CCTGACCGGCAAGCTGGACGCCCGCAAGATCCGGGAGATCCTGA |
| | | TCAAGGCCAAGAAGGGCGGGAAGATCGCCGTG |
| 156 | Non-coding | TGATAATAGGCTGGAGCCTCGGTGGCCTAGCTTCTTGCCCCTTGG |
| | region in NpiLic | GCCTCCCCCCAGCCCCTCCTCCCCTTCCTGCACCCGTACCCCCGT |
| | circle (nt) | GGTCTTTGAATAAAGTCTGAGTGGGCGGCAAAAAAAAAAAAAA |
| | | AAAAAAAAAAAAAAAAAAAAAAAAAAAAAAAAAAAAAAAAAA |
| | | AAAAAAAAAAAAAAAAAAAAAAAAAAAAAAAAAAAAAAAAAA |
| | | AATCTAGGGGTATAATTCTCATTTCCACCTCTTTTCATCTGATTCA |
| | | CGACTACGGAATCGTTGTTCTAGGACACTTCCGAAACCAAAATC |
| | | TCGCC |
| 157 | La protein | MAENGDNEKMAALEAKICHQIEYYFGDFNLPRDKFLKEQIKLDEG |
| | | WVPLEIMIKFNRLNRLTTDFNVIVEALSKSKAELMEISEDKTKIRRSP |
| | | SKPLPEVTDEYKNDVKNRSVYIKGFPTDATLDDIKEWLEDKGQVLN |
| | | IQMRRTLHKAFKGSIFVVFDSIESAKKFVETPGQKYKETDLLILFKD |
| | | DYFAKKNEERKQNKVEAKLRAKQEQEAKQKLEEDAEMKSLEEKIG |
| | | CLLKFSGDLDDQTCREDLHILFSNHGEIKWIDFVRGAKEGIILFKEKA |
| | | KEALGKAKDANNGNLQLRNKEVTWEVLEGEVEKEALKKIIEDQQE |
| | | SLNKWKSKGRRFKGKGKGNKAAQPGSGKGKVQFQGKKTKFASDD |
| | | EHDEHDENGATGPVKRAREETDKEEPASKQQKTENGAGD |
| 158 | La protein without | MAENGDNEKMAALEAKICHQIEYYFGDFNLPRDKFLKEQIKLDEG |
| | NLS | WVPLEIMIKFNRLNRLTTDFNVIVEALSKSKAELMEISEDKTKIRRSP |
| | | SKPLPEVTDEYKNDVKNRSVYIKGFPTDATLDDIKEWLEDKGQVLN |
| | | IQMRRTLHKAFKGSIFVVFDSIESAKKFVETPGQKYKETDLLILFKD |
| | | DYFAKKNEERKQNKVEAKLRAKQEQEAKQKLEEDAEMKSLEEKIG |
| | | CLLKFSGDLDDQTCREDLHILFSNHGEIKWIDFVRGAKEGIILFKEKA |
| | | KEALGKAKDANNGNLQLRNKEVTWEVLEGEVEKEALKKIIEDQQE |
| | | SLNKWKSKGRRFKGKGKGNKAAQPGSGKGKVQFQ |

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A composition comprising:
(a) a first polynucleotide comprising (i) a repressor binding element and (ii) an open reading frame encoding a polypeptide; and
(b) a second polynucleotide comprising (i) a sequence encoding a repressor that binds to the repressor binding element and (ii) a recognition site, wherein modification of the recognition site reduces translation of the repressor from the second polynucleotide,
wherein binding of the repressor to the repressor binding element reduces translation of the polypeptide from the first polynucleotide.

2. The composition of claim 1, wherein the first polynucleotide is mRNA and comprises a polyA tail or is a DNA.

3. The composition of claim 1 or 2, wherein the second polynucleotide is (a) an mRNA and comprises a 5′ cap, a start codon, and a polyA tail or (b) a DNA.

4. The composition of claim 2 or 3, wherein when the first and second polynucleotides are DNA, they are encoded in a single plasmid.

5. The composition of claim 3, wherein the recognition site in the second polynucleotide which is an mRNA is (a) positioned between the sequence encoding the repressor and the polyA tail; or (b) positioned between the 5′ cap and the start codon.

6. The composition of any one of claims 1-5, wherein the repressor binding element comprises a kink-turn forming sequence.

7. The composition of claim 6, wherein the repressor binding element is selected from the group consisting of PRE, PRE2, MS2, PP7, BoxB, U1A hairpin, and 7SK.

8. The composition of any one of claims 1-7, wherein the repressor is selected from the group consisting of 50S ribosomal L7Ae protein, Pumilio and FBF (PUF) protein, PUF2 protein, MBP-LacZ, MBP, PCP, Lambda N, U1A, 15.5 kd, LARP7, and other RNA-binding proteins.

9. The composition of any one of claims 1-8, wherein the recognition site is a microRNA target site or an endonuclease recognition site.

10. The composition of claim 9, wherein the microRNA target site comprises a sequence selected from a group consisting of SEQ ID NOs: 75-80.

11. The composition of any one of claims 1-10, wherein the polypeptide is selected from the group consisting of a secreted protein, a membrane-bound protein, an intercellular protein, or peptides, polypeptides or biologically active fragments thereof.

12. The composition of any one of claims 1-11, wherein the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

13. The composition of any one of claims 1-12, wherein (a) and (b) are in separate dosage forms packaged together.

14. The composition of any one of claims 1-12, wherein (a) and (b) are in a unit dosage form.

15. A method of expressing a polypeptide in a cell, the method comprising contacting the cell with the composition of any one of claims 1-14, wherein the cell expresses a microRNA or endonuclease that binds to the recognition site and reduces translation of the repressor from the second polynucleotide.

16. A method of expressing a polypeptide in a cell, the method comprising contacting the cell with
(a) a first polynucleotide comprising (i) a repressor binding element and (ii) an open reading frame encoding a polypeptide; and
(b) a second polynucleotide comprising (i) a sequence encoding a repressor that binds to the repressor binding element and (ii) a recognition site,
wherein the cell expresses a microRNA or an endonuclease that binds to the recognition site and reduces translation of the repressor from the second polynucleotide.

17. A method of expressing a polypeptide in a cell in a subject, the method comprising administering to the subject:
(a) a first polynucleotide comprising (i) a repressor binding element and (ii) an open reading frame encoding a polypeptide; and
(b) a second polynucleotide comprising (i) a sequence encoding a repressor that binds to the repressor binding element and (ii) a recognition site,
wherein the cell expresses a microRNA or an endonuclease that binds to the recognition site and reduces translation of the repressor from the second polynucleotide.

18. The method of any one of claims 15-17, wherein the cell is a liver cell.

19. The method of any one of claims 15-17, wherein the first polynucleotide is DNA or RNA and the second polynucleotide is DNA or RNA.

20. A composition comprising:
(a) a first polynucleotide comprising (i) an open reading frame encoding a first polypeptide, (ii) an effector binding element, and optionally (iii) a recognition site, wherein the first polynucleotide is mRNA; and
(b) a second polynucleotide comprising a sequence encoding a second polypeptide, wherein the second polypeptide comprises an effector, wherein binding of the effector to the effector binding element enables and/or increases translation of the first polypeptide from the first polynucleotide.

21. The composition of claim 20, wherein the second polynucleotide is an mRNA and comprises a polyA tail or is a DNA.

22. The composition of claim 20 or 21, wherein the effector binding element comprises a sequence selected from the group consisting of MS2, PRE, PRE2, PP7, BoxB, U1A hairpin and 7SK.

23. The composition of any one of claims 20-22, wherein the second polypeptide comprises a sequence selected from the group consisting of Pumilio and FBF (PUF) protein, PUF2 protein, MBP-LacZ, 50S ribosomal L7Ae protein, PCP, Lambda N, U1A, 15.5 kd, LARP7, and an MBP polypeptide set forth in Table 4, wherein the second polypeptide binds to the respective effector binding element set forth in Table 1.

24. The composition of any one of claims 20-23, wherein the recognition site is (a) between the open reading frame encoding the first polypeptide and the effector binding element in the first polynucleotide; or
(b) located after the effector binding element in the first polynucleotide that
(i) is linear and 5′-3′ oriented, or
(ii) has a circular clockwise orientation.

25. The composition of any one of claims 20-24, wherein the first polynucleotide (a) has no polyA tail; (b) is circular; (c) has no 5′ cap; (d) has no 5′ cap and no polyA tail; or (e) has a 5′ and/or 3′ end that is unsuitable for canonical translation.

26. The composition of any one of claims 20-25, wherein the effector is the Eukaryotic translation initiation factor 4 G (eIF4G) or a fragment thereof.

27. The composition of any one of claims 20-25, wherein the effector is an internal ribosome entry site (IRES)-Trans activating factor (ITAF) protein or a variant thereof.

28. The composition of any one of claims 20-25, wherein the effector is La protein or a variant thereof.

29. The composition of any one of claims 20-28, wherein the recognition site is a microRNA target site or an endonuclease cleavage site.

30. The composition of any one of claim 29, wherein the microRNA target site is selected from a group consisting of SEQ ID NOs: 75-80.

31. The composition of any one of claims 20-30, wherein the composition comprises one or more delivery agents selected from a group consisting of a lipid nanoparticle, a liposome, a lipoplex, a polyplex, a lipidoid, a polymer, a microvesicle, an exosome, a peptide, a protein, cells transfected with polynucleotides, hyaluronidase, nanoparticle mimics, nanotubes, and conjugates.

32. The composition of any one of claims 20-31, wherein (a) and (b) are in separate dosage forms packaged together.

33. The composition of any one of claims 20-31, wherein (a) and (b) are in a unit dosage form.

34. A method of expressing a polypeptide in a cell, the method comprising contacting the cell with the composition of any one of claims 20-31, wherein the cell expresses a microRNA or an endonuclease that binds to the recognition site of the first polynucleotide and enhances the ability of the effector to bind to the effector binding element and promote translation of the first polynucleotide.

35. A method of expressing a polypeptide in a cell, the method comprising contacting the cell with
(a) a first polynucleotide comprising (i) an open reading frame encoding a first polypeptide, and (ii) an effector binding element, and optionally (iii) a recognition site, wherein the first polynucleotide is mRNA; and
(b) a second polynucleotide comprising a sequence encoding a second polypeptide, wherein the second polypeptide comprises an effector, wherein binding of the effector to the effector binding element increases translation of the first polypeptide from the first polynucleotide,
optionally wherein the cell expresses a microRNA or an endonuclease that binds to the recognition site of the first polynucleotide and enhances the ability of the effector to bind to the effector binding element and promote translation of the first polynucleotide.

36. A composition comprising:
(a) a first polynucleotide comprising (i) an open reading frame encoding a first polypeptide, and (ii) an effector binding element, wherein the first polynucleotide is mRNA which lacks a polyA tail; and
(b) a second polynucleotide comprising a sequence encoding a second polypeptide, wherein the second polypeptide comprises an effector, wherein binding of the effector to the effector binding element enables and/or increases translation of the first polypeptide from the first polynucleotide.

37. The composition of claim 36, wherein the second polynucleotide is an mRNA and comprises a polyA tail or is a DNA.

38. The composition of claim 37, wherein when the second polynucleotide is a DNA, the sequence encoding the effector is under the control of a tissue-specific promoter.

39. The composition of claim 35, wherein expression and/or recruitment of the effector is under the control of a trigger in a specific microenvironment or specific cell-type.

40. The composition of claim 39, wherein the trigger is microRNA, receptor-mediated activation, and/or a change in pH and/or hypoxia.

41. The composition of claim 36, wherein the effector is a stabilizer and effector binding element is a destabilizing sequence or structure.

42. A composition comprising:
(a) an RNA molecule comprising in order from the 5′ to 3′ end of the RNA (i) an open reading frame encoding a polypeptide, (ii) a polyA tail, (iii) a cleavage site, and (iv) a destabilizing sequence; and
(b) a DNA molecule comprising a sequence encoding an endonuclease that binds to the cleavage site, wherein the endonuclease sequence is under the control of a tissue-specific promoter, wherein binding of the endonuclease to the cleavage site cleaves the destabilizing sequence and enhances translation of the polypeptide from the first polynucleotide.
